SEQUENCE LISTING (31 sequences)

SEQ ID NO: 1 - DNA, 4107 bp, Homo sapiens
  CDS (54)..(2294)
gtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatagtgtaataattgtgcaggtacaggaattgttccaccagcattaggaactttagcatgtcaaaatgaatgtttacttgtgaactcgatagagcaaggaaaccagaaaggtgtaatatttataggttggtaaaatagattgtttttcatggataatttttaacttcattatttctgtacttgtacaaactcaacactaacttttttttttttaaaaaaaaaaaggtactaagtatcttcaatcagctgttggtcaagactaactttcttttaaaggttcatttgtatgataaattcatatgtgtatatataattttttttgttttgtctagtgagtttcaacatttttaaagttttcaaaaagccatcggaatgttaaattaatgtaaagggacagctaatctagaccaaagaatggtattttcacttttctttgtaacattgaatggtttgaagtactcaaaatctgttacgctaaacttttgattctttaacacaattatttttaaacactggcattttccaaaactgtggcagctaactttttaaaatctcaaatgacatgcagtgtgagtagaaggaagtcaacaatatgtggggagagcactcggttgtctttacttttaaaagtaatacttggtgctaagaatttcaggattattgtatttacgttcaaatgaagatggcttttgtacttcctgtggacatgtagtaatgtctatattggctcataaaactaacctgaaaaacaaataaatgctttggaaatgtttcagttgctttagaaacattagtgcctgcctggatccccttagttttgaaatatttgccattgttgtttaaatacctatcactgtggtagagcttgcattgatcttttccacaagtattaaactgccaaaatgtgaatatgcaaagcctttctgaatctataataatggtacttctactggggagagtgtaatattttggactgctgttttccattaatgaggagagcaacaggcccctgattatacagttccaaagtaataagatgttaattgtaattcagccagaaagtacatgtctcccattgggaggatttggtgttaaataccaaactgctagccctagtattatggagatgaacatgatgatgtaacttgtaatagcagaatagttaatgaatgaaactagttcttataatttatctttatttaaaagcttagcctgccttaaaactagagatcaactttctcagctgcaaaagcttctagtctttcaagaagttcatactttatgaaattgcacagtaagcatttatttttcagaccatttttgaacatcactcctaaattaataaagtattcctctgttgctttagtatttattacaataaaaagggtttgaaatatagctgttctttatgcataaaacacccagctaggaccattactgccagagaaaaaaatcgtattgaatggccatttccctacttataagatgtctcaatctgaatttatttggctacactaaagaatgcagtatatttagttttccatttgcatgatgtttgtgtgctatagatgatattttaaattgaaaagtttgttttaaattatttttacagtgaagactgttttcagctctttttatattgtacatagtcttttatgtaatttactggcatatgttttgtagactgtttaatgactggatatcttccttcaacttttgaaatacaaaaccagtgttttttacttgtacactgttttaaagtctattaaaattgtcatttgacttttttctgttaaaaaaaaaaaaaaaaaaaa

SEQ ID NO: 2 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 3 - DNA, 1963 bp, Homo sapiens
  CDS (201)..(1367)
gtgttgtacgaaagcgcgtctgcggccgcaatgtctgctgagagttgtagttctgtgccctatcacggccactcccatttctggtgccgtcacgggacagagcagtcggtgacaggacagagcagtcggtgacgggacacagtggttggtgacgggacagagcggtcggtgacagcctcaagggcttcagcaccgcgcccatggcagagccagacccctctcaccctctggagacccaggcagggaaggtgcaggaggctcaggactcagattcagactctgagggaggagccgctggtggagaagcagacatggacttcctgcggaacttattctcccagacgctcagcctgggcagccagaaggagcgtctgctggacgagctgaccttggaaggggtggcccggtacatgcagagcgaacgctgtcgcagagtcatctgtttggtgggagctggaatctccacatccgcaggcatccccgactttcgctctccatccaccggcctctatgacaacctagagaagtaccatcttccctacccagaggccatctttgagatcagctatttcaagaaacatccggaacccttcttcgccctcgccaaggaactctatcctgggcagttcaagccaaccatctgtcactacttcatgcgcctgctgaaggacaaggggctactcctgcgctgctacacgcagaacatagataccctggagcgaatagccgggctggaacaggaggacttggtggaggcgcacggcaccttctacacatcacactgcgtcagcgccagctgccggcacgaatacccgctaagctggatgaaagagaagatcttctctgaggtgacgcccaagtgtgaagactgtcagagcctggtgaagcctgatatcgtcttttttggtgagagcctcccagcgcgtttcttctcctgtatgcagtcagacttcctgaaggtggacctcctcctggtcatgggtacctccttgcaggtgcagccctttgcctccctcatcagcaaggcacccctctccacccctcgcctgctcatcaacaaggagaaagctggccagtcggaccctttcctggggatgattatgggcctcggaggaggcatggactttgactccaagaaggcctacagggacgtggcctggctgggtgaatgcgaccagggctgcctggcccttgctgagctccttggatggaagaaggagctggaggaccttgtccggagggagcacgccagcatagatgcccagtcgggggcgggggtccccaaccccagcacttcagcttcccccaagaagtccccgccacctgccaaggacgaggccaggacaacagagagggagaaaccccagtgacagctgcatctcccaggcgggatgccgagctcctcagggacagctgagccccaaccgggcctggccccctcttaaccagcagttcttgtctggggagctcagaacatcccccaatctcttacagctccctccccaaaactggggtcccagcaaccctggcccccaaccccagcaaatctctaacacctcctagaggccaaggcttaaacaggcatctctaccagccccactgtctctaaccactcctgggctaaggagtaacctccctcatctctaactgcccccacggggccagggctaccccagaacttttaactcttccaggacagggagcttcgggcccccactctgtctcctgcccccgggggcctgtggctaagtaaaccatacctaacctaccccagtgtgggtgtgggcctctgaatataacccacacccagcgtagggggagtctgagccgggagggctcccgagtctctgccttcagctcccaaagtgggtggtgggcccccttcacgtgggacccacttcccatgctggatgggcagaagacattgcttattggagacaaattaaaaacaaaaacaactaac

SEQ ID NO: 4 - protein, 389 aa, Homo sapiens
MAEPDPSHPLETQAGKVQEAQDSDSDSEGGAAGGEADMDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 5 - DNA, 1931 bp, Homo sapiens
  CDS (257)..(1312)
cgaaagcgcgtctgcggccgcaatgtctgctgagagttgtagttctgtgccctatcacggccactcccatttctggtgccgtcacgggacagagcagtcggtgacaggacagagcagtcggtgacgggacacagtggttggtgacgggacagagcggtcggtgacagcctcaagggcttcagcaccgcgcccatggcagagccagaccgactcagattcagactctgagggaggagccgctggtggagaagcagacatggacttcctgcggaacttattctcccagacgctcagcctgggcagccagaaggagcgtctgctggacgagctgaccttggaaggggtggcccggtacatgcagagcgaacgctgtcgcagagtcatctgtttggtgggagctggaatctccacatccgcaggcatccccgactttcgctctccatccaccggcctctatgacaacctagagaagtaccatcttccctacccagaggccatctttgagatcagctatttcaagaaacatccggaacccttcttcgccctcgccaaggaactctatcctgggcagttcaagccaaccatctgtcactacttcatgcgcctgctgaaggacaaggggctactcctgcgctgctacacgcagaacatagataccctggagcgaatagccgggctggaacaggaggacttggtggaggcgcacggcaccttctacacatcacactgcgtcagcgccagctgccggcacgaatacccgctaagctggatgaaagagaagatcttctctgaggtgacgcccaagtgtgaagactgtcagagcctggtgaagcctgatatcgtcttttttggtgagagcctcccagcgcgtttcttctcctgtatgcagtcagacttcctgaaggtggacctcctcctggtcatgggtacctccttgcaggtgcagccctttgcctccctcatcagcaaggcacccctctccacccctcgcctgctcatcaacaaggagaaagctggccagtcggaccctttcctggggatgattatgggcctcggaggaggcatggactttgactccaagaaggcctacagggacgtggcctggctgggtgaatgcgaccagggctgcctggcccttgctgagctccttggatggaagaaggagctggaggaccttgtccggagggagcacgccagcatagatgcccagtcgggggcgggggtccccaaccccagcacttcagcttcccccaagaagtccccgccacctgccaaggacgaggccaggacaacagagagggagaaaccccagtgacagctgcatctcccaggcgggatgccgagctcctcagggacagctgagccccaaccgggcctggccccctcttaaccagcagttcttgtctggggagctcagaacatcccccaatctcttacagctccctccccaaaactggggtcccagcaaccctggcccccaaccccagcaaatctctaacacctcctagaggccaaggcttaaacaggcatctctaccagccccactgtctctaaccactcctgggctaaggagtaacctccctcatctctaactgcccccacggggccagggctaccccagaacttttaactcttccaggacagggagcttcgggcccccactctgtctcctgcccccgggggcctgtggctaagtaaaccatacctaacctaccccagtgtgggtgtgggcctctgaatataacccacacccagcgtagggggagtctgagccgggagggctcccgagtctctgccttcagctcccaaagtgggtggtgggcccccttcacgtgggacccacttcccatgctggatgggcagaagacattgcttattggagacaaattaaaaacaaaaacaactaacaaaaaaaaaaaaaaaaaaaaaaa

SEQ ID NO: 6 - protein, 352 aa, Homo sapiens
MDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 7 - DNA, 1927 bp, Homo sapiens
  CDS (103)..(1299)
ggcgccgggggcgggggtgggaggcggaggcggggccggggcgccgcgggcggggcgccgggggcggggcgagtccggaggactcctcggactgcgcggaacatggcgttctggggttggcgcgccgcggcagccctccggctgtggggccgggtagttgaacgggtcgaggccgggggaggcgtggggccgtttcaggcctgcggctgtcggctggtgcttggcggcagggacgatgtgagtgcggggctgagaggcagccatggggcccgcggtgagcccttggacccggcgcgccccttgcagaggcctcccagacccgaggtgcccagggcattccggaggcagccgagggcagcagctcccagtttcttcttttcgagtattaaaggtggaagaaggtccatatctttttctgtgggtgcttcaagtgttgttggaagtggaggcagcagtgacaaggggaagctttccctgcaggatgtagctgagctgattcgggccagagcctgccagagggtggtggtcatggtgggggccggcatcagcacacccagtggcattccagacttcagatcgccggggagtggcctgtacagcaacctccagcagtacgatctcccgtaccccgaggccatttttgaactcccattcttctttcacaaccccaagccctttttcactttggccaaggagctgtaccctggaaactacaagcccaacgtcactcactactttctccggctgcttcatgacaaggggctgcttctgcggctctacacgcagaacatcgatgggcttgagagagtgtcgggcatccctgcctcaaagctggttgaagctcatggaacctttgcctctgccacctgcacagtctgccaaagacccttcccaggggaggacattcgggctgacgtgatggcagacagggttccccgctgcccggtctgcaccggcgttgtgaagcccgacattgtgttctttggggagccgctgccccagaggttcttgctgcatgtggttgatttccccatggcagatctgctgctcatccttgggacctccctggaggtggagccttttgccagcttgaccgaggccgtgcggagctcagttccccgactgctcatcaaccgggacttggtggggcccttggcttggcatcctcgcagcagggacgtggcccagctgggggacgtggttcacggcgtggaaagcctagtggagcttctgggctggacagaagagatgcgggaccttgtgcagcgggaaactgggaagcttgatggaccagacaaataggatgatggctgcccccacacaataaatggtaacataggagacatccacatcccaattctgacaagacctcatgcctgaagacagcttgggcaggtgaaaccagaatatgtgaactgagtggacacccgaggctgccactggaatgtcttctcaggccatgagctgcagtgactggtagggctgtgtttacagtcagggccaccccgtcacatatacaaaggagctgcctgcctgtttgctgtgttgaactcttcactctgctgaagctcctaatggaaaaagctttcttctgactgtgaccctcttgaactgaatcagaccaactggaatcccagaccgagtctgctttctgtgcctagttgaacggcaagctcggcatctgttggttacaagatccagacttgggccgagcggtccccagccctcttcatgttccgaagtgtagtcttgaggccctggtgccgcacttctagcatgttggtctcctttagtggggctatttttaatgagagaaaatctgttctttccagcatgaaatacatttagtctcctcaaagggaaaaaaaaaaaaaaaaaaaaaaaaaaaaaaaaaaaaaaaaaaaaaaaaaaaaaaaaaaaaaaaaaa

SEQ ID NO: 8 - protein, 399 aa, Homo sapiens
MAFWGWRAAAALRLWGRVVERVEAGGGVGPFQACGCRLVLGGRDDVSAGLRGSHGARGEPLDPARPLQRPPRPEVPRAFRRQPRAAAPSFFFSSIKGGRRSISFSVGASSVVGSGGSSDKGKLSLQDVAELIRARACQRVVVMVGAGISTPSGIPDFRSPGSGLYSNLQQYDLPYPEAIFELPFFFHNPKPFFTLAKELYPGNYKPNVTHYFLRLLHDKGLLLRLYTQNIDGLERVSGIPASKLVEAHGTFASATCTVCQRPFPGEDIRADVMADRVPRCPVCTGVVKPDIVFFGEPLPQRFLLHVVDFPMADLLLILGTSLEVEPFASLTEAVRSSVPRLLINRDLVGPLAWHPRSRDVAQLGDVVHGVESLVELLGWTEEMRDLVQRETGKLDGPDK

SEQ ID NO: 9 - DNA, 1174 bp, Homo sapiens
  CDS (21)..(962)
gtccgtagagctgtgagagaatgaagatgagctttgcgttgactttcaggtcagcaaaaggccgttggatcgcaaaccccagccagccgtgctcgaaagcctccattgggttatttgtgccagcaagtcctcctctggaccctgagaaggtcaaagagttacagcgcttcatcaccctttccaagagactccttgtgatgactggggcaggaatctccaccgaatcggggataccagactacaggtcagaaaaagtggggctttatgcccgcactgaccgcaggcccatccagcatggtgattttgtccggagtgccccaatccgccagcggtactgggcgagaaacttcgtaggctggcctcaattctcctcccaccagcctaaccctgcacactgggctttgagcacctgggagaaactcggaaagctgtactggttggtgacccaaaatgtggatgctttgcacaccaaggcggggagtcggcgcctgacagagctccacggatgcatggacagggtcctgtgcttggattgtggggaacagactccccggggggtgctgcaagagcgtttccaagtcctgaaccccacctggagtgctgaggcccatggcctggctcctgatggtgacgtctttctctcagaggagcaagtccggagctttcaggtcccaacctgcgttcaatgtggaggccatctgaaaccagatgtcgttttcttcggggacacagtgaaccctgacaaggttgattttgtgcacaagcgtgtaaaagaagccgactccctcttggtggtgggatcatccttgcaggtatactctggttacaggtttatcctcactgcctgggagaagaagctcccgattgcaatactgaacattgggcccacacggtcggatgacttggcgtgtctgaaactgaattctcgttgtggagagttgctgcctttgatagacccatgctgaccacagcctgatattccagaacctggaacagggactttcacttgaatcttgctgctaaatgtaaatgccttctcaaatgacagattccagttcccattcaacagagtagggtgcactgacaaagtatagaaggttctaggtatcttaatgtgtggatattcttaattaaaactcattttttttaaataaaaaattgttcagctttaaaa

SEQ ID NO: 10 - protein, 314 aa, Homo sapiens
MKMSFALTFRSAKGRWIANPSQPCSKASIGLFVPASPPLDPEKVKELQRFITLSKRLLVMTGAGISTESGIPDYRSEKVGLYARTDRRPIQHGDFVRSAPIRQRYWARNFVGWPQFSSHQPNPAHWALSTWEKLGKLYWLVTQNVDALHTKAGSRRLTELHGCMDRVLCLDCGEQTPRGVLQERFQVLNPTWSAEAHGLAPDGDVFLSEEQVRSFQVPTCVQCGGHLKPDVVFFGDTVNPDKVDFVHKRVKEADSLLVVGSSLQVYSGYRFILTAWEKKLPIAILNIGPTRSDDLACLKLNSRCGELLPLIDPC

SEQ ID NO: 11 - DNA, 2350 bp, Homo sapiens
  CDS (219)..(1115)
attcgggggcgcgagctgccccagtaaatggaaatgttttctaacatataaaaacctacagaagaagaaaataattttctggatcaaattagaagtctgtattatattgatgtctccagattcaaatatattagaaagcagccgtggagacaaccatcttcattttgggcgaaataactaaagcccgcctcaagcattagaactacagacaaaccctgatgcgacctctccagattgtcccaagtcgattgatttcccagctatattgtggcctgaagcctccagcgtccacacgaaaccagatttgcctgaaaatggctcggccaagttcaagtatggcagattttcgaaagttttttgcaaaagcaaagcacatagtcatcatctcaggagctggtgttagtgcagaaagtggtgttccgaccttcagaggagctggaggttattggagaaaatggcaagcccaggacctggcgactcccctggcctttgcccacaacccgtcccgggtgtgggagttctaccactaccggcgggaggtcatggggagcaaggagcccaacgccgggcaccgcgccatagccgagtgtgagacccggctgggcaagcagggccggcgagtcgtggtcatcacccagaacatcgatgagctgcaccgcaaggctggcaccaagaaccttctggagatccatggtagcttatttaaaactcgatgtacctcttgtggagttgtggctgagaattacaagagtccaatttgtccagctttatcaggaaaaggtgctccagaacctggaactcaagatgccagcatcccagttgagaaacttccccggtgtgaagaggcaggctgcgggggcttgctgcgacctcacgtcgtgtggtttggagaaaacctggatcctgccattctggaggaggttgacagagagctcgcccactgtgatttatgtctagtggtgggcacttcctctgtggtgtacccagcagccatgtttgccccccaggtggctgccaggggcgtgccagtggctgaatttaacacggagaccaccccagctacgaacagattcagtcatttgatctccatctcatctctaattattataaagaattaaaacaagtcatcattgtagaaaagcaagaaaatgcagatagagaaaaagaagaaaataaaactggagtatttccacaacccaagtttagagttggcccccacctcccatgccatggactgagcagcaggggcccagcatcccttggatatggtggctgtgtcttcatgtgaaagaaactgaacttggtggtttttcctgccagttcaggagagattcttggcatgtaatatatatcactgctcaagtcaagcctcctaaaaccacagacctgtttcagctgctacttcagccaaaattcttcagcttcatattgtcttgaaaacctatgattgtctctaacaaacaggctacttgctagttagaaattcttatcaatttggcaagctacttatcaaccagactgaccacaagaactgtcatctcatcaatgaaggagtaactgatcaatgaagccagcaatgcttttttcttggcatcatcaaagctgacatttagaagagatgctggtgatagtcatctcatcctactcaatttttcaaaggcagaaaccaaccctggagcaattgagaggactgtttaaacacagagcttaacaatggcagaattgtatatctcgtgcttaacagattttggttgaactttaccctaggtcaggggtcagcaaactactgcctgtgggccaaatttgcccaccacctgtatctgtaaataaggtttcattggaacacagctgtggccatatgtttgtatattgtgtgtggctgcttttgcattaggatgacagaggtgaatagttgcaacagagactggctggtctgcaaagcctaaaatatgtcctgtgtggccctttacagaaaaagttttctaacccctgctctaggttacggagaaaaaaaaatggaataatgttctctgctacttttaacctgattttctttgtacctaaataggcagctagaatgctgcctatattttaataaggatttggatctcacaagacaccttaggcctacacaagttgttcagattctttgccccagttctaatctagtgacaaaggcatagaattctcctcccacaggaatgtatttctattttcaaggtgttaattagttccagttttggttttgtcgttttccccatgtccgatgcttatattggatgatttctgataaacctgactattccaataaaccctaggcatttttgaatttaaaaaaaaaaaaaaaaaa

SEQ ID NO: 12 - protein, 299 aa, Homo sapiens
MRPLQIVPSRLISQLYCGLKPPASTRNQICLKMARPSSSMADFRKFFAKAKHIVIISGAGVSAESGVPTFRGAGGYWRKWQAQDLATPLAFAHNPSRVWEFYHYRREVMGSKEPNAGHRAIAECETRLGKQGRRVVVITQNIDELHRKAGTKNLLEIHGSLFKTRCTSCGVVAENYKSPICPALSGKGAPEPGTQDASIPVEKLPRCEEAGCGGLLRPHVVWFGENLDPAILEEVDRELAHCDLCLVVGTSSVVYPAAMFAPQVAARGVPVAEFNTETTPATNRFSHLISISSLIIIKN

SEQ ID NO: 13 - DNA, 1670 bp, Homo sapiens
  CDS (297)..(1226)
ccggagcgcggtcgggacacagcgcctctaggagaaagcctggaaggcgctccgggggtacccagagctcttagcgggccggcagcatgtgcggggcccaagtaaatggaaatgttttctaacatataaaaacctacagaagaagaaaataattttctggatcaaattagaagtctgtattatattgatgtctccagattcaaatatattagaaagcagccgtggagacaaccatcttcattttgggagaaataactaaagcccgcctcaagcattagaactacagacaaaccctgatgcgacctctccagattgtcccaagtcgattgatttcccagctatattgtggcctgaagcctccagcgtccacacgaaaccagatttgcctgaaaatggctcggccaagttcaagtatggcagattttcgaaagttttttgcaaaagcaaagcacatagtcatcatctcaggagctggtgttagtgcagaaagtggtgttccgaccttcagaggagctggaggttattggagaaaatggcaagcccaggacctggcgactcccctggcctttgcccacaacccgtcccgggtgtgggagttctaccactaccggcgggaggtcatggggagcaaggagcccaacgccgggcaccgcgccatagccgagtgtgagacccggctgggcaagcagggccggcgagtcgtggtcatcacccagaacatcgatgagctgcaccgcaaggctggcaccaagaaccttctggagatccatggtagcttatttaaaactcgatgtacctcttgtggagttgtggctgagaattacaagagtccaatttgtccagctttatcaggaaaaggtgctccagaacctggaactcaagatgccagcatcccagttgagaaacttccccggtgtgaagaggcaggctgcgggggcttgctgcgacctcacgtcgtgtggtttggagaaaacctggatcctgccattctggaggaggttgacagagagctcgcccactgtgatttatgtctagtggtgggcacttcctctgtggtgtacccagcagccatgtttgccccccaggtggctgccaggggcgtgccagtggctgaatttaacacggagaccaccccagctacgaacagattcaggtttcatttccagggaccctgtggaacgactcttcctgaagcccttgcctgtcatgaaaatgaaactgtttcttaagtgtcctggggaagaaagaaattacagtatatctaagaactaggccacacgcagaggagaaatggtcttatgggtggtgagctgagtactgaacaatctaaaaatagcctctgattccctcgctggaatccaacctgttgataagtgatgggggtttagaagtagcaaagagcacccacattcaaaagtcacagaactggaaagttaattcatattatttggtttgaactgaaacgtgaggtatctttgatgtgtatggttggttattgggagggaaaaattttgtaaattagattgtctaaaaaaaatagttattctgattatatttttgttatctgggcaaagtagaagtcaaggggtaaaaaccctactattctgatttttgcacaagttttagtggaaaataaaatcacactctacagtaaaaaaaaaaaaaaaaaa

SEQ ID NO: 14 - protein, 310 aa, Homo sapiens
MRPLQIVPSRLISQLYCGLKPPASTRNQICLKMARPSSSMADFRKFFAKAKHIVIISGAGVSAESGVPTFRGAGGYWRKWQAQDLATPLAFAHNPSRVWEFYHYRREVMGSKEPNAGHRAIAECETRLGKQGRRVVVITQNIDELHRKAGTKNLLEIHGSLFKTRCTSCGVVAENYKSPICPALSGKGAPEPGTQDASIPVEKLPRCEEAGCGGLLRPHVVWFGENLDPAILEEVDRELAHCDLCLVVGTSSVVYPAAMFAPQVAARGVPVAEFNTETTPATNRFRFHFQGPCGTTLPEALACHENETVS

SEQ ID NO: 15 - DNA, 1638 bp, Homo sapiens
  CDS (61)..(1125)
gcttccggcggaagcggcctcaacaagggaaactttattgttcccgtggggcagtcgaggatgtcggtgaattacgcggcggggctgtcgccgtacgcggacaagggcaagtgcggcctcccggagatcttcgaccccccggaggagctggagcggaaggtgtgggaactggcgaggctggtctggcagtcttccagtgtggtgttccacacgggtgccggcatcagcactgcctctggcatccccgacttcaggggtccccacggagtctggaccatggaggagcgaggtctggcccccaagttcgacaccacctttgagagcgcgcggcccacgcagacccacatggcgctggtgcagctggagcgcgtgggcctcctccgcttcctggtcagccagaacgtggacgggctccatgtgcgctcaggcttccccagggacaaactggcagagctccacgggaacatgtttgtggaagaatgtgccaagtgtaagacgcagtacgtccgagacacagtcgtgggcaccatgggcctgaaggccacgggccggctctgcaccgtggctaaggcaagggggctgcgagcctgcaggggagagctgagggacaccatcctagactgggaggactccctgcccgaccgggacctggcactcgccgatgaggccagcaggaacgccgacctgtccatcacgctgggtacatcgctgcagatccggcccagcgggaacctgccgctggctaccaagcgccggggaggccgcctggtcatcgtcaacctgcagcccaccaagcacgaccgccatgctgacctccgcatccatggctacgttgacgaggtcatgacccggctcatggagcacctggggctggagatccccgcctgggacggcccccgtgtgctggagagggcgctgccacccctgccccgcccgcccacccccaagctggagcccaaggaggaatctcccacccggatcaacggctctatccccgccggccccaagcaggagccctgcgcccagcacaacggctcagagcccgccagccccaaacgggagcggcccaccagccctgccccccacagaccccccaaaagggtgaaggccaaggcggtccccagctgaccagggtgcttggggagggtggggctttttgtagaaactgtggattctttttctctcgtggtctcactttgttacttgtttctgtccccgggagcctcagggctctgagagctgtgctccaggccaggggttacacctgccctccgtggtccctccctgggctccaggggcctctggtgcggttccgggaagaagccacaccccagaggtgacagctgagcccctgccacaccccagcctctgacttgctgtgttgtccagaggtgaggctgggccctccctggtctccagcttaaacaggagtgaactccctctgtccccagggcctcccttctgggccccctacagcccaccctacccctcctccatgggccctgcaggaggggagacccaccttgaagtgggggatcagtagaggcttgcactgcctttggggctggagggagacgtgggtccaccaggcttctggaaaagtcctcaatgcaataaaaacaatttctttcttgcaaa

SEQ ID NO: 16 - protein, 355 aa, Homo sapiens
MSVNYAAGLSPYADKGKCGLPEIFDPPEELERKVWELARLVWQSSSVVFHTGAGISTASGIPDFRGPHGVWTMEERGLAPKFDTTFESARPTQTHMALVQLERVGLLRFLVSQNVDGLHVRSGFPRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMEHLGLEIPAWDGPRVLERALPPLPRPPTPKLEPKEESPTRINGSIPAGPKQEPCAQHNGSEPASPKRERPTSPAPHRPPKRVKAKAVPS

SEQ ID NO: 17 - DNA, 1718 bp, Homo sapiens
  CDS (34)..(1233)
gcggaagcggaagagcaggtctccaggggagcgatggcagccgggggtctgagccgctccgagcgcaaagcggcggagcgggtccggaggttgcgggaggagcagcagagggagcgcctccgccaggtgtcgcgcatcctgaggaaggcggcggcggagcgcagcgccgaggagggccggctgctggccgagagcgcggacctggtaacggagctgcagggccggagccggcggcgcgagggcctgaagcggcggcaggaggaggtgtgcgacgacccggaggagctgcgggggaaggtccgggagctggccagcgccgtccggaacgccaaatacttggtcgtctacacaggcgcgggaatcagcacggcagcgtctatcccagactaccggggccctaatggagtgtggacactgcttcagaaagggagaagcgttagtgctgccgacctgagcgaggccgagccaaccctcacccacatgagcatcacccgtctgcatgagcagaagctggtgcagcatgtggtgtctcagaactgtgacgggctccacctgaggagtgggctgccgcgcacggccatctccgagctccacgggaacatgtacattgaagtctgtacctcctgcgttcccaacagggagtacgtgcgggtgttcgatgtgacggagcgcactgccctccacagacaccagacaggccggacctgccacaagtgtgggacccagctgcgggacaccattgtgcactttggggagagggggacgttggggcagcctctgaactgggaagcggcgaccgaggctgccagcagagcagacaccatcctgtgtctagggtccagcctgaaggttctaaagaagtacccacgcctctggtgcatgaccaagccccctagccggcggccgaagctttacatcgtgaacctgcagtggaccccgaaggatgactgggctgccctgaagctacatgggaagtgtgatgacgtcatgcggctcctcatggccgagctgggcttggagatccccgcctatagcaggtggcaggatcccattttctcactggcgactcccctgcgtgctggtgaagaaggcagccacagtcggaagtcgctgtgcagaagcagagaggaggccccgcctggggaccggggtgcaccgcttagctcggcccccatcctagggggctggtttggcaggggctgcacaaaacgcacaaaaaggaagaaagtgacgtaatcacgtgctcgatgaagaacagttggcactttgcagatggccagtgtcacggtgaaggctgggttgcccccacgggtctagggagaacgaactctttggggatgacattttcaccgtgacatttttagccatttgtccttgaggaagccccttgcactgctgcggttgtaccctgatacggcctggccatcgaggacacctgcccatccggcctctgtgtcaagaggtggcagccgcacctttctgtgagaacggaactcgggttatttcagccccggcctgcagagtggaagcgcccagcggcctttcctcgctcaccaggccagtctcagggcctcaccgtatttctactactacttaatgaaaaagtgtgaactttatagaatcctctctgtactggatgtgcggcagaggggtggctccgagcctcggctctatgcagacctttttatttctattaaacgtttctgcactggcaaaaa

SEQ ID NO: 18 - protein, 400 aa, Homo sapiens
MAAGGLSRSERKAAERVRRLREEQQRERLRQVSRILRKAAAERSAEEGRLLAESADLVTELQGRSRRREGLKRRQEEVCDDPEELRGKVRELASAVRNAKYLVVYTGAGISTAASIPDYRGPNGVWTLLQKGRSVSAADLSEAEPTLTHMSITRLHEQKLVQHVVSQNCDGLHLRSGLPRTAISELHGNMYIEVCTSCVPNREYVRVFDVTERTALHRHQTGRTCHKCGTQLRDTIVHFGERGTLGQPLNWEAATEAASRADTILCLGSSLKVLKKYPRLWCMTKPPSRRPKLYIVNLQWTPKDDWAALKLHGKCDDVMRLLMAELGLEIPAYSRWQDPIFSLATPLRAGEEGSHSRKSLCRSREEAPPGDRGAPLSSAPILGGWFGRGCTKRTKRKKVT

SEQ ID NO: 19 - protein, 6 aa, Homo sapiens
  MOD_RES (1): Lys(Ac)
KQTARK

SEQ ID NO: 20 - protein, 6 aa, Homo sapiens
  MOD_RES (1): Lys(Ac)
KSTGGK

SEQ ID NO: 21 - protein, 6 aa, Homo sapiens
  MOD_RES (1): Lys(Ac)
KSTGGK

SEQ ID NO: 22 - protein, 5 aa, Homo sapiens
  MOD_RES (1): Lys(Ac)
KAPRK

SEQ ID NO: 23 - protein, 5 aa, Homo sapiens
  MOD_RES (5): Lys(Ac)
SGRGK

SEQ ID NO: 24 - protein, 5 aa, Homo sapiens
  MOD_RES (5): Lys(Ac)
KGGAK

SEQ ID NO: 25 - protein, 5 aa, Homo sapiens
  MOD_RES (1): Lys(Ac)
KGGAK

SEQ ID NO: 26 - protein, 4 aa, Homo sapiens
  MOD_RES (4): Lys(Ac)
QPKK

SEQ ID NO: 27 - protein, 4 aa, Homo sapiens
  MOD_RES (1): Lys(Ac)
KSKK

SEQ ID NO: 28 - protein, 4 aa, Homo sapiens
  MOD_RES (4): Lys(Ac)
RHKK

SEQ ID NO: 29 - protein, 4 aa, Homo sapiens
  MOD_RES (3): Lys(Ac)
RHKK

SEQ ID NO: 30 - protein, 369 aa, Homo sapiens [sequence omitted]

SEQ ID NO: 31 - protein, 562 aa, Saccharomyces cerevisiae
MTIPHMKYAVSKTSENKVSNTVSPTQDKDAIRKQPDDIINNDEPSHKKIKVAQPDSLRETNTTDPLGHTKAALGEVASMELKPTNDMDPLAVSAASVVSMSNDVLKPETPKGPIIISKNPSNGIFYGPSFTKRESLNARMFLKYYGAHKFLDTYLPEDLNSLYIYYLIKLLGFEVKDQALIGTINSIVHINSQERVQDLGSAISVTNVEDPLAKKQTVRLIKDLQRAINKVLCTRLRLSNFFTIDHFIQKLHTARKILVLTGAGVSTSLGIPDFRSSEGFYSKIKHLGLDDPQDVFNYNIFMHDPSVFYNIANMVLPPEKIYSPLHSFIKMLQMKGKLLRNYTQNIDNLESYAGISTDKLVQCHGSFATATCVTCHWNLPGERIFNKIRNLELPLCPYCYKKRREYFPEGYNNKVGVAASQGSMSERPPYILNSYGVLKPDITFFGEALPNKFHKSIREDILECDLLICIGTSLKVAPVSEIVNMVPSHVPQVLINRDPVKHAEFDLSLLGYCDDIAAMVAQKCGWTIPHKKWNDLKNKNFKCQEKDKGVYVVTSDEHPKTL

(19) United States
(12) Patent Application Publication
(10) Pub. No.: US 2006/0025337 A1
(43) Pub. Date: Feb. 2, 2006

(54) Sirtuin related therapeutics and diagnostics for neurodegenerative diseases

(75) Inventors: David A. Sinclair, West Roxbury, MA (US); Li-Huei Tsai, Cambridge, MA (US); Minh Dang Nguyen, Boston, MA (US); Konrad T. Howitz, Allentown, PA (US); Robert E. Zipkin, Wynnewood, PA (US); Kevin J. Bitterman, Boston, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: President and Fellows of Harvard College, Cambridge, MA (US)

(21) Appl. No.: 11/074,374

(22) Filed: Mar. 7, 2005

Related U.S. Application Data
(63) Continuation-in-part of application No. 10/884,022, filed on Jul. 1, 2004.
(63) Continuation-in-part of application No. 10/884,879, filed on Jul. 1, 2004.
(60) Provisional application No. 60/483,949, filed on Jul. 1, 2003.
(60) Provisional application No. 60/532,158, filed on Dec. 23, 2003.
(60) Provisional application No. 60/483,949, filed on Jul. 1, 2003.
(60) Provisional application No. 60/532,158, filed on Dec. 23, 2003.

(51) Int. Cl. A61K 38/17; A61K 48/00; C12Q 1/02

(52) U.S. Cl. 514/12; 435/29; 514/44

(57) ABSTRACT

Provided herein are methods and compositions for modulating the activity of sirtuin deacetylase protein family members; p53 activity; apoptosis; lifespan and sensitivity to stress of cells and organisms. Exemplary methods comprise contacting a cell with an activating compound, such as a flavone, stilbene, flavanone, isoflavone, catechin, chalcone, tannin or anthocyanidin; or an inhibitory compound, such as a sphingolipid, e.g., sphingosine. Also disclosed herein are methods for treating, preventing or diagnosing a disease associated with neuronal cell death, e.g., a neurodegenerative disease.

### CROSS-REFERENCE TO RELATED APPLICATIONS

This application is a continuation-in-part application of U.S. application Ser. No. 10/884,022, filed Jul. 1, 2004 and U.S. application Ser. No. 10/884,879, filed Jul. 1, 2004, both of which claim the benefit of U.S. Provisional Application No. 60/483,949, filed Jul. 1, 2003 and U.S. Provisional Application No. 60/532,158, filed Dec. 23, 2003. The content of all of these applications is specifically incorporated by reference herein.

### GOVERNMENT SUPPORT

This invention was made with government support under Grant numbers GM53049, AG19719 and AG19972 awarded by the National Institutes of Health. The government has certain rights in this invention.

### BACKGROUND

There is now good evidence from model organisms that the pace of aging can be regulated¹. Longevity regulatory genes have been identified in many eukaryotes, including rodents, flies, nematode worms and even single-celled organisms such as baker's yeast (reviewed in^{2,3}). These genes appear to be part of an evolutionarily conserved longevity pathway that evolved to promote survival in response to deteriorating environmental conditions^{1,4}. The yeast S. cerevisiae has proven a particularly useful model in which to study cell autonomous pathways of longevity regulation². In this organism, replicative lifespan is defined as the number of daughter cells an individual mother cell produces before dying. Yeast lifespan extension is governed by PNC1, a calorie restriction (CR)- and stress-responsive gene that depletes nicotinamide, a potent inhibitor of the longevity protein Sir2. Both PNC1 and SIR2 are required for lifespan extension by CR or mild stress^{5,6} and additional copies of these genes extend lifespan 30-70%⁵⁻⁷. Based on these results we proposed that CR may confer health benefits in a variety of species because it is a mild stress that induces a sirtuin-mediated organismal defense response⁶.

Sir2, a histone deacetylase (HDAC), is the founding member of the sirtuin deacetylase family, which is characterized by a requirement for NAD⁺ as a co-substrate⁸⁻¹³. SIR2 was originally identified as a gene required for the formation of transcriptionally silent heterochromatin at yeast mating-type loci¹⁴. Subsequent studies have shown that Sir2 suppresses recombination between repetitive DNA sequences at ribosomal RNA genes (rDNA)¹⁵⁻¹⁷. Sir2 has also been implicated in the partitioning of carbonylated proteins to yeast mother cells during budding¹⁸. Studies in C. elegans, mammalian cells, and the single-celled parasite Leishmania, indicate that the survival and longevity functions of sirtuins are conserved¹⁹⁻²². In C. elegans additional copies of sir-2.1 extend lifespan by 50% via the insulin/IGF-1 signalling pathway, the same pathway recently shown to regulate lifespan in rodents²³⁻²⁵.

### SUMMARY

Provided herein are methods for activating a sirtuin deacetylase protein family member. The method may comprise contacting a sirtuin deacetylase protein family member with a compound having a structure selected from the group consisting of formulas 1-25, 30 and 32-65. Compounds falling within formulas 1-25, 30 and 32-65 and activating a sirtuin protein are referred to herein as “activating compounds.” The activating compound may be a polyphenol compound, such as a plant polyphenol or an analog or derivative thereof. Exemplary compounds are selected from the group consisting of flavones, stilbenes, flavanones, isoflavones, catechins, chalcones, tannins and anthocyanidins or analog or derivative thereof. In illustrative embodiments, compounds are selected from the group consisting of resveratrol, butein, piceatannol, isoliquiritgenin, fisetin, luteolin, 3,6,3′,4′-tetrahydroxyfalvone, quercetin, and analogs and derivatives thereof. In certain embodiments, if the activating compound is a naturally occurring compound, it may not in a form in which it is naturally occurring.

The sirtuin deacetylase protein family member may be the human SIRT1 protein or the yeast Sir2 protein.

The sirtuin deacetylase protein family member may be in a cell, in which case the method may comprise contacting the cell with an activating compound or introducing a compound into the cell. The cell may be in vitro. The cell may be a cell of a subject. The cell may be in a subject and the method may comprise administering the activating compound to the subject. Methods may further comprise determining the activity of the sirtuin deacetylase protein family member.

A cell may be contacted with an activating compound at a concentration of about 0.1-100 μM. In certain embodiments, a cell is further contacted with an additional activating compound. In other embodiments, a cell is contacted with a least three different activating compounds.

Other methods encompassed herein include methods for inhibiting the activity of p53 in a cell and optionally protecting the cell against apoptosis, e.g., comprising contacting the cell with an activating compound at a concentration of less than about 0.5 μM. Another method comprises stimulating the activity of p53 in a cell and optionally inducing apoptosis in the cell, comprising contacting the cell with an activating compound at a concentration of at least about 50 μM.

Also provided herein is a method for extending the lifespan of a eukaryotic cell, such as by increasing its resistance to stress, comprising contacting the cell with a compound selected from the group consisting of stilbene, flavone and chalcone family members. Such compounds are referred to as “lifespan extending compounds.” The compound may have the structure set forth in formula 7. Other compounds may be activating compounds having a structure set forth in any of formulas 1-25, 30 and 32-65, provided they extend lifespan or increase resistance to stress. The compound may be selected from the group consisting of resveratrol, butein and fisetin and analogs and derivatives thereof. In certain embodiments, if the lifespan extending compound is a naturally occurring compound, it is not in a form in which it is naturally occurring. The method may further comprise determining the lifespan of the cell. The method may also further comprise contacting the cell with an additional compound or with at least three compounds selected from the group consisting of stilbene, flavone and chalcone family members or other lifespan extending compound. The cell may be contacted with a compound at a concentration of less than about 10 μM or at a concentration of about 10-100 μM. The cell may be in vitro or in vivo, it may be a yeast cell or a mammalian cell. If the cell is in a subject, the method may comprise administering the compound to the subject.

Methods for inhibiting sirtuins; inhibiting deacetylation of p53; stimulating apoptosis; shorting lifespan and rendering cells and organisms sensitive to stress are also encompassed. One method comprises contacting a sirtuin or cell or organism comprising such with an inhibitory compound having a formula selected from the group of formulas 26-29, 31 and 66-68.

Also provided herein are compositions comprising, e.g., at least one or at least two compounds each having a formula selected from the group consisting of formulas 1-68. Further provided herein are screening methods for identifying compounds, e.g., small molecules, that modulate sirtuins and/or modulate the life span or resistance to stress of cells. Methods may comprise (i) contacting a cell comprising a SIRT1 protein with a peptide of p53 comprising an acetylated residue 382 in the presence of an inhibitor of class I and class II HDAC under conditions appropriate for SIRT1 to deacetylate the peptide and (ii) determining the level of acetylation of the peptide, wherein a different level of acetylation of the peptide in the presence of the test compound relative to the absence of the test compound indicates that the test compound modulates SIRT1 in vivo.

Also provided are methods for treating or preventing a neurodegenerative disease in a subject, comprising administering to a subject in need thereof a therapeutically effective amount of an agent that increases the activity or protein level of a sirtuin, e.g., SIRT1, in a cell. The agent may be a nucleic acid encoding a sirtuin or a sirtuin-activating compound, salt or prodrug thereof. The neurodegenerative disease may be a disease selected from the group consisting of Alzheimer's disease, amyotrophic lateral sclerosis (ALS), Parkinson's disease, Huntington's disease, and multiple sclerosis. The methods may further comprise administering to the subject a therapeutically effective amount of a second therapeutic agent for treating the neurodegenerative disease. The subject may be a subject who has been diagnosed with a neurodegenerative disease and wherein the diagnosis may comprise determining the level or activity of a sirtuin in the subject. The methods may further comprise monitoring the progression of the neurodegenerative disease, such as by determining the level or activity of a sirtuin in the subject.

Compositions comprising a compound having a formula selected from the group of formulas 1-25, 30 and 32-65 and a second agent, e.g., a therapeutic agent for treating a neurodegenerative disease, are also disclosed.

Other methods include methods for determining whether a subject has or is likely to develop a neurodegenerative disease, comprising (i) obtaining a biological sample from a subject; and (ii) determining the level or activity of a sirtuin in the biological sample, wherein a higher level or activity of the sirtuin the biological sample of the subject relative to a control level indicates that the subject has or is likely to develop a neurodegenerative disease. A diagnostic method may further be followed by a method of treatment or prevention.

### BRIEF DESCRIPTION OF THE DRAWINGS

FIG. 1 shows the effects of resveratrol on the kinetics of recombinant human SIRT1. a, Resveratrol dose-response of SIRT1 catalytic rate at 25 μM NAD⁺, 25 μM p53-382 acetylated peptide. Relative initial rates are the mean of two determinations, each derived from the slopes of fluorescence (arbitrary fluorescence units, AFU) vs. time plots with data obtained at 0, 5, 10 and 20 min. of deacetylation. b, SIRT1 initial rate at 3 mM NAD⁺, as a function of p53-382 acetylated peptide concentration in the presence (Δ) or absence (ν) of 100 μM resveratrol. Lines represent non-linear least-squares fits to the Michaelis-Menten equation. Kinetic constants: Kₘ(control, ν)=64 μM, Kₘ (+resveratrol, Δ)=1.8 μM; Vₘₐₓ(control, ν)=1107 AFU/min., Vₘₐₓ(+resveratrol, Δ)=926 AFU/min. c, SIRT1 initial rate at 1 mM p53-382 acetylated peptide, as a function of NAD⁺ concentration, in the presence (Δ) or absence (ν) of 100 μM resveratrol. Lines represent non-linear least-squares fits to the Michaelis-Menten equation. Kinetic constants: Kₘ(control, ν)=558 μM, Kₘ(+resveratrol, Δ)=101 μM; Vₘₐₓ(control, ν)=1863 AFU/min., Vₘₐₓ(+resveratrol, Δ)=1749 AFU/min. d, Effects of resveratrol on nicotinamide inhibition of SIRT1. Kinetic constants are shown relative to those of the control (no nicotinamide, no resveratrol) and represent the mean of two determinations. Error bars are standard errors of the mean. The variable substrate in each experiment (N=NAD⁺, P=p53 acetylated peptide), the presence/absence of nicotinamide (+/−) and the resveratrol concentration (μM) are indicated beneath each pair of Kₘ-Vₘₐₓ bars.

FIG. 2 shows the effects of polyphenols on Sir2 and S. cerevisiae lifespan. a, Initial deacetylation rate of recombinant GST-Sir2 as a function of resveratrol concentration. Rates were determined at the indicated resveratrol concentrations, either with 100 μM ‘Fluor de Lys’ acetylated lysine substrate (FdL) plus 3 mM NAD⁺ (Δ) or with 200 μM p53-382 acetylated peptide substrate plus 200 μM NAD⁺ (ν). b, Lifespan analyses were determined by micro-manipulating individual yeast cells as described³⁷ on complete 2% glucose medium with 10 μM of each compound, unless otherwise stated. Average lifespan for wild type, 22.9 generations, quercetin, 23.4; piceatannol. 24.0. c, Average lifespan for wild type, 22.9 generations; fisetin, 30.0; butein, 35.5; resveratrol, 36.8. d, Average lifespan for wild type untreated, 21.0 generations; growth on resveratrol, 10 μM, 35.7; 100 μM, 29.4; 500 μM, 29.3.

FIG. 3 shows that resveratrol extends lifespan by mimicking CR and suppressing rDNA recombination. Yeast lifespans were determined as in FIG. 2. a, Average lifespan for wild type (wt) untreated, 19.0 generations; wild type+resveratrol (wt+R) 37.8; glucose-restricted+resveratrol (CR+R), 39.9. b, Average lifespans for wild type sir2Δ, 9.9; sir2Δ+resveratrol, 10.0; pnc1Δ, 19.2; pnc1Δ+resveratrol, 33.1. c, Resveratrol suppresses the frequency of ribosomal DNA recombination in the presence and absence of nicotinamide (NAM). Frequencies were determined by loss of the ADE2 marker gene from the rDNA locus (RDN1). d, Resveratrol does not suppress rDNA recombination in a sir2 strain. e, Resveratrol and other sirtuin activators do not significantly increase rDNA silencing compared to a 2×SIR2 strain. Pre-treated cells (RDN1:: URA3) were harvested and spotted as 10-fold serial dilutions on either SC or SC with 5-fluororotic acid (5-FOA). In this assay, increased rDNA silencing results in increased survival on 5-FOA medium. f, Quantitation of the effect of resveratrol on rDNA silencing by counting numbers of surviving cells on FOA/total plated.

FIG. 4 shows that resveratrol and other polyphenols stimulate SIRT1 activity in human cells. a, Method for assaying intracellular deacetylase activity with a fluorogenic, cell-permeable substrate, FdL (‘Fluor de Lys’, BIOMOL). FdL (200 μM) is added to growth media and cells incubated for 1-3 hours to allow FdL to enter the cells and the lysine-deacetylated product (deAc-FdL) to accumulate intracellularly. Cells are lysed with detergent in the presence of 1 μM TSA, 1 mM nicotinamide. Addition of the non-cell-permeable Developer (BIOMOL) releases a fluorophor, specifically from deAc-FdL. b, SIRT1 activating polyphenols can stimulate TSA-insensitive, FdL deacetylation by HeLa S3 cells. Cells were grown adherently in DMEM/10% FCS and treated for 1 hour with 200 μM FdL, 1 μM TSA and either vehicle (0.5% final DMSO, Control) or 500 μM of the indicated compound. Intracellular accumulation of deAc-FdL was then determined as described briefly in a. The intracellular deAc-FdL level for each compound (mean of six replicates) are plotted against the ratios to the control rate obtained in the in vitro SIRT1 polyphenol screen (see Table 1, Supplementary Tables 1 and 3). c, U2OS osteosarcoma cells grown to ≧90% confluence in DMEM/10% FCS were exposed to 0 or 10 grays of gamma irradiation (IR). Whole cell lysates were prepared 4 hours post-irradiation and were probed by Western blotting with indicated antibodies. d, U2OS cells cultured as above were pre-treated with the indicated amounts of resveratrol or a 0.5% DMSO blank for 4 hours after which cells were exposed to 0 or 50 J/cm² of UV radiation. Lysates were prepared and analyzed by Western blot as in c. e, Human embryonic kidney cells (HEK 293) expressing wild type SIRT1 or dominant negative SIRT1-H363Y (SIRT1-HY) protein were cultured as above, pre-treated with the indicated amounts of resveratrol or a 0.5% DMSO blank for 4 hours and exposed to 50 J/cm² of UV radiation as above. Lysates were prepared and analyzed as above.

FIG. 5 shows that intracellular deacetylation activity may be measured with a cell-permeable, fluorogenic HDAC and sirtuin substrate. HeLa S3 cells were grown to confluence in DMEM/10% FCS and then incubated with fresh medium containing 200 μM FdL for the indicated times, 37° C. Intracellular and medium levels of deacetylated substrate (deAc-FdL) were determined according to the manufacturer's instructions (HDAC assay kit, BIOMOL). All data points represent the mean of two determinations. a, Concentration ratio of intracellular ([deAc-FdL]ᵢ) to medium ([deAc-FdL]ₒ) concentrations in the presence (Δ) or absence (ν) of 1 μM trichostatin A (TSA). b, Total accumulation of deacetylated substrate (deAc-FdL) in the presence (Δ) or absence (ν) of 1 μM TSA. c, Intracellular accumulation of deacetylated substrate (deAc-FdL) in the presence (Δ) or absence (ν) of 1 μM TSA.

FIG. 6 shows that deacetylation site preferences of recombinant SIRT1. Initial rates of deacetylation were determined for a series of fluorogenic acetylated peptide substrates based on short stretches of human histone H3, H4 and p53 sequence (see key to substrate name and single letter peptide sequence below the bar graph). Recombinant human SIRT1 (1 μg, BIOMOL), was incubated 10 min, 37° C., with 25 μM of the indicated fluorogenic acetylated peptide substrate and 500 μM NAD⁺. Reactions were stopped by the addition of 1 mM nicotinamide and the deacetylation-dependent fluorescent signal was determined.

FIG. 7 is a graph representing SIRT2 activity as a function of resveratrol concentration.

FIG. 8 shows an alignment of the amino acid sequences of hSIRT2, hSIRT1 and S. cerevisiae Sir2.

FIG. 9A shows resveratrol and BML-230 dose responses of SIRT1 catalytic rate. Points represent the mean of three determinations and error bars are standard errors of the mean.

FIG. 9B shows the ratio of BML-230-activated to resveratrol-activated SIRT1 rates as a function of activator concentration (the ratios were calculated from data of FIG. 9A).

FIG. 10 shows the effect of polyphenolic STACs on metazoan sirtuins. a, Schematic of Sir2 polypeptides from human, yeast, C. elegans and D. melanogaster aligned to show conserved regions. Amino acids forming the NAD⁺-binding pocket (grey) and substrate binding groove (black) are indicated. Percentages refer to the homology to SIRT1. b, Effect of polyphenolic STACs (500 μM) on NAD⁺-dependent, trichostatin A (TSA)-insensitive deacetylase activity in Drosophila S2 cells. c, Fold stimulation of recombinant SIR-2.1 by STACs (10 μM). d, Fold stimulation of recombinant dSir2 by STACs (10 μM). Values are the mean of at least three determinations (+/−standard error). e, Dose-dependent activation of C. elegans SIR-2.1 by resveratrol. Rates were determined using a fluorigenic acetylated lysine substrate (Fluor de Lys). f, Dose-dependent activation of Drosophila dSir2 by resveratrol. g, SIR-2.1 initial rate at 10 μM Fluor de Lys as a function of NAD⁺ concentration, in the presence or absence of 100 μM resveratrol. AFU, arbitrary fluorescence units.

FIG. 11 shows the C. elegans survival on resveratrol. a, Survivorship of adult wild-type N2 C. elegans treated with 100 μM resveratrol fed with heat-killed OP50 E. coli. Mean lifespan relative to control (triangles, n=47) was increased by 14.5% (Log-Rank test, P<0.0001) by 100 μM resveratrol (squares, n=46). b, Survivorship of sir-2.1 mutants treated with resveratrol fed with heat-killed OP50. Adult lifespan of sir-2.1 animals does not differ significantly from N2 controls (Log-Rank, P=0.68) and the effect on lifespan of 100□M resveratrol on sir-2.1 mutant animals was not statistically significant (5.2% extension, Log-Rank P=0.058; n=60 control, 58 treated). c, Survivorship of wild-type N2 C. elegans on 100 μM resveratrol fed with live OP50 (12.6% extension, P<0.0001; n=47 control, 67 treated). d, Survivorship of sir-2.1 mutants on 100 μM resveratrol fed with live OP50 (3.3% extension, P=0.81; n=57 control, 51 treated) e, Fecundity of adult hermaphrodites treated with 100 μM resveratrol. Controls: 106 eggs/5 worms/5 hours (s.d. 10.0); resveratrol-treated: 99 eggs/5 worms/5 hours (s.d. 13.0). f, Feeding rates of L4 larval and adult hermaphrodites treated with 100 μM resveratrol. L4 on live OP50: control 310±10.2 pumps/min, resveratrol 315±9.8; Adult on dead OP50: control 228±26.2, resveratrol 283±31.9; Adult on live OP50: control 383±16.0, resveratrol 383±22.7.

FIG. 12 shows wild-type female D. melanogaster survival with adults fed resveratrol or fisetin. a, Canton-S on 15% SY media. b, Canton-S on 5% SY media with resveratrol at two concentrations. c, Strain yw on 3% CSY media. d, Strain yw on 2% CSY media with resveratol at two concentrations. e, Strain yw on 3% CSY media with 100 μM resveratrol or fisetin. f, Strain yw on 2% CSY media with 100 μM resveratrol or fisetin. Life table statistics for this figure, for males and for additional trials are in Table 20. g, Mean daily fecundity per female (s.e.) estimated over 5-day intervals of Canton-S on 15% SY media with 0 or 10 μM resveratrol. h, Proportion (s.e.) of yw females feeding on diet with and without resveratrol in crop-filling assay. i, Mean (s.e.) body mass of Canton-S males and females feeding on diet without and with resveratrol (10 μM).

FIG. 13 shows the survivorship of D. melanogaster adults with mutant alleles of dSir2 when fed resveratrol (100 μM). Females (a) and males (b) with loss-of-function genotype dSir2^{4.5}/dSir2^{5.26}. Females (c) and males (d) with strong hypomorphic genotype dSir2¹⁷/dSir2^{KG00871}.

FIG. 14 shows the mortality rates of control and resveratrol treated adults. Mortality was estimated as ln(−ln(pₓ)) where pₓ is the survival probability at day x to x+1. a, C. elegans wild-type N2 on heat-killed OP50 E. coli. b, C. elegans wild-type N2 on live OP50 E. coli. In a and b mortality is plotted only at days with observed mortality. c, D. melanogaster wildtype females of Trial 1 at effective doses of resveratrol on 15% SY diet. d, D. melanogaster wildtype males of Trial 1 at effective doses of resveratrol on 15% SY diet. In c and d mortality is smoothed from 3-day running average of pₓ.

FIG. 15 shows the stimulation of SIRT 1 catalytic rate by 100 μM plant polyphenols (Table 1).

FIG. 16 shows the effect of 100 μM stilbenes and chalcones on SIRT 1 catalytic rate (Supplementary Table 1).

FIG. 17 shows the effect of 100 μM flavones on SIRT 1 catalytic rate (Supplementary Table 2).

FIG. 18 shows the effect of 100 μM flavones on SIRT 1 catalytic rate (Supplementary Table 3).

FIG. 19 shows the effect of 100 μM isoflavones, flavanones and anthocyanidins on SIRT 1 catalytic rate (Supplementary Table 4).

FIG. 20 shows the effect of 100 μM catechins (Flavan-3-ols) on SIRT 1 catalytic rate (Supplementary Table 5).

FIG. 21 shows the effect of 100 μM free radical protective compounds on SIRT 1 catalytic rate (Supplementary Table 6).

FIG. 22 shows the effect of 100 μM miscellaneous compounds on SIRT 1 catalytic rate (Supplementary Table 7).

FIG. 23 shows the effect of 100 μM of various modulators on SIRT 1 catalytic rate (Supplementary Table 8).

FIG. 24 shows the effect of 100 μM of new resveratrol analogs on SIRT 1 catalytic rate (Table 9).

FIG. 25 shows the effect of 100 μM of new resveratrol analogs on SIRT 1 catalytic rate (Table 10).

FIG. 26 shows the effect of 100 μM of new resveratrol analogs on SIRT 1 catalytic rate (Table 11).

FIG. 27 shows the effect of 100 μM of new resveratrol analogs on SIRT 1 catalytic rate (Table 12).

FIG. 28 shows the effect of 100 μM of new resveratrol analogs on SIRT 1 catalytic rate (Table 13).

FIG. 29 shows synthetic intermediates of resveratrol analog synthesis (Table 14).

FIG. 30 shows synthetic intermediates of resveratrol analog synthesis (Table 15).

FIG. 31 shows synthetic intermediates of resveratrol analog synthesis (Table 16).

FIG. 32 shows synthetic intermediates of resveratrol analog synthesis (Table 17).

FIG. 33 shows synthetic intermediates of resveratrol analog synthesis (Table 18).

FIG. 34 shows the effect of resveratrol on Drosophila melanogaster (Table 20).

FIGS. 35A-G shows sirtuin activators and the fold activation of SIRT1 (Table 21).

FIG. 36 shows sirtuin inhibitors and the fold inhibition of SIRT1 (Table 22).

FIG. 37 shows the upregulation of SIRT1 in mouse models displaying progressive and severe neurodegeneration. (A) is an immunoblot showing the upregulation of SIRT1 in p25 transgenic (Tg) mice during progressive neurodegeneration (after 2-12 weeks of induction). (B) is a graph showing the quantification of levels of SIRT1 in p25 Tg mice. ** P(T<=t) two tails: 0.004. (C) is a graph showing mRNA levels of Sirtuin family members in p25 Tg mice revealed by microarrays. ** P(T<=t) two tails: 0.003. (D) is an immunoblot showing the progressive increase of SIRT1 in mutant SOD1G37R (line 29) peaking at stage of massive neurodegeneration (10-12 month). (E) is a graph showing the quantification of levels of SIRT1 in SOD1G37R mice. ** P(T<=t) two tails: 0.005. *** P(T<=t) two tails: 0.002. (F) in an immunoblot showing the treatment of primary cortical neurons with low concentrations of ionomycin (1 μm) and hydrogen peroxide (25 μm) induces the rapid upregulation of SIRT associated with generation of p25. Time expressed in minutes.

FIG. 38 provides a western blot and a graph showing the levels of SIRT1 in forbrains of PDAPP-V717F mice. There is no increase of SIRT1 in brains of PDAPP-V717F and TauP301L mice.

FIG. 39 shows primary neurons treated with resveratrol are protected against either p25 or mutant SOD1 toxicity. (A) is a series of immunofluorescence images showing no toxicity observed in GFP-transfected neurons treated with resveratrol for 48 hours (50 to 500 nm). For all experiments (A to E), primary rat neurons were transfected at DIV1 with plasmids and resveratrol was added to the medium at 2-3 hours after transfection. Characterization of neuronal integrity was performed 24 to 48 hours after transfection. Bar: 40 μm. (B) shows representative confocal images of dying and healthy neurons transfected with p25-GFP and treated with DMSO (control) or resveratrol (250 nm) for 24 hours, respectively. Bar: 20 μm. (C) is a graph showing the quantification of surviving p25-GFP expressing neurons after treatment with DMSO (control) or 250 nm of resveratrol for 24 hours expressed in % (49+/−2% vs 72+/−8%; ** P(T<=t) two tails: 0.01). (D) shows representative confocal images of neurons transfected with SOD1G93A-FLAG and treated with DMSO (Control) or 500 nm of resveratrol for 48 hours. Bar: 25 μm. (E) is a graph showing the quantification of surviving SOD1G93A-FLAG-expressing neurons after treatment with control (DMSO) or resveratrol (500 μm) for 48 hours expressed in %. (28+/−3% vs 41+/−3%; ** P(T<=t) two tails: 0.01).

FIG. 40 shows that overexpression of SIRT1 protects against p25 and mutant SOD1 toxicity. (A) is a series of immunofluorescence images of primary neurons transfected with p25-GFP and the effects of overexpression of SIRT1 or SIRT1 lacking deacetylase activity (H363Y) on p25 GFP toxicity. Arrows point to neurons with ectopic expression of SIRT1. Bar: 20 μm. p25-GFP is shown in green and SIRT1 in red. (B) is a graph showing the quantification of surviving p25-GFP expressing neurons with or without ectopic expression of SIRT1 or H363Y. ** P(T<=t) two tails: 0.001. Non-significant (NS); P(T<=t) two tails: 0.27. (C) is an immunoblot showing unchanged levels of p25-GFP following expression of SIRT1 or H363Y. h: human; m: mouse. Comparison of HEK and CAD cells for SIRT1 expression. (D) is a series of immunofluorescence images showing primary neurons transfected with wild type hSOD1 or mutant hSOD1G93A and the effects of overexpression of SIRT1 or H363Y on SOD1G93A toxicity. Arrows point to SOD1 aggregates as detected with FLAG Ab. WTSOD1 is not toxic. Bar: 25 μm. hSOD1 is in red; Flag is in green; DAPI in blue. (E) is a graph showing the quantification of surviving SOD193A and WT SOD1-expressing neurons with or without ectopic expression of SIRT1 or H363Y. ** P(T<=t) two tails: 0.001. Non-significant (NS); P(T<=t) two tails: 0.33.

FIG. 41 is a series of immunofluorescence images showing the effects of SIRT1 overexpression on p25-GFP expressing neurons. Intact neuronal processes and nuclear morphology in primary neurons co-transfected with p25-GFP and SIRT1 (white arrow). No protection is observed in singly p25-transfected neurons (white arrowheads). Bar: 10 μm.

FIG. 42 is a series of immunofluorescence images showing the subcellular localization of SIRT1 in CNS neurons. SIRT1 localizes in nucleus and cell bodies of spinal motor neurons in wild-type (WT) and SOD1G93A mice. Bar: 15 μm.

FIG. 43 shows the upregulation of SIRT1 in prefontal cortex of post-mortem AD samples. (A) is an immunoblot and a graph showing the levels of SIRT1 in post-mortem prefontal cortex of control (n=9) and AD (n=11) patients scored by graph (average+/−standard error). Two sets of samples were processed independently. P(T<=t) two tails: 0.004. (B) is a series of confocal images of prefontal cortex SIRT1-positive neurons from control #1 and AD patients #1 and 2 in 1^{st} set. Bars: 120 μm and 40 μm. (C) shows the absence of correlation between levels of SIRT1-expressing neurons and proximity to β-amyloid plaques (stars). White arrows point to SIRT1-expressing neurons distant from a β-amyloid plaque stained with 4G8 Abs. White arrowheads point to SIRT1-expressing neurons in close proximity of β-amyloid plaques. AD samples 2 and 3 were used. Bar: 20 μm.

### DETAILED DESCRIPTION

Definitions

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

“Activating a sirtuin protein” refers to the action of producing an activated sirtuin protein, i.e., a sirtuin protein that is capable of performing at least one of its biological activities to at least some extent, e.g., with an increase of activity of at least about 10%, 50%, 2 fold or more. Biological activities of sirtuin proteins include deacetylation, e.g., of histones and p53; extending lifespan; increasing genomic stability; silencing transcription; and controlling the segregation of oxidized proteins between mother and daughter cells.

An “activating compound” or a “sirtuin activating compound” refers to a compound that activates a sirtuin protein or stimulates or increases at least one of its activities. Activating compounds may have a formula selected from the group of formulas 1-25, 30 and 32-65.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

A “form that is naturally occurring” when referring to a compound means a compound that is in a form, e.g., a composition, in which it can be found naturally. For example, since resveratrol can be found in red wine, it is present in red wine in a form that is naturally occurring. A compound is not in a form that is naturally occurring if, e.g., the compound has been purified and separated from at least some of the other molecules that are found with the compound in nature.

“Inhibiting a sirtuin protein” refers to the action of reducing at least one of the biological activities of a sirtuin protein to at least some extent, e.g., at least about 10%, 50%, 2 fold or more.

An “inhibitory compound” or “inhibiting compound” or “sirtuin inhibitory compound” refers to a compound that inhibits a sirtuin protein. Inhibitory compounds may have a formula selected from the group of formulas 26-29, 31 and 66-68.

A “naturally occurring compound” refers to a compound that can be found in nature, i.e., a compound that has not been designed by man. A naturally occurring compound may have been made by man or by nature. For example, resveratrol is a naturally-occurring compound. A “non-naturally occurring compound” is a compound that is not known to exist in nature or that does not occur in nature.

“Replicative lifespan” of a cell refers to the number of daughter cells produced by an individual “mother cell.” “Chronological aging” or “chronological lifespan,” on the other hand, refers to the length of time a population of non-dividing cells remains viable when deprived of nutrients. “Increasing the lifespan of a cell” or “extending the lifespan of a cell,” as applied to cells or organisms, refers to increasing the number of daughter cells produced by one cell; increasing the ability of cells or organisms to cope with stresses and combat damage, e.g., to DNA, proteins; and/or increasing the ability of cells or organisms to survive and exist in a living state for longer under a particular condition, e.g., stress. Lifespan can be increased by at least about 20%, 30%, 40%, 50%, 60% or between 20% and 70%, 30% and 60%, 40% and 60% or more using methods described herein.

“Sirtuin deacetylase protein family members;” “Sir2 family members;” “Sir2 protein family members;” or “sirtuin proteins” includes yeast Sir2, Sir-2.1, and human SIRT1 and SIRT2 proteins. The nucleotide and amino acid sequences of the human sirtuin, SIRT1 (silent mating type information regulation 2 homolog), are set forth as SEQ ID NOs: 1 and 2, respectively (corresponding to GenBank Accession numbers NM_{—}012238 and NP_{—}036370, respectively). The mouse homolog of SIRT1 is Sirt2α. Human Sirt2 corresponds to Genbank Accession numbers NM_{—}012237 and NP_{—}036369 (for variant 1; SEQ ID NOs: 3 and 4, respectively) and NM_{—}030593 and NP_{—}085096 (for variant 2; SEQ ID NOs: 5 and 6, respectively). Other family members include the four additional yeast Sir2-like genes termed “HST genes” (homologues of Sir two) HST1, HST2, HST3 and HST4, and the five other human homologues hSIRT3 (corresponding to Genbank Accession numbers NM_{—}012239 and NP_{—}036371; SEQ ID NOs: 7 and 8, respectively), hSIRT4 (corresponding to Genbank Accession numbers NM_{—}012240 and NP_{—}036372; SEQ ID NOs: 9 and 10, respectively), hSIRT5 (corresponding to Genbank Accession numbers NM_{—}012241 and NP_{—}036373 for variant 1 (SEQ ID NOs: 11 and 12, respectively) and NM_{—}031244 and NP_{—}112534 for variant 2 (SEQ ID NOs: 13 and 14, respectively)), hSIRT6 (corresponding to Genbank Accession numbers NM_{—}016539 and NP_{—}057623; SEQ ID NOs: 15 and 16, respectively) and hSIRT7 (corresponding to Genbank Accession numbers NM_{—}016538 and NP_{—}057622; SEQ ID NOs: 17 and 18, respectively) (Brachmann et al. (1995) Genes Dev. 9:2888 and Frye et al. (1999) BBRC 260:273). Preferred sirtuins are those that share more similarities with SIRT1, i.e., hSIRT1, and/or Sir2 than with SIRT2, such as those members having at least part of the N-terminal sequence present in SIRT1 and absent in SIRT2 such as SIRT3 has.

“Biologically active portion of a sirtuin” refers to a portion of a sirtuin protein having a biological activity, such as the ability to deacetylate. Biologically active portions of sirtuins may comprise the core domain of sirtuins. For example, amino acids 62-293 of SIRT1 having SEQ ID NO: 2, which are encoded by nucleotides 237 to 932 of SEQ ID NO: 1, encompass the NAD⁺ binding domain and the substrate binding domain. Therefore, this region is sometimes referred to as the core domain. Other biologically active portions of SIRT1, also sometimes referred to as core domains, include about amino acids 261 to 447 of SEQ ID NO: 2, which are encoded by nucleotides 834 to 1394 of SEQ ID NO: 1; about amino acids 242 to 493 of SEQ ID NO: 2, which are encoded by nucleotides 777 to 1532 of SEQ ID NO: 1; or about amino acids 254 to 495 of SEQ ID NO: 2, which are encoded by nucleotides 813 to 1538 of SEQ ID NO: 1.

The terms “comprise” and “comprising” are used in the inclusive, open sense, meaning that additional elements may be included.

A “direct activator” of a sirtuin is a molecule that activates a sirtuin by binding to it. A “direct inhibitor” of a sirtuin is a molecule that inhibits a sirtuin by binding to it.

The term “including” is used to mean “including but not limited to”. “Including” and “including but not limited to” are used interchangeably.

“Neurodegenerative disorder” or “neurodegenerative disease” or “neuropathology” refers to a wide range of diseases and/or disorders of the central and peripheral nervous system, such as Parkinson's disease, Alzheimer's disease (AD), amyotrophic lateral sclerosis (ALS), denervation atrophy, otosclerosis, stroke, dementia, multiple sclerosis, Huntington's disease, encephalopathy associated with acquired immunodeficiency disease (AIDS), and other diseases associated with neuronal cell toxicity and cell death.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The term “cis” is art-recognized and refers to the arrangement of two atoms or groups around a double bond such that the atoms or groups are on the same side of the double bond. Cis configurations are often labeled as (Z) configurations.

The term “trans” is art-recognized and refers to the arrangement of two atoms or groups around a double bond such that the atoms or groups are on the opposite sides of a double bond. Trans configurations are often labeled as (E) configurations.

The term “covalent bond” is art-recognized and refers to a bond between two atoms where electrons are attracted electrostatically to both nuclei of the two atoms, and the net effect of increased electron density between the nuclei counterbalances the internuclear repulsion. The term covalent bond includes coordinate bonds when the bond is with a metal ion.

The term “therapeutic agent” is art-recognized and refers to any chemical moiety that is a biologically, physiologically, or pharmacologically active substance that acts locally or systemically in a subject. The term thus means any substance intended for use in the diagnosis, cure, mitigation, treatment or prevention of disease or in the enhancement of desirable physical or mental development and/or conditions in an animal or human.

The term “therapeutic effect” is art-recognized and refers to a local or systemic effect in animals, particularly mammals, and more particularly humans caused by a pharmacologically active substance. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions described herein may be administered in a sufficient amount to produce a at a reasonable benefit/risk ratio applicable to such treatment.

The term “synthetic” is art-recognized and refers to production by in vitro chemical or enzymatic synthesis.

The term “meso compound” is art-recognized and refers to a chemical compound which has at least two chiral centers but is achiral due to a plane or point of symmetry.

The term “chiral” is art-recognized and refers to molecules which have the property of non-superimposability of the mirror image partner, while the term “achiral” refers to molecules which are superimposable on their mirror image partner. A “prochiral molecule” is a molecule which has the potential to be converted to a chiral molecule in a particular process.

The term “stereoisomers” is art-recognized and refers to compounds which have identical chemical constitution, but differ with regard to the arrangement of the atoms or groups in space. In particular, “enantiomers” refer to two stereoisomers of a compound which are non-superimposable mirror images of one another. “Diastereomers”, on the other hand, refers to stereoisomers with two or more centers of dissymmetry and whose molecules are not mirror images of one another.

Furthermore, a “stereoselective process” is one which produces a particular stereoisomer of a reaction product in preference to other possible stereoisomers of that product. An “enantioselective process” is one which favors production of one of the two possible enantiomers of a reaction product.

The term “regioisomers” is art-recognized and refers to compounds which have the same molecular formula but differ in the connectivity of the atoms. Accordingly, a “regioselective process” is one which favors the production of a particular regioisomer over others, e.g., the reaction produces a statistically significant increase in the yield of a certain regioisomer.

The term “epimers” is art-recognized and refers to molecules with identical chemical constitution and containing more than one stereocenter, but which differ in configuration at only one of these stereocenters.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

The term “structure-activity relationship” or “(SAR)” is art-recognized and refers to the way in which altering the molecular structure of a drug or other compound alters its biological activity, e.g., its interaction with a receptor, enzyme, nucleic acid or other target and the like.

The term “aliphatic” is art-recognized and refers to a linear, branched, cyclic alkane, alkene, or alkyne. In certain embodiments, aliphatic groups in the present compounds are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure. The term “alkyl” is also defined to include halosubstituted alkyls.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “heteroatom” is art-recognized and refers to an atom of any element other than carbon or hydrogen. Illustrative heteroatoms include boron, nitrogen, oxygen, phosphorus, sulfur and selenium.

The term “aryl” is art-recognized and refers to 5-, 6- and 7-membered single-ring aromatic groups that may include from zero to four heteroatoms, for example, benzene, naphtalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂—. “Halide” designates the corresponding anion of the halogens, and “pseudohalide” has the definition set forth on 560 of “Advanced Inorganic Chemistry” by Cotton and Wilkinson.

The terms “amine” and “amino” are art-recognized and refer to both unsubstituted and substituted amines, e.g., a moiety that may be represented by the general formulas: 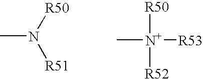
wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula: 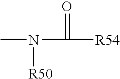
wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula: 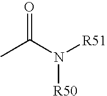
wherein R50 and R51 are as defined above. Certain embodiments of amides may not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R₆₁ are defined above. Representative alkylthio groups include methylthio, ethyl thio, and the like.

The term “carbonyl” is art recognized and includes such moieties as may be represented by the general formulas: 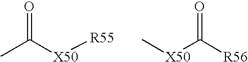
wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula: 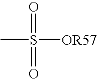
in which R57 is an electron pair, hydrogen, alkyl, cycloalkyl, or aryl.

The term “sulfate” is art recognized and includes a moiety that may be represented by the general formula: 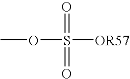
in which R57 is as defined above.

The term “sulfonamido” is art recognized and includes a moiety that may be represented by the general formula: 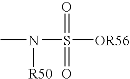
in which R50 and R56 are as defined above.

The term “sulfamoyl” is art-recognized and refers to a moiety that may be represented by the general formula: 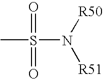
in which R50 and R51 are as defined above.

The term “sulfonyl” is art-recognized and refers to a moiety that may be represented by the general formula: 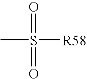
in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula: 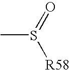
in which R58 is defined above.

The term “phosphoryl” is art-recognized and may in general be represented by the formula: 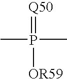
wherein Q50 represents S or O, and R59 represents hydrogen, a lower alkyl or an aryl. When used to substitute, e.g., an alkyl, the phosphoryl group of the phosphorylalkyl may be represented by the general formulas: 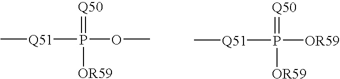
wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas: 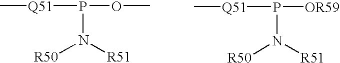
wherein Q51, R50, R51 and R59 are as defined above.

The term “phosphonamidite” is art-recognized and may be represented in the general formulas: 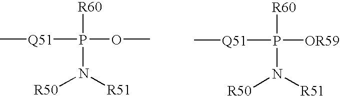
wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, and the like, when it occurs more than once in any structure, is intended to be independent of its definition elsewhere in the same structure.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions described herein may exist in particular geometric or stereoisomeric forms. In addition, compounds may also be optically active. Contemplated herein are all such compounds, including cis- and trans-isomers, R- and S-enantiomers, diastereomers, (D)-isomers, (L)-isomers, the racemic mixtures thereof, and other mixtures thereof. Additional asymmetric carbon atoms may be present in a substituent such as an alkyl group. All such isomers, as well as mixtures thereof, are encompassed herein.

If, for instance, a particular enantiomer of a compound is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. Heteroatoms such as nitrogen may have hydrogen substituents and/or any permissible substituents of organic compounds described herein which satisfy the valences of the heteroatoms. Compounds are not intended to be limited in any manner by the permissible substituents of organic compounds.

The chemical elements are identified in accordance with the Periodic Table of the Elements, CAS version, Handbook of Chemistry and Physics, 67th Ed., 1986-87, inside cover.

The term “protecting group” is art-recognized and refers to temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed by Greene and Wuts in Protective Groups in Organic Synthesis (2^{nd} ed., Wiley: New York, 1991).

The term “hydroxyl-protecting group” is art-recognized and refers to those groups intended to protect a hydroxyl group against undesirable reactions during synthetic procedures and includes, for example, benzyl or other suitable esters or ethers groups known in the art.

The term “carboxyl-protecting group” is art-recognized and refers to those groups intended to protect a carboxylic acid group, such as the C-terminus of an amino acid or peptide or an acidic or hydroxyl azepine ring substituent, against undesirable reactions during synthetic procedures and includes. Examples for protecting groups for carboxyl groups involve, for example, benzyl ester, cyclohexyl ester, 4-nitrobenzyl ester, t-butyl ester, 4-pyridylmethyl ester, and the like.

The term “amino-blocking group” is art-recognized and refers to a group which will prevent an amino group from participating in a reaction carried out on some other functional group, but which can be removed from the amine when desired. Such groups are discussed by in Ch. 7 of Greene and Wuts, cited above, and by Barton, Protective Groups in Organic Chemistry ch. 2 (McOmie, ed., Plenum Press, New York, 1973). Examples of suitable groups include acyl protecting groups such as, to illustrate, formyl, dansyl, acetyl, benzoyl, trifluoroacetyl, succinyl, methoxysuccinyl, benzyl and substituted benzyl such as 3,4-dimethoxybenzyl, o-nitrobenzyl, and triphenylmethyl; those of the formula —COOR where R includes such groups as methyl, ethyl, propyl, isopropyl, 2,2,2-trichloroethyl, 1-methyl-1-phenylethyl, isobutyl, t-butyl, t-amyl, vinyl, allyl, phenyl, benzyl, p-nitrobenzyl, o-nitrobenzyl, and 2,4-dichlorobenzyl; acyl groups and substituted acyl such as formyl, acetyl, chloroacetyl, dichloroacetyl, trichloroacetyl, trifluoroacetyl, benzoyl, and p-methoxybenzoyl; and other groups such as methanesulfonyl, p-toluenesulfonyl, p-bromobenzenesulfonyl, p-nitrophenylethyl, and p-toluenesulfonyl-aminocarbonyl. Preferred amino-blocking groups are benzyl (—CH₂C₆H₅), acyl [C(O)R1] or SiR1₃ where R1 is C₁-C₄ alkyl, halomethyl, or 2-halo-substituted-(C₂-C₄ alkoxy), aromatic urethane protecting groups as, for example, carbonylbenzyloxy (Cbz); and aliphatic urethane protecting groups such as t-butyloxycarbonyl (Boc) or 9-fluorenylmethoxycarbonyl (FMOC).

The definition of each expression, e.g. lower alkyl, m, n, p and the like, when it occurs more than once in any structure, is intended to be independent of its definition elsewhere in the same structure.

The term “electron-withdrawing group” is art-recognized, and refers to the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, March, Advanced Organic Chemistry 251-59 (McGraw Hill Book Company: New York, 1977). The Hammett constant values are generally negative for electron donating groups (σ(P)=−0.66 for NH₂) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

The term “small molecule” is art-recognized and refers to a composition which has a molecular weight of less than about 2000 amu, or less than about 1000 amu, and even less than about 500 amu. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays described herein. The term “small organic molecule” refers to a small molecule that is often identified as being an organic or medicinal compound, and does not include molecules that are exclusively nucleic acids, peptides or polypeptides.

The term “modulation” is art-recognized and refers to up regulation (i.e., activation or stimulation), down regulation (i.e., inhibition or suppression) of a response, or the two in combination or apart.

The term “treating” is art-recognized and refers to curing as well as ameliorating at least one symptom of any condition or disease or preventing a condition or disease from worsening.

The term “prophylactic” or “therapeutic” treatment is art-recognized and refers to administration of a drug to a host. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

A “patient,” “subject” or “host” to be treated by the subject method may mean either a human or non-human animal.

The term “mammal” is known in the art, and exemplary mammals include humans, primates, bovines, porcines, canines, felines, and rodents (e.g., mice and rats).

The term “bioavailable” when referring to a compound is art-recognized and refers to a form of a compound that allows for it, or a portion of the amount of compound administered, to be absorbed by, incorporated to, or otherwise physiologically available to a subject or patient to whom it is administered.

The term “pharmaceutically-acceptable salts” is art-recognized and refers to the relatively non-toxic, inorganic and organic acid addition salts of compounds, including, for example, those contained in compositions described herein.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “systemic administration,” “administered systemically,” “peripheral administration” and “administered peripherally” are art-recognized and refer to the administration of a subject composition, therapeutic or other material other than directly into the central nervous system, such that it enters the patient's system and, thus, is subject to metabolism and other like processes.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection and infusion.

Exemplary Methods and Compositions

Provided herein are methods and compounds for activating a sirtuin deacetylase protein family member (referred to as a “sirtuin protein”). The methods may comprise contacting the sirtuin deacetylase protein family member with a compound, such as a polyphenol, e.g. a plant polyphenol, and referred to herein as “activation compound” or “activating compound.” Exemplary sirtuin deacetylase proteins include the yeast silent information regulator 2 (Sir2) and human SIRT1. Other family members include proteins having a significant amino acid sequence homology and biological activity, e.g., the ability to deacetylate target proteins, such as histones and p53, to those of Sir2 and SIRT1.

Exemplary activating compounds are those selected from the group consisting of flavones, stilbenes, flavanones, isoflavanones, catechins, chalcones, tannins and anthocyanidins. Exemplary stilbenes include hydroxystilbenes, such as trihydroxystilbenes, e.g., 3,5,4′-trihydroxystilbene (“resveratrol”). Resveratrol is also known as 3,4′,5-stilbenetriol. Tetrahydroxystilbenes, e.g., piceatannol, are also encompassed. Hydroxychalones including trihydroxychalones, such as isoliquiritigenin, and tetrahydroxychalones, such as butein, can also be used. Hydroxyflavones including tetrahydroxyflavones, such as fisetin, and pentahydroxyflavones, such as quercetin, can also be used. Exemplary compounds are set forth in Tables 1-13 and 21 (compounds for which the ratio to control rate is >1). The compounds of Tables 1-8 may be obtained from Biomol, Sigma/Aldrich or Indofine.

In one embodiment, methods for activating a sirtuin protein comprise using an activating compound that is a stilbene or chalcone compound of formula 1: 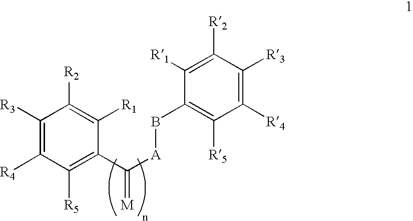
wherein, independently for each occurrence,
R₁, R₂, R₃, R₄, R₅, R′₁, R′₂, R′₃, R′₄, and R′₅ represent H, alkyl, aryl, heteroaryl, aralkyl, alkaryl, heteroaralkyl, halide, NO₂, SR, OR, N(R)₂, or carboxyl;
R represents H, alkyl, aryl, heteroaryl, aralkyl, —SO₃H, monosaccharide, oligosaccharide, glycofuranosyl, glycopyranosyl, glucuronosyl, or glucuronide;
M represents O, NR, or S;
A-B represents a bivalent alkyl, alkenyl, alkynyl, amido, sulfonamido, diazo, ether, alkylamino, alkylsulfide, hydroxylamine, or hydrazine group; and
n is 0 or 1.

In a further embodiment, the methods comprise a compound of formula 1 and the attendant definitions, wherein n is 0. In a further embodiment, the methods comprise a compound of formula 1 and the attendant definitions, wherein n is 1. In a further embodiment, the methods comprise a compound of formula 1 and the attendant definitions, wherein A-B is ethenyl. In a further embodiment, the methods comprise a compound of formula 1 and the attendant definitions, wherein A-B is —CH₂CH(Me)CH(Me)CH₂—. In a further embodiment, the methods comprise a compound of formula 1 and the attendant definitions, wherein M is O. In a further embodiment, the methods comprises a compound of formula 1 and the attendant definitions, wherein R₁, R₂, R₃, R₄, R₅, R′₁, R′₂, R′₃, R′₄, and R′₅ are H. In a further embodiment, the methods comprise a compound of formula 1 and the attendant definitions, wherein R₂, R₄, and R′₃ are OH. In a further embodiment, the methods comprise a compound of formula 1 and the attendant definitions, wherein R₂, R₄, R′₂ and R′₃ are OH. In a further embodiment, the methods comprise a compound of formula 1 and the attendant definitions, wherein R₃, R₅, R′₂ and R′₃ are OH. In a further embodiment, the methods comprise a compound of formula 1 and the attendant definitions, wherein R₁, R₃, R₅, R′₂ and R′₃ are OH. In a further embodiment, the methods comprise a compound of formula 1 and the attendant definitions, wherein R₂ and R′₂ are OH; R₄ is O-β-D-glucoside; and R′₃ is OCH₃. In a further embodiment, the methods comprise a compound of formula 1 and the attendant definitions, wherein R₂ is OH; R₄ is O-β-D-glucoside; and R′₃ is OCH₃.

In a further embodiment, the methods comprise a compound of formula 1 and the attendant definitions, wherein n is 0; A-B is ethenyl; and R₁, R₂, R₃, R₄, R₅, R′₁, R′₂, R′₃, R′₄, and R′₅ are H (trans stilbene). In a further embodiment, the methods comprise a compound of formula 1 and the attendant definitions, wherein n is 1; A-B is ethenyl; M is O; and R₁, R₂, R₃, R₄, R₅, R′₁, R′₂, R′₃, R′₄, and R′₅ are H (chalcone). In a further embodiment, the methods comprise a compound of formula 1 and the attendant definitions, wherein n is 0; A-B is ethenyl; R₂, R₄, and R′₃ are OH; and R₁, R₃, R₅, R′₁, R′₂, R′₄, and R′₅ are H (resveratrol). In a further embodiment, the methods comprise a compound of formula 1 and the attendant definitions, wherein n is 0; A-B is ethenyl; R₂, R₄, R′₂ and R′₃ are OH; and R₁, R₃, R₅, R′₁, R′₄ and R′₅ are H (piceatannol). In a further embodiment, the methods comprise a compound of formula 1 and the attendant definitions, wherein n is 1; A-B is ethenyl; M is O; R₃, R₅, R′₂ and R′₃ are OH; and R₁, R₂, R₄, R′₁, R′₄, and R′₅ are H (butein). In a further embodiment, the methods comprise a compound of formula 1 and the attendant definitions, wherein n is 1; A-B is ethenyl; M is O; R₁, R₃, R₅, R′₂ and R′₃ are OH; and R₂, R₄, R′₁, R′₄, and R′₅ are H (3,4,2′,4′,6′-pentahydroxychalcone). In a further embodiment, the methods comprise a compound of formula 1 and the attendant definitions, wherein n is 0; A-B is ethenyl; R₂ and R′₂ are OH, R₄ is O-β-D-glucoside, R′₃ is OCH₃; and R₁, R₃, R₅, R′₁, R′₄, and R′₅ are H (rhapontin). In a further embodiment, the methods comprise a compound of formula 1 and the attendant definitions, wherein n is 0; A-B is ethenyl; R₂ is OH, R₄ is O-β-D-glucoside, R′₃ is OCH₃; and R₁, R₃, R₅, R′₁, R′₂, R′₄, and R′₅ are H (deoxyrhapontin). In a further embodiment, the methods comprise a compound of formula 1 and the attendant definitions, wherein n is 0; A-B is —CH₂CH(Me)CH(Me)CH₂—; R₂, R₃, R′₂, and R′₃ are OH; and R₁, R₄, R₅, R′₁, R′₄, and R′₅ are H (NDGA).

In another embodiment, methods for activating a sirtuin protein comprise using an activating compound that is a flavanone compound of formula 2: 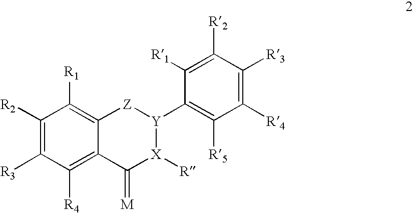
wherein, independently for each occurrence,
R₁, R₂, R₃, R₄, R′₁, R′₂, R′₃, R′₄, R′₅, and R″ represent H, alkyl, aryl, heteroaryl, aralkyl, alkaryl, heteroaralkyl, halide, NO₂, SR, OR, N(R)₂, or carboxyl;
R represents H, alkyl, aryl, heteroaryl, aralkyl, —SO₃H, monosaccharide, oligosaccharide, glycofuranosyl, glycopyranosyl, glucuronosyl, or glucuronide;
M represents H₂, O, NR, or S;
Z represents CR, O, NR, or S;
X represents CR or N; and
Y represents CR or N.

In a further embodiment, the methods comprise a compound of formula 2 and the attendant definitions, wherein X and Y are both CH. In a further embodiment, the methods comprise a compound of formula 2 and the attendant definitions, wherein M is O. In a further embodiment, the methods comprise a compound of formula 2 and the attendant definitions, wherein M is H₂. In a further embodiment, the methods comprise a compound of formula 2 and the attendant definitions, wherein Z is O. In a further embodiment, the methods comprise a compound of formula 2 and the attendant definitions, wherein R″ is H. In a further embodiment, the methods comprise a compound of formula 2 and the attendant definitions, wherein R″ is OH. In a further embodiment, the methods comprise a compound of formula 2 and the attendant definitions, wherein R″ is an alkoxycarbonyl. In a further embodiment, the methods comprise a compound of formula 2 and the attendant definitions, wherein R₁ is 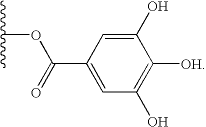
In a further embodiment, the methods comprise a compound of formula 2 and the attendant definitions, wherein R₁, R₂, R₃, R₄, R′₁, R′₂, R′₃, R′₄, R′₅ and R″ are H. In a further embodiment, the methods comprise a compound of formula 2 and the attendant definitions, wherein R₂, R₄, and R′₃ are OH. In a further embodiment, the methods comprise a compound of formula 2 and the attendant definitions, wherein R₄, R′₂, R′₃, and R″ are OH. In a further embodiment, the methods comprise a compound of formula 2 and the attendant definitions, wherein R₂, R₄, R′₂, R′₃, and R″ are OH. In a further embodiment, the methods comprise a compound of formula 2 and the attendant definitions, wherein R₂, R₄, R′₂, R′₃, R′₄, and R″ are OH.

In a further embodiment, the methods comprise a compound of formula 2 and the attendant definitions, wherein X and Y are CH; M is O; Z and O; R″ is H; and R₁, R₂, R₃, R₄, R′₁, R′₂, R′₃, R′₄, R′₅ and R″ are H (flavanone). In a further embodiment, the methods comprise a compound of formula 2 and the attendant definitions, wherein X and Y are CH; M is O; Z and O; R″ is H; R₂, R₄, and R′₃ are OH; and R₁, R₃, R′₁, R′₂, R′₄, and R′₅ are H (naringenin). In a further embodiment, the methods comprise a compound of formula 2 and the attendant definitions, wherein X and Y are CH; M is O; Z and O; R″ is OH; R₂, R₄, R′₂, and R′₃ are OH; and R₁, R₃, R′₁, R′₄, and R′₅ are H (3,5,7,3′,4′-pentahydroxyflavanone). In a further embodiment, the methods comprise a compound of formula 2 and the attendant definitions, wherein X and Y are CH; M is H₂; Z and O; R″ is OH; R₂, R₄, R′₂, and R′₃, are OH; and R₁, R₃, R′₁, R′₄ and R′₅ are H (epicatechin). In a further embodiment, the methods comprise a compound of formula 2 and the attendant definitions, wherein X and Y are CH; M is H₂; Z and O; R″ is OH; R₂, R₄, R′₂, R′₃, and R′₄ are OH; and R₁, R₃, R′₁, and R′₅ are H (gallocatechin). In a further embodiment, the methods comprise a compound of formula 2 and the attendant definitions, wherein X and Y are CH; M is H₂; Z and O; R″ is 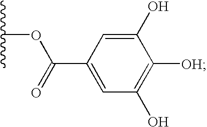
R₂, R₄, R′₂, R′₃, R′₄, and R″ are OH; and R₁, R₃, R′₁, and R′₅ are H (epigallocatechin gallate).

In another embodiment, methods for activating a sirtuin protein comprise using an activating compound that is an isoflavanone compound of formula 3: 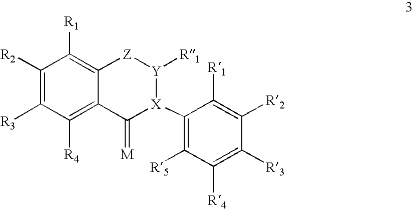
wherein, independently for each occurrence,
R₁, R₂, R₃, R₄, R′₁, R′₂, R′₃, R′₄, R′₅, and R″, represent H, alkyl, aryl, heteroaryl, aralkyl, alkaryl, heteroaralkyl, halide, NO₂, SR, OR, N(R)₂, or carboxyl;
R represents H, alkyl, aryl, heteroaryl, aralkyl, —SO₃H, monosaccharide, oligosaccharide, glycofuranosyl, glycopyranosyl, glucuronosyl, or glucuronide;
M represents H₂, O, NR, or S;
Z represents C(R)₂, O, NR, or S;
X represents CR or N; and
Y represents CR or N.

In another embodiment, methods for activating a sirtuin protein comprise using an activating compound that is a flavone compound of formula 4: 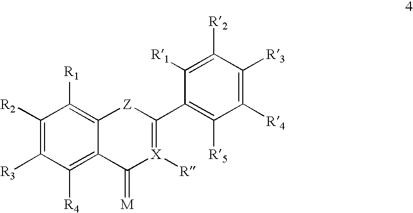
wherein, independently for each occurrence,
R₁, R₂, R₃, R₄, R′₁, R′₂, R′₃, R′₄, and R′₅, represent H, alkyl, aryl, heteroaryl, aralkyl, alkaryl, heteroaralkyl, halide, NO₂, SR, OR, N(R)₂, or carboxyl;
R represents H, alkyl, aryl, heteroaryl, aralkyl, —SO₃H, monosaccharide, oligosaccharide, glycofuranosyl, glycopyranosyl, glucuronosyl, or glucuronide;
M represents H₂, O, NR, or S;
Z represents CR, O, NR, or S; and
X represents CR″ or N, wherein
R″ is H, alkyl, aryl, heteroaryl, alkaryl, heteroaralkyl, halide, NO₂, SR, OR, N(R)₂, or carboxyl.

In a further embodiment, the methods comprise a compound of formula 4 and the attendant definitions, wherein X is C. In a further embodiment, the methods comprise a compound of formula 4 and the attendant definitions, wherein X is CR. In a further embodiment, the methods comprise a compound of formula 4 and the attendant definitions, wherein Z is O. In a further embodiment, the methods comprise a compound of formula 4 and the attendant definitions, wherein M is O. In a further embodiment, the methods comprise a compound of formula 4 and the attendant definitions, wherein R″ is H. In a further embodiment, the methods comprise a compound of formula 4 and the attendant definitions, wherein R″ is OH. In a further embodiment, the methods comprise a compound of formula 4 and the attendant definitions, wherein R₁, R₂, R₃, R₄, R′₁, R′₂, R′₃, R′₄, and R′₅ are H. In a further embodiment, the methods comprise a compound of formula 4 and the attendant definitions, wherein R₂, R′₂, and R′₃ are OH. In a further embodiment, the methods comprise a compound of formula 4 and the attendant definitions, wherein R₂, R₄, R′₂, R′₃, and R′₄ are OH. In a further embodiment, the methods comprise a compound of formula 4 and the attendant definitions, wherein R₂, R₄, R′₂, and R′₃ are OH. In a further embodiment, the methods comprise a compound of formula 4 and the attendant definitions, wherein R₃, R′₂, and R′₃ are OH. In a further embodiment, the methods comprise a compound of formula 4 and the attendant definitions, wherein R₂, R₄, R′₂, and R′₃ are OH. In a further embodiment, the methods comprise a compound of formula 4 and the attendant definitions, wherein R₂, R′₂, R′₃, and R′₄ are OH. In a further embodiment, the methods comprise a compound of formula 4 and the attendant definitions, wherein R₂, R₄, and R′₃ are OH. In a further embodiment, the methods comprise a compound of formula 4 and the attendant definitions, wherein R₂, R₃, R₄, and R′₃ are OH. In a further embodiment, the methods comprise a compound of formula 4 and the attendant definitions, wherein R₂, R₄, and R′₃ are OH. In a further embodiment, the methods comprise a compound of formula 4 and the attendant definitions, wherein R₃, R′₁, and R′₃ are OH. In a further embodiment, the methods comprise a compound of formula 4 and the attendant definitions, wherein R₂ and R′₃ are OH. In a further embodiment, the methods comprise a compound of formula 4 and the attendant definitions, wherein R₁, R₂, R′₂, and R′₃ are OH. In a further embodiment, the methods comprise a compound of formula 4 and the attendant definitions, wherein R₃, R′₁, and R′₂ are OH. In a further embodiment, the methods comprise a compound of formula 4 and the attendant definitions, wherein R′₃ is OH. In a further embodiment, the methods comprise a compound of formula 4 and the attendant definitions, wherein R₄ and R′₃ are OH. In a further embodiment, the methods comprise a compound of formula 4 and the attendant definitions, wherein R₂ and R₄ are OH. In a further embodiment, the methods comprise a compound of formula 4 and the attendant definitions, wherein R₂, R₄, R′₁, and R′₃ are OH. In a further embodiment, the methods comprise a compound of formula 4 and the attendant definitions, wherein R₄ is OH. In a further embodiment, the methods comprise a compound of formula 4 and the attendant definitions, wherein R₂, R₄, R′₂, R′₃, and R′₄ are OH. In a further embodiment, the methods comprise a compound of formula 4 and the attendant definitions, wherein R₂, R′₂, R′₃, and R′₄ are OH. In a further embodiment, the methods comprise a compound of formula 4 and the attendant definitions, wherein R₁, R₂, R₄, R′₂, and R′₃ are OH.

In a further embodiment, the methods comprise a compound of formula 4 and the attendant definitions, wherein X is CH; Z is O; M is O; and R₁, R₂, R₃, R₄, R′₁, R′₂, R′₃, R′₄, and R′₅ are H (flavone). In a further embodiment, the methods comprise a compound of formula 4 and the attendant definitions, wherein X is COH; Z is O; M is O; R₂, R′₂, and R′₃ are OH; and R₁, R₃, R₄, R′₁, R′₄, and R′₅ are H (fisetin). In a further embodiment, the methods comprise a compound of formula 4 and the attendant definitions, wherein X is CH; Z is O; M is O; R₂, R₄, R′₂, R′₃, and R′₄ are OH; and R₁, R₃, R′₁, and R′₅ are H (5,7,3′,4′,5′-pentahydroxyflavone). In a further embodiment, the methods comprise a compound of formula 4 and the attendant definitions, wherein X is CH; Z is O; M is O; R₂, R₄, R′₂, and R′₃ are OH; and R₁, R₃, R′₁, R′₄, and R′₅ are H (luteolin). In a further embodiment, the methods comprise a compound of formula 4 and the attendant definitions, wherein X is COH; Z is O; M is O; R₃, R′₂, and R′₃ are OH; and R₁, R₂, R₄, R′, R′₄, and R′₅ are H (3,6,3′,4′-tetrahydroxyflavone). In a further embodiment, the methods comprise a compound of formula 4 and the attendant definitions, wherein X is COH; Z is O; M is O; R₂, R₄, R′₂, and R′₃ are OH; and R₁, R₃, R′, R′₄, and R′₅ are H (quercetin). In a further embodiment, the methods comprise a compound of formula 4 and the attendant definitions, wherein X is CH; Z is O; M is O; R₂, R′₂, R′₃, and R′₄ are OH; and R₁, R₃, R₄, R′₁, and R′₅ are H. In a further embodiment, the methods comprise a compound of formula 4 and the attendant definitions, wherein X is COH; Z is O; M is O; R₂, R₄, and R′₃ are OH; and R₁, R₃, R′₁, R′₂, R′₄, and R′₅ are H. In a further embodiment, the methods comprise a compound of formula 4 and the attendant definitions, wherein X is CH; Z is O; M is O; R₂, R₃, R₄, and R′₃ are OH; and R₁, R′₁, R′₂, R′₄, and R′₅ are H. In a further embodiment, the methods comprise a compound of formula 4 and the attendant definitions, wherein X is CH; Z is O; M is 0; R₂, R₄, and R′₃ are OH; and R₁, R₃, R′₁, R′₂, R′₄, and R′₅ are H. In a further embodiment, the methods comprise a compound of formula 4 and the attendant definitions, wherein X is COH; Z is O; M is O; R₃, R′₁, and R′₃ are OH; and R₁, R₂, R₄, R′₂, R′₄, and R′₅ are H. In a further embodiment, the methods comprise a compound of formula 4 and the attendant definitions, wherein X is CH; Z is O; M is O; R₂ and R′₃ are OH; and R₁, R₃, R₄, R′, R′₂, R′₄, and R′₅ are H. In a further embodiment, the methods comprise a compound of formula 4 and the attendant definitions, wherein X is COH; Z is O; M is O; R₁, R₂, R′₂, and R′₃ are OH; and R₁, R₂, R₄, R′₃, R′₄, and R′₅ are H. In a further embodiment, the methods comprise a compound of formula 4 and the attendant definitions, wherein X is COH; Z is O; M is O; R₃, R′₁, and R′₂ are OH; and R₁, R₂, R₄; R′₃, R′₄, and R′₅ are H. In a further embodiment, the methods comprise a compound of formula 4 and the attendant definitions, wherein X is CH; Z is O; M is O; R′₃ is OH; and R₁, R₂, R₃, R₄, R′₁, R′₂, R′₄, and R′₅ are H. In a further embodiment, the methods comprise a compound of formula 4 and the attendant definitions, wherein X is CH; Z is O; M is O; R₄ and R′₃ are OH; and R₁, R₂, R₃, R′₁, R′₂, R′₄, and R′₅ are H. In a further embodiment, the methods comprise a compound of formula 4 and the attendant definitions, wherein X is CH; Z is O; M is 0; R₂ and R₄ are OH; and R₁, R₃, R′₁, R′₂, R′₃, R′₄, and R′₅ are H. In a further embodiment, the methods comprise a compound of formula 4 and the attendant definitions, wherein X is COH; Z is O; M is O; R₂, R₄, R′₁, and R′₃ are OH; and R₁, R₃, R′₂, R′₄, and R′₅ are H. In a further embodiment, the methods comprise a compound of formula 4 and the attendant definitions, wherein X is CH; Z is O; M is O; R₄ is OH; and R, R₂, R₃, R′₁, R′₂, R′₃, R′₄, and R′₅ are H. In a further embodiment, the methods comprise a compound of formula 4 and the attendant definitions, wherein X is COH; Z is O; M is O; R₂, R₄, R′₂, R′₃, and R′₄ are OH; and R₁, R₃, R′, and R′₅ are H. In a further embodiment, the methods comprise a compound of formula 4 and the attendant definitions, wherein X is COH; Z is O; M is O; R₂, R′₂, R′₃, and R′₄ are OH; and R₁, R₃, R₄, R′₁, and R′5 are H. In a further embodiment, the methods comprise a compound of formula 4 and the attendant definitions, wherein X is COH; Z is O; M is O; R₁, R₂, R₄, R′₂, and R′₃ are OH; and R₃, R′₁, R′₄, and R′₅ are H.

In another embodiment, methods for activating a sirtuin protein comprise using an activating compound that is an isoflavone compound of formula 5: 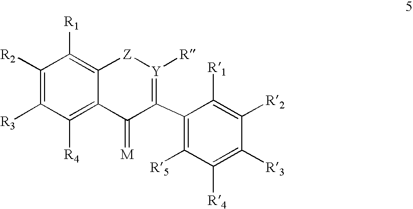
wherein, independently for each occurrence,
R₁, R₂, R₃, R₄, R′₁, R′₂, R′₃, R′₄, and R′₅, represent H, alkyl, aryl, heteroaryl, aralkyl, alkaryl, heteroaralkyl, halide, NO₂, SR, OR, N(R)₂, or carboxyl;
R represents H, alkyl, aryl, heteroaryl, aralkyl, —SO₃H, monosaccharide, oligosaccharide, glycofuranosyl, glycopyranosyl, glucuronosyl, or glucuronide;
M represents H₂, O, NR, or S;
Z represents C(R)₂, O, NR, or S; and
Y represents CR″ or N, wherein
R″ represents H, alkyl, aryl, heteroaryl, alkaryl, heteroaralkyl, halide, NO₂, SR, OR, N(R)₂, or carboxyl.

In a further embodiment, the methods comprise a compound of formula 5 and the attendant definitions, wherein Y is CR″. In a further embodiment, the methods comprise a compound of formula 5 and the attendant definitions, wherein Y is CH. In a further embodiment, the methods comprise a compound of formula 5 and the attendant definitions, wherein Z is O. In a further embodiment, the methods comprise a compound of formula 5 and the attendant definitions, wherein M is O. In a further embodiment, the methods comprise a compound of formula 5 and the attendant definitions, wherein R₂ and R′₃ are OH. In a further embodiment, the methods comprise a compound of formula 5 and the attendant definitions, wherein R₂, R₄, and R′₃ are OH.

In a further embodiment, the methods comprise a compound of formula 5 and the attendant definitions, wherein Y is CH; Z is O; M is O; R₂ and R′₃ are OH; and R₁, R₃, R₄, R′₁, R′₂, R′₄, and R′₅ are H. In a further embodiment, the methods comprise a compound of formula 5 and the attendant definitions, wherein Y is CH; Z is O; M is O; R₂, R₄, and R′₃ are OH; and R₁, R₃, R′₁, R′₂, R′₄, and R′₅ are H.

In another embodiment, methods for activating a sirtuin protein comprise using an activating compound that is an anthocyanidin compound of formula 6: 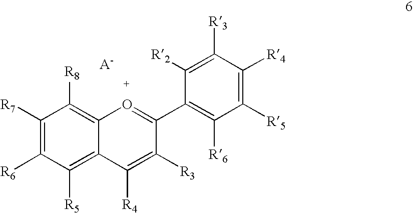
wherein, independently for each occurrence,
R₃, R₄, R₅, R₆, R₇, R₈, R′₂, R′₃, R′₄, R′₅, and R′₆ represent H, alkyl, aryl, heteroaryl, aralkyl, alkaryl, heteroaralkyl, halide, NO₂, SR, OR, N(R)₂, or carboxyl;
R represents H, alkyl, aryl, heteroaryl, aralkyl, —SO₃H, monosaccharide, oligosaccharide, glycofuranosyl, glycopyranosyl, glucuronosyl, or glucuronide; and
A⁻ represents an anion selected from the following: Cl⁻, Br⁻, or I⁻.

In a further embodiment, the methods comprise a compound of formula 6 and the attendant definitions, wherein A⁻ is Cl⁻. In a further embodiment, the methods comprise a compound of formula 6 and the attendant definitions, wherein R₃, R₅, R₇, and R′₄ are OH. In a further embodiment, the methods comprise a compound of formula 6 and the attendant definitions, wherein R₃, R₅, R₇, R′₃, and R′₄ are OH. In a further embodiment, the methods comprise a compound of formula 6 and the attendant definitions, wherein R₃, R₅, R₇, R′₃, R′₄, and R′₅ are OH.

In a further embodiment, the methods comprise a compound of formula 6 and the attendant definitions, wherein A⁻ is Cl⁻; R₃, R₅, R₇, and R′₄ are OH; and R₄, R₆, R₈, R′₂, R′₃, R′₅, and R′₆ are H. In a further embodiment, the methods comprise a compound of formula 6 and the attendant definitions, wherein A⁻ is Cl⁻; R₃, R₅, R₇, R′₃, and R′₄ are OH; and R₄, R₆, R₈, R′₂, R′₅, and R′₆ are H. In a further embodiment, the methods comprise a compound of formula 6 and the attendant definitions, wherein A⁻ is Cl⁻; R₃, R₅, R₇, R′₃, R′₄, and R′₅ are OH; and R₄, R₆, R₈, R′₂, and R′₆ are H.

Methods for activating a sirtuin protein may also comprise using a stilbene, chalcone, or flavone compound represented by formula 7: 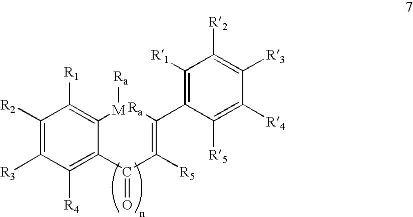
wherein, independently for each occurrence:
M is absent or O;
R₁, R₂, R₃, R₄, R₅, R′₁, R′₂, R′₃, R′₄, and R′₅ represent H, alkyl, aryl, heteroaryl, aralkyl, alkaryl, heteroaralkyl, halide, NO₂, SR, OR, N(R)₂, or carboxyl;
Rₐ represents H or the two instances of Rₐ form a bond;
R represents H, alkyl, aryl, heteroaryl, aralkyl, —SO₃H, monosaccharide, oligosaccharide, glycofuranosyl, glycopyranosyl, glucuronosyl, or glucuronide; and
n is 0 or 1.

In a further embodiment, the methods comprise an activating compound represented by formula 7 and the attendant definitions, wherein n is 0. In a further embodiment, the methods comprise an activating compound represented by formula 7 and the attendant definitions, wherein n is 1. In a further embodiment, the methods comprise an activating compound represented by formula 7 and the attendant definitions, wherein M is absent. In a further embodiment, the methods comprise an activating compound represented by formula 7 and the attendant definitions, wherein M is O. In a further embodiment, the methods comprise an activating compound represented by formula 7 and the attendant definitions, wherein Rₐ is H. In a further embodiment, the methods comprise an activating compound represented by formula 7 and the attendant definitions, wherein M is O and the two Rₐ form a bond.

In a further embodiment, the methods comprise an activating compound represented by formula 7 and the attendant definitions, wherein R₅ is H. In a further embodiment, the methods comprise an activating compound represented by formula 7 and the attendant definitions, wherein R₅ is OH. In a further embodiment, the methods comprise an activating compound represented by formula 7 and the attendant definitions, wherein R₁, R₃, and R′₃ are OH. In a further embodiment, the methods comprise an activating compound represented by formula 7 and the attendant definitions, wherein R₂, R₄, R′₂, and R′₃ are OH. In a further embodiment, the methods comprise an activating compound represented by formula 7 and the attendant definitions, wherein R₂, R′₂, and R′₃ are OH. In a further embodiment, the methods comprise an activating compound represented by formula 7 and the attendant definitions, wherein R₂ and R₄ are OH.

In a further embodiment, the methods include contacting a cell with an activating compound represented by formula 7 and the attendant definitions, wherein n is 0; M is absent; Rₐ is H; R₅ is H; R₁, R₃, and R′₃ are OH; and R₂, R₄, R′₁, R′₂, R′₄, and R′₅ are H. In a further embodiment, the methods comprise an activating compound represented by formula 7 and the attendant definitions, wherein n is 1; M is absent; Rₐ is H; R₅ is H; R₂, R₄, R′₂, and R′₃ are OH; and R₁, R₃, R′₁, R′₄, and R′₅ are H. In a further embodiment, the methods comprise an activating compound represented by formula 7 and the attendant definitions, wherein n is 1; M is O; the two Rₐ form a bond; R₅ is OH; R₂, R′₂, and R′₃ are OH; and R₁, R₃, R₄, R′, R′₄, and R′₅ are H.

Other compounds for activating sirtuin deacetylase protein family members include compounds having a formula selected from the group consisting of formulas 8-25 and 30 set forth below: 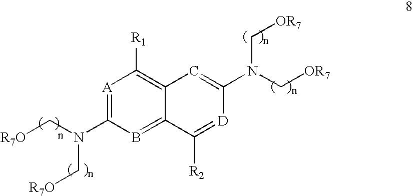
wherein, independently for each occurrence:
R₁ and R₂ represent H, aryl, heterocycle, or small alkyl;
R₇ represents H, alkyl, aryl, heteroaryl, aralkyl, —SO₃H, monosaccharide, oligosaccharide, glycofuranosyl, glycopyranosyl, glucuronosyl, or glucuronide;
A, B, C, and D represent CR₁ or N; and
n is 0, 1, 2, or 3; 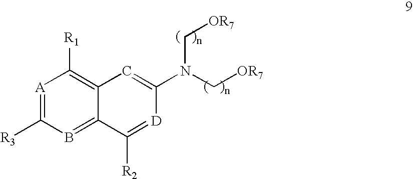
wherein, independently for each occurrence:
R₁ and R₂ represent H, aryl, heterocycle, or small alkyl;
R₃ represents small alkyl;
R₇ represents H, alkyl, aryl, heteroaryl, aralkyl, —SO₃H, monosaccharide, oligosaccharide, glycofuranosyl, glycopyranosyl, glucuronosyl, or glucuronide;
A, B, C, and D represent CR₁ or N; and
n is 0, 1, 2, or 3; 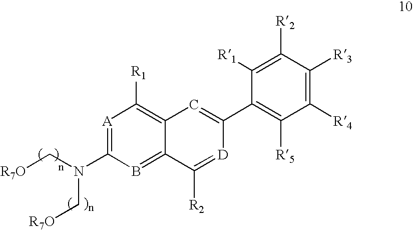
wherein, independently for each occurrence,
R₁ and R₂ represent H, aryl, heterocycle, or small alkyl;
R′₁, R′₂, R′₃, R′₄, and R′₅ represent H or OR₇;
R₇ represents H, alkyl, aryl, heteroaryl, aralkyl, —SO₃H, monosaccharide, oligosaccharide, glycofuranosyl, glycopyranosyl, glucuronosyl, or glucuronide;
A, B, C, and D represent CR₁ or N; and
n is 0, 1, 2, or 3; 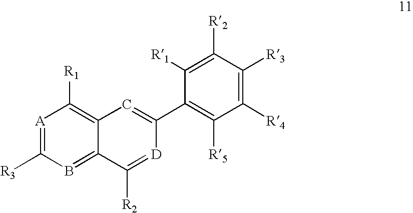
wherein, independently for each occurrence:
R₁ and R₂ represent H, aryl, heterocycle, or small alkyl;
R₃ represents small alkyl;
R′₁, R′₂, R′₃, R′₄, and R′₅ represent H or OR₇;
R₇ represents H, alkyl, aryl, heteroaryl, aralkyl, —SO₃H, monosaccharide, oligosaccharide, glycofuranosyl, glycopyranosyl, glucuronosyl, or glucuronide;
A, B, C, and D represent CR₁ or N; and
n is 0, 1, 2, or 3; 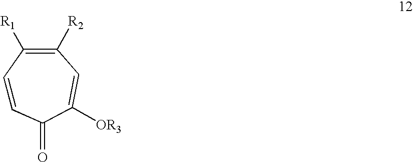
wherein, independently for each occurrence:
R₁ and R₂ represent H, aryl, or alkenyl; and
R₇ represents H, —SO₃H, monosaccharide, oligosaccharide, glycofuranosyl, glycopyranosyl, glucuronosyl, or glucuronide; 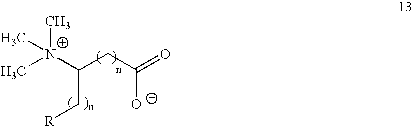
wherein, independently for each occurrence:
R represents heterocycle or aryl; and
n is 0 to 10 inclusive; 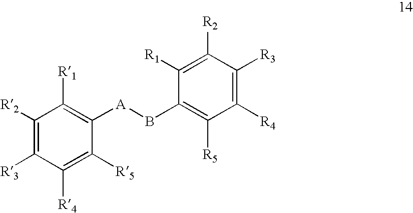
wherein, independently for each occurrence:
R₁, R₂, R₃, R₄, R₅, R′₁, R′₂, R′₃, R′₄, and R′₅ represents H, halogen, NO₂, SH, SR, OH, OR, NRR′, alkyl, aryl or carboxy;
R represents H, alkyl, aryl, heteroaryl, aralkyl, —SO₃H, monosaccharide, oligosaccharide, glycofuranosyl, glycopyranosyl, glucuronosyl, or glucuronide;
R′ represents H, alkyl, aryl, heteroaryl, aralkyl, —SO₃H, monosaccharide, oligosaccharide, glycofuranosyl, glycopyranosyl, glucuronosyl, or glucuronide; and
A-B represents ethene, ethyne, amide, sulfonamide, diazo, alkyl, ether, alkyl amine, alkyl sulfide, hydroxyamine, or hydrazine; 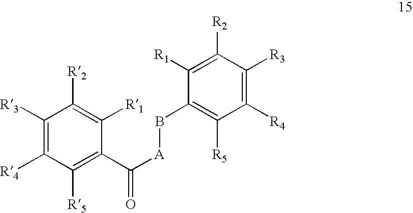
wherein, independently for each occurrence:
R₁, R₂, R₃, R₄, R₅, R′₁, R′₂, R′₃, R′₄, and R′₅ represents H, halogen, NO₂, SH, SR, OH, OR, NRR′, alkyl, aryl or carboxy;
R represents H, alkyl, aryl, heteroaryl, aralkyl, —SO₃H, monosaccharide, oligosaccharide, glycofuranosyl, glycopyranosyl, glucuronosyl, or glucuronide;
R′ represents H, alkyl, aryl, heteroaryl, aralkyl, —SO₃H, monosaccharide, oligosaccharide, glycofuranosyl, glycopyranosyl, glucuronosyl, or glucuronide; and
A-B represents ethene, ethyne, amide, sulfonamide, diazo, alkyl, ether, alkyl amine, alkyl sulfide, hydroxyamine, or hydrazine; 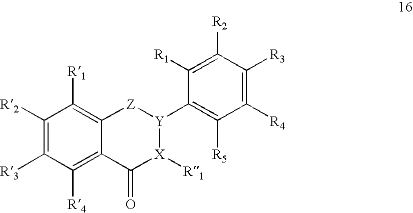
wherein, independently for each occurrence:
R₁, R₂, R₃, R₄, R₅, R′₁, R′₂, R′₃, R′₄, and R′₅ represents H, halogen, NO₂, SH, SR, OH, OR, NRR′, alkyl, aryl or carboxy;
R represents H, alkyl, aryl, heteroaryl, aralkyl, —SO₃H, monosaccharide, oligosaccharide, glycofuranosyl, glycopyranosyl, glucuronosyl, or glucuronide;
R′ represents H, alkyl, aryl, heteroaryl, aralkyl, —SO₃H, monosaccharide, oligosaccharide, glycofuranosyl, glycopyranosyl, glucuronosyl, or glucuronide;
X represents CR₈ or N;
Y represents CR₈ or N;
Z represents O, S, C(R₈)₂, or NR₈; and
R₈ represents alkyl, aryl or aralkyl; 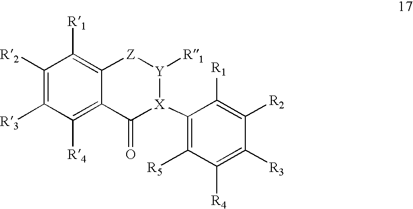
wherein, independently for each occurrence:
R₁, R₂, R₃, R₄, R₅, R′₁, R′₂, R′₃, R′₄, and R′₅ represents H, halogen, NO₂, SH, SR, OH, OR, NRR′, alkyl, aryl or carboxy;
R represents H, alkyl, aryl, heteroaryl, aralkyl, —SO₃H, monosaccharide, oligosaccharide, glycofuranosyl, glycopyranosyl, glucuronosyl, or glucuronide;
R′ represents H, alkyl, aryl, heteroaryl, aralkyl, —SO₃H, monosaccharide, oligosaccharide, glycofuranosyl, glycopyranosyl, glucuronosyl, or glucuronide;
X represents CR₈ or N;
Y represents CR₈ or N;
Z represents O, S, C(R₈)₂, or NR₈; and
R₈ represents alkyl, aryl or aralkyl; 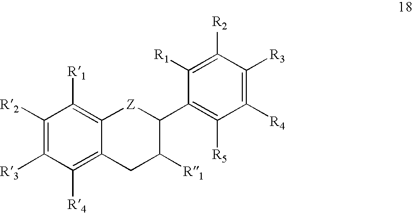
wherein, independently for each occurrence:
R₁, R₂, R₃, R₄, R₅, R′₁, R′₂, R′₃, R′₄, and R′₅ represents H, halogen, NO₂, SH, SR, OH, OR, NRR′, alkyl, aryl or carboxy;
R represents H, alkyl, aryl, heteroaryl, aralkyl, —SO₃H, monosaccharide, oligosaccharide, glycofuranosyl, glycopyranosyl, glucuronosyl, or glucuronide;
R′ represents H, alkyl, aryl, heteroaryl, aralkyl, —SO₃H, monosaccharide, oligosaccharide, glycofuranosyl, glycopyranosyl, glucuronosyl, or glucuronide;
Z represents O, S, C(R₈)₂, or NR₈; and
R₈ represents alkyl, aryl or aralkyl; 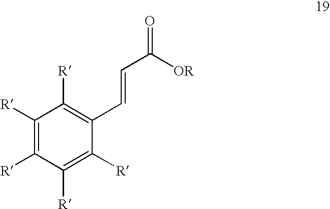
wherein, independently for each occurrence:
R is H, alkyl, aryl, heterocycyl, heteroaryl, aralkyl, —SO₃H, monosaccharide, oligosaccharide, glycofuranosyl, glycopyranosyl, glucuronosyl, or glucuronide; and
R′ is H, halogen, NO₂, SR, OR, NR₂, alkyl, aryl, or carboxy; 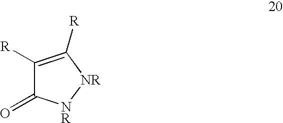
wherein, independently for each occurrence:
R is H, alkyl, aryl, heterocycyl, heteroaryl, aralkyl, —SO₃H, monosaccharide, oligosaccharide, glycofuranosyl, glycopyranosyl, glucuronosyl, or glucuronide; 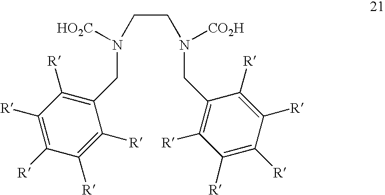
wherein, independently for each occurrence:
R′ is H, halogen, NO₂, SR, OR, NR₂, alkyl, aryl, aralkyl, or carboxy; and
R is H, alkyl, aryl, heterocycyl, heteroaryl, aralkyl, —SO₃H, monosaccharide, oligosaccharide, glycofuranosyl, glycopyranosyl, glucuronosyl, or glucuronide; 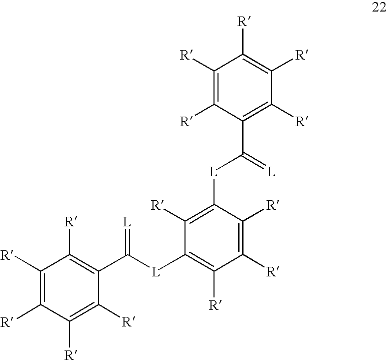
wherein, independently for each occurrence:
L represents CR₂, O, NR, or S;
R represents H, alkyl, aryl, aralkyl, heteroaralkyl, —SO₃H, monosaccharide, oligosaccharide, glycofuranosyl, glycopyranosyl, glucuronosyl, or glucuronide; and
R′ represents H, halogen, NO₂, SR, OR, NR₂, alkyl, aryl, aralkyl, or carboxy; 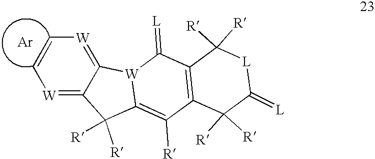
wherein, independently for each occurrence:
L represents CR₂, O, NR, or S;
W represents CR or N;
R represents H, alkyl, aryl, aralkyl, heteroaralkyl, —SO₃H, monosaccharide, oligosaccharide, glycofuranosyl, glycopyranosyl, glucuronosyl, or glucuronide; and
Ar represents a fused aryl or heteroaryl ring; and
R′ represents H, halogen, NO₂, SR, OR, NR₂, alkyl, aryl, aralkyl, or carboxy; 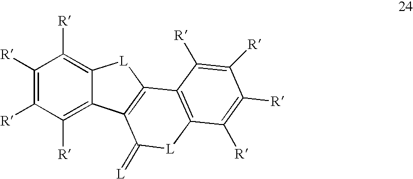
wherein, independently for each occurrence:
L represents CR₂, O, NR, or S;
R represents H, alkyl, aryl, aralkyl, heteroaralkyl, —SO₃H, monosaccharide, oligosaccharide, glycofuranosyl, glycopyranosyl, glucuronosyl, or glucuronide; and
R′ represents H, halogen, NO₂, SR, OR, NR₂, alkyl, aryl, aralkyl, or carboxy; 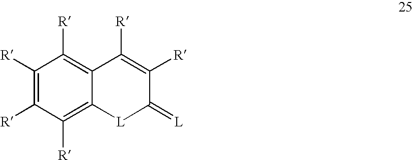
wherein, independently for each occurrence:
L represents CR₂, O, NR, or S;
R represents H, alkyl, aryl, aralkyl, heteroaralkyl, —SO₃H, monosaccharide, oligosaccharide, glycofuranosyl, glycopyranosyl, glucuronosyl, or glucuronide; and
R′ represents H, halogen, NO₂, SR, OR, NR₂, alkyl, aryl, aralkyl, or carboxy.

Methods for activating a sirtuin protein may also comprise using a stilbene, chalcone, or flavone compound represented by formula 30: 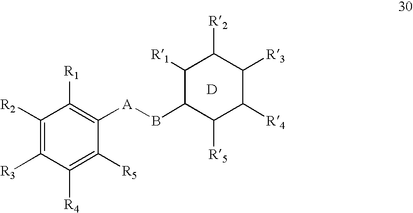
wherein, independently for each occurrence:
D is a phenyl or cyclohexyl group;
R₁, R₂, R₃, R₄, R₅, R′₁, R′₂, R′₃, R′₄, and R′₅ represent H, alkyl, aryl, heteroaryl, alkaryl, heteroaralkyl, halide, NO₂, SR, OR, N(R)₂, carboxyl, azide, ether; or any two adjacent R₁, R₂, R₃, R₄, R₅, R′₁, R′₂, R′₃, R′₄, or R′₅ groups taken together form a fused benzene or cyclohexyl group;
R represents H, alkyl, aryl, aralkyl, —SO₃H, monosaccharide, oligosaccharide, glycofuranosyl, glycopyranosyl, glucuronosyl, or glucuronide; and
A-B represents an ethylene, ethenylene, or imine group;
provided that when A-B is ethenylene, D is phenyl, and R′₃ is H: R₃ is not OH when R₁, R₂, R₄, and R₅ are H; and R₂ and R₄ are not OMe when R₁, R₃, and R₅ are H; and R₃ is not OMe when R₁, R₂, R₄, and R₅ are H.

In a further embodiment, the methods include contacting a cell with an activating compound represented by formula 30 and the attendant definitions, wherein D is a phenyl group.

In a further embodiment, the methods include contacting a cell with an activating compound represented by formula 30 and the attendant definitions, wherein A-B is an ethenylene or imine group.

In a further embodiment, the methods include contacting a cell with an activating compound represented by formula 30 and the attendant definitions, wherein A-B is an ethenylene group.

In a further embodiment, the methods include contacting a cell with an activating compound represented by formula 30 and the attendant definitions, wherein R₂ is OH.

In a further embodiment, the methods include contacting a cell with an activating compound represented by formula 30 and the attendant definitions, wherein R₄ is OH.

In a further embodiment, the methods include contacting a cell with an activating compound represented by formula 30 and the attendant definitions, wherein R₂ and R₄ are OH.

In a further embodiment, the methods include contacting a cell with an activating compound represented by formula 30 and the attendant definitions, wherein D is a phenyl group; and A-B is an ethenylene group.

In a further embodiment, the methods include contacting a cell with an activating compound represented by formula 30 and the attendant definitions, wherein D is a phenyl group; A-B is an ethenylene group; and R₂ and R₄ are OH.

In a further embodiment, the methods include contacting a cell with an activating compound represented by formula 30 and the attendant definitions, wherein A-B is ethenylene; D is a phenyl ring; R₂ and R₄ are OH; and R′₃ is Cl.

In a further embodiment, the methods include contacting a cell with an activating compound represented by formula 30 and the attendant definitions, wherein A-B is ethenylene; D is a phenyl ring; R₂ and R₄ are OH; and R′₃ is OH.

In a further embodiment, the methods include contacting a cell with an activating compound represented by formula 30 and the attendant definitions, wherein A-B is ethenylene; D is a phenyl ring; R₂ and R₄ are OH; and R′₃ is H.

In a further embodiment, the methods include contacting a cell with an activating compound represented by formula 30 and the attendant definitions, wherein A-B is ethenylene; D is a phenyl ring; R₂ and R₄ are OH; and R′₃ is CH₂CH₃.

In a further embodiment, the methods include contacting a cell with an activating compound represented by formula 30 and the attendant definitions, wherein A-B is ethenylene; D is a phenyl ring; R₂ and R₄ are OH; and R′₃ is F.

In a further embodiment, the methods include contacting a cell with an activating compound represented by formula 30 and the attendant definitions, wherein A-B is ethenylene; D is a phenyl ring; R₂ and R₄ are OH; and R′₃ is Me.

In a further embodiment, the methods include contacting a cell with an activating compound represented by formula 30 and the attendant definitions, wherein A-B is ethenylene; D is a phenyl ring; R₂ and R₄ are OH; and R′₃ is an azide.

In a further embodiment, the methods include contacting a cell with an activating compound represented by formula 30 and the attendant definitions, wherein A-B is ethenylene; D is a phenyl ring; R₂ and R₄ are OH; and R′₃ is SMe.

In a further embodiment, the methods include contacting a cell with an activating compound represented by formula 30 and the attendant definitions, wherein A-B is ethenylene; D is a phenyl ring; R₂ and R₄ are OH; and R′₃ is NO₂.

In a further embodiment, the methods include contacting a cell with an activating compound represented by formula 30 and the attendant definitions, wherein A-B is ethenylene; D is a phenyl ring; R₂ and R₄ are OH; and R′₃ is CH(CH₃)₂.

In a further embodiment, the methods include contacting a cell with an activating compound represented by formula 30 and the attendant definitions, wherein A-B is ethenylene; D is a phenyl ring; R₂ and R₄ are OH; and R′₃ is OMe.

In a further embodiment, the methods include contacting a cell with an activating compound represented by formula 30 and the attendant definitions, wherein A-B is ethenylene; D is a phenyl ring; R₂ and R₄ are OH; R′₂ is OH; and R′₃ is OMe.

In a further embodiment, the methods include contacting a cell with an activating compound represented by formula 30 and the attendant definitions, wherein A-B is ethenylene; D is a phenyl ring; R₂ is OH; R₄ is carboxyl; and R′₃ is OH.

In a further embodiment, the methods include contacting a cell with an activating compound represented by formula 30 and the attendant definitions, wherein A-B is ethenylene; D is a phenyl ring; R₂ and R₄ are OH; and R′₃ is carboxyl.

In a further embodiment, the methods include contacting a cell with an activating compound represented by formula 30 and the attendant definitions, wherein A-B is ethenylene; D is a phenyl ring; R₂ and R₄ are OH; and R′₃ and R′₄ taken together form a fused benzene ring.

In a further embodiment, the methods include contacting a cell with an activating compound represented by formula 30 and the attendant definitions, wherein A-B is ethenylene; D is a phenyl ring; and R₄ is OH.

In a further embodiment, the methods include contacting a cell with an activating compound represented by formula 30 and the attendant definitions, wherein A-B is ethenylene; D is a phenyl ring; R₂ and R₄ are OCH₂OCH₃; and R′₃ is SMe.

In a further embodiment, the methods include contacting a cell with an activating compound represented by formula 30 and the attendant definitions, wherein A-B is ethenylene; D is a phenyl ring; R₂ and R₄ are OH; and R′₃ is carboxyl.

In a further embodiment, the methods include contacting a cell with an activating compound represented by formula 30 and the attendant definitions, wherein A-B is ethenylene; D is a cyclohexyl ring; and R₂ and R₄ are OH.

In a further embodiment, the methods include contacting a cell with an activating compound represented by formula 30 and the attendant definitions, wherein A-B is ethenylene; D is a phenyl ring; and R₃ and R₄ are OMe.

In a further embodiment, the methods include contacting a cell with an activating compound represented by formula 30 and the attendant definitions, wherein A-B is ethenylene; D is a phenyl ring; R₂ and R₄ are OH; and R′₃ is OH.

In another embodiment, methods for activating a sirtuin protein comprise using an activating compound of formula 32: 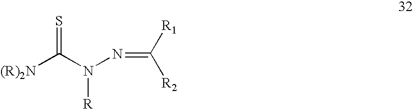
wherein, independently for each occurrence,
R is H, or a substituted or unsubstituted alkyl, aryl, aralkyl, heterocyclyl, heterocyclylalkyl, heteroaryl, or heteroaralkyl; and
R₁ and R₂ are a substituted or unsubstituted alkyl, aryl, aralkyl, heterocyclyl, heterocyclylalkyl, heteroaryl, or heteroaralkyl.

In a further embodiment, the methods comprise a compound of formula 32 and the attendant definitions wherein R is H.

In a further embodiment, the methods comprise a compound of formula 32 and the attendant definitions wherein R₁ is 3-hydroxyphenyl.

In a further embodiment, the methods comprise a compound of formula 32 and the attendant definitions wherein R₂ is methyl.

In a further embodiment, the methods comprise a compound of formula 32 and the attendant definitions wherein R is H and R₁ is 3-hydroxyphenyl.

In a further embodiment, the methods comprise a compound of formula 32 and the attendant definitions wherein R is H, R₁ is 3-hydroxyphenyl, and R₂ is methyl.

In another embodiment, methods for activating a sirtuin protein comprise using an activating compound of formula 33: 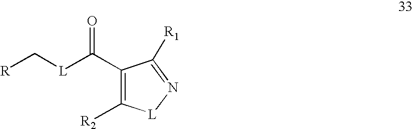
wherein, independently for each occurrence:
R is H, or a substituted or unsubstituted alkyl, alkenyl, or alkynyl;
R₁ and R₂ are a substituted or unsubstituted alkyl, aryl, aralkyl, heterocyclyl, heterocyclylalkyl, heteroaryl, or heteroaralkyl; and
L is O, S, or NR.

In a further embodiment, the methods comprise a compound of formula 33 and the attendant definitions wherein R is alkynyl.

In a further embodiment, the methods comprise a compound of formula 33 and the attendant definitions wherein R₁ is 2,6-dichlorophenyl.

In a further embodiment, the methods comprise a compound of formula 33 and the attendant definitions wherein R₂ is methyl.

In a further embodiment, the methods comprise a compound of formula 33 and the attendant definitions wherein L is O.

In a further embodiment, the methods comprise a compound of formula 33 and the attendant definitions wherein R is alkynyl and R₁ is 2,6-dichlorophenyl.

In a further embodiment, the methods comprise a compound of formula 33 and the attendant definitions wherein R is alkynyl, R₁ is 2,6-dichlorophenyl, and R₂ is methyl.

In a further embodiment, the methods comprise a compound of formula 33 and the attendant definitions wherein R is alkynyl, R₁ is 2,6-dichlorophenyl, R₂ is methyl, and L is O.

In another embodiment, methods for activating a sirtuin protein comprise using an activating compound of formula 34: 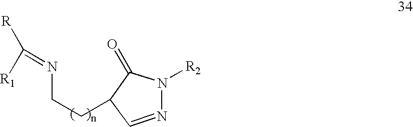
wherein, independently for each occurrence:
R, R₁, and R₂ are H, or a substituted or unsubstituted alkyl, aryl, aralkyl, heterocyclyl, heterocyclylalkyl, heteroaryl, or heteroaralkyl; and
n is an integer from 0 to 5 inclusive.

In a further embodiment, the methods comprise a compound of formula 34 and the attendant definitions wherein R is 3,5-dichloro-2-hydroxyphenyl.

In a further embodiment, the methods comprise a compound of formula 34 and the attendant definitions wherein R₁ is H.

In a further embodiment, the methods comprise a compound of formula 34 and the attendant definitions wherein R₂ is H.

In a further embodiment, the methods comprise a compound of formula 34 and the attendant definitions wherein n is 1.

In a further embodiment, the methods comprise a compound of formula 34 and the attendant definitions wherein R is 3,5-dichloro-2-hydroxyphenyl and R₁ is H.

In a further embodiment, the methods comprise a compound of formula 34 and the attendant definitions wherein R is 3,5-dichloro-2-hydroxyphenyl, R₁ is H, and R₂ is H.

In a further embodiment, the methods comprise a compound of formula 34 and the attendant definitions wherein R is 3,5-dichloro-2-hydroxyphenyl, R₁ is H, R₂ is H, and n is 1.

In another embodiment, methods for activating a sirtuin protein comprise using an activating compound of formula 35: 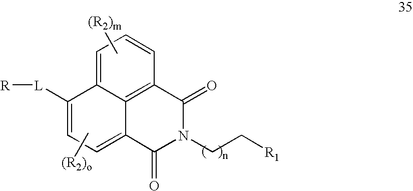
wherein, independently for each occurrence:
R is H or a substituted or unsubstituted alkyl, aryl, aralkyl, heterocyclyl, heterocyclylalkyl, heteroaryl, or heteroaralkyl;
R₁ is a substituted or unsubstituted alkyl, aryl, aralkyl, heterocyclyl, heterocyclylalkyl, heteroaryl, or heteroaralkyl;
R₂ is hydroxy, amino, cyano, halide, OR₃, ether, ester, amido, ketone, carboxylic acid, nitro, or a substituted or unsubstituted alkyl, aryl, aralkyl, heterocyclyl, heterocyclylalkyl, heteroaryl, heteroaralkyl;
R₃ is alkyl, —SO₃H, monosaccharide, oligosaccharide, glycofuranosyl, glycopyranosyl, glucuronosyl, or glucuronide;
L is O, NR, or S;
m is an integer from 0 to 3 inclusive;
n is an integer from 0 to 5 inclusive; and
o is an integer from 0 to 2 inclusive.

In a further embodiment, the methods comprise a compound of formula 35 and the attendant definitions wherein R is phenyl.

In a further embodiment, the methods comprise a compound of formula 35 and the attendant definitions wherein R₁ is pyridine.

In a further embodiment, the methods comprise a compound of formula 35 and the attendant definitions wherein L is S.

In a further embodiment, the methods comprise a compound of formula 35 and the attendant definitions wherein m is 0.

In a further embodiment, the methods comprise a compound of formula 35 and the attendant definitions wherein n is 1.

In a further embodiment, the methods comprise a compound of formula 35 and the attendant definitions wherein o is 0.

In a further embodiment, the methods comprise a compound of formula 35 and the attendant definitions wherein R is phenyl and R₁ is pyridine.

In a further embodiment, the methods comprise a compound of formula 35 and the attendant definitions wherein R is phenyl, R₁ is pyridine, and L is S.

In a further embodiment, the methods comprise a compound of formula 35 and the attendant definitions wherein R is phenyl, R₁ is pyridine, L is S, and m is 0.

In a further embodiment, the methods comprise a compound of formula 35 and the attendant definitions wherein R is phenyl, R₁ is pyridine, L is S, m is 0, and n is 1.

In a further embodiment, the methods comprise a compound of formula 35 and the attendant definitions wherein R is phenyl, R₁ is pyridine, L is S, m is 0, n is 1, and o is 0.

In another embodiment, methods for activating a sirtuin protein comprise using an activating compound of formula 36: 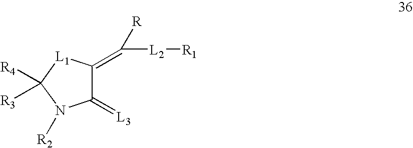
wherein, independently for each occurrence:
R, R₃, and R₄ are H, hydroxy, amino, cyano, halide, OR₅, ether, ester, amido, ketone, carboxylic acid, nitro, or a substituted or unsubstituted alkyl, aryl, aralkyl, heterocyclyl, heterocyclylalkyl, heteroaryl, heteroaralkyl;
R₅ is alkyl, —SO₃H, monosaccharide, oligosaccharide, glycofuranosyl, glycopyranosyl, glucuronosyl, or glucuronide;
R₁ and R₂ are H or a substituted or unsubstituted alkyl, aryl, aralkyl, heterocyclyl, heterocyclylalkyl, heteroaryl, heteroaralkyl;
L₁ is O, NR₁, S, C(R)₂, or SO₂; and
L₂ and L₃ are O, NR₁, S, or C(R)₂.

In a further embodiment, the methods comprise a compound of formula 36 and the attendant definitions wherein R is H.

In a further embodiment, the methods comprise a compound of formula 36 and the attendant definitions wherein R₁ is 4-chlorophenyl.

In a further embodiment, the methods comprise a compound of formula 36 and the attendant definitions wherein R₂ is 4-chlorophenyl.

In a further embodiment, the methods comprise a compound of formula 36 and the attendant definitions wherein R₃ is H.

In a further embodiment, the methods comprise a compound of formula 36 and the attendant definitions wherein R₄ is H.

In a further embodiment, the methods comprise a compound of formula 36 and the attendant definitions wherein L₁ is SO₂.

In a further embodiment, the methods comprise a compound of formula 36 and the attendant definitions wherein L₂ is NH.

In a further embodiment, the methods comprise a compound of formula 36 and the attendant definitions wherein L₃ is 0.

In a further embodiment, the methods comprise a compound of formula 36 and the attendant definitions wherein R is H and R₁ is 4-chlorophenyl.

In a further embodiment, the methods comprise a compound of formula 36 and the attendant definitions wherein R is H, R₁ is 4-chlorophenyl, and R₂ is 4-chlorophenyl.

In a further embodiment, the methods comprise a compound of formula 36 and the attendant definitions wherein R is H, R₁ is 4-chlorophenyl, R₂ is 4-chlorophenyl, and R₃ is H.

In a further embodiment, the methods comprise a compound of formula 36 and the attendant definitions wherein R is H, R₁ is 4-chlorophenyl, R₂ is 4-chlorophenyl, R₃ is H, and R₄ is H.

In a further embodiment, the methods comprise a compound of formula 36 and the attendant definitions wherein R is H, R₁ is 4-chlorophenyl, R₂ is 4-chlorophenyl, R₃ is H, R₄ is H, and L₁ is SO₂.

In a further embodiment, the methods comprise a compound of formula 36 and the attendant definitions wherein R is H, R₁ is 4-chlorophenyl, R₂ is 4-chlorophenyl, R₃ is H, R₄ is H, L₁ is SO₂, and L₂ is NH.

In a further embodiment, the methods comprise a compound of formula 36 and the attendant definitions wherein R is H, R₁ is 4-chlorophenyl, R₂ is 4-chlorophenyl, R₃ is H, R₄ is H, L₁ is SO₂, L₂ is NH, and L₃ is O.

In another embodiment, methods for activating a sirtuin protein comprise using an activating compound of formula 37: 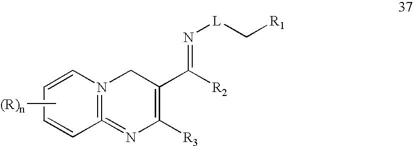
wherein, independently for each occurrence:
R is hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, heteroaralkyl;
R₁ is H or a substituted or unsubstituted alkyl, aryl, aralkyl, heterocyclyl, heterocyclylalkyl, heteroaryl, heteroaralkyl;
R₂ and R₃ are H or a substituted or unsubstituted alkyl, aryl, aralkyl, heterocyclyl, heterocyclylalkyl, heteroaryl, heteroaralkyl;
R₄ is alkyl, —SO₃H, monosaccharide, oligosaccharide, glycofuranosyl, glycopyranosyl, glucuronosyl, or glucuronide;
L is O, NR₁, or S; and
n is an integer from 0 to 4 inclusive.

In a further embodiment, the methods comprise a compound of formula 37 and the attendant definitions wherein R is methyl.

In a further embodiment, the methods comprise a compound of formula 37 and the attendant definitions wherein n is 1.

In a further embodiment, the methods comprise a compound of formula 37 and the attendant definitions wherein R₁ is 3-fluorophenyl.

In a further embodiment, the methods comprise a compound of formula 37 and the attendant definitions wherein R₂ is H.

In a further embodiment, the methods comprise a compound of formula 37 and the attendant definitions wherein R₃ is 4-chlorophenyl.

In a further embodiment, the methods comprise a compound of formula 37 and the attendant definitions wherein L is O.

In a further embodiment, the methods comprise a compound of formula 37 and the attendant definitions wherein R is methyl and n is 1.

In a further embodiment, the methods comprise a compound of formula 37 and the attendant definitions wherein R is methyl, n is 1, and R₁ is 3-fluorophenyl.

In a further embodiment, the methods comprise a compound of formula 37 and the attendant definitions wherein R is methyl, n is 1, R₁ is 3-fluorophenyl, and R₂ is H.

In a further embodiment, the methods comprise a compound of formula 37 and the attendant definitions wherein R is methyl, n is 1, R₁ is 3-fluorophenyl, R₂ is H, and R₃ is 4-chlorophenyl.

In another embodiment, methods for activating a sirtuin protein comprise using an activating compound of formula 38: 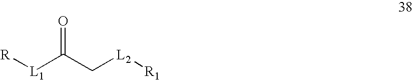
wherein, independently for each occurrence:
R and R₁ are H or a substituted or unsubstituted alkyl, aryl, aralkyl, heterocyclyl, heterocyclylalkyl, heteroaryl, or heteroaralkyl; and
L₁ and L₂ are O, NR, or S.

In a further embodiment, the methods comprise a compound of formula 38 and the attendant definitions wherein R is 3-methoxyphenyl.

In a further embodiment, the methods comprise a compound of formula 38 and the attendant definitions wherein R₁ is 4-t-butylphenyl.

In a further embodiment, the methods comprise a compound of formula 38 and the attendant definitions wherein L₁ is NH.

In a further embodiment, the methods comprise a compound of formula 38 and the attendant definitions wherein L₂ is O.

In a further embodiment, the methods comprise a compound of formula 38 and the attendant definitions wherein R is 3-methoxyphenyl and R₁ is 4-t-butylphenyl.

In a further embodiment, the methods comprise a compound of formula 38 and the attendant definitions wherein R is 3-methoxyphenyl, R₁ is 4-t-butylphenyl, and L₁ is NH.

In a further embodiment, the methods comprise a compound of formula 38 and the attendant definitions wherein R is 3-methoxyphenyl, R₁ is 4-t-butylphenyl, L₁ is NH, and L₂ is O.

In another embodiment, methods for activating a sirtuin protein comprise using an activating compound of formula 39: 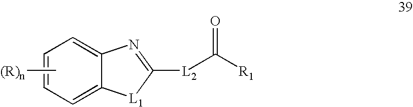
wherein, independently for each occurrence:
R is H, hydroxy, amino, cyano, halide, OR₂, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₁ is H or a substituted or unsubstituted alkyl, aryl, alkaryl, heterocyclyl, heterocyclylalkyl, heteroaryl, or heteroaralkyl;
R₂ is alkyl, —SO₃H, monosaccharide, oligosaccharide, glycofuranosyl, glycopyranosyl, glucuronosyl, or glucuronide;
L₁ and L₂ are O, NR, or S; and
n is an integer from 0 to 4 inclusive.

In a further embodiment, the methods comprise a compound of formula 39 and the attendant definitions wherein R is methyl.

In a further embodiment, the methods comprise a compound of formula 39 and the attendant definitions wherein n is 1.

In a further embodiment, the methods comprise a compound of formula 39 and the attendant definitions wherein R₁ is 3,4,5-trimethoxyphenyl.

In a further embodiment, the methods comprise a compound of formula 39 and the attendant definitions wherein L₁ is S.

In a further embodiment, the methods comprise a compound of formula 39 and the attendant definitions wherein L₂ is NH.

In a further embodiment, the methods comprise a compound of formula 39 and the attendant definitions wherein R is methyl and n is 1.

In a further embodiment, the methods comprise a compound of formula 39 and the attendant definitions wherein R is methyl, n is 1, and R₁ is 3,4,5-trimethoxyphenyl.

In a further embodiment, the methods comprise a compound of formula 39 and the attendant definitions wherein R is methyl, n is 1, R₁ is 3,4,5-trimethoxyphenyl, and L₁ is S.

In a further embodiment, the methods comprise a compound of formula 39 and the attendant definitions wherein R is methyl, n is 1, R₁ is 3,4,5-trimethoxyphenyl, L₁ is S, and L₂ is NH.

In another embodiment, methods for activating a sirtuin protein comprise using an activating compound of formula 40: 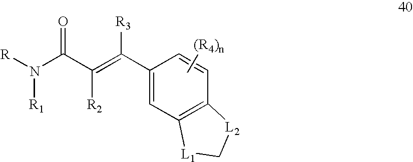
wherein, independently for each occurrence:
R, R₁, R₂, R₃ are H or a substituted or unsubstituted alkyl, aryl, alkaryl, heterocyclyl, heterocyclylalkyl, heteroaryl, or heteroaralkyl;
R₄ is hydroxy, amino, cyano, halide, OR₅, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₅ is alkyl, —SO₃H, monosaccharide, oligosaccharide, glycofuranosyl, glycopyranosyl, glucuronosyl, or glucuronide;
L₁ and L₂ are O, NR, or S; and
n is an integer from 0 to 3 inclusive.

In a further embodiment, the methods comprise a compound of formula 40 and the attendant definitions wherein R is H.

In a further embodiment, the methods comprise a compound of formula 40 and the attendant definitions wherein R₁ is perfluorophenyl.

In a further embodiment, the methods comprise a compound of formula 40 and the attendant definitions wherein R₂ is H.

In a further embodiment, the methods comprise a compound of formula 40 and the attendant definitions wherein R₃ is H.

In a further embodiment, the methods comprise a compound of formula 40 and the attendant definitions wherein L₁ is O.

In a further embodiment, the methods comprise a compound of formula 40 and the attendant definitions wherein L₂ is O.

In a further embodiment, the methods comprise a compound of formula 40 and the attendant definitions wherein n is 0.

In a further embodiment, the methods comprise a compound of formula 40 and the attendant definitions wherein R is H and R₁ is perfluorophenyl.

In a further embodiment, the methods comprise a compound of formula 40 and the attendant definitions wherein R is H, R₁ is perfluorophenyl, and R₂ is H.

In a further embodiment, the methods comprise a compound of formula 40 and the attendant definitions R is H, R₁ is perfluorophenyl, R₂ is H, and R₃ is H.

In a further embodiment, the methods comprise a compound of formula 40 and the attendant definitions wherein R is H, R₁ is perfluorophenyl, R₂ is H, R₃ is H, and L₁ is O.

In a further embodiment, the methods comprise a compound of formula 40 and the attendant definitions wherein R is H, R₁ is perfluorophenyl, R₂ is H, R₃ is H, L₁ is O, and L₂ is O.

In a further embodiment, the methods comprise a compound of formula 40 and the attendant definitions wherein R is H, R₁ is perfluorophenyl, R₂ is H, R₃ is H, L₁ is O, L₂ is O, and n is 0.

In another embodiment, methods for activating a sirtuin protein comprise using an activating compound of formula 41: 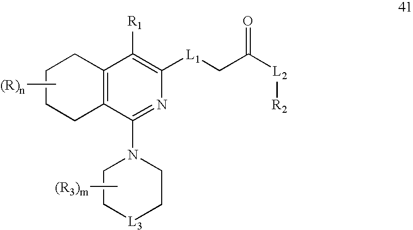
wherein, independently for each occurrence:
R, R₁, and R₃ are hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₂ is H or a substituted or unsubstituted alkyl, aryl, aralkyl, heterocyclyl, heterocyclylalkyl, heteroaryl, or heteroaralkyl;
R₄ is alkyl, —SO₃H, monosaccharide, oligosaccharide, glycofuranosyl, glycopyranosyl, glucuronosyl, or glucuronide;
L₁, L₂, and L₃ are O, NR₂, or S; and
m and n are integers from 0 to 8 inclusive.

In a further embodiment, the methods comprise a compound of formula 41 and the attendant definitions wherein n is 0.

In a further embodiment, the methods comprise a compound of formula 41 and the attendant definitions wherein R₁ is cyano.

In a further embodiment, the methods comprise a compound of formula 41 and the attendant definitions wherein R₂ is ethyl.

In a further embodiment, the methods comprise a compound of formula 41 and the attendant definitions wherein m is 0.

In a further embodiment, the methods comprise a compound of formula 41 and the attendant definitions wherein L₁ is S.

In a further embodiment, the methods comprise a compound of formula 41 and the attendant definitions wherein L₂ is O.

In a further embodiment, the methods comprise a compound of formula 41 and the attendant definitions wherein L₃ is O.

In a further embodiment, the methods comprise a compound of formula 41 and the attendant definitions wherein n is 0 and R₁ is cyano.

In a further embodiment, the methods comprise a compound of formula 41 and the attendant definitions wherein n is 0, R₁ is cyano, and R₂ is ethyl.

In a further embodiment, the methods comprise a compound of formula 41 and the attendant definitions wherein n is 0, R₁ is cyano, R₂ is ethyl, and m is 0.

In a further embodiment, the methods comprise a compound of formula 41 and the attendant definitions wherein n is 0, R₁ is cyano, R₂ is ethyl, m is 0, and L₁ is S.

In a further embodiment, the methods comprise a compound of formula 41 and the attendant definitions wherein n is 0, R₁ is cyano, R₂ is ethyl, m is 0, L₁ is S, and L₂ is O.

In a further embodiment, the methods comprise a compound of formula 41 and the attendant definitions wherein n is 0, R₁ is cyano, R₂ is ethyl, m is 0, L₁ is S, L₂ is O, and L₃ is O.

In another embodiment, methods for activating a sirtuin protein comprise using an activating compound of formula 42: 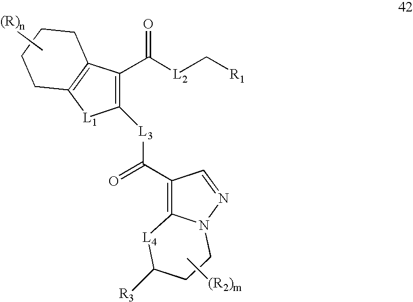
wherein, independently for each occurrence:
R and R₂ are H, hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₁ and R₃ are H or a substituted or unsubstituted alkyl, aryl, aralkyl, heterocyclyl, heterocyclylalkyl, heteroaryl, or heteroaralkyl;
R₄ is alkyl, —SO₃H, monosaccharide, oligosaccharide, glycofuranosyl, glycopyranosyl, glucuronosyl, or glucuronide;
L₁, L₂, L₃, and L₄ are O, NR₁, or S;
m is an integer from 0 to 6 inclusive; and
n is an integer from 0 to 8 inclusive.

In a further embodiment, the methods comprise a compound of formula 42 and the attendant definitions wherein n is 0.

In a further embodiment, the methods comprise a compound of formula 42 and the attendant definitions wherein R₁ is methyl.

In a further embodiment, the methods comprise a compound of formula 42 and the attendant definitions wherein R₂ is CF₃ and m is 1.

In a further embodiment, the methods comprise a compound of formula 42 and the attendant definitions wherein R₃ is 4-methylphenyl.

In a further embodiment, the methods comprise a compound of formula 42 and the attendant definitions wherein L₁ is S.

In a further embodiment, the methods comprise a compound of formula 42 and the attendant definitions wherein L₂ is O.

In a further embodiment, the methods comprise a compound of formula 42 and the attendant definitions wherein L₃ is NR₁.

In a further embodiment, the methods comprise a compound of formula 42 and the attendant definitions wherein L₄ is NR₁.

In a further embodiment, the methods comprise a compound of formula 42 and the attendant definitions wherein n is 0 and R₁ is methyl.

In a further embodiment, the methods comprise a compound of formula 42 and the attendant definitions wherein n is 0, R₁ is methyl, R₂ is CF₃, and m is 1.

In a further embodiment, the methods comprise a compound of formula 42 and the attendant definitions wherein n is 0, R₁ is methyl, R₂ is CF₃, m is 1; and R₃ is 4-methylphenyl.

In a further embodiment, the methods comprise a compound of formula 42 and the attendant definitions wherein n is 0, R₁ is methyl, R₂ is CF₃, m is 1; R₃ is 4-methylphenyl; and L₁ is S.

In a further embodiment, the methods comprise a compound of formula 42 and the attendant definitions wherein n is 0, R₁ is methyl, R₂ is CF₃, m is 1; R₃ is 4-methylphenyl; L₁ is S, and L₂ is O.

In a further embodiment, the methods comprise a compound of formula 42 and the attendant definitions wherein n is 0, R₁ is methyl, R₂ is CF₃, m is 1; R₃ is 4-methylphenyl; L₁ is S, L₂ is O; and L₃ is NR₁.

In a further embodiment, the methods comprise a compound of formula 42 and the attendant definitions wherein n is 0, R₁ is methyl, R₂ is CF₃, m is 1; R₃ is 4-methylphenyl; L₁ is S, L₂ is O; L₃ is NR₁, and L₄ is NR₁.

In another embodiment, methods for activating a sirtuin protein comprise using an activating compound of formula 43: 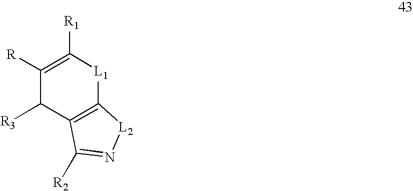
wherein, independently for each occurrence:
R and R₁ are hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₂ and R₃ are H or a substituted or unsubstituted alkyl, aryl, aralkyl, heterocyclyl, heterocyclylalkyl, heteroaryl, or heteroaralkyl;
R₄ is alkyl, —SO₃H, monosaccharide, oligosaccharide, glycofuranosyl, glycopyranosyl, glucuronosyl, or glucuronide; and
L₁ and L₂ are O, NR₂, or S.

In a further embodiment, the methods comprise a compound of formula 43 and the attendant definitions wherein R is cyano.

In a further embodiment, the methods comprise a compound of formula 43 and the attendant definitions wherein R₁ is NH₂.

In a further embodiment, the methods comprise a compound of formula 43 and the attendant definitions wherein R₂ is 4-bromophenyl.

In a further embodiment, the methods comprise a compound of formula 43 and the attendant definitions wherein R₃ is 3-hydroxy-4-methoxyphenyl.

In a further embodiment, the methods comprise a compound of formula 43 and the attendant definitions wherein L₁ is O.

In a further embodiment, the methods comprise a compound of formula 43 and the attendant definitions wherein L₂ is NR₂.

In a further embodiment, the methods comprise a compound of formula 43 and the attendant definitions wherein R is cyano and R₁ is NH₂.

In a further embodiment, the methods comprise a compound of formula 43 and the attendant definitions wherein R is cyano, R₁ is NH₂, and R₂ is 4-bromophenyl.

In a further embodiment, the methods comprise a compound of formula 43 and the attendant definitions wherein R is cyano, R₁ is NH₂, R₂ is 4-bromophenyl, and R₃ is 3-hydroxy-4-methoxyphenyl.

In a further embodiment, the methods comprise a compound of formula 43 and the attendant definitions wherein R is cyano, R₁ is NH₂, R₂ is 4-bromophenyl, R₃ is 3-hydroxy-4-methoxyphenyl, and L₁ is O.

In a further embodiment, the methods comprise a compound of formula 43 and the attendant definitions wherein R is cyano, R₁ is NH₂, R₂ is 4-bromophenyl, R₃ is 3-hydroxy-4-methoxyphenyl, L₁ is O, and L₂ is NR₂.

In another embodiment, methods for activating a sirtuin protein comprise using an activating compound of formula 44: 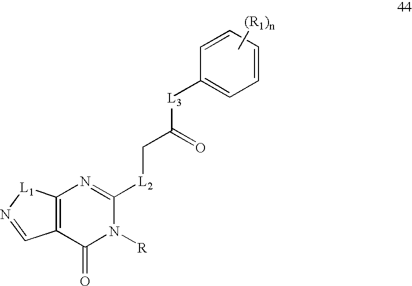
wherein, independently for each occurrence:
R is H or a substituted or unsubstituted alkyl, aryl, aralkyl, heterocyclyl, heterocyclylalkyl, heteroaryl, or heteroaralkyl;
R₁ is hydroxy, amino, cyano, halide, OR₂, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₂ is alkyl, —SO₃H, monosaccharide, oligosaccharide, glycofuranosyl, glycopyranosyl, glucuronosyl, or glucuronide;
L₁, L₂, and L₃ are O, NR, or S; and
n is an integer from 0 to 5 inclusive.

In a further embodiment, the methods comprise a compound of formula 44 and the attendant definitions wherein R is 3-trifluoromethylphenyl.

In a further embodiment, the methods comprise a compound of formula 44 and the attendant definitions wherein R₁ is C(O)OCH₃.

In a further embodiment, the methods comprise a compound of formula 44 and the attendant definitions wherein L₁ is NR.

In a further embodiment, the methods comprise a compound of formula 44 and the attendant definitions wherein L₂ is S.

In a further embodiment, the methods comprise a compound of formula 44 and the attendant definitions wherein L₃ is NR.

In a further embodiment, the methods comprise a compound of formula 44 and the attendant definitions wherein n is 2.

In a further embodiment, the methods comprise a compound of formula 44 and the attendant definitions wherein R is 3-trifluoromethylphenyl and R₁ is C(O)OCH₃.

In a further embodiment, the methods comprise a compound of formula 44 and the attendant definitions wherein R is 3-trifluoromethylphenyl, R₁ is C(O)OCH₃, and L₁ is NR.

In a further embodiment, the methods comprise a compound of formula 44 and the attendant definitions wherein R is 3-trifluoromethylphenyl, R₁ is C(O)OCH₃, L₁ is NR, and L₂ is S.

In a further embodiment, the methods comprise a compound of formula 44 and the attendant definitions wherein R is 3-trifluoromethylphenyl, R₁ is C(O)OCH₃, L₁ is NR, L₂ is S, and L₃ is NR.

In a further embodiment, the methods comprise a compound of formula 44 and the attendant definitions wherein R is 3-trifluoromethylphenyl, R₁ is C(O)OCH₃, L₁ is NR, L₂ is S, L₃ is NR, and n is 2.

In another embodiment, methods for activating a sirtuin protein comprise using an activating compound of formula 45: 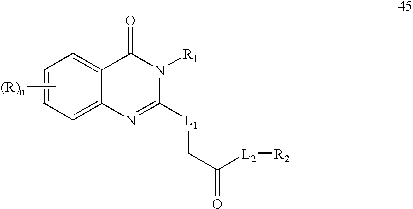
wherein, independently for each occurrence:
R is hydroxy, amino, cyano, halide, OR₃, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₁ and R₂ are H or a substituted or unsubstituted alkyl, aryl, aralkyl, heterocyclyl, heterocyclylalkyl, heteroaryl, or heteroaralkyl;
R₃ is alkyl, —SO₃H, monosaccharide, oligosaccharide, glycofuranosyl, glycopyranosyl, glucuronosyl, or glucuronide;
L₁ and L₂ are O, NR₁, or S; and
n is an integer from 0 to 4 inclusive.

In a further embodiment, the methods comprise a compound of formula 45 and the attendant definitions wherein n is 0.

In a further embodiment, the methods comprise a compound of formula 45 and the attendant definitions wherein R₁ is 2-tetrahydrofuranylmethyl.

In a further embodiment, the methods comprise a compound of formula 45 and the attendant definitions wherein R₂ is —CH₂CH₂C₆H₄SO₂NH₂.

In a further embodiment, the methods comprise a compound of formula 45 and the attendant definitions wherein L₁ is S.

In a further embodiment, the methods comprise a compound of formula 45 and the attendant definitions wherein L₂ is NR₁.

In a further embodiment, the methods comprise a compound of formula 45 and the attendant definitions wherein n is 0 and R₁ is 2-tetrahydrofuranylmethyl.

In a further embodiment, the methods comprise a compound of formula 45 and the attendant definitions wherein n is 0, R₁ is 2-tetrahydrofuranylmethyl, and R₂ is —CH₂CH₂C₆H₄SO₂NH₂.

In a further embodiment, the methods comprise a compound of formula 45 and the attendant definitions wherein n is 0, R₁ is 2-tetrahydrofuranylmethyl, R₂ is —CH₂CH₂C₆H₄SO₂NH₂, and L₁ is S.

In a further embodiment, the methods comprise a compound of formula 45 and the attendant definitions wherein n is 0, R₁ is 2-tetrahydrofuranylmethyl, R₂ is —CH₂CH₂C₆H₄SO₂NH₂, L₁ is S, and L₂ is NR₁.

In another embodiment, methods for activating a sirtuin protein comprise using an activating compound of formula 46: 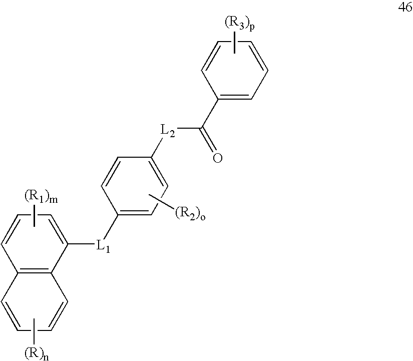
wherein, independently for each occurrence:
R, R₁, R₂, and R₃ are hydroxy, amino, cyano, halide, OR₅, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₅ is alkyl, —SO₃H, monosaccharide, oligosaccharide, glycofuranosyl, glycopyranosyl, glucuronosyl, or glucuronide;
L₁ and L₂ are O, NR₄, or S;
R₄ is H or a substituted or unsubstituted alkyl, aryl, aralkyl, heterocyclyl, heterocyclylalkyl, heteroaryl, or heteroaralkyl;
n is an integer from 0 to 4 inclusive;
m is an integer from 0 to 3 inclusive;
o is an integer from 0 to 4 inclusive; and
p is an integer from 0 to 5 inclusive.

In a further embodiment, the methods comprise a compound of formula 46 and the attendant definitions wherein n is 0.

In a further embodiment, the methods comprise a compound of formula 46 and the attendant definitions wherein m is 1.

In a further embodiment, the methods comprise a compound of formula 46 and the attendant definitions wherein R₁ is Cl.

In a further embodiment, the methods comprise a compound of formula 46 and the attendant definitions wherein o is 1.

In a further embodiment, the methods comprise a compound of formula 46 and the attendant definitions wherein R₂ is Cl.

In a further embodiment, the methods comprise a compound of formula 46 and the attendant definitions wherein p is 3.

In a further embodiment, the methods comprise a compound of formula 46 and the attendant definitions wherein R₃ is OH or I.

In a further embodiment, the methods comprise a compound of formula 46 and the attendant definitions wherein n is 0 and m is 1.

In a further embodiment, the methods comprise a compound of formula 46 and the attendant definitions wherein n is 0, m is 1, and o is 1.

In a further embodiment, the methods comprise a compound of formula 46 and the attendant definitions wherein n is 0, m is 1, o is 1, and R₁ is Cl.

In a further embodiment, the methods comprise a compound of formula 46 and the attendant definitions wherein n is 0, m is 1, o is 1, R₁ is Cl, and p is 3.

In a further embodiment, the methods comprise a compound of formula 46 and the attendant definitions wherein n is 0, m is 1, o is 1, R₁ is Cl, p is 3, and R₂ is OH or I.

In another embodiment, methods for activating a sirtuin protein comprise using an activating compound of formula 47: 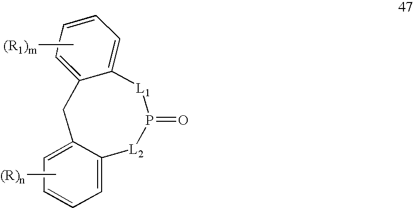
wherein, independently for each occurrence:
R and R₁ are hydroxy, amino, cyano, halide, OR₅, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
L₁ and L₂ are O, NR₄, or S;
R₄ is H or a substituted or unsubstituted alkyl, aryl, aralkyl, heterocyclyl, heterocyclylalkyl, heteroaryl, or heteroaralkyl;
R₅ is alkyl, —SO₃H, monosaccharide, oligosaccharide, glycofuranosyl, glycopyranosyl, glucuronosyl, or glucuronide; and
m and n are integers from 0 to 4 inclusive.

In a further embodiment, the methods comprise a compound of formula 47 and the attendant definitions wherein n is 2.

In a further embodiment, the methods comprise a compound of formula 47 and the attendant definitions wherein R is methyl or t-butyl.

In a further embodiment, the methods comprise a compound of formula 47 and the attendant definitions wherein m is 2.

In a further embodiment, the methods comprise a compound of formula 47 and the attendant definitions wherein R₁ is methyl or t-butyl.

In a further embodiment, the methods comprise a compound of formula 47 and the attendant definitions wherein L₁ is O.

In a further embodiment, the methods comprise a compound of formula 47 and the attendant definitions wherein L₂ is O.

In a further embodiment, the methods comprise a compound of formula 47 and the attendant definitions wherein n is 2 and R is methyl or t-butyl.

In a further embodiment, the methods comprise a compound of formula 47 and the attendant definitions wherein n is 2, R is methyl or t-butyl, and m is 2.

In a further embodiment, the methods comprise a compound of formula 47 and the attendant definitions wherein n is 2, R is methyl or t-butyl, m is 2, and R₁ is methyl or t-butyl.

In a further embodiment, the methods comprise a compound of formula 47 and the attendant definitions wherein n is 2, R is methyl or t-butyl, m is 2, R₁ is methyl or t-butyl, and L₁ is O.

In a further embodiment, the methods comprise a compound of formula 47 and the attendant definitions wherein n is 2, R is methyl or t-butyl, m is 2, R₁ is methyl or t-butyl, L₁ is O, and L₂ is O.

In another embodiment, methods for activating a sirtuin protein comprise using an activating compound of formula 48: 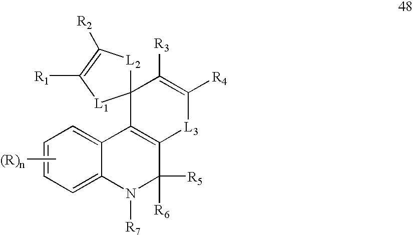
wherein, independently for each occurrence:
R, R₁, R₂, R₃, R₄, R₅, and R₆ are hydroxy, amino, cyano, halide, OR₈, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₇ is H or a substituted or unsubstituted alkyl, acyl, aryl, aralkyl, heterocyclyl, heterocyclylalkyl, heteroaryl, or heteroaralkyl;
R₈ is alkyl, —SO₃H, monosaccharide, oligosaccharide, glycofuranosyl, glycopyranosyl, glucuronosyl, or glucuronide;
L₁, L₂, and L₃ are O, NR₇, or S and
n is an integer from 0 to 4 inclusive.

In a further embodiment, the methods comprise a compound of formula 48 and the attendant definitions wherein n is 1.

In a further embodiment, the methods comprise a compound of formula 48 and the attendant definitions wherein R is methyl.

In a further embodiment, the methods comprise a compound of formula 48 and the attendant definitions wherein R₁ is C(O)OCH₃.

In a further embodiment, the methods comprise a compound of formula 48 and the attendant definitions wherein R₂ is C(O)OCH₃.

In a further embodiment, the methods comprise a compound of formula 48 and the attendant definitions wherein R₃ is C(O)OCH₃.

In a further embodiment, the methods comprise a compound of formula 48 and the attendant definitions wherein R₄ is C(O)OCH₃.

In a further embodiment, the methods comprise a compound of formula 48 and the attendant definitions wherein R₅ is methyl.

In a further embodiment, the methods comprise a compound of formula 48 and the attendant definitions wherein R₆ is methyl.

In a further embodiment, the methods comprise a compound of formula 48 and the attendant definitions wherein R₇ is C(O)CF₃.

In a further embodiment, the methods comprise a compound of formula 48 and the attendant definitions wherein L₁ is S.

In a further embodiment, the methods comprise a compound of formula 48 and the attendant definitions wherein L₂ is S.

In a further embodiment, the methods comprise a compound of formula 48 and the attendant definitions wherein L₃ is S.

In a further embodiment, the methods comprise a compound of formula 48 and the attendant definitions wherein n is 1 and R is methyl.

In a further embodiment, the methods comprise a compound of formula 48 and the attendant definitions wherein n is 1, R is methyl, and R₁ is C(O)OCH₃.

In a further embodiment, the methods comprise a compound of formula 48 and the attendant definitions wherein n is 1, R is methyl, R₁ is C(O)OCH₃, and R₂ is C(O)OCH₃.

In a further embodiment, the methods comprise a compound of formula 48 and the attendant definitions wherein n is 1, R is methyl, R₁ is C(O)OCH₃, R₂ is C(O)OCH₃, and R₃ is C(O)OCH₃.

In a further embodiment, the methods comprise a compound of formula 48 and the attendant definitions wherein n is 1, R is methyl, R₁ is C(O)OCH₃, R₂ is C(O)OCH₃, R₃ is C(O)OCH₃, and R₄ is C(O)OCH₃.

In a further embodiment, the methods comprise a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, and R₅ is methyl.

In a further embodiment, the methods comprise a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, and R₆ is methyl.

In a further embodiment, the methods comprise a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, and R₇ is C(O)CF₃.

In a further embodiment, the methods comprise a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, R₇ is C(O)CF₃, and L₁ is S.

In a further embodiment, the methods comprise a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, R₇ is C(O)CF₃, L₁ is S, and L₂ is S.

In a further embodiment, the methods comprise a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, R₇ is C(O)CF₃, L₁ is S, L₂ is S, and L₃ is S.

In another embodiment, methods for activating a sirtuin protein comprise using an activating compound of formula 49: 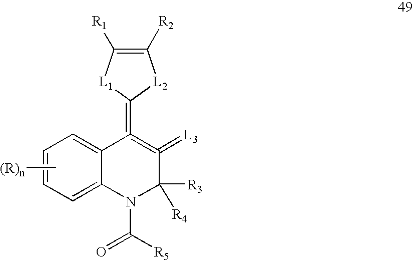
wherein, independently for each occurrence:
R, R₁, R₂, R₃, R₄, and R₅ are hydroxy, amino, cyano, halide, OR₇, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
L₁, L₂, and L₃ are O, NR₆, or S;
R₆ is H or a substituted or unsubstituted alkyl, aryl, aralkyl, heterocyclyl, heterocyclylalkyl, heteroaryl, or heteroaralkyl;
R₇ is alkyl, —SO₃H, monosaccharide, oligosaccharide, glycofuranosyl, glycopyranosyl, glucuronosyl, or glucuronide; and
n is an integer from 0 to 4 inclusive.

In a further embodiment, the methods comprise a compound of formula 49 and the attendant definitions wherein n is 1.

In a further embodiment, the methods comprise a compound of formula 49 and the attendant definitions wherein R is methyl.

In a further embodiment, the methods comprise a compound of formula 49 and the attendant definitions wherein R₁ is C(O)OCH₃.

In a further embodiment, the methods comprise a compound of formula 49 and the attendant definitions wherein R₂ is C(O)OCH₃.

In a further embodiment, the methods comprise a compound of formula 49 and the attendant definitions wherein R₃ is methyl.

In a further embodiment, the methods comprise a compound of formula 49 and the attendant definitions wherein R₄ is methyl.

In a further embodiment, the methods comprise a compound of formula 49 and the attendant definitions wherein R₅ is CH₂CH(CH₃)₂.

In a further embodiment, the methods comprise a compound of formula 49 and the attendant definitions wherein L₁ is S.

In a further embodiment, the methods comprise a compound of formula 49 and the attendant definitions wherein L₂ is S.

In a further embodiment, the methods comprise a compound of formula 49 and the attendant definitions wherein L₃ is S.

In a further embodiment, the methods comprise a compound of formula 49 and the attendant definitions wherein n is 1 and R is methyl.

In a further embodiment, the methods comprise a compound of formula 49 and the attendant definitions wherein n is 1, R is methyl, and R₁ is C(O)OCH₃.

In a further embodiment, the methods comprise a compound of formula 49 and the attendant definitions wherein n is 1, R is methyl, R₁ is C(O)OCH₃, and R₂ is C(O)OCH₃.

In a further embodiment, the methods comprise a compound of formula 49 and the attendant definitions wherein n is 1, R is methyl, R₁ is C(O)OCH₃, R₂ is C(O)OCH₃, and R₃ is methyl.

In a further embodiment, the methods comprise a compound of formula 49 and the attendant definitions wherein n is 1, R is methyl, R₁ is C(O)OCH₃, R₂ is C(O)OCH₃, R₃ is methyl, and R₄ is methyl.

In a further embodiment, the methods comprise a compound of formula 49 and the attendant definitions wherein n is 1, R is methyl, R₁ is C(O)OCH₃, R₂ is C(O)OCH₃, R₃ is methyl, R₄ is methyl, and R₅ is CH₂CH(CH₃)₂.

In a further embodiment, the methods comprise a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, and L₁ is S.

In a further embodiment, the methods comprise a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, and L₁ is S.

In a further embodiment, the methods comprise a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, L₁ is S, and L₂ is S.

In a further embodiment, the methods comprise a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, L₁ is S, and L₂ is S.

In a further embodiment, the methods comprise a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, L₁ is S, L₂ is S, and L₃ is S.

In another embodiment, methods for activating a sirtuin protein comprise using an activating compound of formula 50: 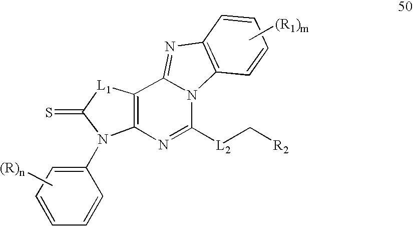
wherein, independently for each occurrence:
R and R₁ are hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₂ is H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₄ is alkyl, —SO₃H, monosaccharide, oligosaccharide, glycofuranosyl, glycopyranosyl, glucuronosyl, or glucuronide;
L₁ and L₂ are O, NR₃, or S;
R₃ is H or a substituted or unsubstituted alkyl, aryl, aralkyl, heterocyclyl, heterocyclylalkyl, heteroaryl, or heteroaralkyl;
n is an integer from 0 to 5 inclusive; and
m is an integer from 0 to 4 inclusive.

In a further embodiment, the methods comprise a compound of formula 50 and the attendant definitions wherein n is 1.

In a further embodiment, the methods comprise a compound of formula 50 and the attendant definitions wherein R is CO₂Et.

In a further embodiment, the methods comprise a compound of formula 50 and the attendant definitions wherein m is 0.

In a further embodiment, the methods comprise a compound of formula 50 and the attendant definitions wherein R₂ is cyano.

In a further embodiment, the methods comprise a compound of formula 50 and the attendant definitions wherein L₁ is S.

In a further embodiment, the methods comprise a compound of formula 50 and the attendant definitions wherein L₂ is S.

In a further embodiment, the methods comprise a compound of formula 50 and the attendant definitions wherein n is 1 and R is CO₂Et.

In a further embodiment, the methods comprise a compound of formula 50 and the attendant definitions wherein n is 1, R is CO₂Et, and m is 0.

In a further embodiment, the methods comprise a compound of formula 50 and the attendant definitions wherein n is 1, R is CO₂Et, m is 0, and R₂ is cyano.

In a further embodiment, the methods comprise a compound of formula 50 and the attendant definitions wherein n is 1, R is CO₂Et, m is 0, R₂ is cyano, and L₁ is S.

In a further embodiment, the methods comprise a compound of formula 50 and the attendant definitions wherein n is 1, R is CO₂Et, m is 0, R₂ is cyano, L₁ is S, and L₂ is S.

In another embodiment, methods for activating a sirtuin protein comprise using an activating compound of formula 51: 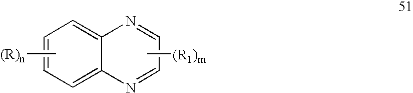
wherein, independently for each occurrence:
R and R₁ are hydroxy, amino, cyano, halide, OR₂, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₂ is alkyl, —SO₃H, monosaccharide, oligosaccharide, glycofuranosyl, glycopyranosyl, glucuronosyl, or glucuronide;
n is an integer from 0 to 4 inclusive; and
m is an integer from 0 to 2 inclusive.

In a further embodiment, the methods comprise a compound of formula 51 and the attendant definitions wherein n is 2.

In a further embodiment, the methods comprise a compound of formula 51 and the attendant definitions wherein R is Cl or trifluoromethyl.

In a further embodiment, the methods comprise a compound of formula 51 and the attendant definitions wherein m is 2.

In a further embodiment, the methods comprise a compound of formula 51 and the attendant definitions wherein R₁ is phenyl.

In a further embodiment, the methods comprise a compound of formula 51 and the attendant definitions wherein n is 2 and R is Cl or trifluoromethyl.

In a further embodiment, the methods comprise a compound of formula 51 and the attendant definitions wherein n is 2, R is Cl or trifluoromethyl, and m is 2.

In a further embodiment, the methods comprise a compound of formula 51 and the attendant definitions wherein n is 2, R is Cl or trifluoromethyl, m is 2, and R₁ is phenyl.

In a further embodiment, the methods comprise a compound of formula 51 and the attendant definitions wherein n is 1.

In a further embodiment, the methods comprise a compound of formula 51 and the attendant definitions wherein R is F.

In a further embodiment, the methods comprise a compound of formula 51 and the attendant definitions wherein R₁ is 4-methylphenyl.

In a further embodiment, the methods comprise a compound of formula 51 and the attendant definitions wherein n is 1 and R is F.

In a further embodiment, the methods comprise a compound of formula 51 and the attendant definitions wherein n is 1, R is F, and m is 2.

In a further embodiment, the methods comprise a compound of formula 51 and the attendant definitions wherein n is 1, R is F, m is 2, and R₁ is 4-methylphenyl.

In another embodiment, methods for activating a sirtuin protein comprise using an activating compound of formula 52: 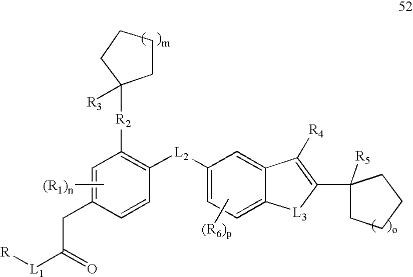
wherein, independently for each occurrence:
R is H or a substituted or unsubstituted alkyl, aryl, aralkyl, heterocyclyl, heterocyclylalkyl, heteroaryl, or heteroaralkyl;
R₁ and R₆ are hydroxy, amino, cyano, halide, OR₇, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₂ is alkylene, alkenylene, or alkynylene;
R₃, R₄, and R₅ are H, hydroxy, amino, cyano, halide, OR₇, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₇ is alkyl, —SO₃H, monosaccharide, oligosaccharide, glycofuranosyl, glycopyranosyl, glucuronosyl, or glucuronide;
L₁, L₂, and L₃ are O, NR, or S;
n and p are integers from 0 to 3 inclusive; and
m and o are integers from 0 to 2 inclusive.

In a further embodiment, the methods comprise a compound of formula 52 and the attendant definitions wherein R is CH₂CH₂OH.

In a further embodiment, the methods comprise a compound of formula 52 and the attendant definitions wherein n is 1.

In a further embodiment, the methods comprise a compound of formula 52 and the attendant definitions wherein R₁ is I.

In a further embodiment, the methods comprise a compound of formula 52 and the attendant definitions wherein R₂ is alkynylene.

In a further embodiment, the methods comprise a compound of formula 52 and the attendant definitions wherein m is 1.

In a further embodiment, the methods comprise a compound of formula 52 and the attendant definitions wherein R₃ is OH.

In a further embodiment, the methods comprise a compound of formula 52 and the attendant definitions wherein R₄ is C(O)OEt.

In a further embodiment, the methods comprise a compound of formula 52 and the attendant definitions wherein o is 1.

In a further embodiment, the methods comprise a compound of formula 52 and the attendant definitions wherein R₅ is OH.

In a further embodiment, the methods comprise a compound of formula 52 and the attendant definitions wherein p is 0.

In a further embodiment, the methods comprise a compound of formula 52 and the attendant definitions wherein L₁ is NH.

In a further embodiment, the methods comprise a compound of formula 52 and the attendant definitions wherein L₂ is O.

In a further embodiment, the methods comprise a compound of formula 52 and the attendant definitions wherein L₃ is O.

In a further embodiment, the methods comprise a compound of formula 52 and the attendant definitions wherein R is CH₂CH₂OH and n is 1.

In a further embodiment, the methods comprise a compound of formula 52 and the attendant definitions wherein R is CH₂CH₂OH, n is 1, and R₁ is I.

In a further embodiment, the methods comprise a compound of formula 52 and the attendant definitions wherein R is CH₂CH₂OH, n is 1, R₁ is I, and R₂ is alkynylene.

In a further embodiment, the methods comprise a compound of formula 52 and the attendant definitions wherein R is CH₂CH₂OH, n is 1, R₁ is I, R₂ is alkynylene, and m is 1.

In a further embodiment, the methods comprise a compound of formula 52 and the attendant definitions wherein R is CH₂CH₂OH, n is 1, R₁ is I, R₂ is alkynylene, m is 1, and R₃ is OH.

In a further embodiment, the methods comprise a compound of formula 52 and the attendant definitions wherein R is CH₂CH₂OH, n is 1, R₁ is I, R₂ is alkynylene, m is 1, R₃ is OH, and R₄ is C(O)OEt.

In a further embodiment, the methods comprise a compound of formula 52 and the attendant definitions wherein R is CH₂CH₂OH, n is 1, R₁ is I, R₂ is alkynylene, m is 1, R₃ is OH, R₄ is C(O)OEt, and o is 1.

In a further embodiment, the methods comprise a compound of formula 52 and the attendant definitions wherein R is CH₂CH₂OH, n is 1, R₁ is I, R₂ is alkynylene, m is 1, R₃ is OH, R₄ is C(O)OEt, o is 1, and R₅ is OH.

In a further embodiment, the methods comprise a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, and p is 0.

In a further embodiment, the methods comprise a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, p is 0, and L₁ is NH.

In a further embodiment, the methods comprise a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, p is 0, L₁ is NH, and L₂ is O.

In a further embodiment, the methods comprise a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, p is 0, L₁ is NH, L₂ is O, and L₃ is O.

In another embodiment, methods for activating a sirtuin protein comprise using an activating compound of formula 53: 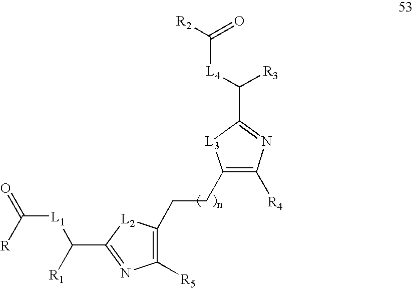
wherein, independently for each occurrence:
R, R₁, R₂, R₃, R₄, and R₅ are H, hydroxy, amino, cyano, halide, OR₇, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
L₁, L₂, L₃, and L₄ are O, NR₆, or S;
R₆ is and H, or a substituted or unsubstituted alkyl, aryl, aralkyl, heterocyclyl, heterocyclylalkyl, heteroaryl, or heteroaralkyl;
R₇ is alkyl, —SO₃H, monosaccharide, oligosaccharide, glycofuranosyl, glycopyranosyl, glucuronosyl, or glucuronide; and
n is an integer from 0 to 5 inclusive.

In a further embodiment, the methods comprise a compound of formula 53 and the attendant definitions wherein R is O-t-butyl.

In a further embodiment, the methods comprise a compound of formula 53 and the attendant definitions wherein R₁ is t-butyl.

In a further embodiment, the methods comprise a compound of formula 53 and the attendant definitions wherein R₂ is O-t-butyl.

In a further embodiment, the methods comprise a compound of formula 53 and the attendant definitions wherein R₃ is t-butyl.

In a further embodiment, the methods comprise a compound of formula 53 and the attendant definitions wherein R₄ is C(O)OMe.

In a further embodiment, the methods comprise a compound of formula 53 and the attendant definitions wherein R₅ is C(O)OMe.

In a further embodiment, the methods comprise a compound of formula 53 and the attendant definitions wherein L₁ is NH.

In a further embodiment, the methods comprise a compound of formula 53 and the attendant definitions wherein L₂ is O.

In a further embodiment, the methods comprise a compound of formula 53 and the attendant definitions wherein L₃ is O.

In a further embodiment, the methods comprise a compound of formula 53 and the attendant definitions wherein L₄ is NH.

In a further embodiment, the methods comprise a compound of formula 53 and the attendant definitions wherein n is 1.

In a further embodiment, the methods comprise a compound of formula 53 and the attendant definitions wherein R is O-t-butyl and R₁ is t-butyl.

In a further embodiment, the methods comprise a compound of formula 53 and the attendant definitions wherein R is O-t-butyl, R₁ is t-butyl, and R₂ is O-t-butyl.

In a further embodiment, the methods comprise a compound of formula 53 and the attendant definitions wherein R is O-t-butyl, R₁ is t-butyl, R₂ is O-t-butyl, and R₃ is t-butyl.

In a further embodiment, the methods comprise a compound of formula 53 and the attendant definitions wherein R is O-t-butyl, R₁ is t-butyl, R₂ is O-t-butyl, R₃ is t-butyl, and R₄ is C(O)OMe.

In a further embodiment, the methods comprise a compound of formula 53 and the attendant definitions wherein R is O-t-butyl, R₁ is t-butyl, R₂ is O-t-butyl, R₃ is t-butyl, R₄ is C(O)OMe, and R₅ is C(O)OMe.

In a further embodiment, the methods comprise a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, and L₁ is NH.

In a further embodiment, the methods comprise a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, and L₂ is O.

In a further embodiment, the methods comprise a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, L₂ is O, and L₃ is O.

In a further embodiment, the methods comprise a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, L₂ is O, L₃ is O, and L₄ is NH.

In a further embodiment, the methods comprise a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, L₂ is O, L₃ is O, L₄ is NH, and n is 1.

In another embodiment, methods for activating a sirtuin protein comprise using an activating compound of formula 54: 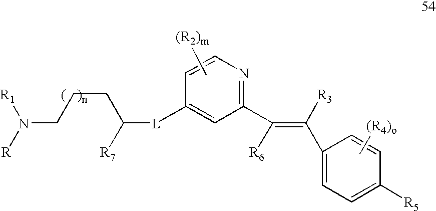
wherein, independently for each occurrence:
R and R₁ are H or a substituted or unsubstituted alkyl, aryl, aralkyl, heterocyclyl, heterocyclylalkyl, heteroaryl, or heteroaralkyl;
R₂, R₄, and R₅ are hydroxy, amino, cyano, halide, OR₈, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₃, R₆, and R₇ are H, hydroxy, amino, cyano, halide, OR₈, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₈ is alkyl, —SO₃H, monosaccharide, oligosaccharide, glycofuranosyl, glycopyranosyl, glucuronosyl, or glucuronide;
L is O, NR, or S;
n and o are integers from 0 to 4 inclusive; and
m is an integer from 0 to 3 inclusive.

In a further embodiment, the methods comprise a compound of formula 54 and the attendant definitions wherein R is ethyl.

In a further embodiment, the methods comprise a compound of formula 54 and the attendant definitions wherein R₁ is ethyl.

In a further embodiment, the methods comprise a compound of formula 54 and the attendant definitions wherein m is 0.

In a further embodiment, the methods comprise a compound of formula 54 and the attendant definitions wherein R₃ is H.

In a further embodiment, the methods comprise a compound of formula 54 and the attendant definitions wherein o is 0.

In a further embodiment, the methods comprise a compound of formula 54 and the attendant definitions wherein R₅ is Cl.

In a further embodiment, the methods comprise a compound of formula 54 and the attendant definitions wherein R₆ is H.

In a further embodiment, the methods comprise a compound of formula 54 and the attendant definitions wherein R₇ is methyl.

In a further embodiment, the methods comprise a compound of formula 54 and the attendant definitions wherein L is NH.

In a further embodiment, the methods comprise a compound of formula 54 and the attendant definitions wherein n is 1.

In a further embodiment, the methods comprise a compound of formula 54 and the attendant definitions wherein R is ethyl and R₁ is ethyl.

In a further embodiment, the methods comprise a compound of formula 54 and the attendant definitions wherein R is ethyl, R₁ is ethyl, and m is 0.

In a further embodiment, the methods comprise a compound of formula 54 and the attendant definitions wherein R is ethyl, R₁ is ethyl, m is 0, and R₃ is H.

In a further embodiment, the methods comprise a compound of formula 54 and the attendant definitions wherein R is ethyl, R is ethyl, m is 0, R₃ is H, and o is 0.

In a further embodiment, the methods comprise a compound of formula 54 and the attendant definitions wherein R is ethyl, R₁ is ethyl, m is 0, R₃ is H, o is 0, and R₅ is Cl.

In a further embodiment, the methods comprise a compound of formula 54 and the attendant definitions wherein R is ethyl, R₁ is ethyl, m is 0, R₃ is H, o is 0, R₅ is Cl, and R₆ is H.

In a further embodiment, the methods comprise a compound of formula 54 and the attendant definitions wherein R is ethyl, R₁ is ethyl, m is 0, R₃ is H, o is 0, R₅ is Cl, R₆ is H, and R₇ is methyl.

In a further embodiment, the methods comprise a compound of formula 54 and the attendant definitions wherein R is ethyl, R₁ is ethyl, m is 0, R₃ is H, o is 0, R₅ is Cl, R₆ is H, R₇ is methyl, and L is NH.

In a further embodiment, the methods comprise a compound of formula 54 and the attendant definitions wherein R is ethyl, R₁ is ethyl, m is 0, R₃ is H, o is 0, R₅ is Cl, R₆ is H, R₇ is methyl, L is NH, and n is 1.

In another embodiment, methods for activating a sirtuin protein comprise using an activating compound of formula 55: 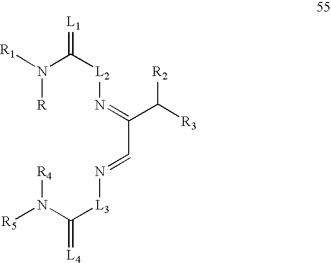
wherein, independently for each occurrence:
R, R₁, R₄, and R₅ are H or a substituted or unsubstituted alkyl, aryl, aralkyl, heterocyclyl, heterocyclylalkyl, heteroaryl, or heteroaralkyl;
R₂ and R₃ are H, hydroxy, amino, cyano, halide, OR₆, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₆ is alkyl, —SO₃H, monosaccharide, oligosaccharide, glycofuranosyl, glycopyranosyl, glucuronosyl, or glucuronide; and
L₁, L₂, L₃, and L₄ are O, NR, or S.

In a further embodiment, the methods comprise a compound of formula 55 and the attendant definitions wherein R is H.

In a further embodiment, the methods comprise a compound of formula 55 and the attendant definitions wherein R₁ is H.

In a further embodiment, the methods comprise a compound of formula 55 and the attendant definitions wherein R₂ is OEt.

In a further embodiment, the methods comprise a compound of formula 55 and the attendant definitions wherein R₃ is methyl.

In a further embodiment, the methods comprise a compound of formula 55 and the attendant definitions wherein R₄ is H.

In a further embodiment, the methods comprise a compound of formula 55 and the attendant definitions wherein R₅ is H.

In a further embodiment, the methods comprise a compound of formula 55 and the attendant definitions wherein L₁ is S.

In a further embodiment, the methods comprise a compound of formula 55 and the attendant definitions wherein L₂ is NH.

In a further embodiment, the methods comprise a compound of formula 55 and the attendant definitions wherein L₃ is NH.

In a further embodiment, the methods comprise a compound of formula 55 and the attendant definitions wherein L₄ is S.

In a further embodiment, the methods comprise a compound of formula 55 and the attendant definitions wherein R is H and R₁ is H.

In a further embodiment, the methods comprise a compound of formula 55 and the attendant definitions wherein R is H, R₁ is H, and R₂ is OEt.

In a further embodiment, the methods comprise a compound of formula 55 and the attendant definitions wherein R is H, R₁ is H, R₂ is OEt, and R₃ is methyl.

In a further embodiment, the methods comprise a compound of formula 55 and the attendant definitions wherein R is H, R₁ is H, R₂ is OEt, R₃ is methyl, and R₄ is H.

In a further embodiment, the methods comprise a compound of formula 55 and the attendant definitions wherein R is H, R₁ is H, R₂ is OEt, R₃ is methyl, R₄ is H, and R₅ is H.

In a further embodiment, the methods comprise a compound of formula 55 and the attendant definitions wherein R is H, R₁ is H, R₂ is OEt, R₃ is methyl, R₄ is H, R₅ is H, and L₁ is S.

In a further embodiment, the methods comprise a compound of formula 55 and the attendant definitions wherein R is H, R₁ is H, R₂ is OEt, R₃ is methyl, R₄ is H, R₅ is H, L₁ is S, and L₂ is NH.

In a further embodiment, the methods comprise a compound of formula 55 and the attendant definitions wherein R is H, R₁ is H, R₂ is OEt, R₃ is methyl, R₄ is H, R₅ is H, L₁ is S, L₂ is NH, and L₃ is NH.

In a further embodiment, the methods comprise a compound of formula 55 and the attendant definitions wherein R is H, R₁ is H, R₂ is OEt, R₃ is methyl, R₄ is H, R₅ is H, L₁ is S, L₂ is NH, L₃ is NH, and L₄ is S.

In another embodiment, methods for activating a sirtuin protein comprise using an activating compound of formula 56: 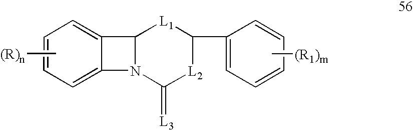
wherein, independently for each occurrence:
R and R₁ are hydroxy, amino, cyano, halide, OR₃, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₃ is alkyl, —SO₃H, monosaccharide, oligosaccharide, glycofuranosyl, glycopyranosyl, glucuronosyl, or glucuronide;
L₁, L₂, and L₃ are O, NR₂, or S;
R₂ is H or a substituted or unsubstituted alkyl, aryl, aralkyl, heterocyclyl, heterocyclylalkyl, heteroaryl, or heteroaralkyl;
n is an integer from 0 to 4 inclusive; and
m is an integer from 0 to 5 inclusive.

In a further embodiment, the methods comprise a compound of formula 56 and the attendant definitions wherein n is 0.

In a further embodiment, the methods comprise a compound of formula 56 and the attendant definitions wherein m is 0.

In a further embodiment, the methods comprise a compound of formula 56 and the attendant definitions wherein L₁ is NH.

In a further embodiment, the methods comprise a compound of formula 56 and the attendant definitions wherein L₂ is S.

In a further embodiment, the methods comprise a compound of formula 56 and the attendant definitions wherein L₃ is S.

In a further embodiment, the methods comprise a compound of formula 56 and the attendant definitions wherein m is 0 and n is 0.

In a further embodiment, the methods comprise a compound of formula 56 and the attendant definitions wherein m is 0, n is 0, and L₁ is NH.

In a further embodiment, the methods comprise a compound of formula 56 and the attendant definitions wherein m is 0, n is 0, L₁ is NH, and L₂ is S.

In a further embodiment, the methods comprise a compound of formula 56 and the attendant definitions wherein m is 0, n is 0, L₁ is NH, L₂ is S, and L₃ is S.

In another embodiment, methods for activating a sirtuin protein comprise using an activating compound of formula 57: 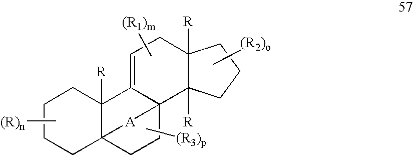
wherein, independently for each occurrence:
R, R₁, R₂, and R₃ are hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₃ is alkyl, —SO₃H, monosaccharide, oligosaccharide, glycofuranosyl, glycopyranosyl, glucuronosyl, or glucuronide;
A is alkylene, alkenylene, or alkynylene;
n is an integer from 0 to 8 inclusive;
m is an integer from 0 to 3 inclusive;
o is an integer from 0 to 6 inclusive; and
p is an integer from 0 to 4 inclusive.

In a further embodiment, the methods comprise a compound of formula 57 and the attendant definitions wherein n is 2.

In a further embodiment, the methods comprise a compound of formula 57 and the attendant definitions wherein R is OH or methyl.

In a further embodiment, the methods comprise a compound of formula 57 and the attendant definitions wherein m is 1.

In a further embodiment, the methods comprise a compound of formula 57 and the attendant definitions wherein R₁ is methyl.

In a further embodiment, the methods comprise a compound of formula 57 and the attendant definitions wherein o is 1.

In a further embodiment, the methods comprise a compound of formula 57 and the attendant definitions wherein R₂ is C(O)CH₃.

In a further embodiment, the methods comprise a compound of formula 57 and the attendant definitions wherein p is 2.

In a further embodiment, the methods comprise a compound of formula 57 and the attendant definitions wherein R₃ is CO₂H.

In a further embodiment, the methods comprise a compound of formula 57 and the attendant definitions wherein A is alkenylene.

In a further embodiment, the methods comprise a compound of formula 57 and the attendant definitions wherein n is 2 and R is OH or methyl.

In a further embodiment, the methods comprise a compound of formula 57 and the attendant definitions wherein n is 2, R is OH or methyl, and m is 1.

In a further embodiment, the methods comprise a compound of formula 57 and the attendant definitions wherein n is 2, R is OH or methyl, m is 1, and R₁ is methyl.

In a further embodiment, the methods comprise a compound of formula 57 and the attendant definitions wherein n is 2, R is OH or methyl, m is 1, R₁ is methyl, and o is 1.

In a further embodiment, the methods comprise a compound of formula 57 and the attendant definitions wherein n is 2, R is OH or methyl, m is 1, R₁ is methyl, o is 1, and R₂ is C(O)CH₃.

In a further embodiment, the methods comprise a compound of formula 57 and the attendant definitions wherein n is 2, R is OH or methyl, m is 1, R₁ is methyl, o is 1, R₂ is C(O)CH₃, and p is 2.

In a further embodiment, the methods comprise a compound of formula 57 and the attendant definitions wherein n is 2, R is OH or methyl, m is 1, R₁ is methyl, o is 1, R₂ is C(O)CH₃, p is 2, and R₃ is CO₂H.

In a further embodiment, the methods comprise a compound of formula 57 and the attendant definitions wherein n is 2, R is OH or methyl, m is 1, R₁ is methyl, o is 1, R₂ is C(O)CH₃, p is 2, R₃ is CO₂H, and A is alkenylene.

In another embodiment, methods for activating a sirtuin protein comprise using an activating compound of formula 58: 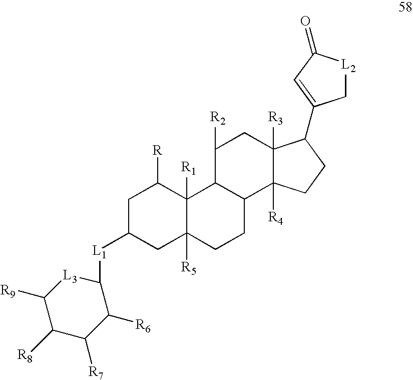
wherein, independently for each occurrence:
R, R₁, R₂, R₃, R₄, R₅, R₆, R₇, R₈, and R₉ are hydroxy, amino, cyano, halide, OR₁₁, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₁₁ is alkyl, —SO₃H, monosaccharide, oligosaccharide, glycofuranosyl, glycopyranosyl, glucuronosyl, or glucuronide;
L₁, L₂, and L₃ are O, NR₁₀, or S; and
R₁₀ is H or a substituted or unsubstituted alkyl, aryl, aralkyl, heterocyclyl, heterocyclylalkyl, heteroaryl, or heteroaralkyl.

In a further embodiment, the methods comprise a compound of formula 58 and the attendant definitions wherein R is OH.

In a further embodiment, the methods comprise a compound of formula 58 and the attendant definitions wherein R₁ is CH₂OH.

In a further embodiment, the methods comprise a compound of formula 58 and the attendant definitions wherein R₂ is OH.

In a further embodiment, the methods comprise a compound of formula 58 and the attendant definitions wherein R₃ is methyl.

In a further embodiment, the methods comprise a compound of formula 58 and the attendant definitions wherein R₄ is OH.

In a further embodiment, the methods comprise a compound of formula 58 and the attendant definitions wherein R₅ is OH.

In a further embodiment, the methods comprise a compound of formula 58 and the attendant definitions wherein R₆ is OH.

In a further embodiment, the methods comprise a compound of formula 58 and the attendant definitions wherein R₇ is OH.

In a further embodiment, the methods comprise a compound of formula 58 and the attendant definitions wherein R₈ is OH.

In a further embodiment, the methods comprise a compound of formula 58 and the attendant definitions wherein R₉ is methyl.

In a further embodiment, the methods comprise a compound of formula 58 and the attendant definitions wherein L₁ is O.

In a further embodiment, the methods comprise a compound of formula 58 and the attendant definitions wherein L₂ is O.

In a further embodiment, the methods comprise a compound of formula 58 and the attendant definitions wherein L₃ is O.

In a further embodiment, the methods comprise a compound of formula 58 and the attendant definitions wherein R is OH and R₁ is CH₂OH.

In a further embodiment, the methods comprise a compound of formula 58 and the attendant definitions wherein R is OH, R₁ is CH₂OH, and R₂ is OH.

In a further embodiment, the methods comprise a compound of formula 58 and the attendant definitions wherein R is OH, R₁ is CH₂OH, R₂ is OH, and R₃ is methyl.

In a further embodiment, the methods comprise a compound of formula 58 and the attendant definitions wherein R is OH, R₁ is CH₂OH, R₂ is OH, R₃ is methyl, and R₄ is OH.

In a further embodiment, the methods comprise a compound of formula 58 and the attendant definitions wherein R is OH, R₁ is CH₂OH, R₂ is OH, R₃ is methyl, R₄ is OH, and R₅ is OH.

In a further embodiment, the methods comprise a compound of formula 58 and the attendant definitions wherein R is OH, R₁ is CH₂OH, R₂ is OH, R₃ is methyl, R₄ is OH, R₅ is OH, and R₆ is OH.

In a further embodiment, the methods comprise a compound of formula 58 and the attendant definitions wherein R is OH, R₁ is CH₂OH, R₂ is OH, R₃ is methyl, R₄ is OH, R₅ is OH, R₆ is OH, and R₇ is OH.

In a further embodiment, the methods comprise a compound of formula 58 and the attendant definitions wherein R is OH, R₁ is CH₂OH, R₂ is OH, R₃ is methyl, R₄ is OH, R₅ is OH, R₆ is OH, R₇ is OH, and R₈ is OH.

In a further embodiment, the methods comprise a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, and R₉ is methyl.

In a further embodiment, the methods comprise a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, R₉ is methyl, and L₁ is O.

In a further embodiment, the methods comprise a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, R₉ is methyl, L₁ is O, and L₂ is O.

In a further embodiment, the methods comprise a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, R₉ is methyl, L₁ is O, L₂ is O, and L₃ is O.

In another embodiment, methods for activating a sirtuin protein comprise using an activating compound of formula 59: 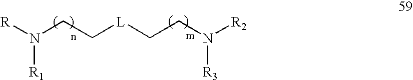
wherein, independently for each occurrence:
R, R₁, R₂, and R₃ are H or a substituted or unsubstituted alkyl, aryl, aralkyl, heterocyclyl, heterocyclylalkyl, heteroaryl, or heteroaralkyl;
L is O, NR, S, or Se; and
n and m are integers from 0 to 5 inclusive.

In a further embodiment, the methods comprise a compound of formula 59 and the attendant definitions wherein R is H.

In a further embodiment, the methods comprise a compound of formula 59 and the attendant definitions wherein R₁ is H.

In a further embodiment, the methods comprise a compound of formula 59 and the attendant definitions wherein R₂ is H.

In a further embodiment, the methods comprise a compound of formula 59 and the attendant definitions wherein R₃ is H.

In a further embodiment, the methods comprise a compound of formula 59 and the attendant definitions wherein L is Se.

In a further embodiment, the methods comprise a compound of formula 59 and the attendant definitions wherein n is 1.

In a further embodiment, the methods comprise a compound of formula 59 and the attendant definitions wherein m is 1.

In a further embodiment, the methods comprise a compound of formula 59 and the attendant definitions wherein R is H and R₁ is H.

In a further embodiment, the methods comprise a compound of formula 59 and the attendant definitions wherein R is H, R₁ is H, and R₂ is H.

In a further embodiment, the methods comprise a compound of formula 59 and the attendant definitions wherein R is H, R₁ is H, R₂ is H, and R₃ is H.

In a further embodiment, the methods comprise a compound of formula 59 and the attendant definitions wherein R is H, R₁ is H, R₂ is H, R₃ is H, and L is Se.

In a further embodiment, the methods comprise a compound of formula 59 and the attendant definitions wherein R is H, R₁ is H, R₂ is H, R₃ is H, L is Se, and n is 1.

In a further embodiment, the methods comprise a compound of formula 59 and the attendant definitions wherein R is H, R₁ is H, R₂ is H, R₃ is H, L is Se, n is 1, and m is 1.

In another embodiment, methods for activating a sirtuin protein comprise using an activating compound of formula 60: 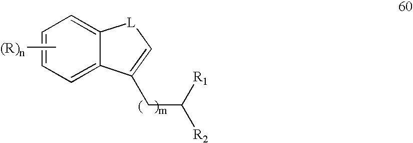
wherein, independently for each occurrence:
R is hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₁ and R₂ are H, hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₄ is alkyl, —SO₃H, monosaccharide, oligosaccharide, glycofuranosyl, glycopyranosyl, glucuronosyl, or glucuronide;
L is O, NR₃, S, or SO₂;
R₃ is H or a substituted or unsubstituted alkyl, aryl, aralkyl, heterocyclyl, heterocyclylalkyl, heteroaryl, or heteroaralkyl;
n is an integer from 0 to 4 inclusive; and
m is an integer from 1 to 5 inclusive.

In a further embodiment, the methods comprise a compound of formula 60 and the attendant definitions wherein n is 1.

In a further embodiment, the methods comprise a compound of formula 60 and the attendant definitions wherein R is Cl.

In a further embodiment, the methods comprise a compound of formula 60 and the attendant definitions wherein R₁ is NH₂.

In a further embodiment, the methods comprise a compound of formula 60 and the attendant definitions wherein R₂ is CO₂H.

In a further embodiment, the methods comprise a compound of formula 60 and the attendant definitions wherein L is SO₂.

In a further embodiment, the methods comprise a compound of formula 60 and the attendant definitions wherein m is 1.

In a further embodiment, the methods comprise a compound of formula 60 and the attendant definitions wherein n is 1 and R is Cl.

In a further embodiment, the methods comprise a compound of formula 60 and the attendant definitions wherein n is 1, R is Cl, and R₁ is NH₂.

In a further embodiment, the methods comprise a compound of formula 60 and the attendant definitions wherein n is 1, R is Cl, R₁ is NH₂, and R₂ is CO₂H.

In a further embodiment, the methods comprise a compound of formula 60 and the attendant definitions wherein n is 1, R is Cl, R₁ is NH₂, R₂ is CO₂H, and L is SO₂.

In a further embodiment, the methods comprise a compound of formula 60 and the attendant definitions wherein n is 1, R is Cl, R₁ is NH₂, R₂ is CO₂H, L is SO₂, and m is 1.

In another embodiment, methods for activating a sirtuin protein comprise using an activating compound of formula 61: 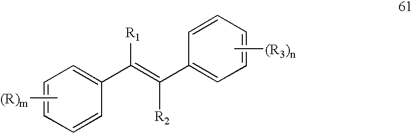
wherein, independently for each occurrence:
R, R₁, R₂, and R₃ are H, hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₄ is alkyl, —SO₃H, monosaccharide, oligosaccharide, glycofuranosyl, glycopyranosyl, glucuronosyl, or glucuronide; and
n and m are integers from 0 to 5 inclusive.

In a further embodiment, the methods comprise a compound of formula 61 and the attendant definitions wherein n is 2.

In a further embodiment, the methods comprise a compound of formula 61 and the attendant definitions wherein R is 3-hydroxy and 5-hydroxy.

In a further embodiment, the methods comprise a compound of formula 61 and the attendant definitions wherein R₁ is H.

In a further embodiment, the methods comprise a compound of formula 61 and the attendant definitions wherein R₂ is H.

In a further embodiment, the methods comprise a compound of formula 61 and the attendant definitions wherein m is 0.

In a further embodiment, the methods comprise a compound of formula 61 and the attendant definitions wherein m is 1.

In a further embodiment, the methods comprise a compound of formula 61 and the attendant definitions wherein R₃ is 4-hydroxy.

In a further embodiment, the methods comprise a compound of formula 61 and the attendant definitions wherein R₃ is 4-methoxy.

In a further embodiment, the methods comprise a compound of formula 61 and the attendant definitions wherein n is 2 and R is 3-hydroxy and 5-hydroxy.

In a further embodiment, the methods comprise a compound of formula 61 and the attendant definitions wherein n is 2, R is 3-hydroxy and 5-hydroxy, and R₁ is H.

In a further embodiment, the methods comprise a compound of formula 61 and the attendant definitions wherein n is 2, R is 3-hydroxy and 5-hydroxy, R₁ is H, and R₂ is H.

In a further embodiment, the methods comprise a compound of formula 61 and the attendant definitions wherein n is 2, R is 3-hydroxy and 5-hydroxy, R₁ is H, R₂ is H, and m is 0.

In a further embodiment, the methods comprise a compound of formula 61 and the attendant definitions wherein n is 2, R is 3-hydroxy and 5-hydroxy, R₁ is H, R₂ is H, and m is 1.

In a further embodiment, the methods comprise a compound of formula 61 and the attendant definitions wherein n is 2, R is 3-hydroxy and 5-hydroxy, R₁ is H, R₂ is H, m is 1, and R₃ is 4-hydroxy.

In a further embodiment, the methods comprise a compound of formula 61 and the attendant definitions wherein n is 2, R is 3-hydroxy and 5-hydroxy, R₁ is H, R₂ is H, m is 1, and R₃ is 4-methoxy.

In another embodiment, methods for activating a sirtuin protein comprise using an activating compound of formula 62: 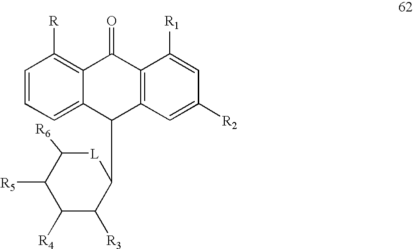
wherein, independently for each occurrence:
R, R₁, R₂, R₃, R₄, R₅, and R₆ are H, hydroxy, amino, cyano, OR₈, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₈ is alkyl, —SO₃H, monosaccharide, oligosaccharide, glycofuranosyl, glycopyranosyl, glucuronosyl, or glucuronide;
L is O, NR₇, or S; and
R₇ is H or a substituted or unsubstituted alkyl, aryl, aralkyl, heterocyclyl, heterocyclylalkyl, heteroaryl, or heteroaralkyl.

In a further embodiment, the methods comprise a compound of formula 62 and the attendant definitions wherein R is OH.

In a further embodiment, the methods comprise a compound of formula 62 and the attendant definitions wherein R₁ is OH.

In a further embodiment, the methods comprise a compound of formula 62 and the attendant definitions wherein R₂ is CH₂OH.

In a further embodiment, the methods comprise a compound of formula 62 and the attendant definitions wherein R₃ is OH.

In a further embodiment, the methods comprise a compound of formula 62 and the attendant definitions wherein R₄ is OH.

In a further embodiment, the methods comprise a compound of formula 62 and the attendant definitions wherein R₅ is OH.

In a further embodiment, the methods comprise a compound of formula 62 and the attendant definitions wherein R₆ is CH₂OH.

In a further embodiment, the methods comprise a compound of formula 62 and the attendant definitions wherein L is O.

In a further embodiment, the methods comprise a compound of formula 62 and the attendant definitions wherein R is OH and R₁ is OH.

In a further embodiment, the methods comprise a compound of formula 62 and the attendant definitions wherein R is OH, R₁ is OH, and R₂ is CH₂OH.

In a further embodiment, the methods comprise a compound of formula 62 and the attendant definitions wherein R is OH, R₁ is OH, R₂ is CH₂OH, and R₃ is OH.

In a further embodiment, the methods comprise a compound of formula 62 and the attendant definitions wherein R is OH, R₁ is OH, R₂ is CH₂OH, R₃ is OH, and R₄ is OH.

In a further embodiment, the methods comprise a compound of formula 62 and the attendant definitions wherein R is OH, R₁ is OH, R₂ is CH₂OH, R₃ is OH, R₄ is OH, and R₅ is OH.

In a further embodiment, the methods comprise a compound of formula 62 and the attendant definitions wherein R is OH, R₁ is OH, R₂ is CH₂OH, R₃ is OH, R₄ is OH, R₅ is OH, and R₆ is CH₂OH.

In a further embodiment, the methods comprise a compound of formula 62 and the attendant definitions wherein R is OH, R₁ is OH, R₂ is CH₂OH, R₃ is OH, R₄ is OH, R₅ is OH, R₆ is CH₂OH, and L is O.

In another embodiment, methods for activating a sirtuin protein comprise using an activating compound of formula 63: 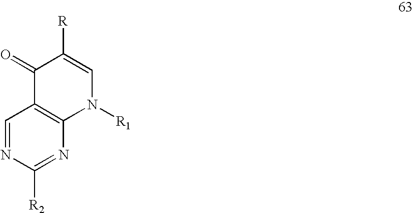
wherein, independently for each occurrence:
R, R₁, and R₂ are H, hydroxy, amino, cyano, halide, OR₃, ether, ester, amido, ketone, carboxylic acid, nitro, or a substituted or unsubstituted alkyl, aryl, aralkyl, heterocyclyl, heterocyclylalkyl, heteroaryl, or heteroaralkyl; and
R₃ is alkyl, —SO₃H, monosaccharide, oligosaccharide, glycofuranosyl, glycopyranosyl, glucuronosyl, or glucuronide.

In a further embodiment, the methods comprise a compound of formula 63 and the attendant definitions wherein R is CO₂H.

In a further embodiment, the methods comprise a compound of formula 63 and the attendant definitions wherein R₁ is ethyl.

In a further embodiment, the methods comprise a compound of formula 63 and the attendant definitions wherein R₂ is N-1-pyrrolidine.

In a further embodiment, the methods comprise a compound of formula 63 and the attendant definitions wherein R is CO₂H and R₁ is ethyl.

In a further embodiment, the methods comprise a compound of formula 63 and the attendant definitions wherein R is CO₂H and R₂ is N-1-pyrrolidine.

In a further embodiment, the methods comprise a compound of formula 63 and the attendant definitions wherein R₁ is ethyl and R₂ is N-1-pyrrolidine.

In a further embodiment, the methods comprise a compound of formula 63 and the attendant definitions wherein R is CO₂H, R₁ is ethyl, and R₂ is N-1-pyrrolidine.

In another embodiment, methods for activating a sirtuin protein comprise using an activating compound of formula 64: 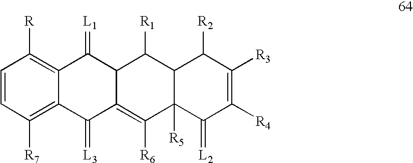
wherein, independently for each occurrence:
R, R₁, R₂, R₃, R₄, R₅, R₆, and R₇ are H, hydroxy, amino, cyano, halide, OR₉, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₉ is alkyl, —SO₃H, monosaccharide, oligosaccharide, glycofuranosyl, glycopyranosyl, glucuronosyl, or glucuronide;
L₁, L₂, and L₃ are CH₂, O, NR₈, or S; and
R₈ is H or a substituted or unsubstituted alkyl, aryl, aralkyl, heterocyclyl, heterocyclylalkyl, heteroaryl, or heteroaralkyl.

In a further embodiment, the methods comprise a compound of formula 64 and the attendant definitions wherein R is Cl.

In a further embodiment, the methods comprise a compound of formula 64 and the attendant definitions wherein R is H.

In a further embodiment, the methods comprise a compound of formula 64 and the attendant definitions wherein R₁ is OH.

In a further embodiment, the methods comprise a compound of formula 64 and the attendant definitions wherein R₂ is N(Me)₂.

In a further embodiment, the methods comprise a compound of formula 64 and the attendant definitions wherein R₃ is OH.

In a further embodiment, the methods comprise a compound of formula 64 and the attendant definitions wherein R₄ is C(O)NH₂.

In a further embodiment, the methods comprise a compound of formula 64 and the attendant definitions wherein R₅ is OH.

In a further embodiment, the methods comprise a compound of formula 64 and the attendant definitions wherein R₆ is OH.

In a further embodiment, the methods comprise a compound of formula 64 and the attendant definitions wherein R₇ is OH.

In a further embodiment, the methods comprise a compound of formula 64 and the attendant definitions wherein L₁ is CH₂.

In a further embodiment, the methods comprise a compound of formula 64 and the attendant definitions wherein L₂ is O.

In a further embodiment, the methods comprise a compound of formula 64 and the attendant definitions wherein L₃ is O.

In a further embodiment, the methods comprise a compound of formula 64 and the attendant definitions wherein R is Cl and R₁ is OH.

In a further embodiment, the methods comprise a compound of formula 64 and the attendant definitions wherein R is Cl, R₁ is OH, and R₂ is N(Me)₂.

In a further embodiment, the methods comprise a compound of formula 64 and the attendant definitions wherein R is Cl, R₁ is OH, R₂ is N(Me)₂, and R₃ is OH.

In a further embodiment, the methods comprise a compound of formula 64 and the attendant definitions wherein R is Cl, R₁ is OH, R₂ is N(Me)₂, R₃ is OH, and R₄ is C(O)NH₂.

In a further embodiment, the methods comprise a compound of formula 64 and the attendant definitions wherein R is Cl, R₁ is OH, R₂ is N(Me)₂, R₃ is OH, R₄ is C(O)NH₂, and R₅ is OH.

In a further embodiment, the methods comprise a compound of formula 64 and the attendant definitions wherein R is Cl, R₁ is OH, R₂ is N(Me)₂, R₃ is OH, R₄ is C(O)NH₂, R₅ is OH, and R₆ is OH.

In a further embodiment, the methods comprise a compound of formula 64 and the attendant definitions wherein R is Cl, R₁ is OH, R₂ is N(Me)₂, R₃ is OH, R₄ is C(O)NH₂, R₅ is OH, R₆ is OH, and R₇ is OH.

In a further embodiment, the methods comprise a compound of formula 64 and the attendant definitions wherein R is Cl, R₁ is OH, R₂ is N(Me)₂, R₃ is OH, R₄ is C(O)NH₂, R₅ is OH, R₆ is OH, R₇ is OH, and L₁ is CH₂.

In a further embodiment, the methods comprise a compound of formula 64 and the attendant definitions wherein R is Cl, R₁ is OH, R₂ is N(Me)₂, R₃ is OH, R₄ is C(O)NH₂, R₅ is OH, R₆ is OH, R₇ is OH, L₁ is CH₂, and L₂ is O.

In a further embodiment, the methods comprise a compound of formula 64 and the attendant definitions wherein R is Cl, R₁ is OH, R₂ is N(Me)₂, R₃ is OH, R₄ is C(O)NH₂, R₅ is OH, R₆ is OH, R₇ is OH, L₁ is CH₂, L₂ is O, and L₃ is O.

In a further embodiment, the methods comprise a compound of formula 64 and the attendant definitions wherein R is H and R₁ is OH.

In a further embodiment, the methods comprise a compound of formula 64 and the attendant definitions wherein R is H, R₁ is OH, and R₂ is N(Me)₂.

In a further embodiment, the methods comprise a compound of formula 64 and the attendant definitions wherein R is H, R₁ is OH, R₂ is N(Me)₂, and R₃ is OH.

In a further embodiment, the methods comprise a compound of formula 64 and the attendant definitions wherein R is H, R₁ is OH, R₂ is N(Me)₂, R₃ is OH, and R₄ is C(O)NH₂.

In a further embodiment, the methods comprise a compound of formula 64 and the attendant definitions wherein R is H, R₁ is OH, R₂ is N(Me)₂, R₃ is OH, R₄ is C(O)NH₂, and R₅ is OH.

In a further embodiment, the methods comprise a compound of formula 64 and the attendant definitions wherein R is H, R₁ is OH, R₂ is N(Me)₂, R₃ is OH, R₄ is C(O)NH₂, R₅ is OH, and R₆ is OH.

In a further embodiment, the methods comprise a compound of formula 64 and the attendant definitions wherein R is H, R₁ is OH, R₂ is N(Me)₂, R₃ is OH, R₄ is C(O)NH₂, R₅ is OH, R₆ is OH, and R₇ is OH.

In a further embodiment, the methods comprise a compound of formula 64 and the attendant definitions wherein R is H, R₁ is OH, R₂ is N(Me)₂, R₃ is OH, R₄ is C(O)NH₂, R₅ is OH, R₆ is OH, R₇ is OH, and L₁ is CH₂.

In a further embodiment, the methods comprise a compound of formula 64 and the attendant definitions wherein R is H, R₁ is OH, R₂ is N(Me)₂, R₃ is OH, R₄ is C(O)NH₂, R₅ is OH, R₆ is OH, R₇ is OH, L₁ is CH₂, and L₂ is O.

In a further embodiment, the methods comprise a compound of formula 64 and the attendant definitions wherein R is H, R₁ is OH, R₂ is N(Me)₂, R₃ is OH, R₄ is C(O)NH₂, R₅ is OH, R₆ is OH, R₇ is OH, L₁ is CH₂, L₂ is O, and L₃ is O.

In another embodiment, methods for activating a sirtuin protein comprise using an activating compound of formula 65: 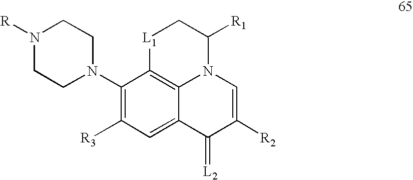
wherein, independently for each occurrence:
R is H or a substituted or unsubstituted alkyl, aryl, aralkyl, heterocyclyl, heterocyclylalkyl, heteroaryl, or heteroaralkyl;
R₁, R₂, and R₃ are hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₄ is alkyl, —SO₃H, monosaccharide, oligosaccharide, glycofuranosyl, glycopyranosyl, glucuronosyl, or glucuronide; and
L₁ and L₂ are O, NR, or S.

In a further embodiment, the methods comprise a compound of formula 65 and the attendant definitions wherein R is methyl.

In a further embodiment, the methods comprise a compound of formula 65 and the attendant definitions wherein R₁ is methyl.

In a further embodiment, the methods comprise a compound of formula 65 and the attendant definitions wherein R₂ is CO₂H.

In a further embodiment, the methods comprise a compound of formula 65 and the attendant definitions wherein R₃ is F.

In a further embodiment, the methods comprise a compound of formula 65 and the attendant definitions wherein L₁ is O.

In a further embodiment, the methods comprise a compound of formula 65 and the attendant definitions wherein L₂ is O.

In a further embodiment, the methods comprise a compound of formula 65 and the attendant definitions wherein R is methyl and R₁ is methyl.

In a further embodiment, the methods comprise a compound of formula 65 and the attendant definitions wherein R is methyl, R₁ is methyl, and R₂ is CO₂H.

In a further embodiment, the methods comprise a compound of formula 65 and the attendant definitions wherein R is methyl, R₁ is methyl, R₂ is CO₂H, and R₃ is F.

In a further embodiment, the methods comprise a compound of formula 65 and the attendant definitions wherein R is methyl, R₁ is methyl, R₂ is CO₂H, R₃ is F, and L₁ is O.

In a further embodiment, the methods comprise a compound of formula 65 and the attendant definitions wherein R is methyl, R₁ is methyl, R₂ is CO₂H, R₃ is F, L₁ is O, and L₂ is O.

Exemplary activating compounds are those listed in the appended Tables having a ratio to control rate of more than one. A preferred compound of formula 8 is Dipyridamole; a preferred compound of formula 12 is Hinokitiol; a preferred compound of formula 13 is L-(+)-Ergothioneine; a preferred compound of formula 19 is Caffeic Acid Phenol Ester; a preferred compound of formula 20 is MCI-186 and a preferred compound of formula 21 is HBED (Supplementary Table 6). Activating compounds may also be oxidized forms of the compounds of Table 21.

Also included are pharmaceutically acceptable addition salts and complexes of the compounds of formulas 1-25, 30, and 32-65. In cases wherein the compounds may have one or more chiral centers, unless specified, the compounds contemplated herein may be a single stereoisomer or racemic mixtures of stereoisomers.

In cases in which the compounds have unsaturated carbon-carbon double bonds, both the cis (Z) and trans (E) isomers are contemplated herein. In cases wherein the compounds may exist in tautomeric forms, such as keto-enol tautomers, such as 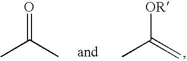
each tautomeric form is contemplated as being included within the methods presented herein, whether existing in equilibrium or locked in one form by appropriate substitution with R′. The meaning of any substituent at any one occurrence is independent of its meaning, or any other substituent's meaning, at any other occurrence.

Also included in the methods presented herein are prodrugs of the compounds of formulas 1-25, 30, and 32-65. Prodrugs are considered to be any covalently bonded carriers that release the active parent drug in vivo. Metabolites, such as in vivo degradation products, of the compounds described herein are also included.

Analogs and derivatives of the above-described compounds can also be used for activating a member of the sirtuin protein family. For example, derivatives or analogs may make the compounds more stable or improve their ability to traverse cell membranes or being phagocytosed or pinocytosed. Exemplary derivatives include glycosylated derivatives, as described, e.g., in U.S. Pat. No. 6,361,815 for resveratrol. Other derivatives of resveratrol include cis- and trans-resveratrol and conjugates thereof with a saccharide, such as to form a glucoside (see, e.g., U.S. Pat. No. 6,414,037). Glucoside polydatin, referred to as piceid or resveratrol 3-O-beta-D-glucopyranoside, can also be used. Saccharides to which compounds may be conjugated include glucose, galactose, maltose, lactose and sucrose. Glycosylated stilbenes are further described in Regev-Shoshani et al. Biochemical J. (published on Apr. 16, 2003 as BJ20030141). Other derivatives of compounds described herein are esters, amides and prodrugs. Esters of resveratrol are described, e.g., in U.S. Pat. No. 6,572,882. Resveratrol and derivatives thereof can be prepared as described in the art, e.g., in U.S. Pat. Nos. 6,414,037; 6,361,815; 6,270,780; 6,572,882; and Brandolini et al. (2002) J. Agric. Food. Chem. 50:7407. Derivatives of hydroxyflavones are described, e.g., in U.S. Pat. No. 4,591,600. Resveratrol and other activating compounds can also be obtained commercially, e.g., from Sigma.

In certain embodiments, if an activating compound occurs naturally, it may be at least partially isolated from its natural environment prior to use. For example, a plant polyphenol may be isolated from a plant and partially or significantly purified prior to use in the methods described herein. An activating compound may also be prepared synthetically, in which case it would be free of other compounds with which it is naturally associated. In an illustrative embodiment, an activating composition comprises, or an activating compound is associated with, less than about 50%, 10%, 1%, 0.1%, 10⁻²% or 10⁻³% of a compound with which it is naturally associated.

Sirtuin proteins may be activated in vitro, e.g., in a solution or in a cell. In one embodiment, a sirtuin protein is contacted with an activating compound in a solution. A sirtuin is activated by a compound when at least one of its biological activities, e.g., deacetylation activity, is higher in the presence of the compound than in its absence. Activation may be by a factor of at least about 10%, 30%, 50%, 100% (i.e., a factor of two), 3, 10, 30, or 100 fold. The extent of activation can be determined, e.g., by contacting the activated sirtuin with a deacetylation substrate and determining the extent of deacetylation of the substrate, as further described herein. The observation of a lower level of acetylation of the substrate in the presence of a test sirtuin relative to the presence of a non activated control sirtuin indicates that the test sirtuin is activated. The solution may be a reaction mixture. The solution may be in a dish, e.g., a multiwell dish. Sirtuin proteins may be prepared recombinantly or isolated from cells according to methods known in the art.

In another embodiment, a cell comprising a sirtuin deacetylase protein is contacted with an activating compound. The cell may be a eukaryotic cell, e.g., a mammalian cell, such as a human cell, a yeast cell, a non-human primate cell, a bovine cell, an ovine cell, an equine cell, a porcine cell, a sheep cell, a bird (e.g., chicken or fowl) cell, a canine cell, a feline cell or a rodent (mouse or rat) cell. It can also be a non-mammalian cell, e.g., a fish cell. Yeast cells include S. cerevesiae and C. albicans. The cell may also be a prokaryotic cell, e.g., a bacterial cell. The cell may also be a single-cell microorganism, e.g., a protozoan. The cell may also be a metazoan cell, a plant cell or an insect cell. The application of the methods decribed herein to a large number of cell types is based at least on the high convervation of sirtuins from humans to fungi, protozoans, metazoans and plants.

In one embodiment, the cells are in vitro. A cell may be contacted with a solution having a concentration of an activating compound of less than about 0.1 μM; 0.5 μM; less than about 1 μM; less than about 10 μM or less than about 100 μM. The concentration of the activating compound may also be in the range of about 0.1 to 1 μM, about 1 to 10 μM or about 10 to 100 μM. The appropriate concentration may depend on the particular compound and the particular cell used as well as the desired effect. F or example, a cell may be contacted with a “sirtuin activating” concentration of an activating compound, e.g., a concentration sufficient for activating the sirtuin by a factor of at least 10%, 30%, 50%, 100%, 3, 10, 30, or 100 fold.

In certain embodiments, a cell is contacted with an activating compound in vivo, such as in a subject. The subject can be a human, a non-human primate, a bovine, an ovine, an equine, a porcine, a sheep, a canine, a feline or a rodent (mouse or rat). For example, an activating compound may be administered to a subject. Administration may be local, e.g., topical, parenteral, oral, or other depending on the desired result of the administration (as further described herein). Administration may be followed by measuring a factor in the subject or the cell, such as the activity of the sirtuin, lifespan or stress resistance. In an illustrative embodiment, a cell is obtained from a subject following administration of an activating compound to the subject, such as by obtaining a biopsy, and the activity of the sirtuin is determined in the biopsy. The cell may be any cell of the subject, but in cases in which an activating compound is administered locally, the cell is preferably a cell that is located in the vicinity of the site of administration.

Also provided are methods for modulating the acetylation level of p53 proteins. As shown herein (see, e.g., the Examples), lysine 382 of p53 proteins in cells is deacetylated following incubation of cells in the presence of low concentrations of resveratrol. Accordingly, “p53 deacetylating concentrations” of compounds include, e.g., concentrations of less than about 0.1 μM, 0.5 μM, 1 μM, 3 μM, 50 μM, 100 μM or 300 μM. It has also been shown herein that p53 proteins in cells are acetylated in the presence of higher concentrations of resveratrol. Accordingly, “p53 acetylating concentrations” of compounds include, e.g., concentrations of at least about 10 μM, 30 μM, 100 μM or 300 μM. The level of acetylation of p53 can be determined by methods known in the art, e.g., as further described in the Examples.

Other methods contemplated are methods for protecting a cell against apoptosis. Without wanting to be limited to a particular mechanism of action, but based at least in part on the fact that acetylation of p53 proteins activates p53 proteins and that activated p53 proteins induce apoptosis, incubating cells comprising p53 proteins in the presence of a p53 deacetylating concentration of an activating compound prevents the induction of apoptosis of the cells. Accordingly, a cell can be protected from apoptosis by activating sirtuins by contacting the cell with an amount of an activating compound sufficient or adequate for protecting against apoptosis, e.g., less than about 0.1 μM, 0.5 μM, 1 μM, 3 μM or 10 μM. An amount sufficient or adequate for protection against apoptosis can also be determined experimentally, such as by incubating a cell with different amounts of an activating compound, subjecting the cell to an agent or condition that induces apoptosis, and comparing the extent of apoptosis in the presence of different concentrations or the absence of an enhancing compound and determining the concentration that provides the desired protection. Determining the level of apoptosis in a population of cells can be performed according to methods known in the art.

Yet other methods contemplated herein are methods for inducing apoptosis in a cell. Without wanting to be limited to a particular mechanism of action, as shown in the Examples, at certain concentrations of compounds, p53 proteins are acetylated rather than deacetylated, thereby activating the p53 proteins, and inducing apoptosis. Apoptosis inducing concentrations of compounds may be, e.g., at least about 10 μM, 30 μM, 100 μM or 300 μM.

Appropriate concentrations for modulating p53 deacetylation and apoptosis can be determined according to methods, e.g., those described herein. Concentrations may vary slightly from one cell to another, from one activating compound to another and whether the cell is isolated or in an organism.

Cells in which p53 acetylation and apoptosis may be modulated can be in vitro, e.g., in cell culture, or in vivo, e.g., in a subject. Administration of an activating compound to a subject can be conducted as further described herein. The level of p53 acetylation and/or apoptosis in cells of the subject can be determined, e.g., by obtaining a sample of cells from the subject and conducting an in vitro analysis of the level of p53 acetylation and/or apoptosis.

Also provided herein are methods for extending the lifespan of a eukaryotic cell and/or increasing its resistance to stress comprising, e.g., contacting the eukaryotic cell with a compound, e.g., a polyphenol compound. Exemplary compounds include the activating compounds described herein, such as compounds of the stilbene, flavone and chalcone families. Although the Examples show that quercetin and piceatannol, which activate sirtuins, were not found to significantly affect the lifespan of eukaryotic cells, it is believed that this may be the result of a lack of entry of the compounds into the cell or potentially the existence of another pathway overriding activation of sirtuins. Derivatives and analogs of these compounds or administration of these compounds to other cells or by other methods are expected to activate sirtuins.

In one embodiment, methods for extending the lifespan of a eukaryotic cell and/or increasing its resistance to stress comprise contacting the cell with a stilbene, chalcone, or flavone compound represented by formula 7: 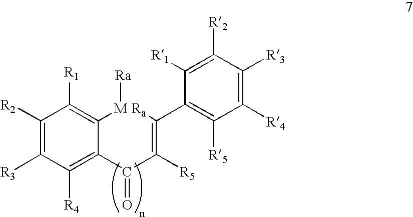
wherein, independently for each occurrence,
M is absent or O;
R₁, R₂, R₃, R₄, R₅, R′₁, R′₂, R′₃, R′₄, and R′₅ represent H, alkyl, aryl, heteroaryl, aralkyl, alkaryl, heteroaralkyl, halide, NO₂, SR, OR, N(R)₂, or carboxyl;
Rₐ represents H or the two instances of Rₐ form a bond;
R represents H, alkyl, aryl, —SO₃H, monosaccharide, oligosaccharide, glycofuranosyl, glycopyranosyl, glucuronosyl, or glucuronide; and
n is 0 or 1.

In a further embodiment, the methods comprise a compound represented by formula 7 and the attendant definitions, wherein n is 0. In a further embodiment, the methods comprise a compound represented by formula 7 and the attendant definitions, wherein n is 1. In a further embodiment, the methods comprise a compound represented by formula 7 and the attendant definitions, wherein M is absent. In a further embodiment, the methods comprise a compound represented by formula 7 and the attendant definitions, wherein M is O. In a further embodiment, the methods comprise a compound represented by formula 7 and the attendant definitions, wherein Rₐ is H. In a further embodiment, the methods comprise a compound represented by formula 7 and the attendant definitions, wherein M is O and the two Rₐ form a bond. In a further embodiment, the methods comprise a compound represented by formula 7 and the attendant definitions, wherein R₅ is H. In a further embodiment, the methods comprise a compound represented by formula 7 and the attendant definitions, wherein R₅ is OH. In a further embodiment, the methods comprise a compound represented by formula 7 and the attendant definitions, wherein R₁, R₃, and R′₃ are OH. In a further embodiment, the methods comprise a compound represented by formula 7 and the attendant definitions, wherein R₂, R₄, R′₂, and R′₃ are OH. In a further embodiment, the methods comprise a compound represented by formula 7 and the attendant definitions, wherein R₂, R′₂, and R′₃ are OH.

In a further embodiment, methods for extending the lifespan of a eukaryotic cell comprise contacting the cell with a compound represented by formula 7 and the attendant definitions, wherein n is 0; M is absent; Rₐ is H; R₅ is H; R₁, R₃, and R′₃ are OH; and R₂, R₄, R′, R′₂, R′₄, and R′₅ are H. In a further embodiment, the methods comprise a compound represented by formula 7 and the attendant definitions, wherein n is 1; M is absent; Rₐ is H; R₅ is H; R₂, R₄, R′₂, and R′₃ are OH; and R₁, R₃, R′₁, R′₄, and R′₅ are H. In a further embodiment, the methods comprise a compound represented by formula 7 and the attendant definitions, wherein n is 1; M is O; the two Rₐ form a bond; R₅ is OH; R₂, R′₂, and R′₃ are OH; and R₁, R₃, R₄, R′₁, R′₄, and R′₅ are H.

The eukaryotic cell whose lifespan may be extended can be a human, a non-human primate, a bovine, an ovine, an equine, a porcine, a sheep, a canine, a feline, a rodent (mouse or rat) or a yeast cell. A yeast cell may be Saccharomyces cerevisiae or Candida albicans. Concentrations of compounds for this purpose may be about 0.1 μM, 0.3 μM, 0.5 μM, 1 μM, 3 μM, 10 μM, 30 μM, 100 μM or 300 μM. Based at least on the high conservation of Sir2 proteins in various organisms, lifespan can also be prolonged in prokaryotes, protozoans, metazoans, insects and plants.

The cell may be in vitro or in vivo. In some embodiments, a life extending compound is administered to an organism (e.g., a subject) such as to induce hormesis, i.e., an increasing resistance to mild stress that results in increasing the lifespan of the organism. In fact, it has been shown that SIR2 is essential for the increased longevity provided by calorie restriction, a mild stress, that extends the lifespan of every organism it has been tested on (Lin et al. (2000) Science 249:2126). For example, overexpression of a Caenorhabditis elegans SIR2 homologue, sir-2.1, increases lifespan via a forkhead transcription factor, DAF-16, and a SIR2 gene has recently been implicated in lifespan regulation in Drosophila melanogaster (Rogina et al. Science (2002) 298:1745). Furthermore, the closest human Sir2 homologue, SIRT1, promotes survival in human cells by down-regulating the activity of the tumor suppressor p53 (Tissenbaum et al. Nature 410, 227-30 (2001); Rogina et al. Science 298:1745 (2002); and Vaziri, H. et al. Cell 107, 149-59. (2001)). The role of SIR2 in stress resistance and cell longevity is further supported by the identification of PNC1 as a calorie restriction- and stress-responsive gene that increases lifespan and stress resistance of cells by depleting intracellular nicotinamide (Anderson et al. (2003) Nature 423:181 and Bitterman et al. (2002) J. Biol. Chem. 277: 45099). Accordingly, compounds may be administered to a subject for protecting the cells of the subject from stresses and thereby extending the lifespan of the cells of the subject.

Also encompassed are methods for inhibiting sirtuins; inhibiting deacetylation of p53, e.g., for stimulating acetylation of p53; stimulating apoptosis; reducing lifespan and/or rendering cells or organisms more sensitive to stresses. Methods may include contacting a cell or a molecule, such as a sirtuin or a p53 protein, with a compound that inhibits sirtuins, i.e., an “inhibiting compound” or “sirtuin inhibitory compound.” Exemplary inhibiting compounds are set forth in Tables 1-13 and 22 (compounds for which the ratio to control rate is <1). Another compound is Mercury, (2-hydroxy-5-nitrophenyl)(6-thioguanosinato-N7,S6). The compounds of Tables 1-8 may be obtained from Biomol, Sigma/Aldrich or Indofine.

A sirtuin inhibitory compound may have a formula selected from the group of formulas 26-29, 31, and 66-68: 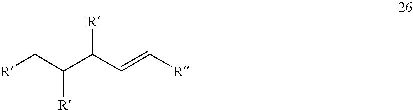
wherein, independently for each occurrence,
R′ represents H, halogen, NO₂, SR, OR, NR₂, alkyl, aryl, aralkyl, or carboxy;
R represents H, alkyl, aryl, aralkyl, heteroaralkyl, —SO₃H, monosaccharide, oligosaccharide, glycofuranosyl, glycopyranosyl, glucuronosyl, or glucuronide; and
R″ represents alkyl, alkenyl, or alkynyl; 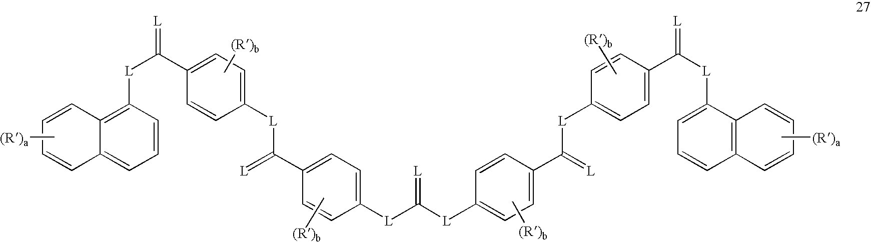
wherein, independently for each occurrence,
L represents O, NR, or S;
R represents H, alkyl, aryl, aralkyl, heteroaralkyl, —SO₃H, monosaccharide, oligosaccharide, glycofuranosyl, glycopyranosyl, glucuronosyl, or glucuronide; and
R′ represents H, halogen, NO₂, SR, SO₃, OR, NR₂, alkyl, aryl, aralkyl, or carboxy;
a represents an integer from 1 to 7 inclusive; and
b represents an integer from 1 to 4 inclusive; 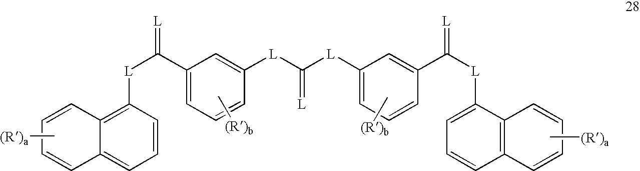
wherein, independently for each occurrence,
L represents O, NR, or S;
R represents H, alkyl, aryl, aralkyl, heteroaralkyl, —SO₃H, monosaccharide, oligosaccharide, glycofuranosyl, glycopyranosyl, glucuronosyl, or glucuronide; and
R′ represents H, halogen, NO₂, SR, SO₃, OR, NR₂, alkyl, aryl, or carboxy;
a represents an integer from 1 to 7 inclusive; and
b represents an integer from 1 to 4 inclusive; 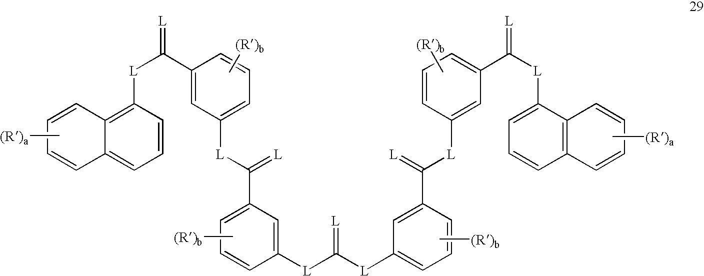
wherein, independently for each occurrence,
L represents O, NR, or S;
R represents H, alkyl, aryl, aralkyl, heteroaralkyl, —SO₃H, monosaccharide, oligosaccharide, glycofuranosyl, glycopyranosyl, glucuronosyl, or glucuronide; and
R′ represents H, halogen, NO₂, SR, SO₃, OR, NR₂, alkyl, aryl, aralkyl, or carboxy;
a represents an integer from 1 to 7 inclusive; and
b represents an integer from 1 to 4 inclusive; 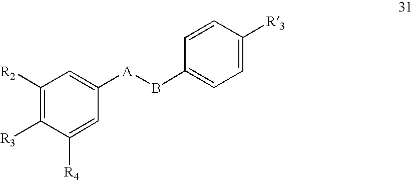
wherein, independently for each occurrence,
R₂, R₃, and R₄ are H, OR, or O-alkyl;
R represents H, —SO₃H, monosaccharide, oligosaccharide, glycofuranosyl, glycopyranosyl, glucuronosyl, or glucuronide; and
R′₃ is H or NO₂; and
A-B is an ethenylene or amido group.

In a further embodiment, the inhibiting compound is represented by formula 31 and the attendant definitions, wherein R₃ is OH, A-B is ethenylene, and R′₃ is H.

In a further embodiment, the inhibiting compound is represented by formula 31 and the attendant definitions, wherein R₂ and R₄ are OH, A-B is an amido group, and R′₃ is H.

In a further embodiment, the inhibiting compound is represented by formula 31 and the attendant definitions, wherein R₂ and R₄ are OMe, A-B is ethenylene, and R′₃ is NO₂.

In a further embodiment, the inhibiting compound is represented by formula 31 and the attendant definitions, wherein R₃ is OMe, A-B is ethenylene, and R′₃ is H.

In another embodiment, methods for activating a sirtuin protein comprise using an activating compound of formula 66: 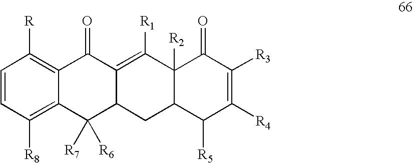
wherein, independently for each occurrence:
R, R₁, R₂, R₃, R₄, R₅, R₆, R₇, and R₈ are H, hydroxy, amino, cyano, halide, OR₉, ether, ester, amido, ketone, carboxylic acid, nitro, or a substituted or unsubstituted alkyl, aryl, aralkyl, heterocyclyl, heterocyclylalkyl, heteroaryl, or heteroaralkyl; and
R₉ represents alkyl, —SO₃H, monosaccharide, oligosaccharide, glycofuranosyl, glycopyranosyl, glucuronosyl, or glucuronide.

In a further embodiment, the methods comprise a compound of formula 66 and the attendant definitions wherein R is OH.

In a further embodiment, the methods comprise a compound of formula 66 and the attendant definitions wherein R₁ is OH.

In a further embodiment, the methods comprise a compound of formula 66 and the attendant definitions wherein R₂ is OH.

In a further embodiment, the methods comprise a compound of formula 66 and the attendant definitions wherein R₃ is C(O)NH₂.

In a further embodiment, the methods comprise a compound of formula 66 and the attendant definitions wherein R₄ is OH.

In a further embodiment, the methods comprise a compound of formula 66 and the attendant definitions wherein R₅ is NMe₂.

In a further embodiment, the methods comprise a compound of formula 66 and the attendant definitions wherein R₆ is methyl.

In a further embodiment, the methods comprise a compound of formula 66 and the attendant definitions wherein R₇ is OH.

In a further embodiment, the methods comprise a compound of formula 66 and the attendant definitions wherein R₈ is Cl.

In a further embodiment, the methods comprise a compound of formula 66 and the attendant definitions wherein R is OH and R₁ is OH.

In a further embodiment, the methods comprise a compound of formula 66 and the attendant definitions wherein R is OH, R₁ is OH, and R₂ is OH.

In a further embodiment, the methods comprise a compound of formula 66 and the attendant definitions wherein R is OH, R₁ is OH, R₂ is OH, and R₃ is C(O)NH₂.

In a further embodiment, the methods comprise a compound of formula 66 and the attendant definitions wherein R is OH, R₁ is OH, R₂ is OH, R₃ is C(O)NH₂, and R₄ is OH.

In a further embodiment, the methods comprise a compound of formula 66 and the attendant definitions wherein R is OH, R₁ is OH, R₂ is OH, R₃ is C(O)NH₂, R₄ is OH, and R₅ is NMe₂.

In a further embodiment, the methods comprise a compound of formula 66 and the attendant definitions wherein R is OH, R₁ is OH, R₂ is OH, R₃ is C(O)NH₂, R₄ is OH, R₅ is NMe₂, and R₆ is methyl.

In a further embodiment, the methods comprise a compound of formula 66 and the attendant definitions wherein R is OH, R₁ is OH, R₂ is OH, R₃ is C(O)NH₂, R₄ is OH, R₅ is NMe₂, R₆ is methyl, and R₇ is OH.

In a further embodiment, the methods comprise a compound of formula 66 and the attendant definitions wherein R is OH, R₁ is OH, R₂ is OH, R₃ is C(O)NH₂, R₄ is OH, R₅ is NMe₂, R₆ is methyl, R₇ is OH, and R₈ is Cl.

In another embodiment, methods for inhibiting a sirtuin protein comprise using an inhibiting compound of formula 67: 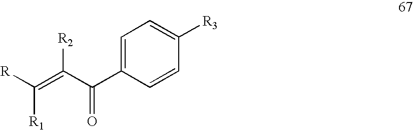
wherein, independently for each occurrence:
R, R₁, R₂, and R₃ are H, hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl; and
R₄ represents alkyl, —SO₃H, monosaccharide, oligosaccharide, glycofuranosyl, glycopyranosyl, glucuronosyl, or glucuronide.

In a further embodiment, the methods comprise a compound of formula 67 and the attendant definitions wherein R is Cl.

In a further embodiment, the methods comprise a compound of formula 67 and the attendant definitions wherein R₁ is H.

In a further embodiment, the methods comprise a compound of formula 67 and the attendant definitions wherein R₂ is H.

In a further embodiment, the methods comprise a compound of formula 67 and the attendant definitions wherein R₃ is Br.

In a further embodiment, the methods comprise a compound of formula 67 and the attendant definitions wherein R is Cl and R₁ is H.

In a further embodiment, the methods comprise a compound of formula 67 and the attendant definitions wherein R is Cl, R₁ is H, and R₂ is H.

In a further embodiment, the methods comprise a compound of formula 67 and the attendant definitions wherein R is Cl, R₁ is H, R₂ is H, and R₃ is Br.

In another embodiment, methods for inhibiting a sirtuin protein comprise using an inhibiting compound of formula 68: 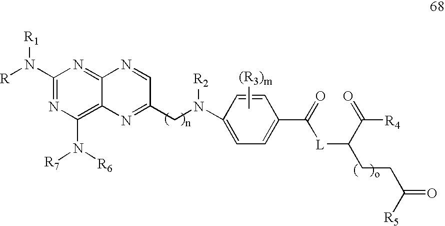
wherein, independently for each occurrence:
R, R₁, R₂, R₆, and R₇ are H or a substituted or unsubstituted alkyl, aryl, aralkyl, heterocyclyl, heterocyclylalkyl, heteroaryl, or heteroaralkyl;
R₃, R₄, and R₅ are H, hydroxy, amino, cyano, halide, OR₆, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₆ represents alkyl, —SO₃H, monosaccharide, oligosaccharide, glycofuranosyl, glycopyranosyl, glucuronosyl, or glucuronide.

L is O, NR, or S;
m is an integer from 0 to 4 inclusive; and
n and o are integers from 0 to 6 inclusive.

In a further embodiment, the methods comprise a compound of formula 68 and the attendant definitions wherein R is H.

In a further embodiment, the methods comprise a compound of formula 68 and the attendant definitions wherein R₁ is H.

In a further embodiment, the methods comprise a compound of formula 68 and the attendant definitions wherein R₂ is methyl.

In a further embodiment, the methods comprise a compound of formula 68 and the attendant definitions wherein m is 0.

In a further embodiment, the methods comprise a compound of formula 68 and the attendant definitions wherein R₄ is OH.

In a further embodiment, the methods comprise a compound of formula 68 and the attendant definitions wherein R₅ is OH.

In a further embodiment, the methods comprise a compound of formula 68 and the attendant definitions wherein R₆ is H.

In a further embodiment, the methods comprise a compound of formula 68 and the attendant definitions wherein R₇ is H.

In a further embodiment, the methods comprise a compound of formula 68 and the attendant definitions wherein L is NH.

In a further embodiment, the methods comprise a compound of formula 68 and the attendant definitions wherein n is 1.

In a further embodiment, the methods comprise a compound of formula 68 and the attendant definitions wherein o is 1.

In a further embodiment, the methods comprise a compound of formula 68 and the attendant definitions wherein R is H and R₁ is H.

In a further embodiment, the methods comprise a compound of formula 68 and the attendant definitions wherein R is H, R₁ is H, and R₂ is methyl.

In a further embodiment, the methods comprise a compound of formula 68 and the attendant definitions wherein R is H, R₁ is H, R₂ is methyl, and m is 0.

In a further embodiment, the methods comprise a compound of formula 68 and the attendant definitions wherein R is H, R₁ is H, R₂ is methyl, m is 0, and R₄ is OH.

In a further embodiment, the methods comprise a compound of formula 68 and the attendant definitions wherein R is H, R₁ is H, R₂ is methyl, m is 0, R₄ is OH, and R₅ is OH.

In a further embodiment, the methods comprise a compound of formula 68 and the attendant definitions wherein R is H, R₁ is H, R₂ is methyl, m is 0, R₄ is OH, R₅ is OH, and R₆ is H.

In a further embodiment, the methods comprise a compound of formula 68 and the attendant definitions wherein R is H, R₁ is H, R₂ is methyl, m is 0, R₄ is OH, R₅ is OH, R₆ is H, and R₇ is H.

In a further embodiment, the methods comprise a compound of formula 68 and the attendant definitions wherein R is H, R₁ is H, R₂ is methyl, m is 0, R₄ is OH, R₅ is OH, R₆ is H, R₇ is H, and L is NH.

In a further embodiment, the methods comprise a compound of formula 68 and the attendant definitions wherein R is H, R₁ is H, R₂ is methyl, m is 0, R₄ is OH, R₅ is OH, R₆ is H, R₇ is H, L is NH, and n is 1.

In a further embodiment, the methods comprise a compound of formula 68 and the attendant definitions wherein R is H, R₁ is H, R₂ is methyl, m is 0, R₄ is OH, R₅ is OH, R₆ is H, R₇ is H, L is NH, n is 1, and o is 1.

Inhibitory compounds may also be oxidized forms of the compounds of Table 22. An oxidized form of chlortetracyclin may be an activator.

Also included are pharmaceutically acceptable addition salts and complexes of the compounds of formulas 26-29, 31 and 66-68. In cases wherein the compounds may have one or more chiral centers, unless specified, the compounds contemplated herein may be a single stereoisomer or racemic mixtures of stereoisomers.

Exemplary inhibitory compounds are those set forth in the appended Tables for which the “ratio to control rate” is lower than one.

In cases in which the compounds have unsaturated carbon-carbon double bonds, both the cis (Z) and trans (E) isomers are contemplated herein. In cases wherein the compounds may exist in tautomeric forms, such as keto-enol tautomers, such as 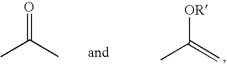
each tautomeric form is contemplated as being included within the methods presented herein, whether existing in equilibrium or locked in one form by appropriate substitution with R′. The meaning of any substituent at any one occurrence is independent of its meaning, or any other substituent's meaning, at any other occurrence.

Also included in the methods presented herein are prodrugs of the compounds of formulas 26-29, 31 and 66-68. Prodrugs are considered to be any covalently bonded carriers that release the active parent drug in vivo. Metabolites, such as in vivo degradation products, of the compounds described herein are also included.

Inhibitory compounds may be contacted with a cell, administered to a subject, or contacted with one or more molecules, such as a sirtuin protein and a p53 protein. Doses of inhibitory compounds may be similar to those of activating compounds.

Whether in vitro or in vivo, a cell may also be contacted with more than one compound (whether an activating compound or an inhibiting compound). A cell may be contacted with at least 2, 3, 5, or 10 different compounds. A cell may be contacted simultaneously or sequentially with different compounds.

Also encompassed are compositions comprising one or more activating or inhibiting compounds having a formula selected from the group of formulas 1-68. Compounds may be in a pharmaceutical composition, such as a pill or other formulation for oral administration, further described herein. Compositions may also comprise or consist of extracts of plants, red wine or other source of the compounds.

In certain embodiments, a certain biological function, e.g., extending lifespan, is modulated by any one of a compound of a genus of compounds (e.g., having formula 1), with the proviso that the genus does not include one or more specific compounds. For example, in certain embodiments, a sirtuin activating compound may be a compound of any one of formulas 1-25, 30 and 32-65 with the proviso that the compound is not resveratrol, flavone or any of the other compounds specifically cited herein. A sirtuin activating compound may also be a compound described herein that is not naturally-occurring.

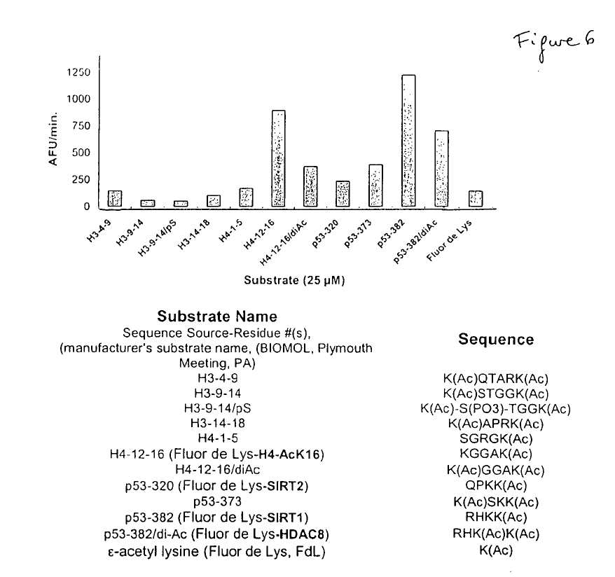

Yet other methods contemplated herein include sceening methods for identifying compounds or agents that modulate sirtuins. An agent may be a nucleic acid, such as an aptamer. Assays may be conducted in a cell based or cell free format. For example, an assay may comprise incubating (or contacting) a sirtuin with a test agent under conditions in which a sirtuin can be activated by an agent known to activate the sirtuin, and monitoring or determining the level of activation of the sirtuin in the presence of the test agent relative to the absence of the test agent. The level of activation of a sirtuin can be determined by determining its ability to deacetylate a substrate. Exemplary substrates are acetylated peptides, e.g., those set forth in FIG. 5, which can be obtained from BIOMOL (Plymouth Meeting, Pa.). Preferred substrates include peptides of p53, such as those comprising an acetylated K382. A particularly preferred substrate is the Fluor de Lys-SIRT1 (BIOMOL), i.e., the acetylated peptide Arg-His-Lys-Lys. Other substrates are peptides from human histones H3 and H4 or an acetylated amino acid (see FIG. 5). Substrates may be fluorogenic. The sirtuin may be SIRT1 or Sir2 or a portion thereof. For example, recombinant SIRT1 can be obtained from BIOMOL. The reaction may be conducted for about 30 minutes and stopped, e.g., with nicotinamide. The HDAC fluorescent activity assay/drug discovery kit (AK-500, BIOMOL Research Laboratories) may be used to determine the level of acetylation. Similar assays are described in Bitterman et al. (2002) J. Biol. Chem. 277:45099. The level of activation of the sirtuin in an assay may be compared to the level of activation of the sirtuin in the presence of one or more (separately or simultaneously) compounds described herein, which may serve as positive or negative controls. Sirtuins for use in the assays may be full length sirtuin proteins or portions thereof. Since it has been shown herein that activating compounds appear to interact with the N-terminus of SIRT1, proteins for use in the assays include N-terminal portions of sirtuins, e.g., about amino acids 1-176 or 1-255 of SIRT1; about amino acids 1-174 or 1-252 of Sir2.

In one embodiment, a screening assay comprises (i) contacting a sirtuin with a test agent and an acetylated substrate under conditions appropriate for the sirtuin to deacetylate the substrate in the absence of the test agent; and (ii) determining the level of acetylation of the substrate, wherein a lower level of acetylation of the substrate in the presence of the test agent relative to the absence of the test agent indicates that the test agent stimulates deacetylation by the sirtuin, whereas a higher level of acetylation of the substrate in the presence of the test agent relative to the absence of the test agent indicates that the test agent inhibits deacetylation by the sirtuin.

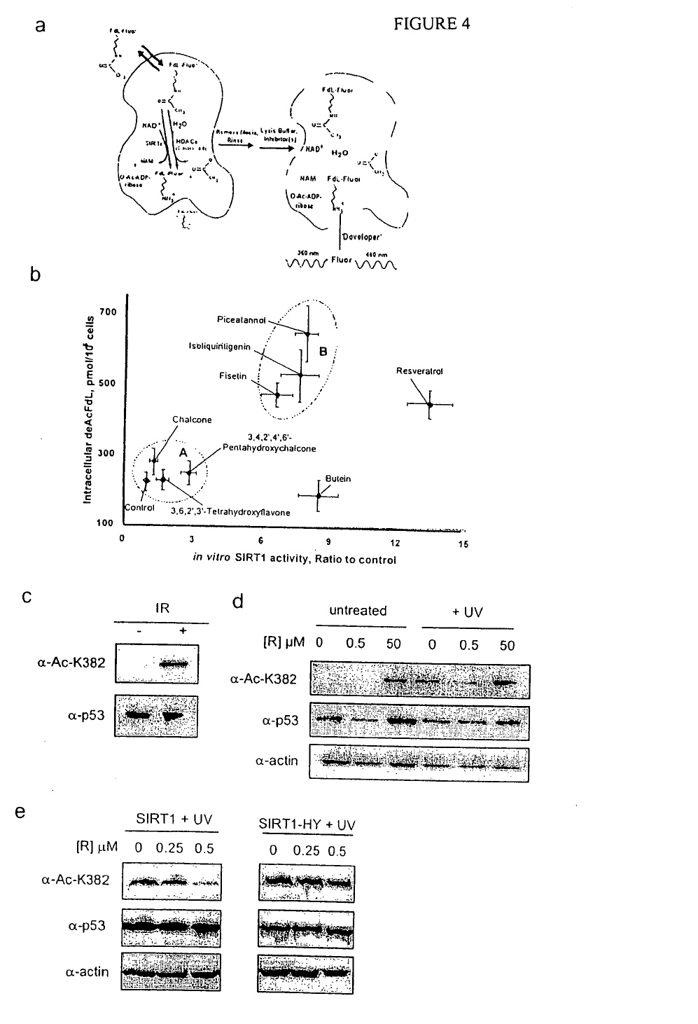

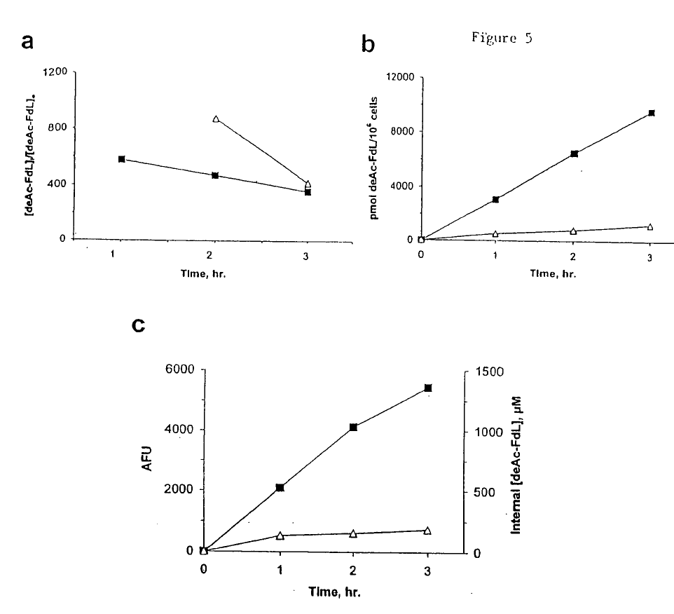

Methods for identifying an agent that modulates, e.g., stimulate or inhibit, sirtuins in vivo may comprise (i) contacting a cell with a test agent and a substrate that is capable of entering a cell in the presence of an inhibitor of class I and class II HDACs under conditions appropriate for the sirtuin to deacetylate the substrate in the absence of the test agent; and (ii) determining the level of acetylation of the substrate, wherein a lower level of acetylation of the substrate in the presence of the test agent relative to the absence of the test agent indicates that the test agent stimulates deacetylation by the sirtuin, whereas a higher level of acetylation of the substrate in the presence of the test agent relative to the absence of the test agent indicates that the test agent inhibits deacetylation by the sirtuin. A preferred substrate is an acetylated peptide, which is also prefeably fluorogenic, as further described herein (Examples). The method may further comprise lysing the cells to determine the level of acetylation of the substrate. Substrates may be added to cells at a concentration ranging from about 1 μM to about 10 mM, preferably from about 10 μM to 1 mM, even more preferably from about 100 μM to 1 mM, such as about 200 μM. A preferred substrate is an acetylated lysine, e.g., ε-acetyl lysine (Fluor de Lys, FdL) or Fluor de Lys-SIRT1. A preferred inhibitor of class I and class II HDACs is trichostatin A (TSA), which may be used at concentrations ranging from about 0.01 to 100 μM, preferably from about 0.1 to 10 μM, such as 1 μM. Incubation of cells with the test compound and the substrate may be conducted for about 10 minutes to 5 hours, preferably for about 1-3 hours. Since TSA inhibits all class I and class II HDACs, and that certain substrates, e.g., Fluor de Lys, is a poor substrate for SIRT2 and even less a substrate for SIRT3-7, such an assay may be used to identify modulators of SIRT1 in vivo. An exemplary assay is further described in the Examples and shown in FIG. 4a.

Also provided herein are assays for identifying agents that are capable of extending or reducing the lifespan of cells and/or increasing or decreasing their resistance to stress. A method may comprise incubating cells with a test agent and determining the effect of the test agent on rDNA silencing and rDNA recombination, wherein an increase in the frequency of rDNA recombination and an absence of effect on rDNA silencing in the presence of the test agent relative to the absence of the test agent indicates that the test agent extends lifespan. This assay is based at least on the observation that resveratrol reduced the frequency of rDNA recombination by about 60% in a SIR2 dependent manner, but did not increase rDNA silencing.

Assays may further comprise a step of determining the effect of a compound on cell death, e.g., neuronal cell death.

Also provided herein are methods for identifying the binding site of activating or inhibitory compounds in sirtuin proteins. In one embodiment, BML-232 (Table 10) is used. BML-232, has very similar SIRT1 activating properties to resveratrol and contains a phenylazide function. Phenylazide groups may be activated by the absorption of ultraviolet light to form reactive nitrenes. When a protein-bound phenylazide is light-activated it can react to form covalent adducts with various protein functional groups in the site to which it is bound. The photo cross-linked protein may then be analyzed by proteolysis/mass spectrometry to identify amino acid residues which may form part of the binding site for the compound. This information, in combination with published three dimensional structural information on SIRT1 homologs could be used to aid the design of new, possibly higher affinity, SIRT1 activating ligands.

Exemplary Uses

In one embodiment, cells are treated in vitro as described herein to mimic caloric restriction, such as to extend their lifespan, e.g., to keep them proliferating longer and/or increasing their resistance to stress or prevent apoptosis. That compounds described herein may increase resistance to stress is based at least on the observation that Sir2 provides stress resistance and that PNC1 modulates Sir2 activity in response to cell stress (Anderson et al. (2003) Nature 423:181). This is particularly useful for primary cell cultures (i.e., cells obtained from an organism, e.g., a human), which are known to have only a limited lifespan in culture. Treating such cells according to methods described herein, e.g., by contacting them with an activating or lifespan extending compound, will result in increasing the amount of time that the cells are kept alive in culture. Cells may be neuronal cells, such as a neural progenitor cell or a neural stem cell. “Neurons,” “neuronal cells” or “neural cells” may be sensory neurons, motor (efferent) neurons or association (connecting or interneuron) neurons, such as stellate cells, cells of Martinotti, horizontal cells of Cajal, pyramidal cells, granule cells and Purkinje cells.

Embryonic stem (ES) cells and pluripotent cells, and cells differentiated therefrom, can also be treated according to the methods described herein such as to keep the cells or progeny thereof in culture for longer periods of time. Primary cultures of cells, ES cells, pluripotent cells and progeny thereof can be used, e.g., to identify compounds having particular biological effects on the cells or for testing the toxicity of compounds on the cells (i.e., cytotoxicity assays). Such cells can also be used for transplantation into a subject, e.g., after ex vivo modification.

In other embodiments, cells that are intended to be preserved for long periods of time are treated as described herein. The cells can be cells in suspension, e.g., blood cells, serum, biological growth media, or tissues or organs. For example, blood collected from an individual for administering to an individual can be treated as described herein, such as to preserve the blood cells for longer periods of time, such as for forensic purposes. Other cells that one may treat for extending their lifespan or protect against apoptosis include cells for consumption, e.g., cells from non-human mammals (such as meat), or plant cells (such as vegetables).

Generally, sirtuin-activating compounds may be used for extending the lifespan of a cell; extending the proliferative capacity of a cell; slowing ageing of a cell; promoting the survival of a cell; delaying cellular senescence in a cell; or mimicking the effects of calorie restriction. In certain embodiments, a sirtuin-activating compound does not significantly increase the resistance of a cell to oxidative stress, although it may increase its resistance to other types of stresses. For example, a compound may increase the resistance of a cell to oxidative stress less than about 2, 5, 10, 30, or 100 fold relative to another compound, e.g., reseveratrol.

Compounds may also be applied during developmental and growth phases in mammals, plants, insects or microorganisms, in order to, e.g., alter, retard or accelerate the developmental and/or growth process.

In another embodiment, cells obtained from a subject, e.g., a human or other mammal, are treated according to methods described herein and then administered to the same or a different subject. Accordingly, cells or tissues obtained from a donor for use as a graft can be treated as described herein prior to administering to the recipient of the graft. For example, bone marrow cells can be obtained from a subject, treated ex vivo, e.g., to extend their lifespan, and then administered to a recipient. The graft can be an organ, a tissue or loose cells.

In yet other embodiments, cells are treated in vivo, e.g., to increase their lifespan or prevent apoptosis. For example, skin can be protected from aging, e.g., developing wrinkles, by treating skin, e.g., epithelial cells, as described herein. In an exemplary embodiment, skin is contacted with a pharmaceutical or cosmetic composition comprising a compound described herein. Exemplary skin afflictions or skin conditions include disorders or diseases associated with or caused by inflammation, sun damage or natural aging. For example, the compositions find utility in the prevention or treatment of contact dermatitis (including irritant contact dermatitis and allergic contact dermatitis), atopic dermatitis (also known as allergic eczema), actinic keratosis, keratinization disorders (including eczema), epidermolysis bullosa diseases (including penfigus), exfoliative dermatitis, seborrheic dermatitis, erythemas (including erythema multiforme and erythema nodosum), damage caused by the sun or other light sources, discoid lupus erythematosus, dermatomyositis, skin cancer and the effects of natural aging. The formulations may be administered topically, to the skin or mucosal tissue, as an ointment, lotion, cream, microemulsion, gel, solution or the like, as further described herein, within the context of a dosing regimen effective to bring about the desired result. A dose of active agent may be in the range of about 0.005 to about 1 micromoles per kg per day, preferably about 0.05 to about 0.75 micromoles per kg per day, more typically about 0.075 to about 0.5 micromoles per kg per day. It will be recognized by those skilled in the art that the optimal quantity and spacing of individual dosages will be determined by the nature and extent of the condition being treated, the site of administration, and the particular individual undergoing treatment, and that such optimums can be determined by conventional techniques. That is, an optimal dosing regimen for any particular patient, i.e., the number and frequency of doses, can be ascertained using conventional course of treatment determination tests. Generally, a dosing regimen involves administration of the topical formulation at least once daily, and preferably one to four times daily, until symptoms have subsided.

Topical formulations may also be used as preventive, e.g., chemopreventive, compositions. When used in a chemopreventive method, susceptible skin is treated prior to any visible condition in a particular individual.

Compounds can also be delivered locally, e.g., to a tissue or organ within a subject, such as by injection, e.g., to extend the lifespan of the cells; protect against apoptosis or induce apoptosis.

Generally, sirtuin-activating compounds may be used in methods for treating or preventing a disease or condition induced or exacerbated by cellular senescence in a subject; methods for decreasing the rate of senescence of a subject, e.g., after onset of senescence; methods for extending the lifespan of a subject; methods for treating or preventing a disease or condition relating to lifespan; methods for treating or preventing a disease or condition relating to the proliferative capacity of cells; and methods for treating or preventing a disease or condition resulting from cell damage or death. In certain embodiments, the disease or condition does not result from oxidative stress. In certain embodiments, a method does not significantly increase the resistance of the subject to oxidative stress. In certain embodiments, the method does not act by decreasing the rate of occurrence of diseases that shorten the lifespan of a subject. In certain embodiments, a method does not act by reducing the lethality caused by a disease, such as cancer.

In yet another embodiment, a sirtuin activating compound is administered to a subject, such as to generally increase the lifespan of its cells and to protect its cells against stress and/or against apoptosis. It is believed that treating a subject with a compound described herein is similar to subjecting the subject to hormesis, i.e., mild stress that is beneficial to organisms and may extend their lifespan. For example, a compound can be taken by subjects as a food or dietary supplement. In one embodiment, such a compound is a component of a multi-vitamin complex. Compounds can also be added to existing formulations that are taken on a daily basis, e.g., statins and aspirin. Compounds may also be used as food additives.

Compounds described herein could also be taken as one component of a multi-drug complex or as a supplement in addition to a multi-drug regimen. In one embodiment, this multi-drug complex or regimen would include drugs or compounds for the treatment or prevention of aging-related diseases, e.g., stroke, heart disease, arthritis, high blood pressure, Alzheimer's disease. In another embodiment, this multi-drug regimen would include chemotherapeutic drugs for the treatment of cancer. In a specific embodiment, a compound could be used to protect non-cancerous cells from the effects of chemotherapy.

Sirtuin-activating compounds may be administered to a subject to prevent or treat aging. Characteristics of aging or of older humans include skin wrinkling, graying of the hair, baldness, and cataracts, as well as hypermelanosis, osteoporosis, cerebral cortical atrophy, lymphoid depletion, thymic atrophy, increased incidence of diabetes type II, atherosclerosis, cancer, and heart disease. Nehlin et al. (2000), Annals NY Acad Sci 980:176-79. Other aspects of mammalian aging include weight loss, lordokyphosis (hunchback spine), absence of vigor, lymphoid atrophy, decreased bone density, dermal thickening and subcutaneous adipose tissue, decreased ability to tolerate stress (including heat or cold, wounding, anesthesia, and hematopoietic precursor cell ablation), liver pathology, atrophy of intestinal villi, skin ulceration, amyloid deposits, and joint diseases. Tyner et al. (2002), Nature 415:45-53.

Careful observation reveals characteristics of aging in other eukaryotes, including invertebrates. For example, characteristics of aging in the model organism C. elegans include slow movement, flaccidity, yolk accumulation, intestinal autofluorescence (lipofuscin), loss of ability to eat food or dispel waste, necrotic cavities in tissues, and germ cell appearance.

Those skilled in the art will recognize that the aging process is also manifested at the cellular level, as well as in mitochondria. Cellular aging is manifested in loss of doubling capacity, increased levels of apoptosis, changes in differentiated phenotype, and changes in metabolism, e.g., decreased levels of protein synthesis and turnover.

Given the programmed nature of cellular and organismal aging, it is possible to evaluate the “biological age” of a cell or organism by means of phenotypic characteristics that are correlated with aging. For example, biological age can be deduced from patterns of gene expression, resistance to stress (e.g., oxidative or genotoxic stress), rate of cellular proliferation, and the metabolic characteristics of cells (e.g., rates of protein synthesis and turnover, mitochondrial function, ubiquinone biosynthesis, cholesterol biosynthesis, ATP levels within the cell, levels of a Krebs cycle intermediate in the cell, glucose metabolism, nucleic acid metabolism, ribosomal translation rates, etc.). As used herein, “biological age” is a measure of the age of a cell or organism based upon the molecular characteristics of the cell or organism. Biological age is distinct from “temporal age,” which refers to the age of a cell or organism as measured by days, months, and years.

The rate of aging of an organism, e.g., an invertebrate (e.g., a worm or a fly) or a vertebrate (e.g., a rodent, e.g., a mouse) may be determined by a variety of methods, e.g., by one or more of: a) assessing the life span of the cell or the organism; (b) assessing the presence or abundance of a gene transcript or gene product in the cell or organism that has a biological age-dependent expression pattern; (c) evaluating resistance of the cell or organism to stress, e.g., genotoxic stress (e.g., etopicide, UV irradition, exposure to a mutagen, and so forth) or oxidative stress; (d) evaluating one or more metabolic parameters of the cell or organism; (e) evaluating the proliferative capacity of the cell or a set of cells present in the organism; and (f) evaluating physical appearance or behavior of the cell or organism. In one example, evaluating the rate of aging includes directly measuring the average life span of a group of animals (e.g., a group of genetically matched animals) and comparing the resulting average to the average life span of a control group of animals (e.g., a group of animals that did not receive the test compound but are genetically matched to the group of animals that did receive the test compound). Alternatively, the rate of aging of an organism can be determined by measuring an age-related parameter. Examples of age-related parameters include: appearance, e.g., visible signs of age; the expression of one or more genes or proteins (e.g., genes or proteins that have an age-related expression pattern); resistance to oxidative stress; metabolic parameters (e.g., protein synthesis or degradation, ubiquinone biosynthesis, cholesterol biosynthesis, ATP levels, glucose metabolism, nucleic acid metabolism, ribosomal translation rates, etc.); and cellular proliferation (e.g., of retinal cells, bone cells, white blood cells, etc.).

Sirtuin-activating compounds may be administered to a subject to treat or prevent aging-related consequences or diseases, such as stroke, heart disease, such as heart failure, arthritis, high blood pressure, and Alzheimer's disease. Other conditions that can be treated include ocular disorders, e.g., associated with the aging of the eye, such as cataracts, glaucoma, and macular degeneration. Sirtuin-activating compounds described herein can also be administered to subjects for treatment of diseases, e.g., chronic diseases, associated with cell death, such as to protect the cells from cell death. Exemplary diseases include those associated with neural cell death or muscular cell death, such as Parkinson's disease, Alzheimer's disease, multiple sclerosis, amniotropic lateral sclerosis (ALS; also known as Lou Gehrig's disease, Motor Neuron Disease and Charcot's disease), and muscular dystrophy; AIDS; fulminant hepatitis; diseases linked to degeneration of the brain, such as Creutzfeld-Jakob disease, retinitis pigmentosa and cerebellar degeneration; myelodysplasis such as aplastic anemia; ischemic diseases such as myocardial infarction and stroke; hepatic diseases such as alcoholic hepatitis, hepatitis B and hepatitis C; joint-diseases such as osteoarthritis; atherosclerosis; alopecia; damage to the skin due to UV light; lichen planus; atrophy of the skin; graft rejections; diabetic neuropathy; and brain or spinal cord injury.

Since age is also associated with decreased neuronal function and protein aggregation, and that calorie restriction retards this, methods that mimic calorie restriction can also be used for treating polyglutamine diseases, i.e., diseases caused by polyglutamine peptides, such as Huntington's disease, Kennedy's disease, dentatorubral-pallidoluysian atrophy, spinocerebellar ataxia, types 1, 2, 3 (Machado-Joseph), 6 and 7, TBP (severe cerebellar atrophy).

Based at least on the examples showing elevated sirtuin levels in an animal model of Alzheimer's disease, which is a neurodegenerative disease characterized by protein aggregation, other diseases in this category, such as Parkinson's disease, Pick's disease, prion disease and other spongiform encephalopathies may also be treated or prevented. Furthermore, since Alzheimer's disease is a Tauopathy, i.e., a neurodegenerative disorder involving deposition of abnormal tau protein isoforms in neurons and glial cells in the brain, diseases showing pathological aggregations of tau proteins, such as those that are associated with mutation of the tau gene on chromosome 17, may also be treated or prevented. Examples of these diseases include dementia (including Lewy Body disease, mild cognitive impairment (MCI), Primary Senile Degenerative Dementia, Alzheimer Type Senile Dementia and Alzheimer Type Dementia), Parkinsonian disorders (including Lewy Body disease and Parkinsonism-linked to chromosome 17 (FTDP-17)), progressive supranuclear palsy (also known as Steele-Richardson-Olszewski Syndrome or Disease, Progressive Supranuclear Ophthalmoplegia), Pick's disease and corticobasal degeneration.

That heart disease, e.g., myocardial infarction, can be treated by activating sirtuins, e.g., as described herein, is further supported by the observation that calorie restriction, as evidenced by a low Body Mass Index (BMI) promotes better survival following myocardial infarction (Abete et al. (2003) Am J. Clin. Nutr. 78:796).

At least based on the facts described in the Examples, additional diseases that can be treated or prevented include diseases associated with expression or over-expression of p25, a toxic co-activator of cyclin-dependent kinase 5 (cdk5), such as Alzheimer's disease, or those that are associated with a mutation in the gene for superoxide dismutatsel (SOD1G37R) and/or SOD1 aggregates encoded on chromosome 21q22.1, such as ALS. Other diseases include those that are associated with and/or caused by neuronal cell death caused by, e.g., a neurotoxic stress, such as disruption of calcium homeostasis and oxidative stress; and those in which neuronal cell death occurs as a result of a defect in cell cycle regulators, e.g., cdk5.

Other diseases that can be prevented or treated include those that relate to inflammation that results in cell death, e.g., neuronal cell death. Other diseases also include those associated with a trinucleotide repeat, such as Huntington's disease.

The agents described herein could also be used to protect non-cancerous cells from the effects of chemotherapy, such as to protect neurons in the case of preventing neuropathies, hematoxicity, renal toxicity, and gastrointestinal toxicity due to chemotherapy. In particular, the methods described herein may be used to prevent or alleviate neurodegeneration and peripheral neurophathies associated with chemotherapy, such as cancer chemotherapy (e.g., taxol or cisplatin treatment).

Cardiovascular diseases that can be treated or prevented include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to the retinal arterioles, the glomerular arterioles, the vasa nervorum, cardiac arterioles, and associated capillary beds of the eye, the kidney, the heart, and the central and peripheral nervous systems. The compounds may also be used for increasing HDL levels in plasma of an individual.

Yet other disorders that may be treated with sirtuin activators include restenosis, e.g., following coronary intervention, and disorders relating to an abnormal level of high density and low density cholesterol. Sirtuin activators may also be used for treating or preventing viral infections, such as infections by influenza, herpes or papilloma virus. They may also be used as antifungal agents, anti-inflammatory agents and neuroprotective agents.

Sirtuin-activating compounds described herein can also be administered to a subject in need of preventing the onset of disease, e.g., a subject possessing an allele linked to a disease, a subject with a genetic predisposition or family history of a disease; or preventing the advanced stages of the disease, e.g, a subject showing early stages of a disease.

Sirtuin-activating compounds described herein can also be administered to a subject suffering from an acute disease, e.g., damage to an organ or tissue, e.g., a subject suffering from stroke or myocardial infarction or a subject suffering from a spinal cord injury. Compounds can also be used to repair an alcoholic's liver.

Sirtuin-activating compounds can also be administered to subjects who have recently received or are likely to receive a dose of radiation. In one embodiment, the dose of radiation is received as part of a work-related or medical procedure, e.g., working in a nuclear power plant, flying an airplane, an X-ray, CAT scan, or the administration of a radioactive dye for medical imaging; in such an embodiment, the compound is administered as a prophylactic measure. In another embodiment, the radiation exposure is received unintentionally, e.g., as a result of an industrial accident, terrorist act, or act of war involving radioactive material. In such a case, the compound is preferably administered as soon as possible after the exposure to inhibit apoptosis and the subsequent development of acute radiation syndrome.

Based at least on the discovery that certain concentrations of activating compounds prevent deacetylation of p53 in cells and thereby may induce apoptosis in cells, the activating compounds can also be administered to a subject in conditions in which apoptosis of certain cells is desired. For example, cancer may be treated or prevented. Exemplary cancers are those of the brain and kidney; hormone-dependent cancers including breast, prostate, testicular, and ovarian cancers; lymphomas, and leukemias. In cancers associated with solid tumors, a activating compound may be administered directly into the tumor. Cancer of blood cells, e.g., leukemia can be treated by administering a activating compound into the blood stream or into the bone marrow. Benign cell growth can also be treated, e.g., warts. Other diseases that can be treated include autoimmune diseases, e.g., systemic lupus erythematosus, scleroderma, and arthritis, in which autoimmune cells should be removed. Viral infections such as herpes, HIV, adenovirus, and HTLV-1 associated malignant and benign disorders can also be treated by administration of compounds. Alternatively, cells can be obtained from a subject, treated ex vivo to remove certain undesirable cells, e.g., cancer cells, and administered back to the same or a different subject.

Chemotherapeutic agents that may be coadministered with compounds described herein as having anti-cancer activity (e.g., compounds that induce apoptosis, compounds that reduce lifespan or compounds that render cells sensitive to stress) include: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic agents may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, docetaxel, doxorubicin, epirubicin, hexamethylmelamineoxaliplatin, iphosphamide, melphalan, merchlorethamine, mitomycin, mitoxantrone, nitrosourea, paclitaxel, plicamycin, procarbazine, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, COX-2 inhibitors, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors, epidermal growth factor (EGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; chromatin disruptors.

These chemotherapeutic agents may be used by themselves with a compound described herein as inducing cell death or reducing lifespan or increasing sensitivity to stress and/or in combination with other chemotherapeutics agents. Many combinatorial therapies have been developed, including but not limited to those listed in Table 23.

TABLE 23
Exemplary conventional combination cancer chemotherapy

| Name | Therapeutic agents |
|---|---|
| ABV | Doxorubicin, Bleomycin, Vinblastine |
| ABVD | Doxorubicin, Bleomycin, Vinblastine, Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, Procarbazine, |
| | Prednisone |
| BEACOPP | Bleomycin, Etoposide, Doxorubicin, Cyclophosphamide, |
| | Vincristine, Procarbazine, Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, Prednisone, |
| | Asparaginase |
| CAMP | Cyclophosphamide, Doxorubicin, Methotrexate, |
| | Procarbazine |
| CAP | Cyclophosphamide, Doxorubicin, Cisplatin |
| CaT | Carboplatin, Paclitaxel |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide, Epirubicin, Fluorouracil |
| CEPP (B) | Cyclophosphamide, Etoposide, Prednisone, with or without/ |
| | Bleomycin |
| CEV | Cyclophosphamide, Etoposide, Vincristine |
| CF | Cisplatin, Fluorouracil or Carboplatin Fluorouracil |
| CHAP | Cyclophosphamide or Cyclophosphamide, Altretamine, |
| | Doxorubicin, Cisplatin |
| ChlVPP | Chlorambucil, Vinblastine, Procarbazine, Prednisone |
| CHOP | Cyclophosphamide, Doxorubicin, Vincristine, Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, Prednisone |
| CMFVP | Cyclophosphamide, Methotrexate, Fluorouracil, Vincristine, |
| | Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, Leucovorin, |
| | Cytarabine |
| COMP | Cyclophosphamide, Vincristine, Methotrexate, Prednisone |
| Cooper Regimen | Cyclophosphamide, Methotrexate, Fluorouracil, Vincristine, |
| | Prednisone |
| COP | Cyclophosphamide, Vincristine, Prednisone |
| COPE | Cyclophosphamide, Vincristine, Cisplatin, Etoposide |
| COPP | Cyclophosphamide, Vincristine, Procarbazine, Prednisone |
| CP (Chronic lymphocytic | Chlorambucil, Prednisone |
| leukemia) | |
| CP (Ovarian Cancer) | Cyclophosphamide, Cisplatin |
| CT | Cisplatin, Paclitaxel |
| CVD | Cisplatin, Vinblastine, Dacarbazine |
| CVI | Carboplatin, Etoposide, Ifosfamide, Mesna |
| CVP | Cyclophosphamide, Vincristine, Prednisome |
| CVPP | Lomustine, Procarbazine, Prednisone |
| CYVADIC | Cyclophosphamide, Vincristine, Doxorubicin, Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, Fluorouracil |
| ICE-T | Ifosfamide, Carboplatin, Etoposide, Paclitaxel, Mesna |
| IDMTX/6-MP | Methotrexate, Mercaptopurine, Leucovorin |
| IE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, Prednisone, |
| | Melphalan |
| MAC-III | Methotrexate, Leucovorin, Dactinomycin, |
| | Cyclophosphamide |
| MACC | Methotrexate, Doxorubicin, Cyclophosphamide, Lomustine |
| MACOP-B | Methotrexate, Leucovorin, Doxorubicin, Cyclophosphamide, |
| | Vincristine, Bleomycin, Prednisone |
| MAID | Mesna, Doxorubicin, Ifosfamide, Dacarbazine |
| m-BACOD | Bleomycin, Doxorubicin, Cyclophosphamide, Vincristine, |
| | Dexamethasone, Methotrexate, Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, Prednisone |
| MOPP/ABV | Mechlorethamine, Vincristine, Procarbazine, Prednisone, |
| | Doxorubicin, Bleomycin, Vinblastine |
| MP (multiple myeloma) | Melphalan, Prednisone |
| MP (prostate cancer) | Mitoxantrone, Prednisone |
| MTX/6-MO | Methotrexate, Mercaptopurine |
| MTX/6-MP/VP | Methotrexate, Mercaptopurine, Vincristine, Prednisone |
| MTX-CDDPAdr | Methotrexate, Leucovorin, Cisplatin, Doxorubicin |
| MV (breast cancer) | Mitomycin, Vinblastine |
| MV (acute myelocytic | Mitoxantrone, Etoposide |
| leukemia) | |
| M-VAC Methotrexate | Vinblastine, Doxorubicin, Cisplatin |
| MVP Mitomycin | Vinblastine, Cisplatin |
| MVPP | Mechlorethamine, Vinblastine, Procarbazine, Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PC | Paclitaxel, Carboplatin or Paclitaxel, Cisplatin |
| PCV | Lomustine, Procarbazine, Vincristine |
| PE | Paclitaxel, Estramustine |
| PFL | Cisplatin, Fluorouracil, Leucovorin |
| POC | Prednisone, Vincristine, Lomustine |
| ProMACE | Prednisone, Methotrexate, Leucovorin, Doxorubicin, |
| | Cyclophosphamide, Etoposide |
| ProMACE/cytaBOM | Prednisone, Doxorubicin, Cyclophosphamide, Etoposide, |
| | Cytarabine, Bleomycin, Vincristine, Methotrexate, |
| | Leucovorin, Cotrimoxazole |
| PRoMACE/MOPP | Prednisone, Doxorubicin, Cyclophosphamide, Etoposide, |
| | Mechlorethamine, Vincristine, Procarbazine, Methotrexate, |
| | Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, Asparaginase |
| SMF | Streptozocin, Mitomycin, Fluorouracil |
| TAD | Mechlorethamine, Doxorubicin, Vinblastine, Vincristine, |
| | Bleomycin, Etoposide, Prednisone |
| TCF | Paclitaxel, Cisplatin, Fluorouracil |
| TIP | Paclitaxel, Ifosfamide, Mesna, Cisplatin |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, Cisplatin, |
| | Bleomycin |
| VAC | Vincristine, Dactinomycin, Cyclophosphamide |
| VACAdr | Vincristine, Cyclophosphamide, Doxorubicin, Dactinomycin, |
| | Vincristine |
| VAD | Vincristine, Doxorubicin, Dexamethasone |
| VATH | Vinblastine, Doxorubicin, Thiotepa, Flouxymesterone |
| VBAP | Vincristine, Carmustine, Doxorubicin, Prednisone |
| VBCMP | Vincristine, Carmustine, Melphalan, Cyclophosphamide, |
| | Prednisone |
| VC | Vinorelbine, Cisplatin |
| VCAP | Vincristine, Cyclophosphamide, Doxorubicin, Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, Prednisone |
| VP | Etoposide, Cisplatin |
| V-TAD | Etoposide, Thioguanine, Daunorubicin, Cytarabine |
| 5 + 2 | Cytarabine, Daunorubicin, Mitoxantrone |
| 7 + 3 | Cytarabine with/, Daunorubicin or Idarubicin or |
| | Mitoxantrone |
| “8 in 1” | Methylprednisolone, Vincristine, Lomustine, Procarbazine, |
| | Hydroxyurea, Cisplatin, Cytarabine, Dacarbazine |

In addition to conventional chemotherapeutics, the compounds described herein as capable of inducing cell death or reducing lifespan can also be used with antisense RNA, RNAi or other polynucleotides to inhibit the expression of the cellular components that contribute to unwanted cellular proliferation that are targets of conventional chemotherapy. Such targets are, merely to illustrate, growth factors, growth factor receptors, cell cycle regulatory proteins, transcription factors, or signal transduction kinases.

The methods may be advantageous over combination therapies known in the art because it allows conventional chemotherapeutic agent to exert greater effect at lower dosage. In a preferred embodiment, the effective dose (ED₅₀) for a chemotherapeutic agent or combination of conventional chemotherapeutic agents when used in combination with a compound described herein is at least 2 fold less than the ED₅₀ for the chemotherapeutic agent alone, and even more preferably at 5 fold, 10 fold or even 25 fold less. Conversely, the therapeutic index (TI) for such chemotherapeutic agent or combination of such chemotherapeutic agent when used in combination with a compound described herein can be at least 2 fold greater than the TI for conventional chemotherapeutic regimen alone, and even more preferably at 5 fold, 10 fold or even 25 fold greater.

Other combination therapies include conjoint administration with nicotinamide, NAD⁺ or salts thereof, other Vitamin B3 analogs, and nicotinamide riboside or analogs thereof. Carnitines, such as L-carnitine, may also be co-administered, particularly for treating cerebral stroke, loss of memory, pre-senile dementia, Alzheimer's disease or preventing or treating disorders elicted by the use of neurotoxic drugs. Cyclooxygenase inhibitors, e.g., a COX-2 inhibitor, may also be co-administered for treating certain conditions described herein, such as an inflammatory condition or a neurologic disease.

A combination drug regimen may also include other agents or compounds for the treatment or prevention of neurodegenerative disorders, including Alzheimer's disease, ALS, Parkinson's disease, Huntington's disease, multiple sclerosis or secondary conditions associated with any of these conditions. For example, compounds described herein may be used in combination with one or more of the following agents used to treat Alzheimer's disease or related symptoms: cholinesterase inhibitors (such as tacrine and donepezil), rivastigmine, galantamine, galanthamine, memantine, metrifonate, bryostain, methylxanthine, non-steroidal anti-inflammatory drugs (rofecoxib, naxopren, celecoxib, aspirin, ibuprofen), vitamin E, selegiline, estrogen, ginkgo biloba extract, antidepressants, neuroleptics or mood stabilizers.

Compounds described herein may also be used in combination with one or more of the following agents used to treat ALS or related symptoms: riluzole, baclofen, tiranadine, dantrolene, benzodiazepines (such as diazepem), gabapentin, non-steroidal anti-inflammatory drugs (rofecoxib, naxopren, celecoxib, aspirin, ibuprofen), tramadol, antidepressants, selective serotonin re-uptake inhibitors, selective dopamine blockers, branch-chain amino acids, phenytoin, quinine, lorazepan, morpine, and chlorpromazine.

Compounds described herein may also be used in combination with one or more of the following agents used to treat Parkinson's disease or related symptoms: levodopa, carbidopa, selegiline, bromocriptine, pergolide, amantadine, trihexphenidyl, benztropine, COMT inhibitors (catechol-O-methyl transferase), anticholinergics, dopamine precursors, dopamine receptor agonists, MAO-B inhibitors, peripheral decarboxylase inhibitors.

Compounds described herein may also be used in combination with one or more of the following agents used to treat Huntington's disease or related symptoms: neuroleptic agents, dopamine receptor blockers (such as haloperidol and perphenazine), presynaptic dopamine depletors (such as reserpine), clozapine, antidepressants, mood stabilizer, and antipsychotic agents.

Compounds described herein may also be used in combination with one or more of the following agents used to treat multiple sclerosis or related symptoms: interferon beta-1a, interferon beta-1b, glatiramer, mitoxantrone, natalizumab, corticosteroids (such as prednisone, methylprednisolone, prednisolone, dexamethasone, adreno-corticotrophic hormone (ATCH), and corticotropin), chemotherapeutic agents (such as azathiprine, cyclophosphamide, cyclosporin, methotrexate, cladribine), amantadine, baclofen, meclizine, carbamazepine, gabapentin, topiramate, zonisamide, phenytoin, desipramine, amitriptyline, imipramine, doxepin, protriptyline, pentoxifylline, ibprofen, aspirin, acetaminophen, hydroxyzine, antidepressants, and antibodies that bind to α4-integrin (b1 and b7), e.g., TYSABRI® (natalizumab).

In certain embodiments, the subject sirtuin activators, such as SIRT1 activators, do not have any substantial ability to inhibit PI3-kinase, inhibit aldoreductase and/or inhibit tyrosine protein kinases at concentrations (e.g., in vivo) effective for activating the deacetylase activity of the sirtuin, e.g., SIRT1. For instance, in preferred embodiments the sirtuin activator is chosen to have an EC₅₀ for activating sirtuin deacetylase activity that is at least 5 fold less than the EC₅₀ for inhibition of one or more of aldoreductase and/or tyrosine protein kinases, and even more preferably at least 10 fold, 100 fold or even 1000 fold less.

In certain embodiments, the subject sirtuin activators do not have any substantial ability to transactivate EGFR tyrosine kinase activity at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for transactivating EGFR tyrosine kinase activity, and even more preferably at least 10 fold, 100 fold or even 1000 fold less.

In certain embodiments, the subject sirtuin activators do not have any substantial ability to cause coronary dilation at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for coronary dilation, and even more preferably at least 10 fold, 100 fold or even 1000 fold less.

In certain embodiments, the subject sirtuin activators do not have any substantial spasmolytic activity at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for spasmolytic effects (such as on gastrointestinal muscle), and even more preferably at least 10 fold, 100 fold or even 1000 fold less.

In certain embodiments, the subject sirtuin activators do not have any substantial ability to inhibit hepatic cytochrome P450 1B1 (CYP) at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for inhibition of P450 1B1, and even more preferably at least 10 fold, 100 fold or even 1000 fold less.

In certain embodiments, the subject sirtuin activators do not have any substantial ability to inhibit nuclear factor-kappaB (NF-κB) at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for inhibition of NF-κB, and even more preferably at least 10 fold, 100 fold or even 1000 fold less.

In certain embodiments, the subject SIRT1 activators do not have any substantial ability to activate SIRT1 orthologs in lower eukaryotes, particularly yeast or human pathogens, at concentrations (e.g., in vivo) effective for activating the deacetylase activity of human SIRT1. For instance, in preferred embodiments the SIRT1 activator is chosen to have an EC50 for activating human SIRT1 deacetylase activity that is at least 5 fold less than the EC50 for activating yeast Sir2 (such as Candida, S. cerevisiae, etc), and even more preferably at least 10 fold, 100 fold or even 1000 fold less.

In other embodiments, the subject sirtuin activators do not have any substantial ability to inhibit protein kinases; to phosphorylate mitogen activated protein (MAP) kinases; to inhibit the catalytic or transcriptional activity of cyclo-oxygenases, such as COX-2; to inhibit nitric oxide synthase (iNOS); or to inhibit platelet adhesion to type I collagen at concentrations (e.g., in vivo) effective for activating the deacetylase activity of the sirtuin. For instance, in preferred embodiments, the sirtuin activator is chosen to have an EC₅₀ for activating sirtuin deacetylase activity that is at least 5 fold less than the EC₅₀ for performing any of these activities, and even more preferably at least 10 fold, 100 fold or even 1000 fold less.

In other embodiments, a compound described herein, e.g., a sirtuin activator or inhibitor, does not have significant or detectable anti-oxidant activities, as determined by any of the standard assays known in the art. For example, a compound does not significantly scavenge free-radicals, such as O₂ radicals. A compound may have less than about 2, 3, 5, 10, 30 or 100 fold anti-oxidant activity relative to another compound, e.g., resveratrol.

A compound may also have a binding affinity for a sirtuin of about 10⁻⁹M, 10⁻¹⁰M, 10⁻¹¹M, 10⁻¹²M or less. A compound may reduce the Kₘ of a sirtuin for its substrate or NAD⁺ by a factor of at least about 2, 3, 4, 5, 10, 20, 30, 50 or 100. A compound may have an EC₅₀ for activating the deacetylase activity of a sirtuin of less than about 1 nM, less than about 10 nM, less than about 100 nM, less than about 1 μM, less than about 10 μM, less than about 100 μM, or from about 1-10 nM, from about 10-100 nM, from about 0.1-1 μM, from about 1-10 μM or from about 10-100 μM. A compound may activate the deacetylase activity of a sirtuin by a factor of at least about 5, 10, 20, 30, 50, or 100, as measured in an acellular assay or in a cell based assay as described in the Examples. A compound may cause at least a 10%, 30%, 50%, 80%, 2 fold, 5 fold, 10 fold, 50 fold or 100 fold greater induction of the deacetylase activity of SIRT1 relative to the same concentration of resveratrol or other compound described herein. A compound may also have an EC₅₀ for activating SIRT5 that is at least about 10 fold, 20 fold, 30 fold, 50 fold greater than that for activating SIRT1.

A compound may traverse the cytoplasmic membrane of a cell. For example, a compound may have a cell-permeability of at least about 20%, 50%, 75%, 80%, 90% or 95%.

Compounds described herein may also have one or more of the following characteristics: the compound may be essentially non-toxic to a cell or subject; the compound may be an organic molecule or a small molecule of 2000 amu or less, 1000 amu or less; a compound may have a half-life under normal atmospheric conditions of at least about 30 days, 60 days, 120 days, 6 months or 1 year; the compound may have a half-life in solution of at least about 30 days, 60 days, 120 days, 6 months or 1 year; a compound may be more stable in solution than resveratrol by at least a factor of about 50%, 2 fold, 5 fold, 10 fold, 30 fold, 50 fold or 100 fold; a compound may promote deacetylation of the DNA repair factor Ku70; a compound may promote deacetylation of RelA/p65; a compound may increase general turnover rates and enhance the sensitivity of cells to TNF-induced apoptosis.

In other embodiments, methods described herein are applied to yeast cells. Situations in which it may be desirable to extend the lifespan of yeast cells include any process in which yeast is used, e.g., the making of beer, yogurt, and bakery items, e.g., bread. Use of yeast having an extended lifespan can result in using less yeast or in having the yeast be active for longer periods of time. Yeast or other mammalian cells used for recombinantly producing proteins may also be treated as described herein.

Sirtuin activators may also be used for treating or preventing viral infections, such as infections by influenz, herpes or papillomavirus. They may also be used as antifungal agents, anti-inflammatory agents and neuroprotective agents.

Subjects that may be treated as described herein include eukaryotes, such as mammals, e.g., humans, ovines, bovines, equines, porcines, canines, felines, non-human primate, mice, and rats. Cells that may be treated include eukaryotic cells, e.g., from a subject described above, or plant cells, yeast cells and prokaryotic cells, e.g., bacterial cells. For example, activating compounds may be administered to farm animals to improve their ability to withstand farming conditions longer.

Compounds may also be used to increase lifespan, stress resistance, and resistance to apoptosis in plants. In one embodiment, a compound is applied to plants, e.g., on a periodic basis, or to fungi. In another embodiment, plants are genetically modified to produce a compound. In another embodiment, plants and fruits are treated with a compound prior to picking and shipping to increase resistance to damage during shipping. Plant seeds may also be contacted with compounds described herein, e.g., to preverse them.

Compounds may also be used to increase lifespan, stress resistance and resistance to apoptosis in insects. In this embodiment, compounds would be applied to useful insects, e.g., bees and other insects that are involved in pollination of plants. In a specific embodiment, a compound would be applied to bees involved in the production of honey. Generally, the methods described herein may be applied to any organism, e.g., eukaryote, that may have commercial importance. For example, they can be applied to fish (aquaculture) and birds (e.g., chicken and fowl).

Higher doses of compounds may also be used as a pesticide by interfering with the regulation of silenced genes and the regulation of apoptosis during development. In this embodiment, a compound may be applied to plants using a method known in the art that ensures the compound is bio-available to insect larvae, and not to plants.

Activated sirtuin proteins that are in vitro outside of a cell may be used, e.g., for deacetylating target proteins, thereby, e.g., activating the target proteins. Activated sirtuins may be used, e.g., for the identification, in vitro, of previously unknown targets of sirtuin deacetylation, for example using 2D electrophoresis of acetyl labeled proteins.

At least in view of the link between reproduction and longevity (Longo and Finch, Science, 2002), the compounds can be applied to affect the reproduction of organisms such as insects, animals and microorganisms.

Methods for preventing aging and aging-related consequences or diseases, such as neurological diseases and cardiovascular diseases, may also comprise increasing the protein level of a sirtuin, such as SIRT1, in a human cell, Sir2 in yeast cell, Sir2.1 in C. elegans or a homologue of any of these sirtuins in other organisms. Increasing protein levels can be achieved by introducing into a cell one or more copies of a nucleic acid that encodes a sirtuin. For example, the level of SIRT1 can be increased in a mammalian cell by introducing into the mammalian cell a nucleic acid encoding SIRT1, e.g., having the amino acid sequence set forth in SEQ ID NO: 2. The nucleic acid may be under the control of a promoter that regulates the expression of the SIRT1 nucleic acid. Alternatively, the nucleic acid may be introduced into the cell at a location in the genome that is downstream of a promoter. Methods for increasing the level of a protein by these ways are well known in the art. Illustrative methods are described in the Examples.

A nucleic acid that is introduced into a cell to increase the protein level of a sirtuin may encode a protein that is at least about 80%, 85%, 90%, 95%, 98%, or 99% identical to the sequence of a sirtuin, e.g., SEQ ID NO: 2. For example, the nucleic acid encoding the protein may be at least about 80%, 85%, 90%, 95%, 98%, or 99% identical to SEQ ID NO: 1. The nucleic acid may also be a nucleic acid that hybridizes, preferably under stringent hybridization conditions, to a nucleic acid encoding a wild-type sirtuin, e.g., SEQ ID NO: 1. Stringent hybridization conditions may include hybridization and a wash in 0.2×SSC at 65° C. When using a nucleic acid that encodes a protein that is different from a wild-type sirtuin protein, such as a protein that is a fragment of a wild-type sirtuin, the protein is preferably biologically active, e.g., is capable of deacetylation. It is only necessary to express in a cell a portion of the sirtuin that is biologically active. For example, a protein that differs from wild-type SIRT1 having SEQ ID NO: 2, preferably contains the core structure thereof. The core structure sometimes refers to amino acids 62-293 of SEQ ID NO: 2, which are encoded by nucleotides 237 to 932 of SEQ ID NO: 1, which encompasses the NAD binding as well as the substrate binding domains. The core domain of SIRT1 may also refer to about amino acids 261 to 447 of SEQ ID NO: 2, which are encoded by nucleotides 834 to 1394 of SEQ ID NO: 1; to about amino acids 242 to 493 of SEQ ID NO: 2, which are encoded by nucleotides 777 to 1532 of SEQ ID NO: 1; or to about amino acids 254 to 495 of SEQ ID NO: 2, which are encoded by nucleotides 813 to 1538 of SEQ ID NO: 1. Whether a protein retains a biological function, e.g., deacetylation capabilities, can be determined according to methods known in the art.

Sirtuin proteins may also be administered to subjects as a method of treatment. Proteins may be modified or packaged in such a way as to increase their passage through a cell membrane.

Methods for increasing sirtuin protein levels also include methods for stimulating the transcription of genes encoding sirtuins, methods for stabilizing the corresponding mRNAs, methods, and other methods known in the art. Upstream activators of sirtuins, e.g., those in the NAD+ salvage pathway, as described, e.g., in WO 04/016726, may also be used. In other embodiments methods of treatment include increasing the flux through the NAD+ salvage pathway, such as described in WO 2004/01676. In yet other embodiments, nicotinamide riboside or analogs thereof are administered. Nicotinamide riboside can be prepared by treating NMN (from, e.g., Sigma) with a phosphatase, as described, e.g., in Bieganowski et al. (2004) Cell 117:495. Nicotinamide riboside can be in the oxidized or reduced form, the latter of which appears to be more stable (Friedlos et al. (1992) Biochem Pharmacol. 44:631. Nicotinamide riboside (1) is depicted below.

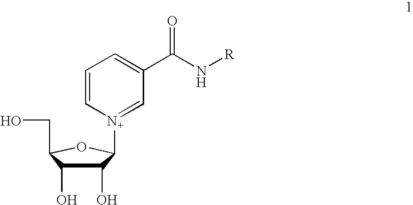

Nicotinamide riboside and some of its analogs are represented by formula A: 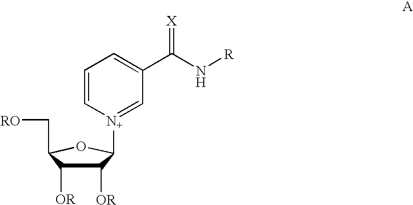
wherein
R represents independently for each occurrence H, acetyl, benzoyl, acyl, phosphate, sulfate, (alkyoxy)methyl, triarylmethyl, (trialkyl)silyl, (dialkyl)(aryl)silyl, (alkyl)(diaryl)silyl, or (triaryl)silyl; and
X represents O or S.

Nicotinamide riboside can be contacted with the cell at a concentration of about 1 nM to 10 μM. A cell may be optionally contacted with an agent that increases protein or activity levels of a nicotinamide riboside kinase (Nrk) enzyme, that phosphorylates nicotinamide riboside to form nicotinamide mononucleotide (NMN). Nrk exits in one form in yeast, Nrk1, and in two forms in humans, Nrk1 (GenBank Accession No. NM_{—}017881.1; NP_{—}060351) and Nrk2 (GenBank Accession Nos. NM_{—}170678; NP_{—}733778).

An exemplary method comprises administering to a subject in need thereof a therapeutically effective amount of an agent that increases the activity or protein level of a sirtuin. An agent may be a small molecule, e.g., as described above, or a nucleic acid encoding a sirtuin or a sirtuin protein.

A subject in need of therapy may be a subject having been diagnosed with a disease, e.g., a neurodegenerative disease. A subject may also be a subject who has been determined as being likely to develop a disease, e.g., a neurodegenerative disease, e.g., a subject having a form of a gene indicating susceptibility of developing the disease, or a subject in whose family the disease is more frequent than normally.

Inhibitory compounds may be used for similar purposes as those described herein for high concentrations of activating compounds. For example, inhibitory compounds may be used to stimulate acetylation of substrates such as p53 and thereby increase apoptosis, as well as to reduce the lifespan of cells and organisms and/or rendering them more sensitive to stress. Thus, inhibitory compounds may be used, e.g., for treating cancer.

Compositions or coformulations comprising a sirtuin activator or inhibitor and another agent, e.g., a chemotherapeutic agent, an antiviral agent, nicotinamide, NAD⁺ or salts thereof, Vitamin B3 analogs, retinoids, alpha-hydroxy acid, ascorbic acid, are also encompassed herein.

Also provided herein are diagnostic methods, e.g., methods for determining whether a subject has or is likely to develop neuronal cell death or a neurodegenerative disorder. A method may comprise determining the level or activity of a sirtuin protein. In one embodiment, a diagnostic method comprises (i) obtaining a biological sample of a subject and (ii) determining the level or activity of a sirtuin protein in the biological sample, wherein a higher level or activity of the sirtuin in the biological sample of the subject relative to a control indicates that the subject has or is likely to develop a neurodegenerative disease. The biological sample may be a cell sample, e.g., a blood sample, a spinal cord sample or a brain sample. Methods for determining the level of a sirtuin protein are known in the art and may involve using an antibody binding to the sirtuin protein. Methods for determining the activity of a sirtuin protein are also known in the art, and may involve determining its deacetylation efficiency. A control in the diagnostic assay may be a value corresponding to the level or activity of a sirtuin protein in an individual who does not have or is not likely to develop a neurodegenerative disease. A control may also be a value corresponding to the average of the level or activity of a sirtuin protein in two or more individuals who do not have or are not likely to develop a neurodegenerative disease. A control value may be an average of at least 5, 10, 50 or 100 individuals. A higher level or activity of a sirtuin in a biological sample of a subject relative to a control may include levels that are at least about 50%, 2, 3, 5, 10, 30, 50 or more fold higher in the subject than in the control.

“A method for diagnosing” includes any immunoassay, such as assays which utilize biotin and avidin or streptavidin, ELISAs, RIAs, Western blots, and immunoprecipitation. Diagnostic methods may use an antibody that specifically binds to a sirtuin. Other diagnostic assays comprise the use of nucleic acids, e.g., for determining the level of RNA, such as mRNA, or for determining the presence of a mutation in a sirtuin gene. The agent that is used in a diagnostic assay, e.g., an antibody or a nucleic acid, may be labeled and/or linked, covalently or not, to a solid surface.

A diagnostic method may comprise determining the level or activity of a sirtuin once or several times, e.g., several times within a determined period of time. For example, a diagnostic method may comprise obtaining a first biological sample of a subject, and obtaining a second biological sample several hours or days (e.g., 1, 2, 3, or 7 days) or weeks (e.g., 1, 2, 3 or 4 weeks) or months (e.g., 1, 2, 3, 6, 10 or more months) or years later. A change in the level of protein or activity of a sirtuin within the two samples may be indicative that a disease, e.g., a neurodegenerative disease, is evolving in the subject. An increase in the level of protein or activity of a sirtuin with time in a subject may indicate that the subject is developing a disease. A decrease in the level of protein or activity of a sirtuin with time in a subject may indicate that the disease or at least one or more symptoms thereof is being treated or prevented effectively.

A diagnosis of a disease may also comprise monitoring another characteristic of the disease, e.g., the presence or absence or level of a marker of the disease. For example, the diagnosis of Alzheimer's disease as described herein may be combined with the detection of β-amyloid plaques.

The diagnosis of a subject as having or being likely to develop a disease, e.g., a neurodegenerative disease, may be followed by the treatment of the subject, such as further described herein. In an illustrative embodiment, a method comprises first determining whether a subject has or is likely to develop a disease and second administering to a subject who was diagnosed as having or likely to develop the disease a therapeutically effective amount of an agent for treating the disease. The agent may be an agent that is known for treating the disease, such as those described herein. Alternatively, the agent may be an agent that increases the level of protein or activity of a sirtuin, such as SIRT1. The two types of agents may also be combined.

In another embodiment, a subject is treated, and the efficacy of the treatment or the progression of the disease is determined. A treatment may be a treatment known in the art or a treatment described herein. The efficacy of the treatment or the progression of the disease may be determined by measuring the level or activity of a sirtuin protein in the subject being treated. Measurements may be conducted on a regular basis, e.g., every day, every other day, once a week, once a month or once a year.

Also provided herein are screening methods for identifying compounds for treating neurodegenerative diseases. A screening method may comprise testing the activity of a compound known to be a sirtuin activator in a cell or animal model of a neurodegenerative disease, e.g., those described herein. A screening method may also comprise first isolating a compound that increases the activity or protein level of a sirtuin in a cell, and then determining its activity in a model of a neurodegenerative disease, e.g., those described herein.

Pharmaceutical Compositions and Methods

Pharmaceutical compositions for use in accordance with the present methods may be formulated in conventional manner using one or more physiologically acceptable carriers or excipients. Thus, activating compounds and their physiologically acceptable salts and solvates may be formulated for administration by, for example, injection, inhalation or insufflation (either through the mouth or the nose) or oral, buccal, parenteral or rectal administration. In one embodiment, the compound is administered locally, at the site where the target cells, e.g., diseased cells, are present, i.e., in the blood or in a joint.

Compounds can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets, lozanges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For administration by inhalation, the compounds may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin, for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions (including cosmetic preparations) may comprise from about 0.00001 to 100% such as from 0.001 to 10% or from 0.1% to 5% by weight of one or more compounds described herein.

In one embodiment, a compound described herein, is incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Formulations may be colorless, odorless ointments, lotions, creams, microemulsions and gels.

Compounds may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington's, cited in the preceding section, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Exemplary water-soluble ointment bases are prepared from polyethylene glycols (PEGs) of varying molecular weight; again, reference may be had to Remington's, supra, for further information.

Compounds may be incorporated into lotions, which generally are preparations to be applied to the skin surface without friction, and are typically liquid or semiliquid preparations in which solid particles, including the active agent, are present in a water or alcohol base. Lotions are usually suspensions of solids, and may comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. An exemplary lotion formulation for use in conjunction with the present method contains propylene glycol mixed with a hydrophilic petrolatum such as that which may be obtained under the trademark Aquaphor®™ from Beiersdorf, Inc. (Norwalk, Conn.).

Compounds may be incorporated into creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic or amphoteric surfactant.

Compounds may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

Compounds may be incorporated into gel formulations, which generally are semisolid systems consisting of either suspensions made up of small inorganic particles (two-phase systems) or large organic molecules distributed substantially uniformly throughout a carrier liquid (single phase gels). Single phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose and gelatin. Although gels commonly employ aqueous carrier liquid, alcohols and oils can be used as the carrier liquid as well.

Various additives, known to those skilled in the art, may be included in formulations, e.g., topical formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients and preservatives. An optimum topical formulation comprises approximately: 2 wt. % to 60 wt. %, preferably 2 wt. % to 50 wt. %, solubilizer and/or skin permeation enhancer; 2 wt. % to 50 wt. %, preferably 2 wt. % to 20 wt. %, emulsifiers; 2 wt. % to 20 wt. % emollient; and 0.01 to 0.2 wt. % preservative, with the active agent and carrier (e.g., water) making of the remainder of the formulation.

A skin permeation enhancer serves to facilitate passage of therapeutic levels of active agent to pass through a reasonably sized area of unbroken skin. Suitable enhancers are well known in the art and include, for example: lower alkanols such as methanol ethanol and 2-propanol; alkyl methyl sulfoxides such as dimethylsulfoxide (DMSO), decylmethylsulfoxide (C.sub.10 MSO) and tetradecylmethyl sulfboxide; pyrrolidones such as 2-pyrrolidone, N-methyl-2-pyrrolidone and N-(-hydroxyethyl)pyrrolidone; urea; N,N-diethyl-m-toluamide; C.sub.2-C.sub.6 alkanediols; miscellaneous solvents such as dimethyl formamide (DMF), N,N-dimethylacetamide (DMA) and tetrahydrofurfuryl alcohol; and the 1-substituted azacycloheptan-2-ones, particularly 1-n-dodecylcyclazacycloheptan-2-one (laurocapram; available under the trademark Azone^{RTM} from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol^{RTM}) and diethylene glycol monoethyl ether oleate (available commercially as Softcutol^{RTM}); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol^{RTM}); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like.

Other active agents may also be included in formulations, e.g., other anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation.

Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507. Accordingly, also provided are closed containers containing a cosmetically acceptable composition as herein defined.

In an alternative embodiment, a pharmaceutical formulation is provided for oral or parenteral administration, in which case the formulation may comprises an activating compound-containing microemulsion as described above, but may contain alternative pharmaceutically acceptable carriers, vehicles, additives, etc. particularly suited to oral or parenteral drug administration. Alternatively, an activating compound-containing microemulsion may be administered orally or parenterally substantially as described above, without modification.

Phospholipids complexes, e.g., resveratrol-phospholipid complexes, and their preparation are described in U.S. 2004116386. Methods for stabilizing active components using polyol/polymer microcapsules, and their preparation are described in U.S. 20040108608. Processes for dissolving lipophilic compounds in aqueous solution with amphiphilic block copolymers are described in WO 04/035013.

Conditions of the eye can be treated or prevented by, e.g., systemic, topical, intraocular injection of a compound described herein, or by insertion of a sustained release device that releases a compound described herein.

Compounds described herein may be stored in oxygen free environment according to methods in the art. For example, resveratrol or analog thereof can be prepared in an airtight capusule for oral administration, such as Capsugel from Pfizer, Inc.

Cells, e.g., treated ex vivo with a compound described herein, can be administered according to methods for administering a graft to a subject, which may be accompanied, e.g., by administration of an immunosuppressant drug, e.g., cyclosporin A. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000.

Toxicity and therapeutic efficacy of compounds can be determined by standard pharmaceutical procedures in cell cultures or experimental animals. The LD₅₀ is the dose lethal to 50% of the population). The ED₅₀ is the dose therapeutically effective in 50% of the population. The dose ratio between toxic and therapeutic effects (LD₅₀/ED₅₀) is the therapeutic index. Compounds that exhibit large therapeutic indexes are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may lie within a range of circulating concentrations that include the ED₅₀ with little or no toxicity. The dosage may vary within this range depending upon the dosage form employed and the route of administration utilized. For any compound, the therapeutically effective dose can be estimated initially from cell culture assays. A dose may b e formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Methods for increasing the protein level of a sirtuin in a cell may also comprise increasing the level of expression of the gene. In addition, one or more nucleic acids encoding a sirtuin may be introduced into a cell to increase the level of the sirtuin protein in the cell. In an exemplary embodiment, a vector encoding a sirtuin is introduced into a cell. A vector may be a viral vector. Viral vectors for administering to subjects are well known in the art, and include adenoviral vectors. For example, the transgene may be incorporated into any of a variety of viral vectors useful in gene therapy, such as recombinant retroviruses, adenovirus, adeno-associated virus (AAV), and herpes simplex virus-1, or recombinant bacterial or eukaryotic plasmids. While various viral vectors may be used in the practice of the methods described herein, AAV- and adenovirus-based approaches are of particular interest. Such vectors are generally understood to be the recombinant gene delivery system of choice for the transfer of exogenous genes in vivo, particularly into humans.

It is possible to limit the infection spectrum of viruses by modifying the viral packaging proteins on the surface of the viral particle (see, for example PCT publications WO93/25234, WO94/06920, and WO94/11524). For instance, strategies for the modification of the infection spectrum of viral vectors include: coupling antibodies specific for cell surface antigens to envelope protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral envelope proteins (Neda et al., (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

Nucleic acids and proteins can also be administered in a form of a complex with other components, e.g., agents facilitating delivery to the target tissue or organ, agents facilitating transport through the cell membrane or the gut. For example proteins may be in the form of fusion proteins, fused, e.g., to transcytosis peptides. Nucleic acids and proteins may be administered with liposomes.

Administration of a sirtuin activator or other agent that increases the activity or protein level of a sirtuin may be followed by measuring a factor in the subject, such as measuring the activity of the sirtuin. In an illustrative embodiment, a cell is obtained from a subject following administration of an activating compound to the subject, such as by obtaining a biopsy, and the activity of the sirtuin or sirtuin expression level is determined in the biopsy. Alternatively, biomarkers, such as plasma biomarkers may be followed. The cell may be any cell of the subject, but in cases in which an activating compound is administered locally, the cell is preferably a cell that is located in the vicinity of the site of administration.

Kits

Also provided herein are kits, e.g., kits for therapeutic purposes or kits for modulating the lifespan of cells or modulating apoptosis. A kit may comprise one or more activating or inhibitory compounds described herein, e.g., in premeasured doses. A kit may optionally comprise devices for contacting cells with the compounds and instructions for use. Devices include syringes, stents and other devices for introducing a compound into a subject or applying it to the skin of a subject.

Further, a kit may also contain components for measuring a factor, e.g., described above, such as the activity of sirtuin proteins, e.g., in tissue samples.

Other kits include kits for diagnosing the likelihood of having or developing a neurodegenerative disorder, precursors thereof or secondary conditions thereof. A kit may comprise an agent for measuring the activity and or expression level of a sirtuin.

Kits for screening assays are also provided. Exemplary kits comprise one or more agents for conducting a screening assay, such as a sirtuin, or a biologically active portion thereof, or a cell or cell extract comprising such. Any of the kits may also comprise instructions for use.

The present description is further illustrated by the following examples, which should not be construed as limiting in any way. The contents of all cited references (including literature references, accession numbers (such as GenBank Accession numbers), issued patents, published patent applications as cited throughout this application) are hereby expressly incorporated by reference.

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

### Example 1

### Small Molecule Activators of SIRT1

To identify compounds that modulate SIRT1 activity, we screened a number of small molecule libraries using a fluorescent deacetylation assay in 96-well plates²⁶. The substrate used in the assay was a fluorogenic peptide based on the sequence encompassing the p53-K382 acetylation site, a known target of SIRT1 in vivo^{20,21,27}. This substrate was preferred over a variety of other fluorogenic peptide substrates that were based on other known HDAC targets (FIG. 5). The small molecule libraries included analogues of nicotinamide, α-acetyl lysine, NAD⁺, nucleotides, nucleotide analogues and purinergic ligands. From the initial screen, several sirtuin inhibitors were found (Supplementary Table 7). However, the most striking outcome was the identification of two compounds, quercetin and piceatannol, that stimulated SIRT1 activity five and eight-fold, respectively (Table 1). Both quercetin and piceatannol have been previously identified as protein kinase inhibitors^{28,29}.

Comparison of the structures of the two activating compounds suggested a possible structure-activity relationship. Piceatannol comprises two phenyl groups trans to one another across a linking ethylene moiety. The trans-stilbene ring structures of piceatannol are superimposable on the flavonoid A and B rings of quercetin, with the ether oxygen and carbon-2 of the C ring aligning with the ethylene carbons in piceatannol (see structures, Table 1). Further, the 5, 7, 3′ and 4′ hydroxyl group positions in quercetin can be aligned, respectively, with the 3, 5, 3′ and 4′ hydroxyls of piceatannol.

Given the demonstrated longevity-enhancing effects of sirtuin activity in S. cerevisiae⁷ and C. elegans¹⁹, it was naturally of interest to further explore the structure-activity relationship among compounds that stimulate SIRT1. Both quercetin and piceatannol are polyphenols, members of a large and diverse group of plant secondary metabolites that includes flavones, stilbenes, flavanones, isoflavones, catechins (flavan-3-ols), chalcones, tannins and anthocyanidins^{30,31}. Polyphenols noteworthy with respect to potential longevity-enhancing effects include resveratrol, a stilbene found in red wine and epigallocatechin gallate (EGCG) from green tea. Both have been suggested on the basis of epidemiological and mechanistic investigations to exert cancer chemopreventive and cardioprotective effects³⁰⁻³². We therefore performed a secondary screen encompassing resveratrol, EGCG and additional representatives from a number of the polyphenol classes listed above. The screen emphasized flavones due to the great number of hydroxyl position variants available in this group³¹. The results of this screen are summarized in Supplementary Tables 1-6. In the tables, a “ratio to control rate” above 1 indicates that a compound with such a rate is an activator of the sirtuin tested and a number under 1 indicates that a compound is an inhibitor.

Additional potent SIRT1 activators were found among the stilbenes, chalcones and flavones (Table 1, Supplementary Tables 1 and 2). The six most active flavones had 3′ and 4′ hydroxyls (Supplementary Table 2), although it should be noted that the most active compound overall, resveratrol (3,5,4′-trihydroxystilbene), was more active than piceatannol, which differs only by its additional 3′-hydroxyl (Table 1). The importance of the 4′-hydroxyl to activity is underscored by the fact that each of the 12 most stimulatory flavones share this feature (Supplementary Tables 1 and 2).

Many, but not all of the most active compounds include hydroxyls in the two meta positions (e.g. 5,7-dihydroxylated flavones) of the ring (A ring), trans to that with the 4′ or 3′,4′ pattern (B ring, see Table 1, Supplementary Tables 1 and 2). A potentially coplanar orientation of the trans phenyl rings may be important for activity since catechins and flavanones, which lack the 2,3 double-bond, have weak activity despite having equivalent hydroxylation patterns to various stimulatory flavones (compare Supplementary Tables 2 and 3 with 4 and 5). The absence of activity in the isoflavone genistein, although hydroxylated in an equivalent way to the stimulatory compounds apigenin and resveratrol (see Supplementary Tables 1, 2 and 4), is consistent with the idea that the trans positioning and spacing of the hydroxylated rings contributes strongly to activity.

The biological effects of polyphenols are frequently attributed to antioxidant, metal ion chelating and/or free-radical scavenging activity^{30,32}. We considered the possibility that the apparent polyphenol stimulation of SIRT1 might simply represent the repair of oxidative and/or metal-ion induced damage incurred during preparation of the recombinant protein. Two features of our results argue against this being the case. First, a variety of free-radical protective compounds, including antioxidants, chelators and radical scavengers, failed to stimulate SIRT1 (see Supplementary Table 6.). Second, among various polyphenols of equivalent antioxidant capacity we observed diverse SIRT1 stimulating activity (e.g. compare resveratrol, quercetin and the epicatechins in Supplementary Tables 1, 2 and 5 and see 33).

### Example 2

### Resveratrol's Effects on SIRT1 Kinetics

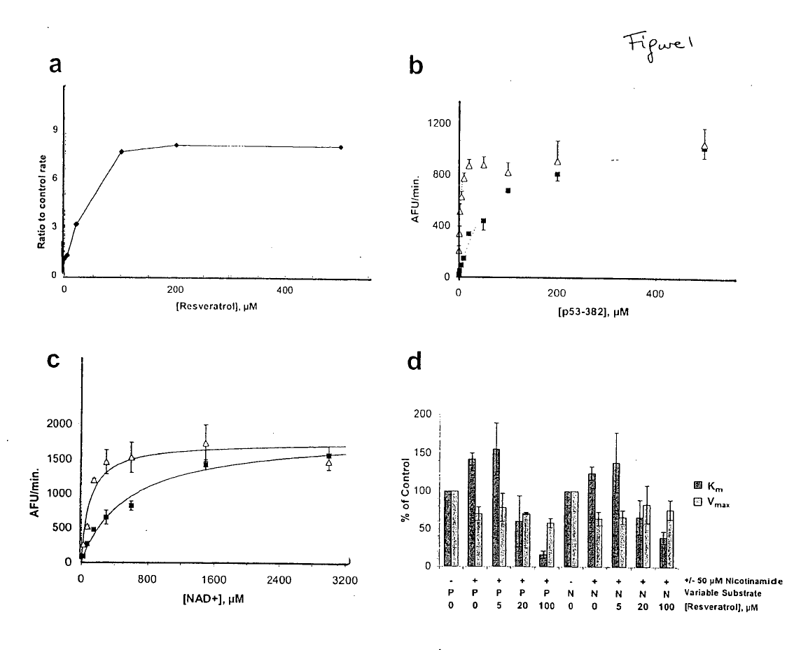

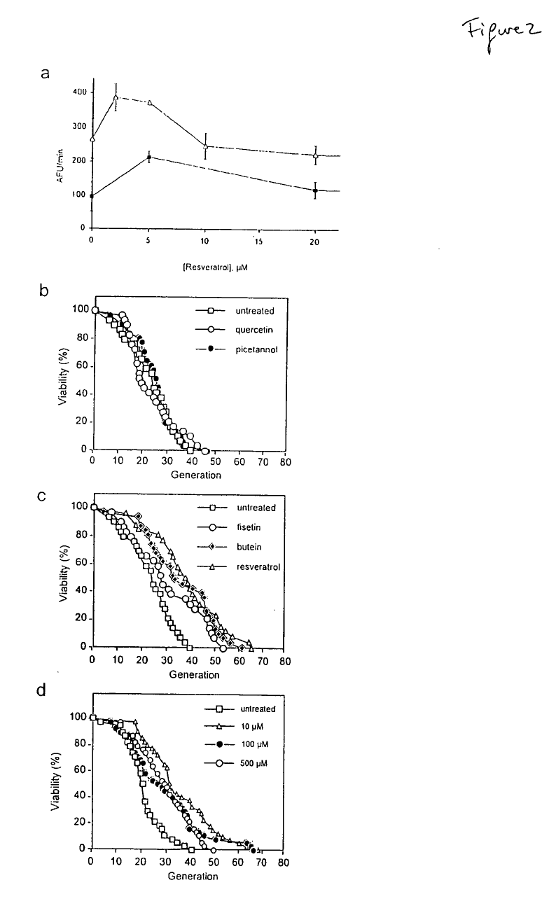

Detailed enzyme kinetic investigations were performed using the most potent activator, resveratrol. Dose-response experiments performed under the conditions of the polyphenol screening assays (25 μM NAD⁺, 25 μM p53-382 acetylated peptide), showed that the activating effect doubled the rate at ˜11 μM and was essentially saturated at 100 μM resveratrol (FIG. 1a). Initial enzyme rates, in the presence or absence of 100 μM resveratrol, were determined either as a function of acetyl-peptide concentration with high NAD⁺ (3 mM NAD⁺, FIG. 1b) or as a function of NAD⁺ concentration with high acetyl-peptide (1 mM p53-382 acetylated peptide, FIG. 1c). Although resveratrol had no significant effect on the two Vₘₐₓ determinations (FIGS. 1b, 1c), it had pronounced effects on the two apparent Kₘs. Its effect on the acetylated peptide Kₘ was particularly striking, amounting to a 35-fold decrease (FIG. 1b). Resveratrol also lowered the Kₘ for NAD⁺ over 5-fold (FIG. 1c). Since resveratrol acts only on Kₘ, it could be classified as an allosteric effector of ‘K system’ type³⁴. This can imply that only the substrate binding affinity of the enzyme has been altered, rather than a rate-limiting catalytic step.

Our previous kinetic analysis of SIRT1 and Sir2²⁶ and our genetic analysis of Sir2's role in yeast lifespan extension^{6,35} have implicated nicotinamide (a product of the sirtuin reaction) as a physiologically important inhibitor of sirtuin activity. Therefore the effects of resveratrol on nicotinamide inhibition were tested. In experiments similar to those of FIGS. 1b and 1c, kinetic constants in the presence of 50 μM nicotinamide were determined either by varying the concentration of NAD⁺ or that of the p53-382 acetylated peptide (FIG. 1d). Nicotinamide, in contrast to resveratrol, affects the SIRT1 Vₘₐₓ (note 30% and 36% Vₘₐₓ decreases in absence of resveratrol, FIG. 1d and see ref.²⁶). In the presence of 50 μM nicotinamide, resveratrol appears to have complex, concentration-dependent effects on the kinetics of SIRT1 (FIG. 1d). Apparent Kₘ for NAD⁺ and acetylated substrate appear to actually be raised by 5 μM resveratrol when nicotinamide is present. At 20 and 100 μM, in the presence of 50 μM nicotinamide, resveratrol lowers the Kₘ for both NAD⁺ and acetylated peptide, without reversing the nicotinamide-induced Vₘₐₓ decrease. It has been proposed that sirtuins may bind nicotinamide at a second site, known as “the C pocket”, distinct from the “B” site that interacts with the nicotinamide moiety of NAD^{+ 26}. In light of this potential complexity, further kinetic studies, supplemented by structural/crystallographic information, will likely be necessary to fully elucidate the interplay between the effects of nicotinamide and polyphenols.

### Example 3

### Activating Compounds Extend Yeast Lifespan

To investigate whether these compounds could stimulate sirtuins in vivo, we utilized S. cerevisiae, an organism in which the upstream regulators and downstream targets of Sir2 are relatively well understood. A resveratrol dose-response study of Sir2 deacetylation rates (FIG. 2a) indeed reveals that resveratrol stimulates Sir2 in vitro, with the optimum concentration of activator being 2-5 μM. Levels of activation were somewhat lower than those for SIRT1, and unlike SIRT1, inhibition was seen at concentrations greater than ˜100 μM.

Resveratrol and four other potent sirtuin activators, representatives of the stilbene, flavone, and chalcone families, were tested for their effect on yeast lifespan. Due to the potential impediment by the yeast cell wall or plasma membrane and suspected slow oxidation of the compound in the medium, we chose to use a concentration (10 μM) slightly higher than the optimal resveratrol concentration in vitro. As shown in FIG. 2b, quercetin and piceatannol had no significant effect on lifespan. In contrast, butein, fisetin and resveratrol increased average lifespan by 31, 55 and 70%, respectively, and all three significantly increased maximum lifespan (FIG. 2c). Concentrations of resveratrol higher than 10 μM provided no added lifespan benefit and there was no lasting effect of the compound on the lifespan of pre-treated young cells (FIG. 2d and data not shown).

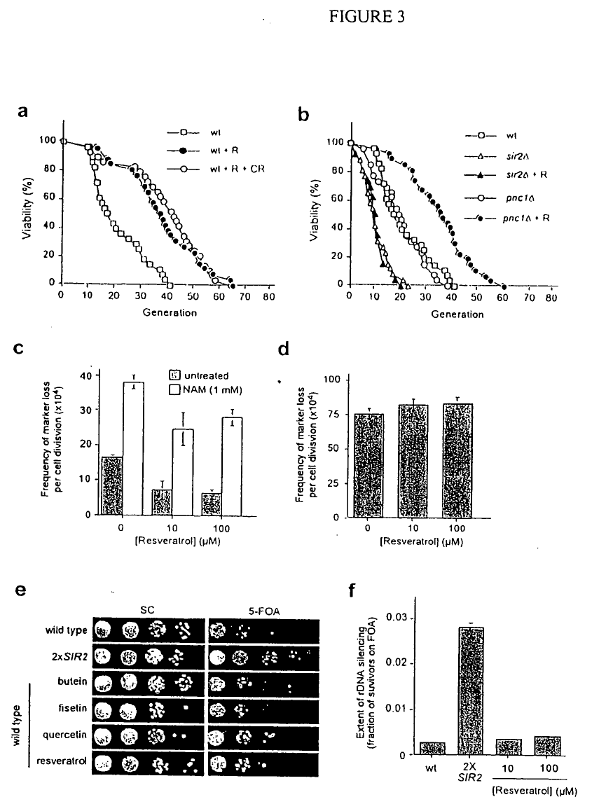

For subsequent yeast genetic experiments we focused on resveratrol because it was the most potent SIRT1 activator and provided the greatest lifespan extension. Glucose restriction, a form of CR in yeast, resulted in no significant extension of the long-lived resveratrol-treated cells (FIG. 3a), indicating that resveratrol likely acts via the same pathway as CR. Consistent with this, resveratrol had no effect on the lifespan of a sir2 null mutant (FIG. 3b). Given that resveratrol is reported to have fungicidal properties at high concentrations³⁶, and that mild stress can extend yeast lifespan by activating PNC1⁶, it was plausible that resveratrol was extending lifespan by inducing PNC1, rather than acting on Sir2 directly. However, resveratrol extended the lifespan of a pnc1 null mutant nearly as well as it did wild type cells (FIG. 3b). Together these data show that resveratrol acts downstream of PNC1 and requires SIR2 for its effect. Thus, the simplest explanation for our observations is that resveratrol increases lifespan by directly stimulating Sir2 activity.

A major cause of yeast aging is thought to stem from the inherent instability of the repetitive rDNA locus^{2,5,37-39}. Homologous recombination between rDNA repeats can generate an extrachromosomal circular form of rDNA (ERC) that is replicated until it reaches toxic levels in old cells. Sir2 is thought to extend lifespan by suppressing recombination at the replication fork barrier of rDNA⁴⁰. Consistent with the lifespan extension we observed for resveratrol, this compound reduced the frequency of rDNA recombination by ˜60% (FIG. 3c), in a SIR2-dependent manner (FIG. 3d). In the presence of the Sir2 inhibitor nicotinamide, recombination was also decreased by resveratrol (FIG. 3c), in agreement with the kinetic data (see FIG. 1d). Interestingly, we found that resveratrol and other sirtuin activators had only minor effects on rDNA silencing (FIGS. 3e and f). Work is underway to elucidate how these various compounds can differentially affect rDNA stability and silencing.

Another measure of lifespan in S. cerevisiae is the length of time cells can survive in a metabolically active but nutrient deprived state. Aging under these conditions (i.e. chronological aging) is primarily due to oxidative damage⁴¹. Resveratrol (10 μM or 100 μM) failed to extend chronological lifespan (not shown), indicating that the sirtuin-stimulatory effect of resveratrol may be more relevant in vivo than its antioxidant activity^{30,31}.

### Example 4

### Effects of Activators in Human Cells

To test whether these compounds could stimulate human SIRT1 in vivo, we first employed a cellular deacetylase assay that we had developed. A schematic of the assay procedure is depicted in FIG. 4a. Cells are incubated with media containing the fluorogenic ε-acetyl-lysine substrate, ‘Fluor de Lys’ (FdL). This substrate, neutral when acetylated, becomes positively charged upon deacetylation and accumulates within cells (see FIG. 6a). Lysis of the cells and addition of the non-cell-permeable ‘Developer’ reagent releases a fluorophor specifically from those substrate molecules that have been deacetylated (FIG. 4a and see Methods). With HeLa cells growing adherently, 5-10% of the signal produced in this assay is insensitive to 1 μM trichostatin A (TSA), a potent inhibitor of class I and II HDACs but not sirtuins (class III)⁴² (FIGS. 6b and 6c).

A selection of SIRT1-stimulatory and non-stimulatory polyphenols were tested for their effects on this TSA-insensitive signal (FIG. 4b). Cellular deacetylation signals in the presence of each compound (y-axis, FIG. 4b) were plotted against their fold-stimulations of SIRT1 in vitro (x-axis, FIG. 4b, data from Supplementary Tables 1-3). For most of the compounds, the in vitro activity roughly corresponded to the cellular signal. Compounds with little or no in vitro activity clustered around the negative control (Group A, FIG. 4b). Another grouping, of strong in vitro activators is clearly distanced from the low activity cluster in both dimensions (Group B, FIG. 4b). A notable outlier was butein, a potent activator of SIRT1 in vitro which had no effect on the cellular signal. With allowances for possible variation among these compounds in properties unrelated to direct sirtuin stimulation, such as cell-permeability and rates of metabolism, these data are consistent with the idea that certain polyphenols can activate native sirtuins in vivo.

One known target of SIRT1 in vivo is lysine 382 of p53. Deacetylation of this residue by SIRT1 decreases the activity and half-life of p53^{20,21,27}. To follow the acetylation status of K382 we generated a rabbit polyclonal antibody that recognizes the acetylated form of K382 (Ac-K382) on Western blots of whole cell lysates. As a control we showed that the signal was specifically detected in extracts from cells exposed to ionizing radiation (FIG. 4c), but not in extracts from cells lacking p53 or where arginine had been substituted for lysine 382 (data not shown). U2OS osteosarcoma cells were pre-treated for 4 hours with resveratrol (0.5 and 50 μM) and exposed to UV radiation. We consistently observed a marked decrease in the level of Ac-K382 in the presence of 0.5 μM resveratrol, compared to untreated cells (FIG. 4d). At higher concentrations of resveratrol (>50 μM) the effect was reversed (FIG. 4d and data not shown), consistent with previous reports of increased p53 activity at such concentrations⁴³. The ability of low concentrations of resveratrol to promote deacetylation of p53 was diminished in cells expressing a dominant-negative SIRT1 allele (H363Y) (FIG. 4e), demonstrating that SIRT1 is necessary for this effect. This biphasic dose-response of resveratrol could explain the dichotomy in the literature regarding the effects of resveratrol on cell survival^{30,43,44}.

Thus, we have discovered the first known class of small molecule sirtuin activators, all of which are plant polyphenols. These compounds can dramatically stimulate sirtuin activity in vitro and promote effects consistent with increased sirtuin activity in vivo. In human cells, resveratrol promotes SIRT1-mediated p53 deacetylation of K382. In yeast, the effect of resveratrol on lifespan is as great as any longevity-promoting genetic manipulation⁶ and has been linked convincingly to the direct activation of Sir2. The correlation between lifespan and rDNA recombination, but not silencing, adds to the body of evidence that yeast aging is due to DNA instability^{2,5,37-39} not gene dysregulation⁴⁵.

How can we explain the activation of the yeast and human sirtuins by so many plant metabolites? Sirtuins have been found in diverse eukaryotes, including fungi, protozoans, metazoans and plants^{46,47}, and likely evolved early in life's history¹. Plants are known to produce a variety of polyphenols, including resveratrol, in response to stresses such as dehydration, nutrient deprivation, UV radiation and pathogens^{48,49}. Therefore it is plausible that these compounds may be synthesized to regulate a sirtuin-mediated plant stress response. This would be consistent with the recently discovered relationship between environmental stress and Sir2 activity in yeast⁶. Perhaps these compounds have stimulatory activity on sirtuins from fungi and animals because they mimic an endogenous activator, as is the case for the opiates/endorphins, cannabinols/endocannabinoids and various polyphenols with estrogen-like activity^{30,31}. Alternatively, animal and fungal sirtuins may have retained or developed an ability to respond to these plant metabolites because they are a useful indicator of a deteriorating environment and/or food supply.

### Example 5

### Materials and Methods for Examples 1-4

Compound Libraries and Deacetylation Assays

His₆-tagged recombinant SIRT1 and GST-tagged recombinant Sir2 were prepared as previously described 26. From 0.1 to 1 μg of SIRT1 and 1.5 μg of Sir2 were used per deacetylation assay (in 50 μl total reaction) as previously described²⁶. SIRT1 assays and certain of those for Sir2 employed the p53-382 acetylated substrate (‘Fluor de Lys-SIRT1’, BIOMOL) rather than FdL.

Themed compound libraries (BIOMOL) were used for primary and secondary screening. Most polyphenol compounds were dissolved at 10 mM in dimethylsulfoxide (DMSO) on the day of the assay. For water soluble compounds and negative controls, 1% v/v DMSO was added to the assay. In vitro fluorescence assay results were read in white 1/2-volume 96-well microplates (Corning Costar 3693) with a CytoFluor™II fluorescence plate reader (PerSeptive Biosystems, Ex. 360 nm, Em. 460 nm, gain=85). HeLa cells were grown and the cellular deacetylation assays were performed and read, as above, but in full-volume 96-well microplates (Corning Costar 3595). Unless otherwise indicated all initial rate measurements were means of three or more replicates, obtained with single incubation times, at which point 5% or less of the substrate initially present had been deacetylated. Calculation of net fluorescence increases included subtraction of a blank value, which in the case of Sir2 was obtained by omitting the enzyme from the reaction and in the case of SIRT1 by adding an inhibitor (200 μM suramin or 1 mM nicotinamide) to the reaction prior to the acetylated substrate. A number of the polyphenols partially quenched the fluorescence produced in the assay and correction factors were obtained by determining the fluorescence increase due to a 3 μM spike of an FdL deacetylated standard (BIOMOL, catalog number KI-142). All error bars represent the standard error of the mean.

Media and Strains

All yeast strains were grown at 30° C. in complete yeast extract/bactopeptone, 2.0% (w/v) glucose (YPD) medium except where stated otherwise. Calorie restriction was induced in 0.5% glucose. Synthetic complete (SC) medium consisted of 1.67% yeast nitrogen base, 2% glucose, 40 mg/litre each of auxotrophic markers. SIR2 was integrated in extra copy and disrupted as described⁵. Other strains are described elsewhere²⁶. For cellular deacetylation assays, HeLa S3 cells were used. U2OS osteosarcoma and human embryonic kidney (HEK 293) cells were cultured adherently in Dulbecco's Modified Eagle's Medium (DMEM) containing 10% fetal calf serum (FCS) with 1.0% glutamine and 1.0% penecillin/streptomycin. HEK 293 overexpressing dominant negative SIRT1 H363Y was a gift of R. Frye (U. Pittsburgh).

Lifespan Determinations

Lifespan measurements were performed using PSY316AT MATα as previously described³⁵. All compounds for lifespan analyses were dissolved in 95% ethanol and plates were dried and used within 24 hours. Prior to lifespan analysis, cells were pre-incubated on their respective media for at least 15 hours. Following transfer to a new plate, cells were equilibrated on the medium for a minimum of 4 hours prior to micro-manipulating them. At least 30 cells were examined per experiment and each experiment was performed at least twice. Statistical significance of lifespan differences was determined using the Wilcoxon rank sum test. Differences are stated to be significant when the confidence is higher than 95%.

Silencing and Recombination Assays

Ribosomal DNA silencing assays using the URA3 reporters were performed as previously described²⁶. Ribosomal DNA recombination frequencies were determined by plating W303AR cells³⁷ on YPD medium with low adenine/histidine and counting the fraction of half-red sectored colonies using Bio-Rad Quantity One software as previously described³⁵. At least 6000 cells were analyzed per experiment and all experiments were performed in triplicate. All strains were pre-grown for 15 hours with the relevant compound prior to plating.

Proteins and Western Analyses

Recombinant Sir2-GST was expressed and purified from E. coli as previously described except that lysates were prepared using sonication²⁶. Recombinant SIRT1 from E. coli was prepared as previously described²⁶. Polyclonal antiserum against p53-AcK382 was generated using an acetylated peptide antigen as previously described²⁰, with the following modifications. Anti-Ac-K382 antibody was affinity purified using non-acetylated p53-K382 peptides and stored in PBS at −70° C. and recognized an acetylated but not a non-acetylated p53 peptide. Western hybridizations using anti-acetylated K382 or anti-actin (Chemicon) antibody were performed at 1:1000 dilution of antibody. Hybridizations with polyclonal p53 antibody (Santa Cruz Biotech.) used 1:500 dilution of antibody. Whole cell extracts were prepared by lysing cells in buffer containing 150 mM NaCl, 1 mM MgCl₂, 10% glycerol, 1% NP40, 1 mM DTT and anti-protease cocktail (Roche).

### REFERENCES FOR EXAMPLES 1-4 AND BACKGROUND

1. Kenyon, C. Cell 105, 165-168 (2001).
2. Sinclair, D. A. Mech Ageing Dev 123, 857-67 (2002).
3. Hekimi, S. & Guarente Science 299, 1351-4 (2003).
4. Guarente, L. & Kenyon, C. Nature 408, 255-62. (2000).
5. Lin et al. Science 289, 2126-8. (2000).
6. Anderson et al. Nature 423, 181-5 (2003).
7. Kaeberlein et al. Genes Dev 13, 2570-80. (1999).
8. Landry et al. Proc Natl Acad Sci USA 97, 5807-11. (2000).
9. Imai et al. Nature 403, 795-800 (2000).
10. Smith et al. Proc Natl Acad Sci USA 97, 6658-63. (2000).
11. Tanner et al. Proc Natl Acad Sci USA 97, 14178-82. (2000).
12. Tanny et al. Cell 99, 735-45. (1999).
13. Tanny et al. Proc Natl Acad Sci USA 98, 415-20. (2001).
14. Laurenson et al. Microbiol Rev 56, 543-60. (1992).
15. Smith et al. Genes Dev 11, 241-54. (1997).
16. Bryk, M. et al. Genes Dev 11, 255-69. (1997).
17. Gottlieb et al. Cell 56, 771-6. (1989).
18. Aguilaniu et al. Science (2003).
19. Tissenbaum et al. Nature 410, 227-30. (2001).
20. Vaziri et al. Cell 107, 149-59. (2001).
21. Luo et al. Cell 107, 137-48. (2001).
22. Vergnes et al. Gene 296, 139-50 (2002).
23. Holzenberger et al. Nature 421, 182-7 (2003).
24. Shimokawa et al. Faseb J 17, 1108-9 (2003).
25. Tatar et al. Science 299, 1346-51 (2003).
26. Bitterman et al. J Biol Chem 277, 45099-107. (2002).
27. Langley et al. EMBO J. 21, 2383-2396 (2002).
28. Glossmann et al. Naunyn Schmiedebergs Arch Pharmacol 317, 100-2 (1981).
29. Oliver et al. J Biol Chem 269, 29697-703 (1994).
30. Ferguson et al. Mutat Res 475, 89-111 (2001).
31. Middleton et al. Pharmacol Rev 52, 673-751 (2000).
32. Jang et al. Science 275, 218-20 (1997).
33. Stojanovic et al. Arch Biochem Biophys 391, 79-89 (2001).
34. Monod et al. J. Mol. Biol. 12, 88-118 (1965).
35. Anderson et al. J Biol Chem 277, 18881-90. (2002).
36. Pont et al. J Phytopathol 130, 1-8 (1990).
37. Sinclair et al. Cell 91, 1033-42. (1997).
38. Defossez et al. Mol Cell 3, 447-55 (1999).
39. Park et al. Mol Cell Biol 19, 3848-56 (1999).
40. Benguria et al. Nucleic Acids Res 31, 893-8 (2003).
41. Longo et al. Science 299, 1342-6 (2003).
42. Denu et al. Trends Biochem Sci 28, 41-8 (2003).
43. Dong et al. Mutat Res 523-524, 145-50 (2003).
44. Nicolini et al. Neurosci Lett 302, 41-4 (2001).
45. Jazwinski, S. M. et al. Ann NY Acad Sci 908, 21-30 (2000).
46. Pandey et al. Nucleic Acids Res 30, 5036-55 (2002).
47. Frye, R. A. Biochem Biophys Res Commun 273, 793-8. (2000).
48. Soleas et al. Clin Biochem 30, 91-113 (1997).
49. Coronado et al. Plant Physiol 108, 533-542 (1995).
50. Masoro, E. J. Exp Gerontol 35, 299-305. (2000).

### Example 6

### Localization of the Activation Domain of Sirtuins to Their N-Terminus

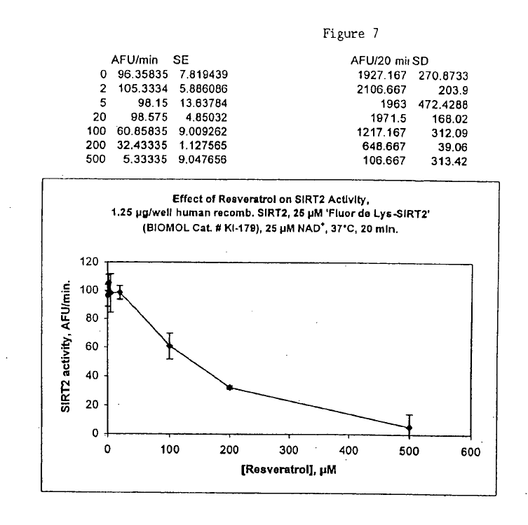

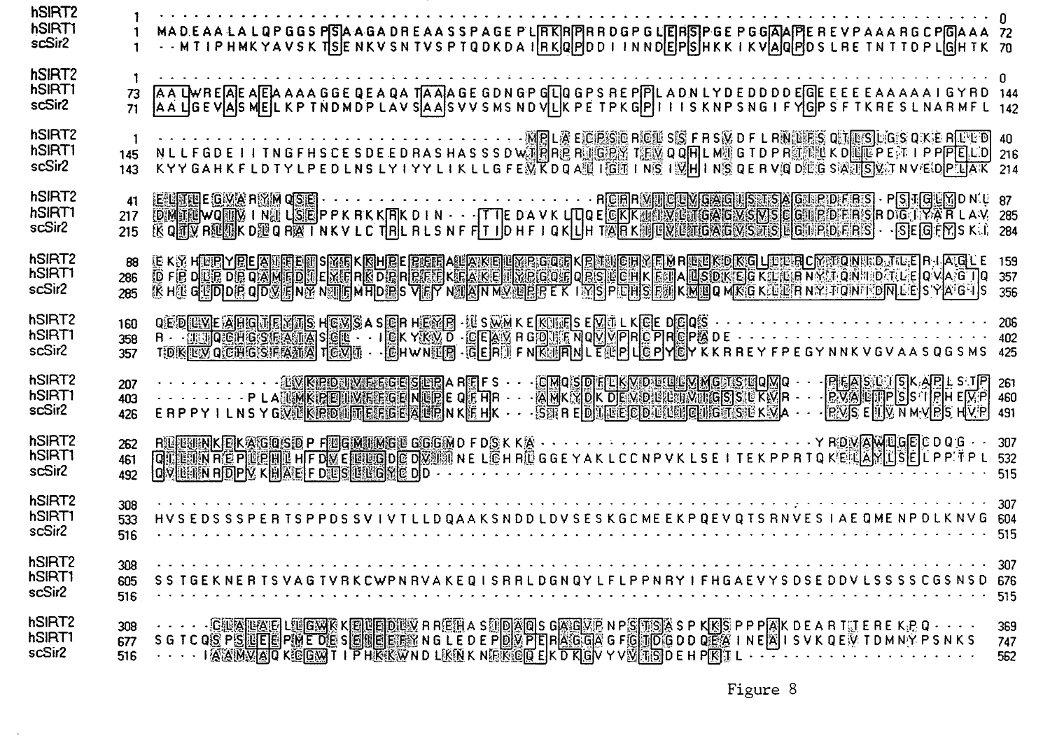

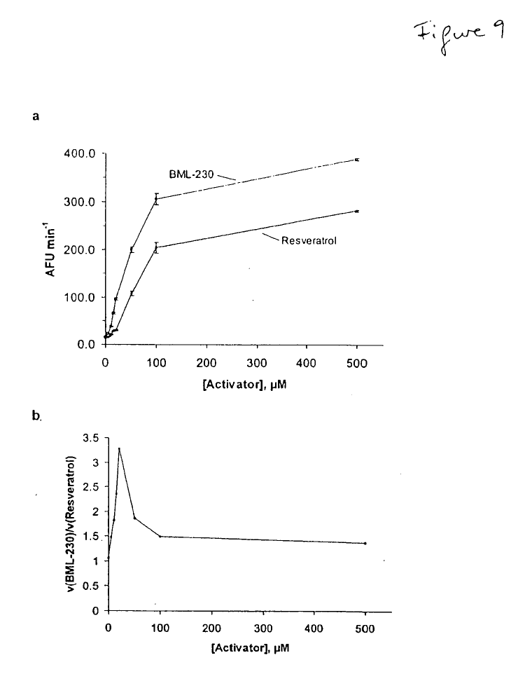

Yeast Sir2 and human SIRT1 are very homologous and differ from human SIRT2 by the addition of an N-terminal domain that is absent in SIRT2. The effect of resveratrol was assayed on human recombinant SIRT2 as follows. Human recombinant SIRT2 was incubated at a concentration of 1.25 μg/well with 25 μM of Fluor de Lys-SIRT2 (BIOMOL cat. # KI-179) and 25 μM NAD+ for 20 minutes at 37° C., as described above. The results, which are shown in FIG. 7, indicate that, in contrast to SIRT1, increasing concentrations of resveratrol decrease SIRT2 activity. Thus, based on the difference in structure of SIRT1 and SIRT2, i.e., the absence of an N-terminal domain (see FIG. 8), it is believed that the N-terminal domain of SIRT1 and Sir2 is necessary for activation by the compounds described herein. In particular, it is likely that the activator compounds described herein interact with the N-terminal portion of sirtuins. The N-terminal portion of SIRT1 that is necessary for the action of the compounds is from about amino acid 1 to about amino acid 176, and that of Sir2 is from about amino acid 1 to about amino acid 175.

### Example 7

### Resveratrol Extends the Lifespan of C. elegans

50 C. elegans worms (strain N2) were grown in the presence or absence of 100 μM resveratrol for 17 days. On day 17, only 5 worms in the control group without resveratrol were alive, whereas 17 worms were alive in the group that was treated with resveratrol. Thus, the presence of resveratrol in the growth media of C. elegans extends their lifespan.

### Example 8

### Identification of Additional Activators of Sirtuins

Using the screening assay described in Example 1, five more sirtuin activators have been identified. These are set forth in supplementary Table 8.

### Example 9

### Identification of Inhibitors of Sirtuins

Using the screening assay described in Example 1, more inhibitors were identified. These are set forth in the appended supplementary Table 8, and correspond to the compounds having a ratio to control rate of less than 1.

### Example 10

### Identification of Further Activators and Inhibitors of Sirtuins

Additional activators and inhibitors of sirtuins were identified, and are listed in Tables 9-13. In these Tables, “SE” stands for Standard error of the mean and N is the number of replicates used to calculate mean ratio to the control rate and standard error.

All SIRT1 rate measurements used in the calculation of “Ratio to Control Rate” were obtained with 25 μM NAD⁺ and 25 μM p53-382 acetylated peptide substrate were performed as described above and in K. T. Howitz et al. Nature (2003) 425: 191. All ratio data were calculated from experiments in which the total deacetylation in the control reaction was 0.25-1.25 μM peptide or 1-5% of the initial concentration of acetylated peptide.

Stability determinations (t_{1/2}) derived from SIRT1 rate measurements performed in a similar way to those described above, except that 5 μM p53-382 acetylated peptide substrate was used rather than 25 μM. The fold-stimulation (ratio to control) obtained with a compound diluted from an aged stock solution was compared to an identical dilution from a stock solution freshly prepared from the solid compound. “t_{1/2}” is defined as the time required for the SIRT1 fold-stimulation of the compound from the aged solution to decay to one-half of that obtained from a freshly prepared solution. Ethanol stocks of resveratrol, BML-212 and BML-221 were prepared at 2.5 mM and the compounds were assayed at a final concentration of 50 μM. The water stock of resveratrol was 100 μM and the assay performed at 10 μM. Stocks were aged by storage at room temperature, in glass vials, under a nitrogen atmosphere.

The effect of some of these compounds on lifespan was determined in yeast and C. elegans, as described above. The results are set forth below in Table 19:

| | % change in yeast | % change in |
|---|---|---|
| | replicative lifespan | C. elegans |
| | relative to untreated | lifespan relative to |
| | organisms | untreated organisms |
| Compound | (10 μM)^{a} | (100/500 μM)^{b} |
| untreated | 100% | 100% |
| Resveratrol | 170-180% | 110% |
| 3,5,4′-Trihydroxy-trans- | | |
| stilbene | | |
| Pinosylvin | 114% | ND |
| 3,5-Dihydroxy-trans- | | |
| stilbene | | |
| BML-212 | 98% | ND |
| 3,5-Dihydroxy-4′-fluoro- | | |
| trans-stilbene | | |
| BML-217 | 90% | ND |
| 3,5-Dihydroxy-4′-chloro- | | |
| trans-stilbene | | |
| BML-221 | 165% | >100% (ongoing) |
| 3,4′-Dihydroxy-5-acetoxy- | | |
| trans-stilbene | | |
| BML-233 | ND | 70% (10) |
| 3,5-Dihydroxy-4′-methoxy- | | 50% (500) |
| trans-stilbene | | |

^{a}Replicative lifespans performed using 2% (w/v) glucose standard yeast compete medium (YPD) under standard conditions.
^{b}Lifespan assays performed on N2 worms using E. coli as food under standard conditions.
ND. Not determined.

The results indicate that resveratrol significantly extends lifespan in yeast and in C. elegans. Since BML-233 was shown to be a strong activator of sirtuins (see above), the results obtained in C. elegans may indicate that the compound is toxic to the cells.

Without wanting to be limited to particular structures, it appears that the following structure/activity relationships exist. SIRT1 activation results from several of these new analogs confirmed the importance of planarity, or at least the potential for planarity, between and within the two rings of the active compounds. Reduction of the double bond of the ethylene function, between, the two rings essentially abolishes activity (compare Resveratrol, Table A and Dihydroresveratrol, Table E). Replacement of a phenyl moiety with a cyclohexyl group is nearly as detrimental to SIRT1 stimulating activity (compare Pinosylvin, Table 9 and BML-224, Table 12). Amide bonds are thought to have a partially double bond character. However, replacement of the ethylene function with a carboxamide abolished activity (compare Pinosylvin, Table 9, with BML-219, Table 13). It is possible that this effect could be due in part to the position that carbonyl oxygen must assume in the conformation that places the two rings trans to one another. If so, a compound in which the positions of the amide nitrogen and carbonyl are reversed might be expected to have greater activity.

In twelve of the analogs resveratrol's 4′-hydroxy was replaced with various functionalities (see Tables 9 and 10, BML-221 in Table 11, BML-222 in Table 12). Although none of the replacements tried led to substantial increases in SIRT1 stimulating activity, this parameter was, in general, remarkably tolerant of substitutions at this position. Small groups (H— in Pinosylvin, Cl— in BML-217, —CH₃ in BML-228) did the least to decrease activity. There is some evidence of a preference in the enzyme's stilbene binding/activation site for unbranched (ethyl in BML-225, azido in BML-232, —SCH₃ in BML-230) and hydrophobic functions (compare isopropyl in BML-231 to acetoxy in BML-221, acetamide in BML-222). Solution stability relative to resveratrol was strongly increased by one of the two 4′-substitutions (acetoxy, BML-221) tested for this so far.

Resveratrol is currently one of the most potent known activator of SIRT1. The collection of analogs described above, particularly the group entailing substitutions at the 4′ position, may be instrumental in informing the design of new SIRT1 ligands with improved pharmacological properties. One parameter that may be of interest in this regard is stability. One 4′-substituted analog, BML-221, displays a vast improvement in solution stability relative to resveratrol and although diminished in in vitro SIRT1 activating ability, retains much of resveratrol's biological activity (see lifespan data). The 4′-hydroxyl of resveratrol is thought to be of primary importance to resveratrol's free-radical scavenging reactivity (S. Stojanovic et al. Arch. Biochem. Biophys. 2001 391 79). Most of the 4′-substituted analogs have yet to be tested for solution stability, but if resveratrol's instability in solution is due to redox reactivity, many of the other analogs would be expected to also exhibit improved stability.

The results obtained with 4′-substituted analogs may indicate promising routes to explore while seeking to increase SIRT1 binding affinity. For example, the efficacy of the 4′-ethyl compound (BML-225) might indicate the presence of a narrow, hydrophobic binding pocket at the SIRT1 site corresponding to the 4′ end of resveratrol. Several new series of 4′-substituted analogs are planned, the simplest comprising straight-chain aliphatic groups of various lengths.

### Example 11

### Methods of Synthesis of the Compounds in Tables 9-13

Most of the resveratrol analogs were synthesized by the same general procedure, from a pair of intermediates, a benzylphosphonate and an aldehyde. The synthesis or sources of these intermediates are described in section II. Section III. describes the procedures for synthesizing the final compounds from any of the benzylphosphonate/aldehyde pairs. The coupling reaction (Section III. A.) is followed by one of two deprotection reactions depending on whether the intermediates contained methoxymethyl (Section III. B.) or methoxy (Section III. C.) protecting groups. Section IV corresponds to Tables 14-18, which list the particular benzylphosphonate and aldehyde used in the synthesis of particular final compounds. Seven of the compounds—Resveratrol, 3,5-Dihydroxy-4′-methoxy-trans-stilbene, Rhapontin aglycone, BML-227, BML-221, Dihydroresveratrol, BML-219—were not synthesized by the general procedure and “N/A” appears next to their entries in the table. Resveratrol was from BIOMOL and the syntheses of the remaining compounds are described in Section V.

II. Synthetic Intermediates

A. Benzylphosphonates (Synthesized)

Synthesis of Diethyl 4-Acetamidobenzylphosphonate: To diethyl 4-aminobenzylphosphonate in 1:1 methylene chloride/pyridine was added catalytic DMAP and acetic anhydride (1.1 eq.). After 3 hours, the reaction was evaporated to dryness and purified via flash chromatography (silica gel).

Synthesis of Diethyl 4-Methylthiobenzylphosphonate: 4-Methylthiobenzyl chloride was heated with triethylphosphite (as solvent) at 120° C. overnight. Excess triethyl phosphite was distilled off under high vacuum and heat. Flash chromatography (silica gel) yielded the desired product.

Synthesis of Diethyl 3,5-Dimethoxybenzylphosphonate: From 3-5-Dimethoxybenzyl bromide. See synthesis of Diethyl 4-Methylthiobenzylphosphonate.

Synthesis of Diethyl 4-Fluorobenzylphosphonate: From 4-Fluorobenzylphosphonate. See synthesis of Diethyl 4-Methylthiobenzylphosphonate.

Synthesis of Diethyl 4-azidobenzylphosphonate: To diethyl 4-aminobenzylphosphonate in acetonitrile (2.5 mL) at 0° C. was added 6M HCl (1 mL). Sodium nitrite (1.12 eq.) in water (1 mL) was added drop wise and the resulting solution stirred at 0° C. for 30 mins. Sodium azide (8 eq.) in water (1 mL) added drop wise (bubbling) and the solution stirred at 0° C. for 30 mins., then at room temperature for 1 hour. The reaction was diluted with ethyl acetate and washed with water and brine and dried over sodium sulfate. Flash chromatography (silica gel) yielded the desired product.

B. Aldehydes (Synthesized)

Synthesis of 3,5-Dimethoxymethoxybenzaldehyde: To 3,5-dihydroxybenzaldehyde in DMF at 0° C. was added sodium hydride (2.2 eq.). The reaction was stirred for 30 min. at 0° C. Chloromethylmethyl ether (2.2 eq.) was added neat, drop wise and the reaction allowed to warm to room temperature over 1.5 hrs. The reaction mixture was diluted with diethyl ether and washed with water (2×) and brine (1×) and dried over sodium sulfate. Flash chromatography (silica gel) yielded the desired product.

C. Purchased Intermediates: Unless listed above, all synthetic intermediates were purchase from Sigma-Aldrich.

III. General Procedure for the Synthesis of Resveratrol Analogues

A. Benzylphosphonate/Aldehyde Coupling Procedure

To the appropriate benzylphosphonate (1.2 eq.) in dimethylformamide (DMF) at room temperature was added sodium methoxide (1.2 eq.). This solution was allowed to stir at room temperature for approximately 45 minutes. The appropriate aldehyde (1 eq.) was then added (neat or in a solution of dimethylformamide). The resulting solution was then allowed to stir overnight at room temperature. Thin layer chromatography (TLC) was used to determine completeness of the reaction. If the reaction was not complete, the solution was heated at 45-50° C. until complete. The reaction mixture was poured into water and extracted with ethyl acetate (2×). The combined organic layers were washed with brine and dried over sodium sulfate. Flash chromatography (silica gel) yielded the desired products.

B. General Procedure for the Deprotection of Methoxymethylresveratrol Analogues

To the appropriate methoxymethylstilbene derivative in methanol was added two drops of concentrated HCl. The resulting solution was heated overnight at 50° C. The solution was evaporated to dryness upon completion of the reaction. Flash chromatography (silica gel) yielded the desired product.

C. General Procedure for the Deprotection of Methoxyresveratrol Analogues

To the appropriate methoxystilbene derivative in methylene chloride was added tetrabutylammonium iodide (1.95 eq. per methoxy group). The reaction was cooled to 0° C. and boron trichloride (1 M in methylene chloride; 2 eq. per methoxy group) was added dropwise. Following the addition of boron trichloride, the cooling bath was removed and the reaction allowed to stir at room temperature until complete (as indicated by TLC). Saturated sodium bicarbonate solution was added and the reaction vigorously stirred for 1 hour. The reaction was poured into cold 1M HCl and extracted with ethyl acetate (3×). The combined organic layers were washed with water (1×) and brine (×) and dried over sodium sulfate. Flash chromatography (silica gel) yielded the desired products.

V. Special Syntheses

Synthesis of BML-219 (N-(3,5-Dihydroxyphenyl)benzamide): To benzoyl chloride (1 eq.) in dry methylene chloride at room temperature was added triethylamine (1.5 eq.) and a catalytic amount of DMAP followed by 3,5-dimethoxyaniline (1 eq.). The reaction was allowed to stir overnight at room temperature. Upon completion, the reaction was diluted with ethyl acetate and washed with 1M HCl, water and brine and dried over sodium sulfate. Flash chromatography (silica gel) yielded the methoxystilbene derivative. To the methoxystilbene in dry methylene chloride at 0° C. was added tetrabutylammonium iodide (3.95 eq.) followed by boron trichloride (4 eq.; 1M in methylene chloride). Upon completion of the reaction (TLC), saturated sodium bicarbonate was added and the mixture was vigorously stirred for 1 hour. The reaction was diluted with ethyl acetate and washed with 1M HCl and brine and dried over sodium sulfate. Flash chromatography (silica gel) yielded the desired product.

Synthesis of BML-220 (3,3′,5-trihydroxy-4′-methoxystilbene): To Rhapontin in methanol was added catalytic p-toluenesulfonic acid. The reaction was refluxed overnight. Upon completion of the reaction (TLC), the reaction mixture was evaporated to dryness and taken up in ethyl acetate. The organics were washed with water and brine and dried over sodium sulfate. Flash chromatography (silica gel) yielded the desired product.

Synthesis of BML-233 (3,5-Dihydroxy-4′-methoxystilbene): To deoxyrhapontin in methanol was added catalytic p-toluenesulfonic acid. The reaction was refluxed overnight. Upon completion of the reaction (TLC), the reaction mixture was evaporated to dryness and taken up in ethyl acetate. The organics were washed with water and brine and dried over sodium sulfate. Flash chromatography (silica gel) yielded the desired product.

Synthesis of BML-221 and 227 (4′ and 3 monoacetylresveratrols): To resveratrol in tetrahydrofuran at room temperature was added pyridine (1 eq.) followed by acetic anhydride (1 eq.). After stirring for 48 hrs., another 0.25 eq. acetic anhydride added followed by 24 hrs. of stirring. The reaction was diluted with methylene chloride (reaction was not complete) and washed with cold 0.5M HCl, water and brine. Organics were dried over sodium sulfate. Flash chromatography yielded a mixture of 4′- and 3-acetyl resveratrols. Preparative HPLC yielded both monoacetyl resveratrols.

Synthesis of Dihydroresveratrol: To resveratrol in argon-purged ethyl acetate in a Parr shaker was added 10% palladium on carbon (10 wt %). The mixture was shaken under an atmosphere of hydrogen (30 psi) for 5 hours. Filtration through a pad of celite yielded the desired material.

### Example 12

### Dose-Response Analysis of SIRT1 Deacetylation by Resveratrol and BML-230

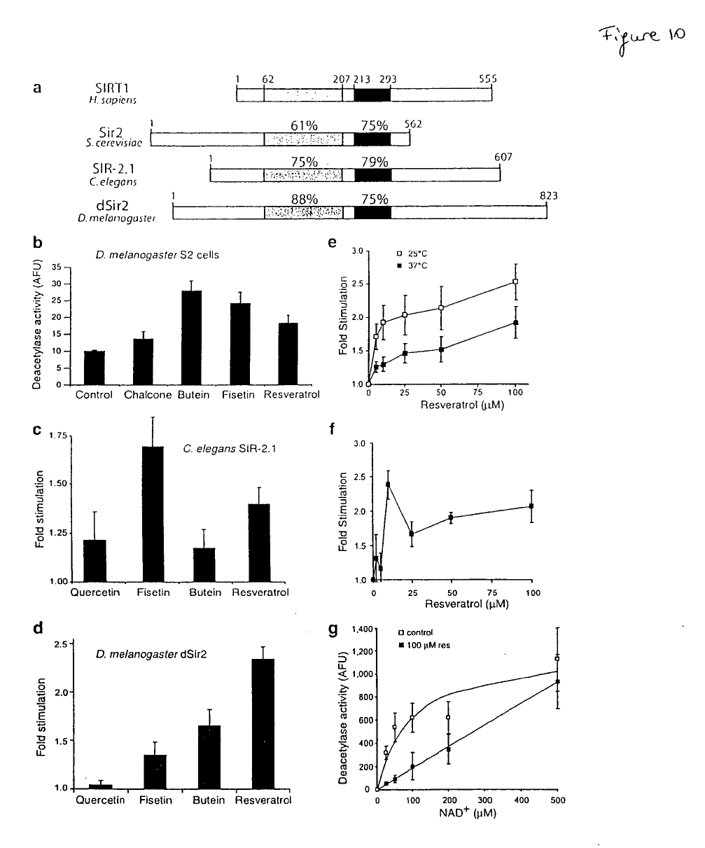

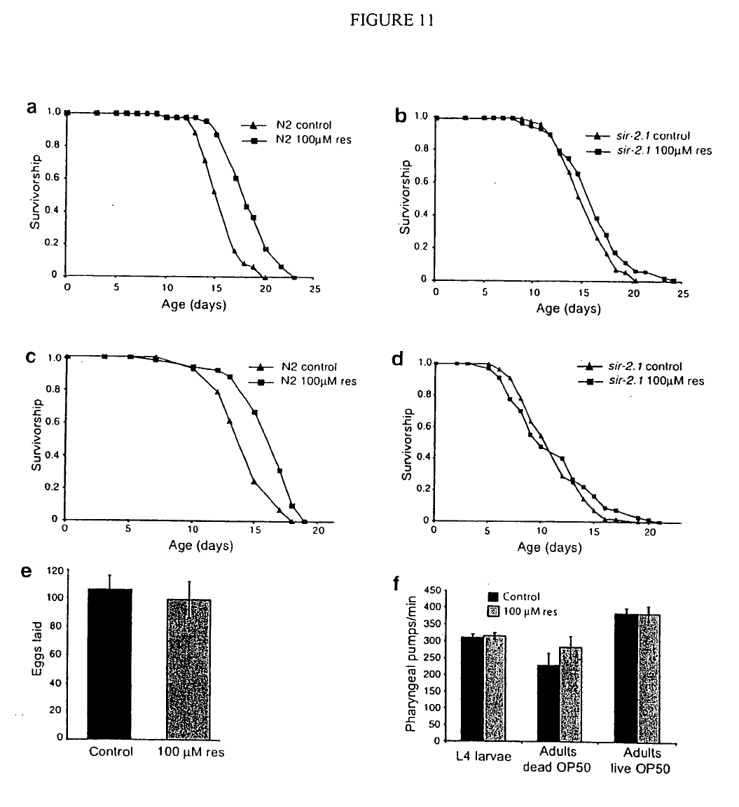

SIRT1 initial rates as a function of activator concentration were determined at 25 μM each of NAD⁺ and p53-382 acetylated peptide, with 20 minutes incubations. Plots of the dose responses of SIRT1 to BML-230 and resveratrol show that the BML-230-stimulated activity exceeds that stimulated by resveratrol at all concentrations tested (FIG. 9a). This could be due to a greater binding affinity of SIRT1 for BML-230, greater activity of the SIRT1/BML-230 complex or some combination of the two. A plot of the ratio of the rates of BML-230-stimulated enzyme to that of resveratrol-stimulated enzyme suggests that increased binding affinity does contribute to the improvement in activity of BML-230 (FIG. 9b). A simple two state model of the binding and activation process assumes that the observed rate (v) is the sum of the fractional contributions of the unliganded and liganded enzymes, where v₀ is the unstimulated rate, v₁ is the rate of the enzyme with bound ligand-1 (L1) and K_{L1} is the dissociation constant of the enzyme/ligand-1 complex:
v=v₀(1−[L1]/(K_{L1}+[L1]))+v₁(−[L1]/(K_{L1}+[L1])
A similar equation can be prepared for ligand-2 and the ratio (R) of the two rates calculated, an equation which will include, given the conditions of FIG. 9, the substitution [L]=[L1]=[L2]. It can be shown that if the two ligand dissociation constants were equal (K_{L1}=K_{L2}=K_{L}), this ratio would be:
R=(v₀K_{L}+v₁[L])/(v₀K_{L}+v₂[L])
If K_{L1}≠ K_{L2}, this ratio would instead be:
R=(v₁[L]²+(v₀K_{L1}+v₁K_{L2})[L]+v₀K_{L1}K_{L2})/(v₂[L]²+(v₀K_{L2}+v₂K_{L1})[L]+v₀K_{L1}K_{L2})
In the first case the plot of R vs. [L] would be a simple hyperbola that monotonically approaches v₁/v₂ as [L] increases. In the second case, as in FIG. 9b, the plot would pass through a maximum before approaching v₁/v₂ at higher [L] values. The data of FIG. 9b would imply that v₁/v₂ (rate for pure SIRT1/BML-230 divided by that for pure SIRT1/resveratrol) is no more than ˜1.4 (R at 500 μM) and that the SIRT1/BML-230 complex indeed has a lower dissociation constant than SIRT1/resveratrol (K_{L1}<K_{L2}).

One of the difficulties in the use of resveratrol as a pharmacologic agent is the relatively low serum concentrations of the aglycone form that can be achieved and maintained when it is administered orally (<<1 μM; see for example D. M Goldberg et al. Clin. Biochem. 2003 36 79). Increasing the SIRT1 binding affinity of synthetic derivatives will improve this aspect of the drug. As sest forth above, various replacements of the resveratrol 4′-hydroxyl, e.g. the H— of pinosylvin or Cl— of BML-217, did not significantly diminish the SIRT1 activating effect. The results obtained with BML-230 indicate that it will be possible to actually increase SIRT1/activator binding affinity by modifications at that site. The 4′-thiomethyl of BML-230 therefore represents a new starting point in seeking further improvements in SIRT1 binding affinity by the synthesis of related derivatives (e.g. 4′-thioethyl etc.).

### Example 13

### Survival Rates

Human 293 were grown to exponential phase under standard conditions and subjected to a dose of compound (50 micromolar) for 96 hours. The number of live cells each time point was counted using a Coulter counter.

TABLE 24
Survival statistics of 293 cells:

| | | Thio- | | | |
|---|---|---|---|---|---|
| | | Methyl | Ethyl | Methyl | Isopropyl |
| Time (h) | Resveratrol | BML-230 | BML-225 | BML-228 | BML-231 |
| 0 | 100% | 100% | 100% | 100% | 100% |
| 48 | 5% | 55% | 5% | 46% | 0% |
| 96 | 0% | 57% | 8% | 32% | 0% |

The results indicate that thiomethyl (BML-230) was the least toxic on 293 cells.

### Example 14

### Sirtuin Activators Mimic Calorie Restriction and Delay Aging in Metazoans

Caloric restriction (CR) extends lifespan in numerous species. In the budding yeast S. cerevisiae, this effect requires Sir2¹, a member of the sirtuin family of NAD⁺-dependent deacetylases^{2,3}. Sirtuin-activating compounds (STACs) can promote the survival of human cells and extend the replicative lifespan of yeast⁴. Here we show that resveratrol and other STACs activate sirtuins from Caenorhabditis elegans and Drosophila melanogaster and extend the lifespan of these animals up to 29% without reducing fecundity. Lifespan extension is dependent on functional Sir2 and is not observed when nutrients are restricted. Together these data indicate that STACs slow metazoan ageing by mechanisms related to CR.

Sir2-like proteins (sirtuins) are a family of NAD⁺-dependent deacetylases conserved from E. coli to humans⁵⁻⁹ (FIG. 10a) that play important roles in gene silencing, DNA repair, rDNA recombination and ageing in model organisms^{2,10-12}. When diet is restricted (calorie restriction, CR), lifespan is extended in diverse species, suggesting there is a conserved mechanism for nutrient regulation of ageing¹³⁻¹⁷. In budding yeast, extra copies this gene extend lifespan by 30% apparently by mimicking CR^{1,18}. We recently described a group of compounds (STACs) that stimulate the catalytic activity of yeast and human sirtuins, and extend the replicative lifespan of yeast cells up to 60%⁴.

To establish whether STACs could activate sirtuins from multicellular animals, we developed a cell-based deacetylation assay for D. melanogaster S2 cells. Several classes of polyphenolic STACs, including chalcones, flavones and stilbenes, increased the rate of deacetylation in an NAD⁺-dependent manner (FIG. 10b). To determine whether this activity was due to direct stimulation of a Sir2 homolog, we purified recombinant SIR-2.1 of C. elegans and dSir2 of D. melanogaster and determined the effect of various STACs on enzymatic activity in vitro (FIG. 10c, d). In a dose-dependent manner, resveratrol stimulated deacetylation up to 2.5-fold for SIR-2.1 (FIG. 10e) and 2.4-fold for dSir2 (FIG. 10f). As previously observed with the yeast and human Sir2 enzymes, resveratrol lowered the Kₘ of SIR-2.1 for the co-substrate NAD⁺ (FIG. 10g).

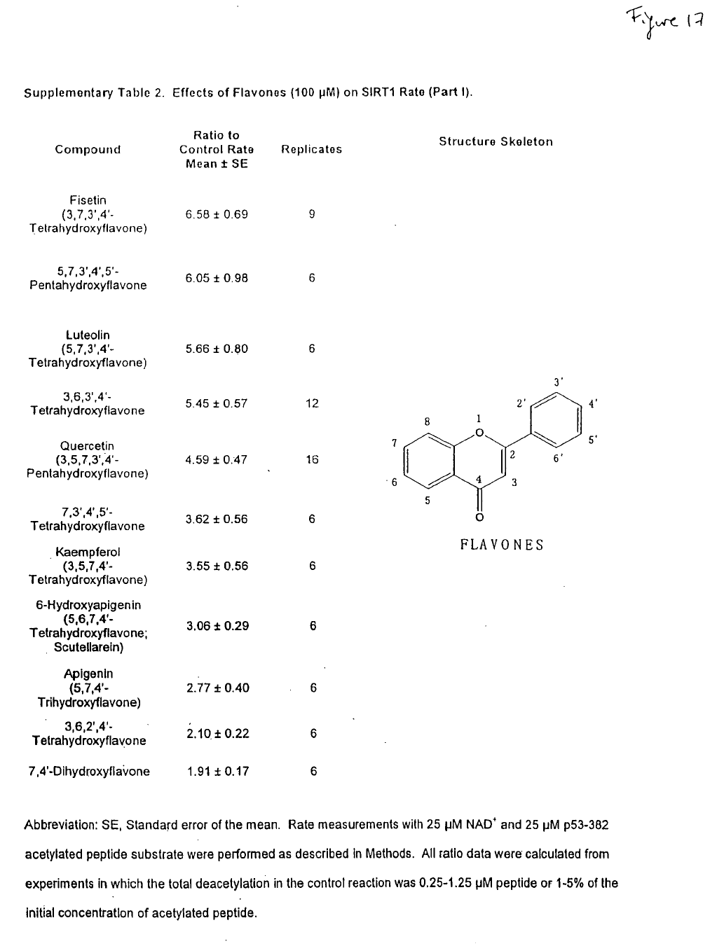

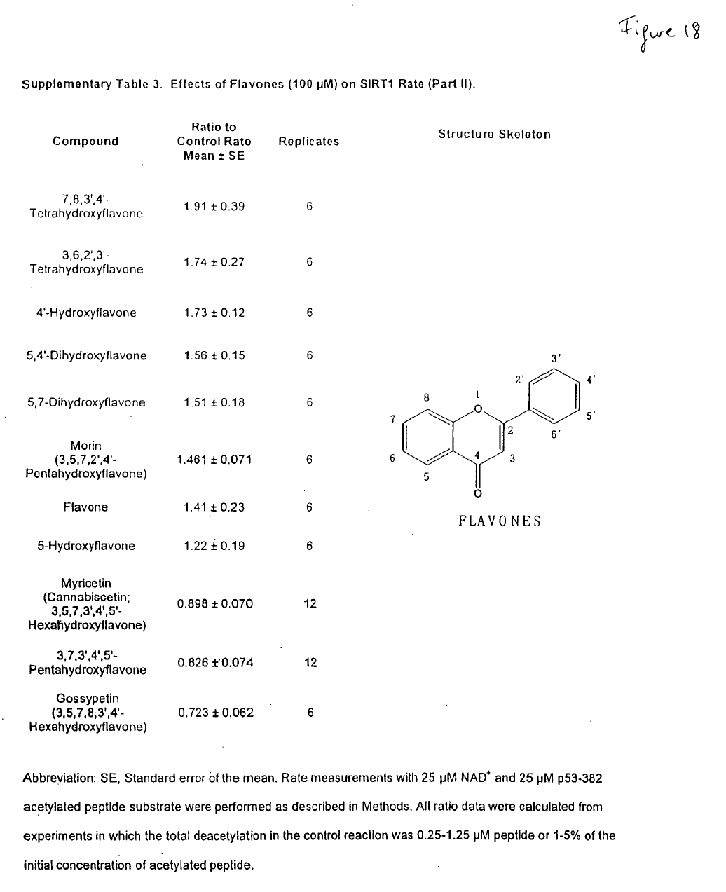

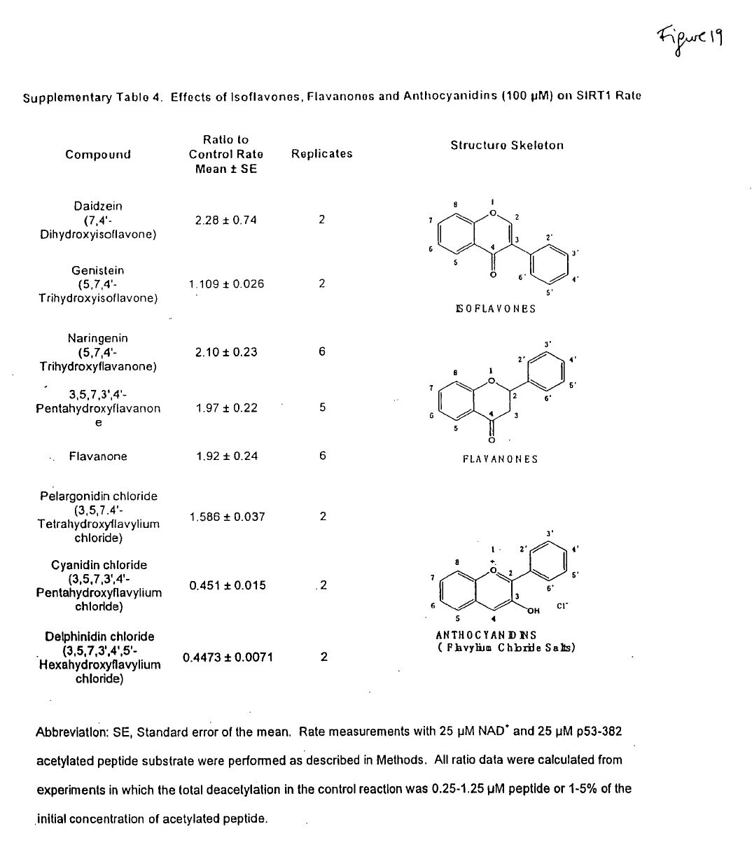

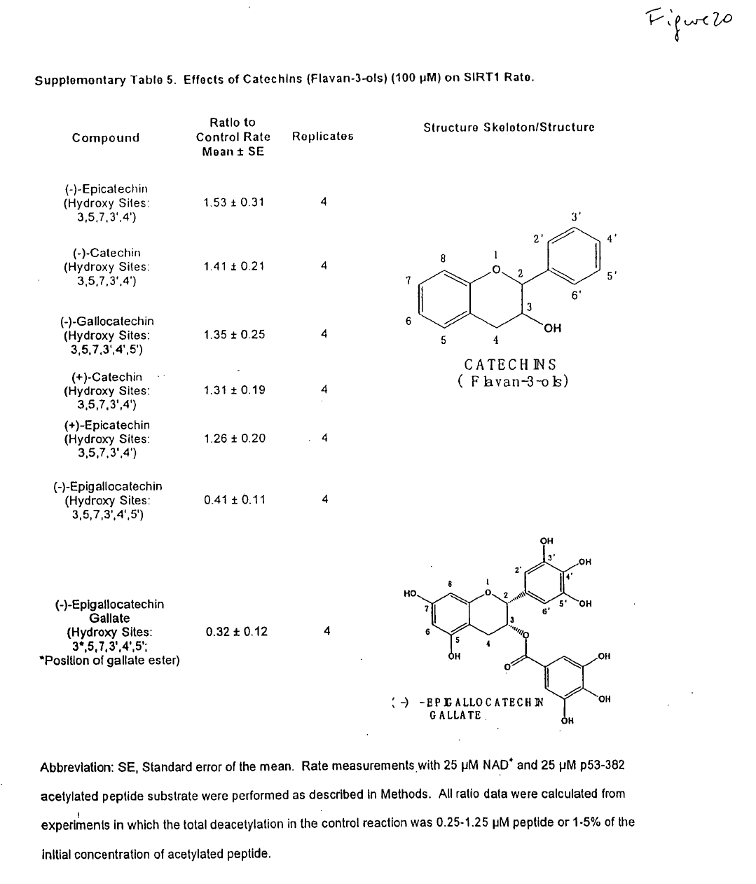

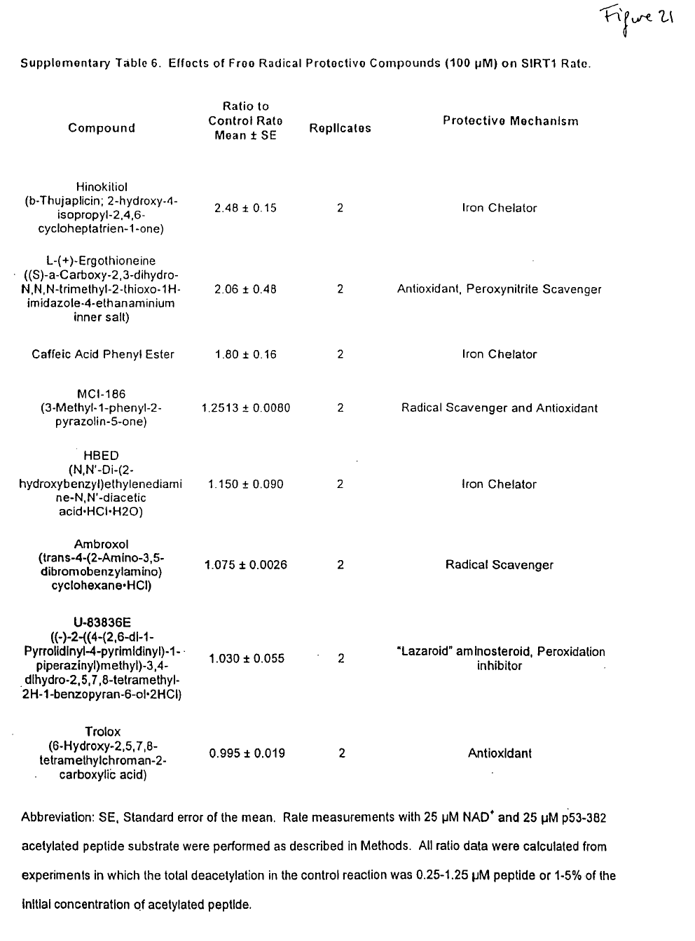

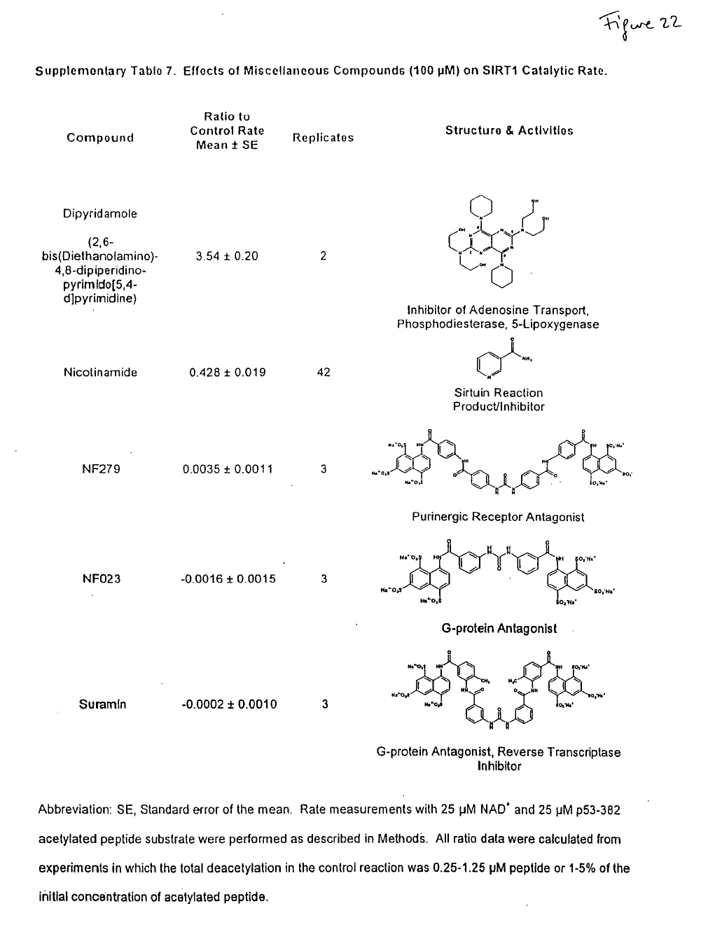

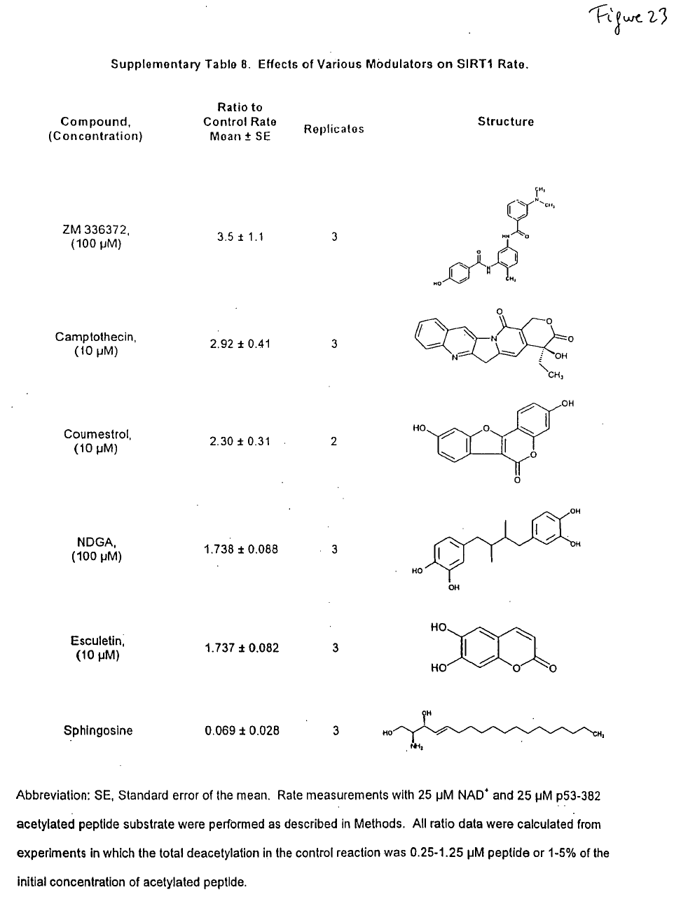

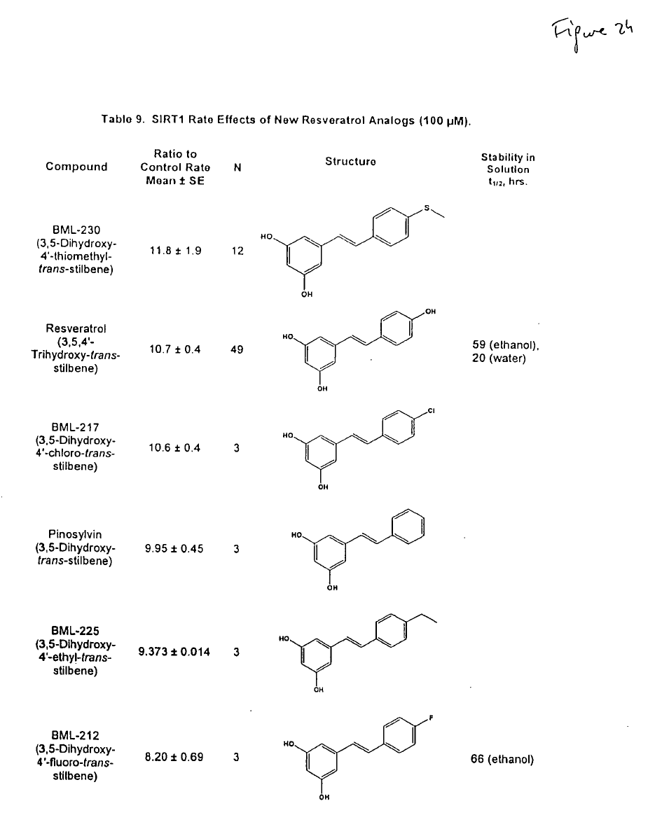

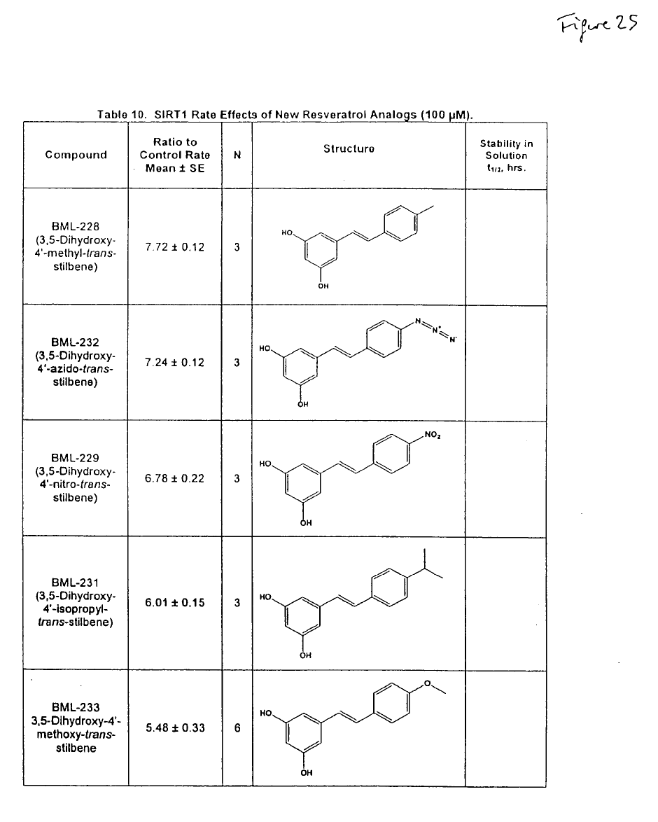

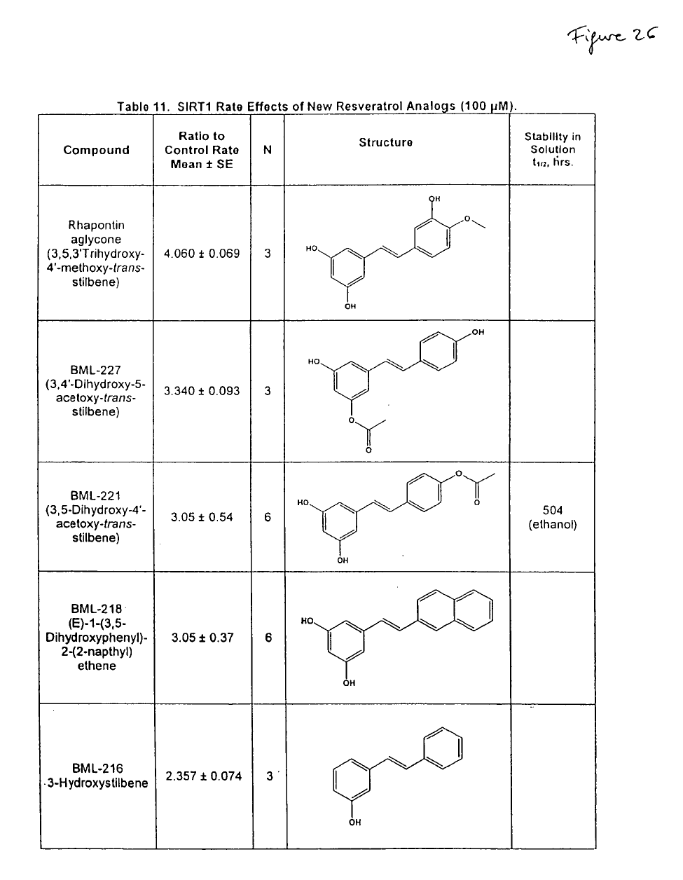

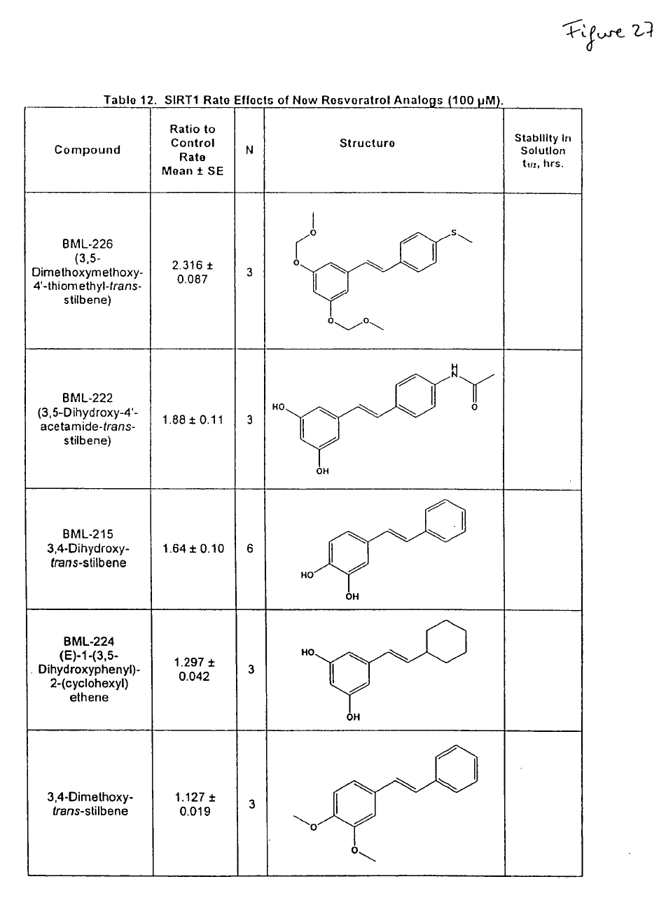

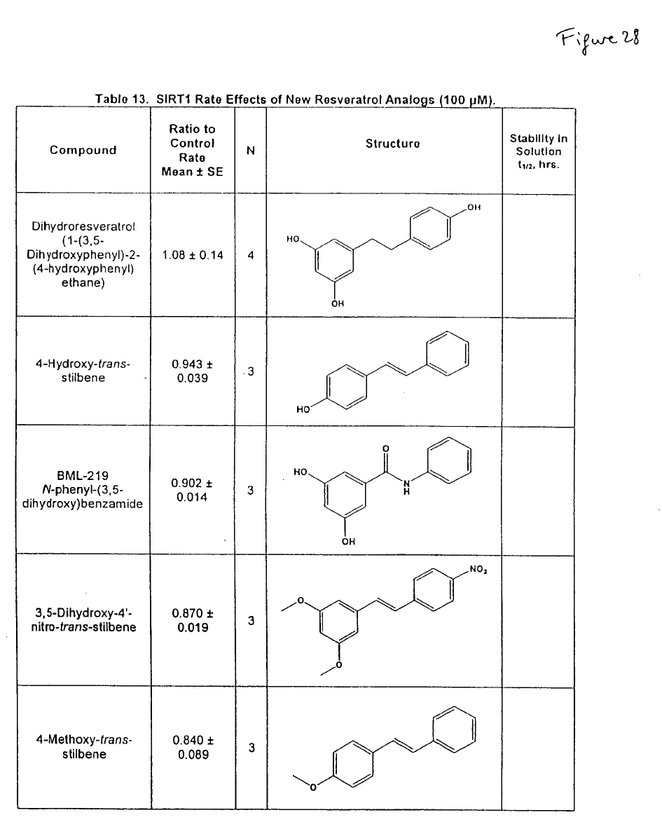

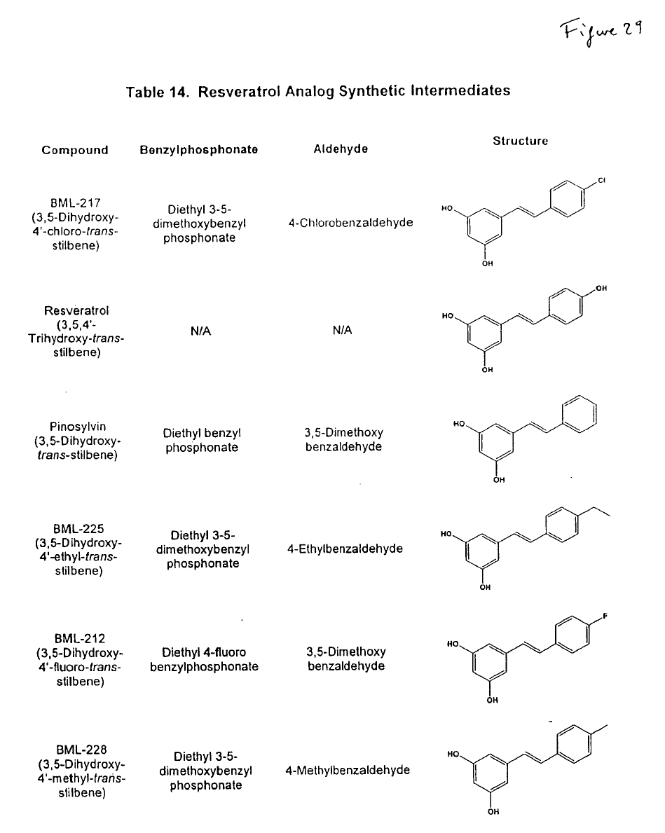

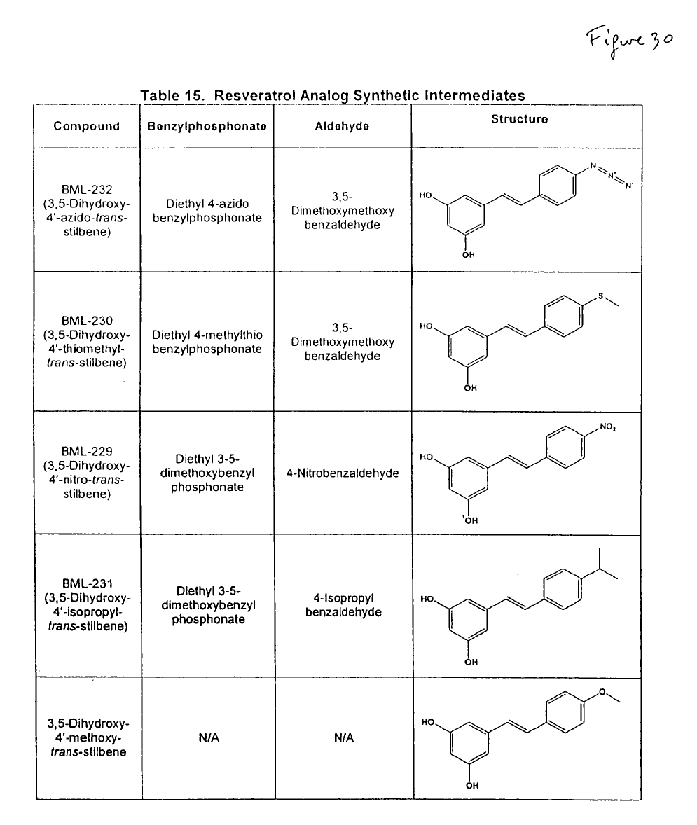

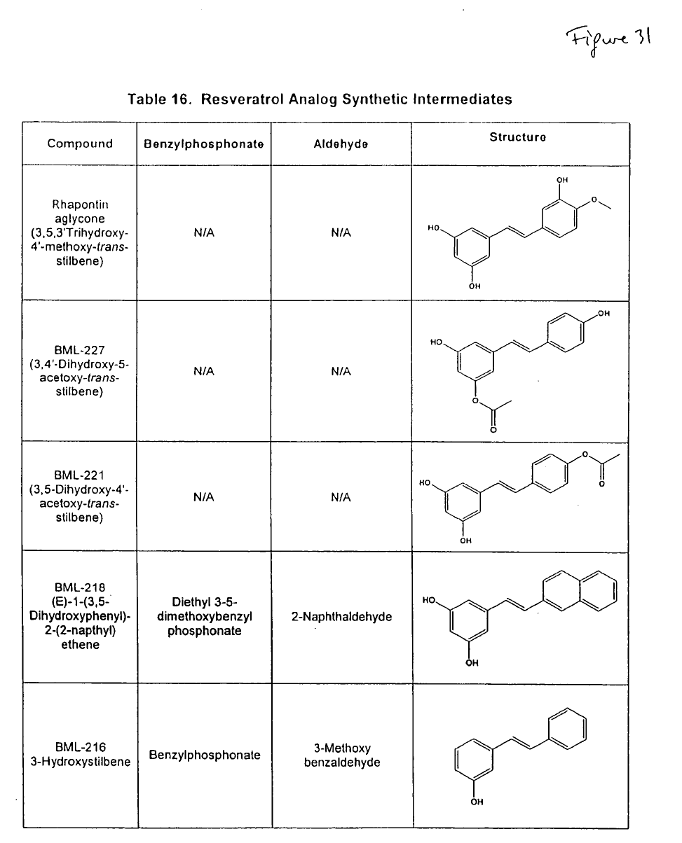

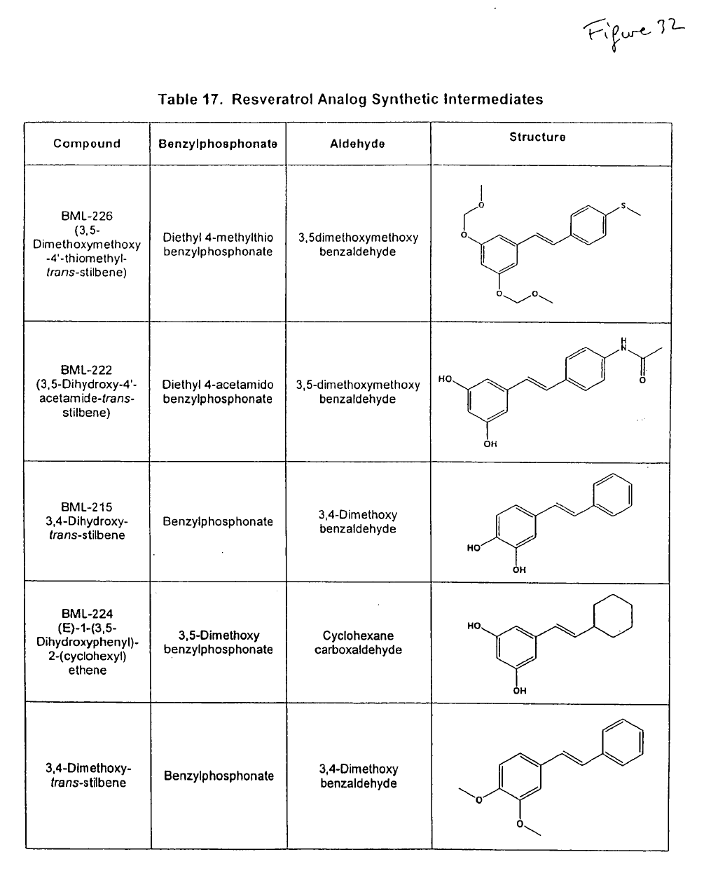

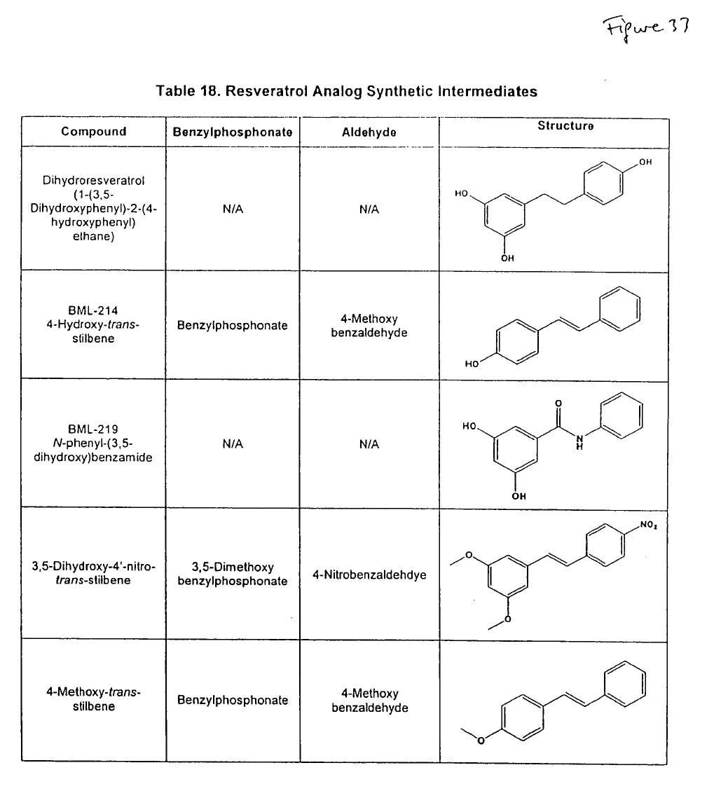

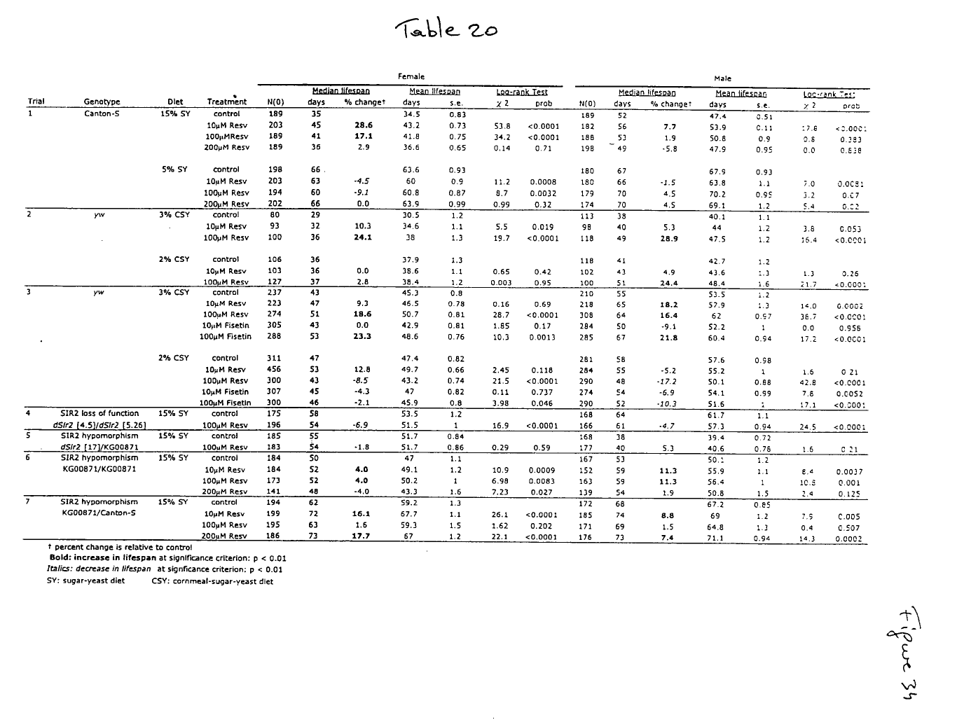

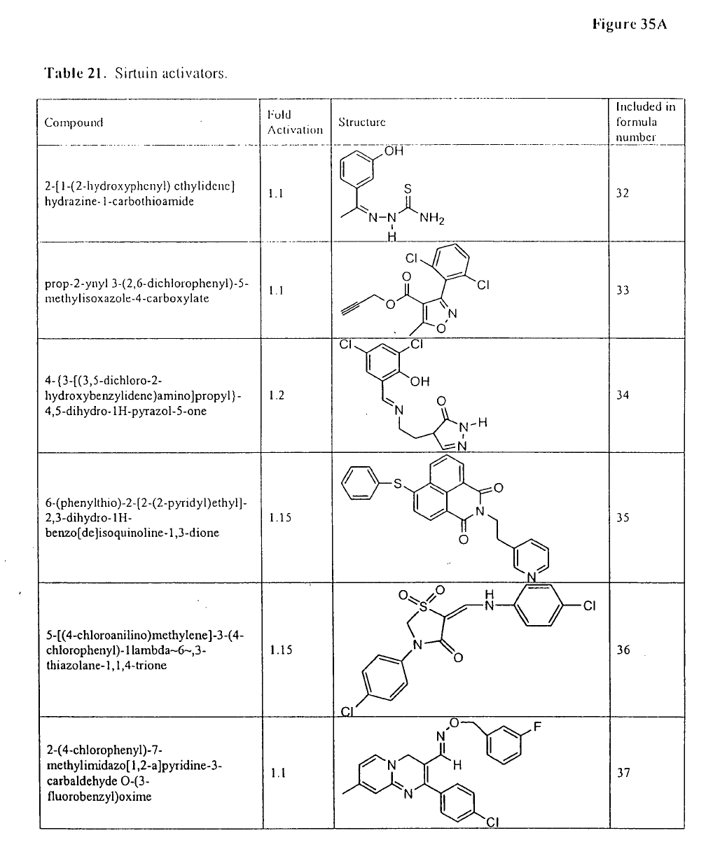

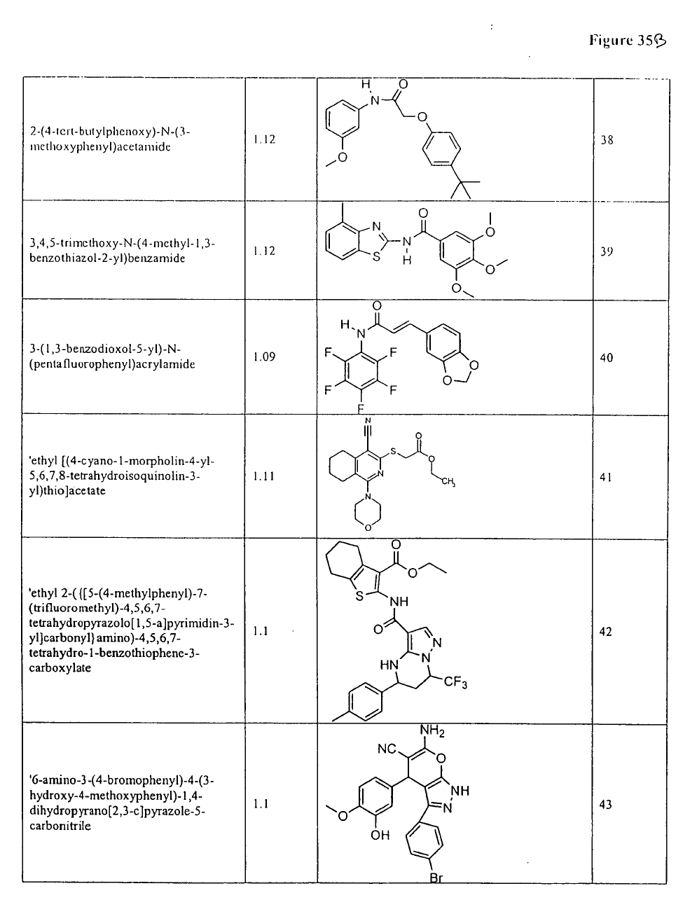

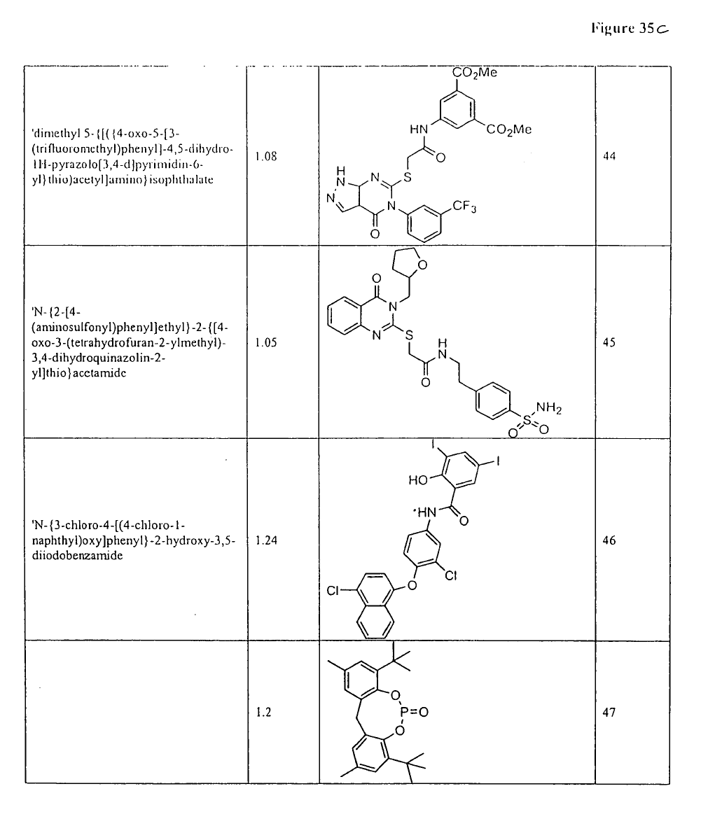

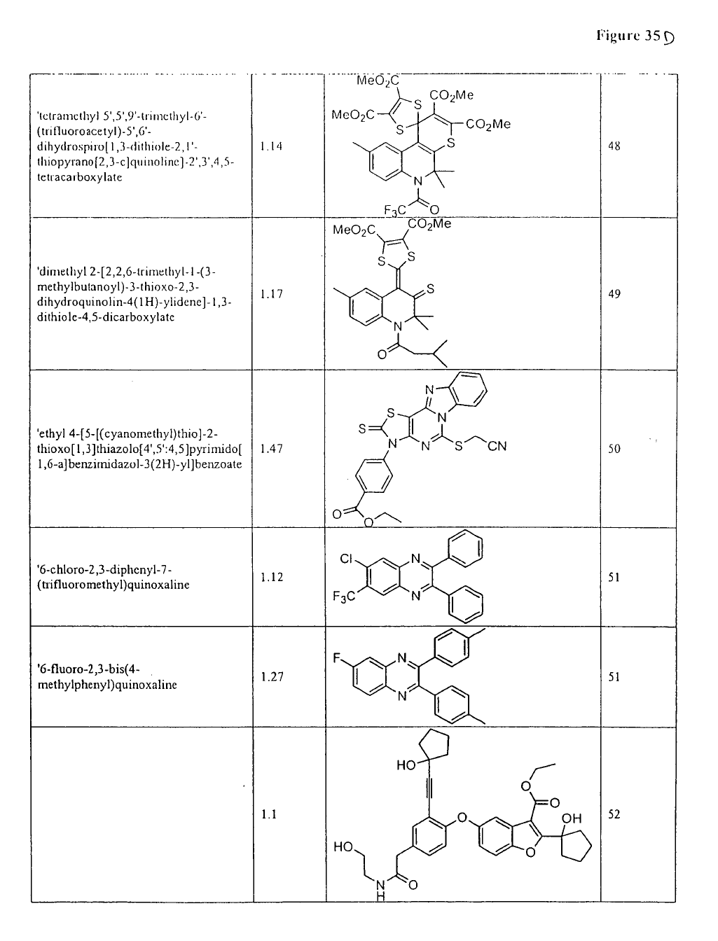

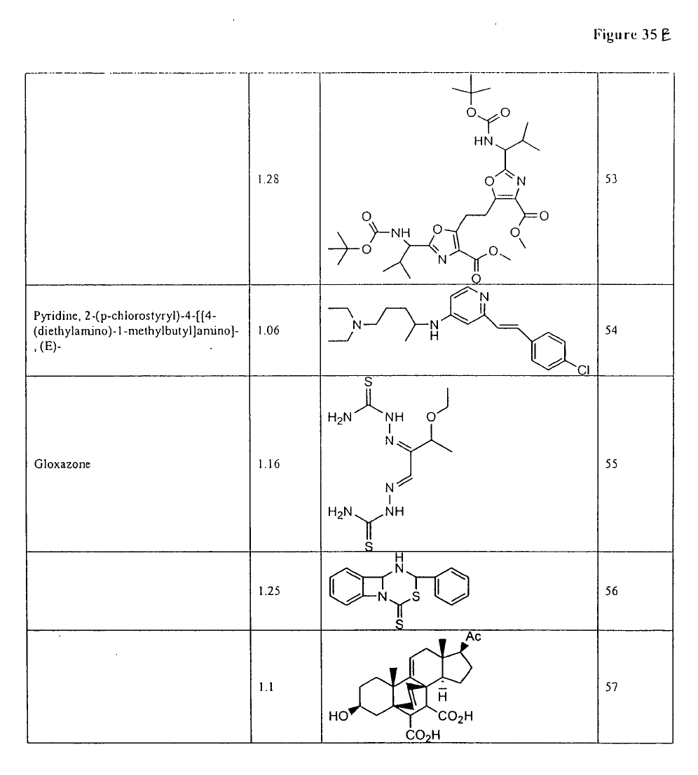

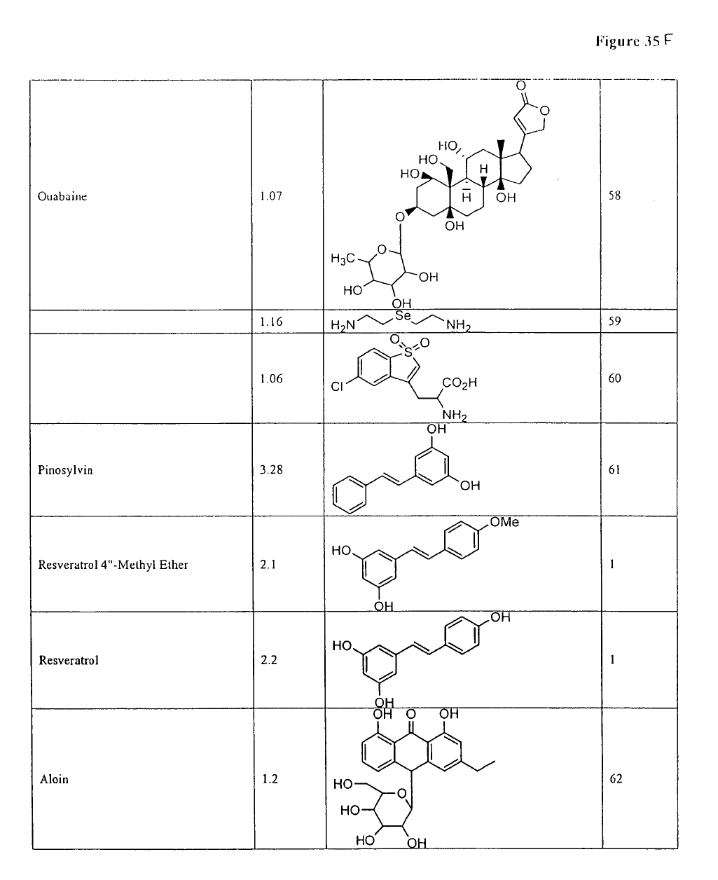

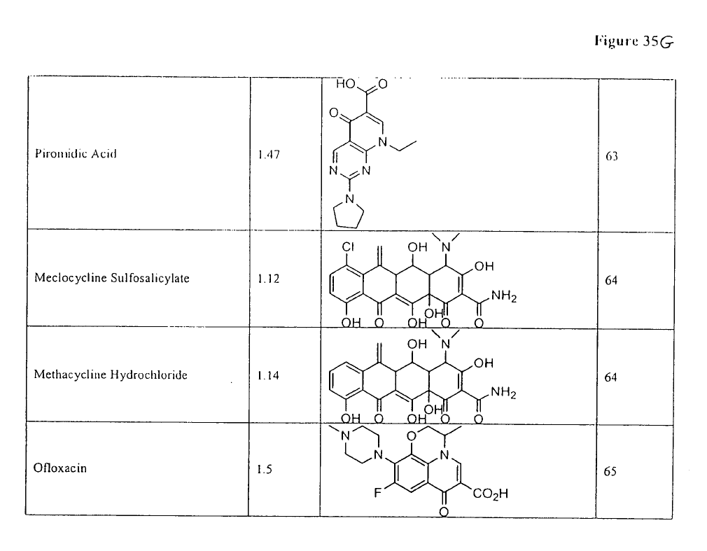

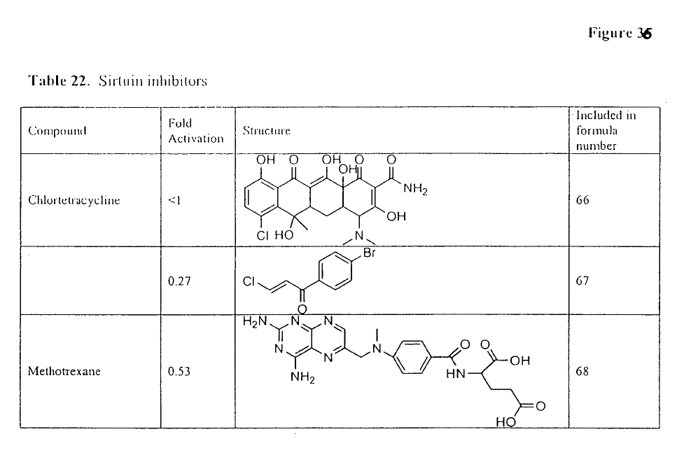

Because resveratrol can significantly extend replicative lifespan in yeast⁴, we asked whether STACs could also extend lifespan in the metazoans C. elegans and D. melanogaster. Wild-type worms were transferred to plates containing 0 or 100 μM of resveratrol shortly after reaching adulthood. Lifespan was reproducibly extended up to 15%, using either heat-killed or live E. coli as food supply (FIG. 11a, c respectively) and mortality was decreased across all adult ages (FIG. 14). To test whether the lifespan extension depends on functional SIR-2.1, we constructed a sir-2.1 null mutant. The lifespan of this strain was not appreciably shorter than the wildtype N2 control and adults treated with resveratrol did not exhibit a significant lifespan extension relative to untreated worms (FIG. 11b, d). There was no decrease in fecundity associated with resveratrol treatment (FIG. 11e). To rule out the possibility that resveratrol was causing the animals to eat less, thereby inducing a CR effect indirectly, we measured feeding rates of both L4 larval and adult worms with or without resveratrol and found no differences (FIG. 11f).

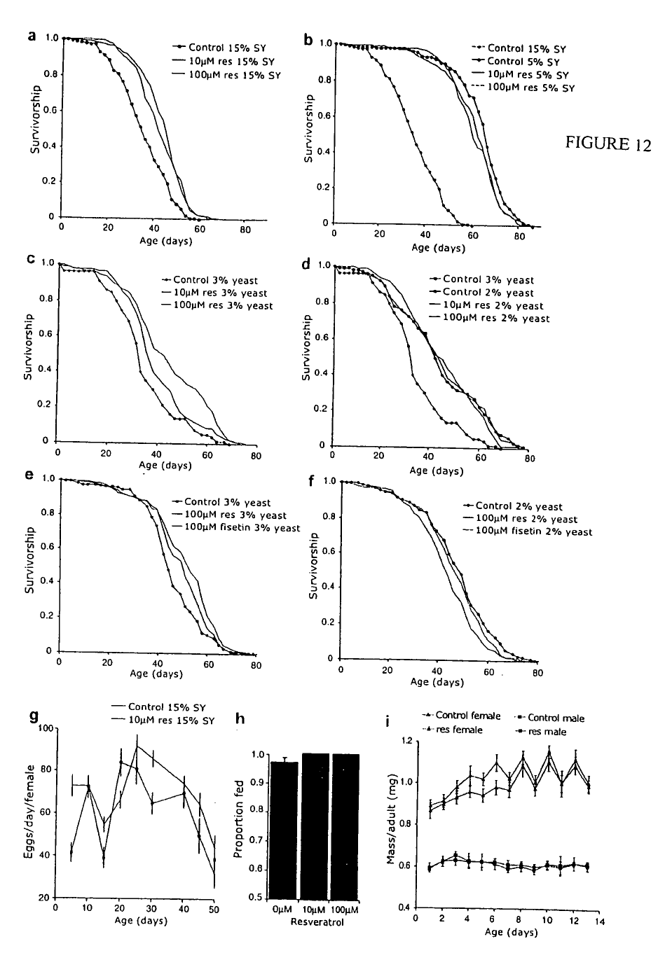

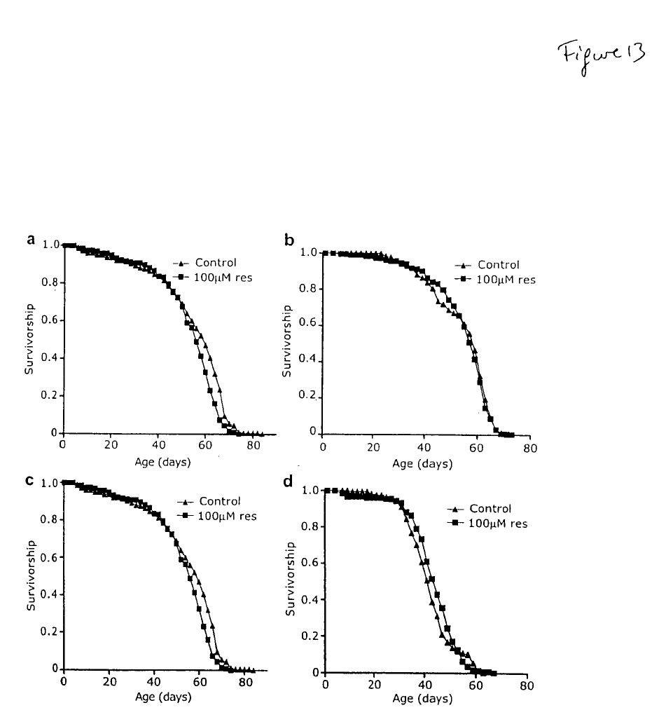

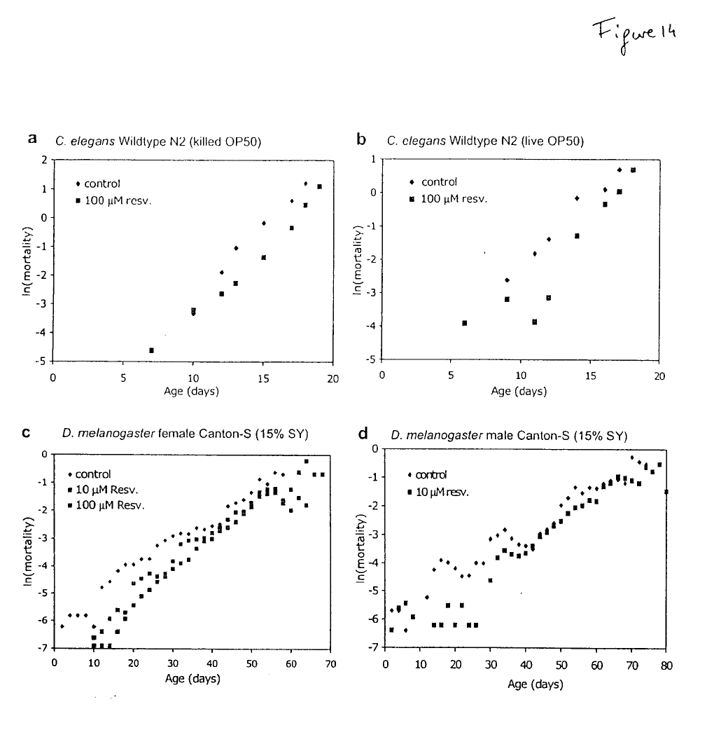

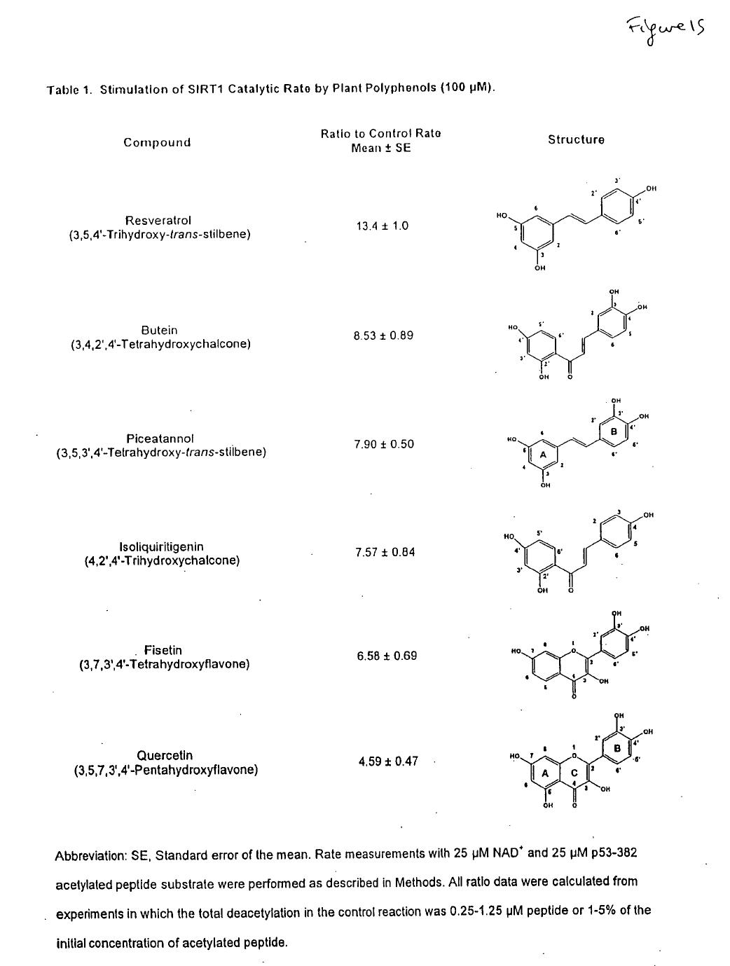

We also tested whether STACs could extend lifespan in D. melanogaster using the standard laboratory wild type strain Canton-S and normal fly culturing conditions (vials), and a yw marked wild type strain and demographic culturing conditions (cages) (Table 20). Across independent tests in males and females, lifespan was extended up to 23% with fisetin and up to 29% with resveratrol (FIG. 12a, c, e). Increased longevity was associated with reduced mortality prior to day 40 (FIG. 14). A restricted diet increased lifespan by 40% in females and by 14% in males (averaged across trials), and under these conditions neither resveratrol nor fisetin further increased longevity (FIG. 12b, d, f), suggesting that resveratrol extends lifespan through a mechanism related to CR.

Surprisingly, while diet manipulations that extend D. melanogaster longevity typically reduce fecundity^{19,20}, longevity-extending doses of resveratrol modestly increased egg production (10 μM resveratrol: 69.8 eggs/5 days, s.e.=2.2; control: 59.9 eggs/5 days, s.e.=2.2; t=3.17, P=0.0017), particularly in the earliest days of adult life (FIG. 12g). The increase in egg production suggests that the lifespan extending effect of resveratrol in D. melanogaster was not due to CR induced by food aversion or lack of appetite. Consistent with this, no decrease in food uptake was seen with resveratrol-fed flies (FIG. 12h). Furthermore, resveratrol-fed flies maintained normal weight (FIG. 12i), except during days 3 through when resveratrol fed females were laying significantly more eggs than control fed females.

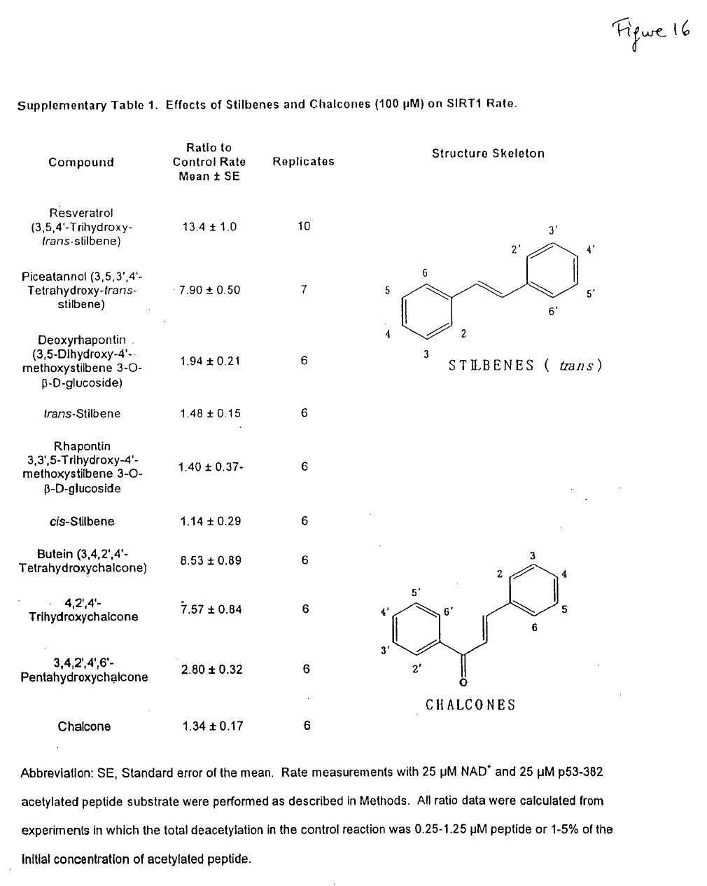

To determine whether resveratrol extends fly lifespan in a Sir2-dependent manner, we analyzed a dSir2 allelic series with increasing amounts of dSir2. Adult offspring from crosses between independently derived alleles of dSir2 were tested. Resveratrol failed to extend lifespan in flies completely lacking functional dSir2 (dSir2^{4.5}/dSir2^{5.26}) (FIG. 13a, b) or in flies in which dSir2 is severely decreased (dSir2¹⁷/dSir2^{KG00871}) (FIG. 13c, d). Resveratrol increased longevity a small but statistically significant amount in flies homozygous for a hypomorphic dSir2 allele (dSir2^{KG0087}/dSir2^{KG0087}) (Table 20, Trial 6) and increased lifespan up to 17% in flies with one copy of the hypomorphic allele and one copy of a wild-type dSir2 (Canton-S/dSir2^{KG0087}) (Table 20, Trial 7). These data demonstrate that the ability of resveratrol to extend fly lifespan requires functional Sir2.

We previously reported that STACs extend the lifespan of replicating yeast cells by mimicking CR⁴. In yeast, chronological and reproductive aging are inseparable in the measure of replicative lifespan. Here we show that STACs can extend lifespan in C. elegans and D. melanogaster, both of which are comprised of primarily non-dividing (post-mitotic) cells as adults, and whose somatic and reproductive aging are independent measures of senescence. In both species, resveratrol increases lifespan in a Sir2-dependent manner and, at least for the fly, this action appears to function through a pathway common to CR.

Our observation that resveratrol can increase longevity without an apparent cost of reproduction is counter to prevalent concepts of senescence evolution. However, STACs may still entail trade-offs under some environmental conditions^{21,22} or in the context of selection acting upon the network of traits that determine fitness^{23,24}. Plants synthesize STACs such as resveratrol in response to stress and nutrient limitation²⁵, possibly to activate their own sirtuin pathways⁴. These molecules may activate animal sirtuins because they serve as plant defense mechanisms against consumers or because they are ancestrally orthologous to endogenous activators within metazoans. Alternatively, animals may use plant stress molecules as a cue to prepare for a decline in their environment or food supply⁴. Understanding the adaptive significance, endogenous function, and evolutionary origin of sirtuin activators will lead to further insights into the underlying mechanisms of longevity regulation and aid in the development of interventions that provide the health benefits of CR.

### Example 15

### Materials and Methods for Example 14

Sirtuin Purification

His₆-tagged recombinant SIR-2.1 and dSir2 were purified from E. coli BL21 (DE3) plysS cells harboring either pET28a-sir-2.1 or pRSETc-dSir2 plasmids. Cells were grown in LB medium containing kanamycin (50 μg/mL) for pET28a-sir-2.1 or ampicillin (100 μg/ml) and chloramphenicol (25 μg/ml) for pRSETc-dSir2 at 30° C. (dSir2) or 37° C. (SIR-2.1) to an OD₆₀₀ of 0.6-0.8. After addition of IPTG (1 mM), flasks were shifted to 16° C. for 20 h. Cell pellets were resuspended in cold PBS buffer containing 300 mM NaCl, 0.5 mM DTT, 0.5 mM PMSF and EDTA-free protease inhibitor tablets and lysed by sonication. Ni²⁺-NTA beads were added to the clarified extract and after 1-3 hours they were loaded on a column, washed with buffer (50 mM Tris. Cl pH 7.4, 200 mM NaCl, 30 mM imidazole) then eluted with the same buffer containing 600 mM imidazole.

Deacetylation Assays

From 0.1 to 1 μg of SIR-2.1 and 1 μg of dSir2 were used per deacetylation assay as previously described with modifications (SIR-2.1: 200 μM NAD⁺, 10 μM Fluor de Lys, FdL; dSir2: 25 μM NAD⁺, 10 μM FdL)²⁶. STACs were dissolved at 10 mM in dimethylsulfoxide (DMSO) the day of the assay. In vitro fluorescence assay results were read in 96-well microplates (Corning Costar 3693) with a Wallac Victor Multilabel counter (Perkin Elmer, excitation at 360 nm, emission at 450 nm). Drosophila S2 cells were grown in Schneider media with fetal calf serum at 23-28° C., seeded at 9×10⁴ cells/well, grown overnight and then exposed to 1 μM TSA, 500 μM polyphenols, and 200 μM FdL for 2 hr. Deacetylation of FdL with lysate from whole cells was determined as described⁴. Unless otherwise indicated all initial rate measurements were means of three or more replicates obtained with single incubation times, at which point 5% or less of the substrate initially present was deacetylated.

C. elegans Media, Strains, Lifespan, and Feeding Assays

Bristol N2 (Caenorhabditis Genetics Center) was used as the wild-type strain. The sir-2.1 mutant strain was generated by backcrossing VC199 (sir-2.1(ok434)) to N2 four times. Cultures were grown on standard NGM media and maintained on E. coli strain OP50. For the lifespan assays, synchronized animals were transferred to treatment plates as young adults (2 d after hatching, day 0 of assay), and were transferred to fresh treatment plates every 2 days for the first 6 to 8 days of the assay. Treatment plates were standard NGM media with the reproductive suppressant FUdR (Sigma; 100 mg/L) containing resveratrol or solvent (DMSO, which does not affect lifespan) added either directly into the agar before pouring (for live OP50 trials) or diluted into PBS and added to the surface of a dry plate to the indicated final concentration (for dead OP50 trials). For some lifespan trials, heat-killed OP50 were used as a food source. OP50 cultures were heated to 65° C. for 30 minutes, then pelleted and resuspended in 1/10 volume in S Basal supplemented with 10 mM MgSO₄. In all assays, worms were monitored daily for mortality by gently probing with a platinum pick. Assays were performed at 24° C.

To assay worm feeding rates, worms at the indicated stages were placed on treatment plates (no FUdR) for 4-5 hours, then videoed for 1 minute using a Pixelink PL-662 camera. The frame rate was slowed and the pumping rate of the pharynx was counted. To assay fecundity, gravid hermaphrodites (5 per plate, raised from synchronized L1s on normal or treatment plates) were allowed to lay eggs on their respective media for 5 hours, and the total number of eggs was counted.

D. melanogaster Media, Strains, Feeding Assay and Lifespan Assays

Survival assays were conducted independently with adult D. melanogaster in two laboratories. In the first laboratory, all trials used an yw marked wild-type strain. Larvae were reared on standard cornmeal-sugar-yeast (CSY) agar diet (cornmeal 5%, sucrose 10.5%, SAF yeast 2%, and agar 0.7%). Newly eclosed adults were placed in 1L demography cages with approximately 75 males and 75 females. Three to four replicate 1L demography cages were used for each treatment group in each trial. Every two days, dead flies were removed and scored, and food vials were replenished. Food vials contained cornmeal-sugar-yeast diet with SAF yeast as either 2% or 3% by weight. Test compounds in 100 μl of EtOH (or blank EtOH in controls) were mixed into melted aliquots of the adult food media to make a final concentration of 0, 10 or 100 μM. Fresh stock solutions and adult media were prepared weekly. In the second laboratory, lifespan trials were conducted with the wild type strain Canton-S, dSir2 ^{4.5} and dSir2 ^{5.26} (S. Smolik, University of Oregon), dSir2¹⁷ (S. Astrom, Stockholm University, Sweden), and dSir2^{KG00871} (Drosophila Stock Center, Bloomington, Ind.). Larvae for all tests were reared on standard cornmeal-sugar-yeast diet. Newly eclosed adults were incubated in plastic shell vials containing 5 ml of 15% sugar-yeast diet (15% SY) or 5% sugar-yeast (5% SY) diet (15% SY: 15% yeast, 15% sucrose, 2% agar; 5% SY: 5% yeast, 5% sucrose, 2% agar as per Ref.²⁰). In all trials, ˜20 males with ˜20 females were placed into each of 10 vials/treatment group. Every two days, flies were passed into new vials and dead flies were counted. Resveratrol in EtOH (or EtOH alone in controls) was added to the media during its preparation after it had cooled to 65° C. and mixed vigorously. Final compound concentrations were 0, 10, 100 or 200 μM. Fresh stock solution and adult media was prepared weekly.

Feeding rate was measured in yw females with the crop-filling assay²⁷. Females were held overnight with water and placed on 2% CSY diet containing food colour (FDA Blue 1) and 0, 10 or 100 μM resveratrol with EtOH. The presence of dye-marked food in the crop was scored in sets of 20 females across five 5-minute intervals. For body mass measurements, 10 vials with 20 males and 20 females each of wild type CS-5 flies were kept on 15% SY diet with EtOH or with resveratrol in EtOH (10 μM). Males and females were weighed daily.

### REFERENCES FOR EXAMPLES 14 AND 15

1. Lin, S. J., Defossez, P. A. & Guarente, L. Requirement of NAD and SIR2 for life-span extension by calorie restriction in Saccharomyces cerevisiae. Science 289, 2126-8. (2000).
2. Gasser, S. C. M. The molecular biology of the SIR proteins. Gene 279, 1-16 (2001).
3. Hekimi, S. & Guarente, L. Genetics and the specificity of the aging process. Science 299, 1351-4 (2003).
4. Howitz, K. T. et al. Small molecule activators of sirtuins extend Saccharomyces cerevisiae lifespan. Nature 425, 191-6 (2003).
5. Landry, J. et al. The silencing protein SIR2 and its homologs are NAD-dependent protein deacetylases. Proc Natl Acad Sci USA 97, 5807-11. (2000).
6. Imai, S., Armstrong, C. M., Kaeberlein, M. & Guarente, L. Transcriptional silencing and longevity protein Sir2 is an NAD-dependent histone deacetylase. Nature 403, 795-800 (2000).
7. Smith, J. S. et al. A phylogenetically conserved NAD+-dependent protein deacetylase activity in the Sir2 protein family. Proc Natl Acad Sci USA 97, 6658-63. (2000).
8. Tanner, K. G., Landry, J., Sternglanz, R. & Denu, J. M. Silent information regulator 2 family of NAD-dependent histone/protein deacetylases generates a unique product, 1-O-acetyl-ADP-ribose. Proc Natl Acad Sci USA 97, 14178-82. (2000).
9. Tanny, J. C., Dowd, G. J., Huang, J., Hilz, H. & Moazed, D. An enzymatic activity in the yeast Sir2 protein that is essential for gene silencing. Cell 99, 735-45. (1999).
10. Guarente, L. Sir2 links chromatin silencing, metabolism, and aging. Genes Dev 14, 1021-6. (2000).
11. Tissenbaum, H. A. & Guarente, L. Increased dosage of a sir-2 gene extends lifespan in Caenorhabditis elegans. Nature 410, 227-30. (2001).
12. Rogina, B., Helfand, S. L. & Frankel, S. Longevity regulation by Drosophila Rpd3 deacetylase and caloric restriction. Science 298, 1745. (2002).
13. Jiang, J. C., Jaruga, E., Repnevskaya, M. V. & Jazwinski, S. M. An intervention resembling caloric restriction prolongs life span and retards aging in yeast. Faseb J 14, 2135-7. (2000).
14. Kenyon, C. A conserved regulatory mechanism for aging. Cell 105, 165-168 (2001).
15. Masoro, E. J. Caloric restriction and aging: an update. Exp Gerontol 35, 299-305. (2000).
16. Koubova, J. & Guarente, L. How does calorie restriction work? Genes Dev 17, 313-21 (2003).
17. Sinclair, D. A. Paradigms and pitfalls of yeast longevity research. Mech Ageing Dev 123, 857-67 (2002).
18. Kaeberlein, M., McVey, M. & Guarente, L. The SIR2/3/4 complex and SIR2 alone promote longevity in Saccharomyces cerevisiae by two different mechanisms. Genes Dev 13, 2570-80. (1999).
19. Chippindale, A. K., Leroi, Armand M., Kim, Sung B., and Rose, Michael R. Phenotypic plasticity and selection in Drosophila life-history evolution. Journal of Evolutionary Biology 6, 171-193 (1993).
20. Chapman, T. & Partridge, L. Female fitness in Drosophila melanogaster: an interaction between the effect of nutrition and of encounter rate with males. Proc R Soc Lond B Biol Sci 263, 755-9 (1996).
21. Walker, D. W., McColl, G., Jenkins, N. L., Harris, J. & Lithgow, G. J. Evolution of lifespan in C. elegans. Nature 405, 296-7 (2000).
22. Marden, J. H., Rogina, B., Montooth, K. L. & Helfand, S. L. Conditional tradeoffs between aging and organismal performance of Indy long-lived mutant flies. Proc Natl Acad Sci USA 100, 3369-73 (2003).
23. Schmid-Hempel, P. On the evolutionary ecology of host-parasite interactions: addressing the question with regard to bumblebees and their parasites. Naturwissenschaften 88, 147-58 (2001).
24. Ebert, D. & Bull, J. J. Challenging the trade-off model for the evolution of virulence: is virulence management feasible? Trends Microbiol 11, 15-20 (2003).
25. Soleas, G. J., Diamandis, E. P. & Goldberg, D. M. Resveratrol: a molecule whose time has come? And gone? Clin Biochem 30, 91-113 (1997).
26. Bitterman, K. J., Anderson, R. M., Cohen, H. Y., Latorre-Esteves, M. & Sinclair, D. A. Inhibition of silencing and accelerated aging by nicotinamide, a putative negative regulator of yeast sir2 and human SIRT1. J Biol Chem 277, 45099-107. (2002).
27. Edgecomb, R. S., Harth, C. E. & Schneiderman, A. M. Regulation of feeding behavior in adult Drosophila melanogaster varies with feeding regime and nutritional state. J Exp Biol 197, 215-35 (1994).

### Example 16

### Identification of Additional Activators and Inhibitors or Sirtuins

The following high-throughput screening protocol was used to identify additional small molecule sirtuin activators and inhibitors from an ICCB library.

The following wells were designated for control reactions: a) with enzyme; DMSO blank, b) with enzyme; with resveratrol (50 μM) positive control. The reaction mixture contains (final): 0.5 units/reaction SIRT1 deacetylase (BIOMOL); 200 μM NAD⁺; 5 μM Fluor de Lys-SIRT1 substrate (BIOMOL); buffer (25 mM Tris/Cl, pH 8.0, 137 mM NaCl, 2.7 mM KCl, 1 mM MgCl₂, and 1 mg/ml BSA). In addition, a reaction mixture containing no enzyme was made so that each well receiving compound has a corresponding “no enzyme control” well. Reactions were performed in black 384 well plates (NUNC) in a final volume of 25 μl/well.

The reactions were started by combining enzyme and substrate in a reaction mixture immediately prior to aliquoting in plates (or substrate only for “no enzyme control” plates). Mixture were aliquoted to plates using Biotek μFill (Biotek Instruments). Control mixtures were manually added to designated wells. A library compound was added at a desired concentration by pin transfer to both “with enzyme” and “no enzyme” plates. Compounds were added in at least triplicate (with enzyme reaction in duplicate and no enzyme controls) at a final concentration of roughly 50 μM. The plates were incubated at 37° C. for 30-60 minutes. Then 25 μl of 1× Developer II (BIOMOL) plus 2 mM nicotinamide were added to all wells to stop the reactions. The reactions were left for at least 30 minutes at 37° C. for the signal to develop. The plates were read in a microplate-reading fluorometer capable of excitation at a wavelength in the range of 350-380 nm and detection of emitted light in the range of 440-460 nm. A read time of 0.1 sec per well was used.

The following positive controls were used: resveratrol, resveratrol 4″-methyl ether (3,5-dihydroxy-4′-methoxy-trans-stilbene, also referred to herein as BML-233, and set forth in Table 10), and pinosylvin, which activated SIRT1 2.2 fold, 2.1 fold and 3.28 fold, respectively. The activators are listed in Table 21 and the inhibitors are listed in Table 22.

### Example 17

### SIRT1 Expression is Upregulated in Mouse Models for Neurodegenerative Disease

For decades, it has been suggested that neurodegenerative disorders are exacerbated by components of the aging process. Nevertheless, the molecules underlying these interconnected phenomena have not been deciphered. Recent studies have revealed that SIR2, the yeast counterpart of mammalian SIRT1, promotes longevity (Anderson, R. M. et al. (2003) Science 302:2124-6; Anderson, R. M. et al. (2003) Nature 423:181-5; Cohen, H. Y. et al. (2004) Science 305:390-2; Howitz, K. T. et al. (2003) Nature 425:191-6; Brunet, A. et al. (2004) Science 303:2011-5; Motta, M. C. et al. (2004) Cell 116:551-63; Langley, E. et al. (2002) Embo J 21:2383-96). This extension in life span is under control of dietary factors (Anderson, R. M. et al. (2003) Science 302:2124-6; Anderson, R. M. et al. (2003) Nature 423:181-5; Cohen, H. Y. et al. (2004) Science 305:390-2; Howitz, K. T. et al. (2003) Nature 425:191-6; Brunet, A. et al. (2004) Science 303:2011-5; Motta, M. C. et al. (2004) Cell 116:551-63; Langley, E. et al. (2002) Embo J 21:2383-96). Here, we show that SIRT1 confers protection against various models of age-dependent neurodegenerative disorders. The upregulation of SIRT1 in post-mortem AD samples further suggests that it is part of a self-defense mechanism that prevents neurodegeneration. This protection conferred by SIRT1 might be mediated through transcriptional silencing in a p53-dependent manner, a key player in neuronal cell death (Mattson, M. P. et al (2001) Apoptosis 6:69-81). SIRT1 may also act to deacetylate Ku70, and thus deactivate the mitochondrial apoptotic machinery (Brunet, A et al. (2004) Science 303:2011-5; Cohen, H. Y. et al. (2004) Mol Cell 13:627-38). Finally, SIRT1 may deacetylate FOXO3/4 transcription factors, thereby enhancing gene expression of anti-oxidative molecules and arresting the cell cycle (Brunet, A et al. (2004) Science 303:2011-5). An involvement of oxidative stress and cell cycle regulators has been reported in a wide diversity of neurodegenerative disorders and conditions (Nguyen, M. D. et al. (2002) Cell Death Differ 9:1294-306; Smith, P. D. et al. (2004) Cell Cycle 3:289-91). In brief, our findings reveal a common molecular pathway in aging and neurodegenerative disorders controlled by SIRT1, the molecular dissection of which may provide novel insights into the aging process and endogenous defense mechanisms.

Here we report that a human homologue of SIR2, SIRT1, is upregulated in brain tissue from Alzheimer's patients and in primary neurons challenged with neurotoxic insults. Using mouse models for AD/Tauopathies and ALS (p25, PDAPP-V717F and mutant SOD1, respectively) we show that expression of SIRT1 correlates with neuronal death and that ectopic expression of SIRT1 or treatment with a SIRT1 activating molecule, resveratrol, protects against neuronal toxicities caused by p25 and mutant SOD1. Thus, SIRT1 provides a unique molecular link between aging and human neurodegenerative disorders.

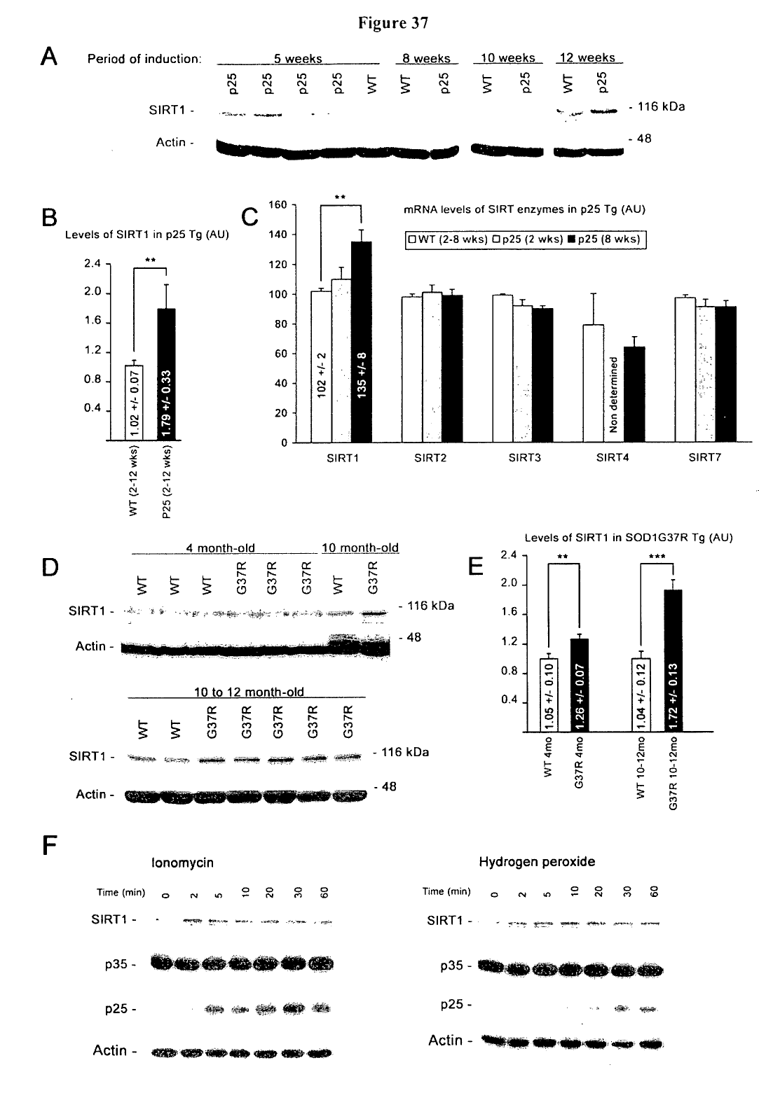

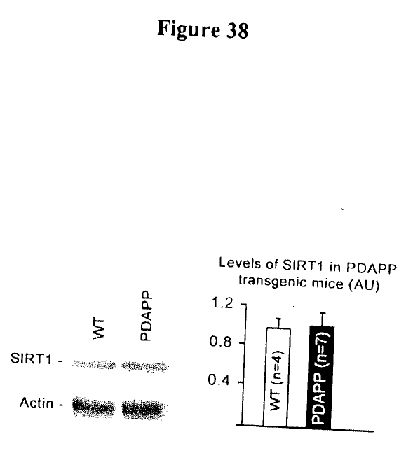

We determined levels of SIRT1 in various mouse models for human age-dependent neurodegeneration. Mice expressing a toxic co-activator of cyclin-dependent kinase 5, p25, display massive degeneration of forebrain with features of AD/Tauopathies (Cruz et al., (2003) Neuron 40: 471-83). Transgenic mice expressing a mutant form of superoxide dismutase 1 (SOD1G37R), which has been linked to human ALS, exhibit severe motor neuron and axon degeneration in spinal cord (Wong, P. C., et al. (1995) Neuron 14: 1105-16; Gurney, M. E., et al. (1994) Science 264: 1772-5). Interestingly, SIRT1 protein levels increase in forebrain of p25 transgenic mice (n=9) as early as two weeks after p25 induction and persists throughout the progression of the pathology to 12 weeks (FIG. 37A-B). Microarray analysis of brain tissue samples from these mice (n=3) showed that SIRT1 mRNA levels progressively increase as the disease progresses, but the levels of the other Sir2 family members, SIRT2-7, do not change (FIG. 37C). In spinal cord of mutant SOD1G37R mice, SIRT1 was only slightly upregulated at 4 months (n=4), a stage with little degeneration, and levels of SIRT1 peaked at 10 to 12 months (n=8) when severe neurodegeneration is evident (FIG. 37D-E) (Nguyen, M. D., et al. (2001) Neuron 30:135-47). Mice expressing a mutant form of Amyloid Precursor Protein (APP) linked to Familial AD (PDAPP-V717F, n=7; 2-12 months) do not exhibit obvious degeneration although they display, in age-dependent manner, β-amyloid plaques, a hallmark of AD (FIG. 38) (Games, D., et al. (1995) Nature 373:523-7). These mice showed no significant increase in SIRT1 in the forebrain (FIG. 38). Together, these results indicate that upregulation of SIRT1 closely correlates with progressive and severe neuronal death in mouse models of human neurodegenerative disorders.

Since both p25 and mutant SOD1 trigger disruption of calcium homeostasis and generate oxidative stress (Cruz, J. C. and Tsai, L. H. (2004) Curr Opin Neurobiol 14:390-4; Bruijin et al. (2004) Annu Rev Neurosci 27:723-49), we tested whether neurons induce SIRT1 in response to these specific stresses. Treatment of primary cortical neurons with either ionomycin (1 μm), a calcium ionophore, or hydrogen peroxide (25 μm), a free-radical generator, rapidly induced SIRT1 protein levels within 5 min and the increased levels lasted up to 1 hour (FIG. 37F). These treatments were paralleled by progressive deterioration of neuronal morphology and the generation of p25 as reported previously (Lee, M. S. et al (2000) Nature 405:360-4; Kusakwa, G. et al. (2000) J Biol Chem 275:17166-72; Nath, R. et al. (2000) Biochem Biophys Res Commun 274:16-21). Thus, SIRT1 is not only induced in mouse models of neurodegeneration, but also in primary cultured neurons under neurotoxic stresses.

### Example 18

### In Cultured Primary Cortical Neurons, SIRT1 Overexpression is Protective Against Neurodegeneration Caused by p25 or the SOD1G39A Mutant

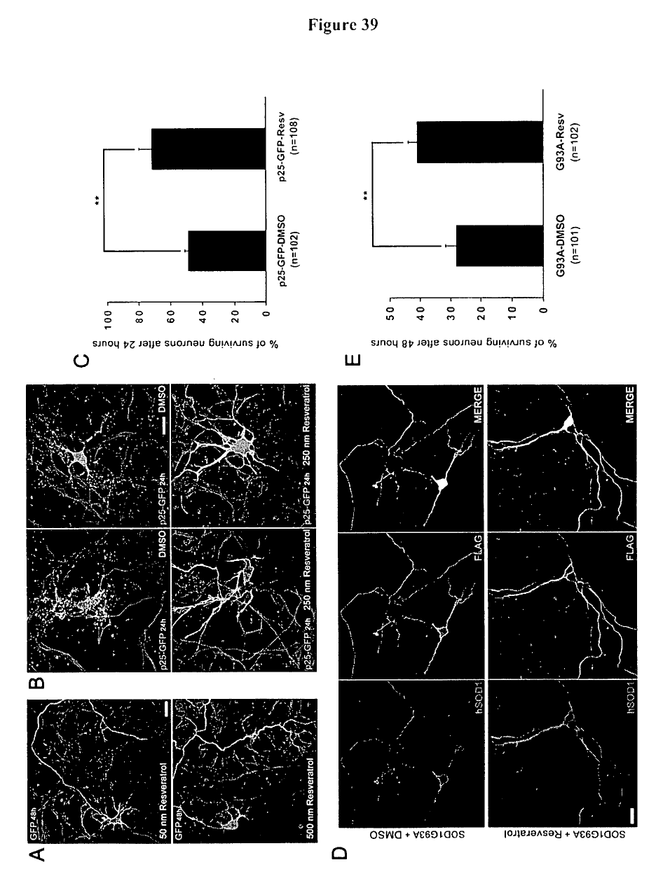

To understand the physiological significance of SIRT1 activation in context of neurodegeneration, we first treated cultured primary hippocampal mouse neurons with resveratrol, a polyphenolic SIRT1 activating compound (STAC) (Howitz, K. T. et al. (2003) Nature 425:191-6) and tested the cells' ability to survive p25-induced toxicity (Patrick, G. N. et al. (1999) Nature 402:615-22). Doses of up to 500 nM resveratrol showed no evidence of toxicity to primary neurons transfected with GFP (FIG. 39A). Cells transfected with p25-GFP showed a high degree of cell death (Lee, M. S. et al. Nature 405:360-4; Patrick, G. N. et al. (1999) Nature 402:615-22; Zhang, J. et al. (1999) J. Neurochem 81:307-13; Hamdane, M. et al. (2003) J Biol Chem 278:34026-34) (50% after 24 hours), based on a decreased integrity of neuronal processes (GFP and Tubulin staining), condensed chromatin and disrupted nuclear morphology with DAPI staining (FIG. 39B-C). Remarkably, resveratrol treatment reduced by 45% the extent of cell death caused by p25 (FIG. 39B-C) (P(T<=t) two tails: 0.01). Resveratrol also provided 45% protection against SOD1G93A toxicity (P(T<=t) two tails: 0.01) (FIG. 39D-E).

### Example 19

### Expression of a Catalytically-Inactive SIRT1 Dominant-Negative Allele in Primary Cortical Neurons Failed to Provide the Same Wild-Type SIRT1

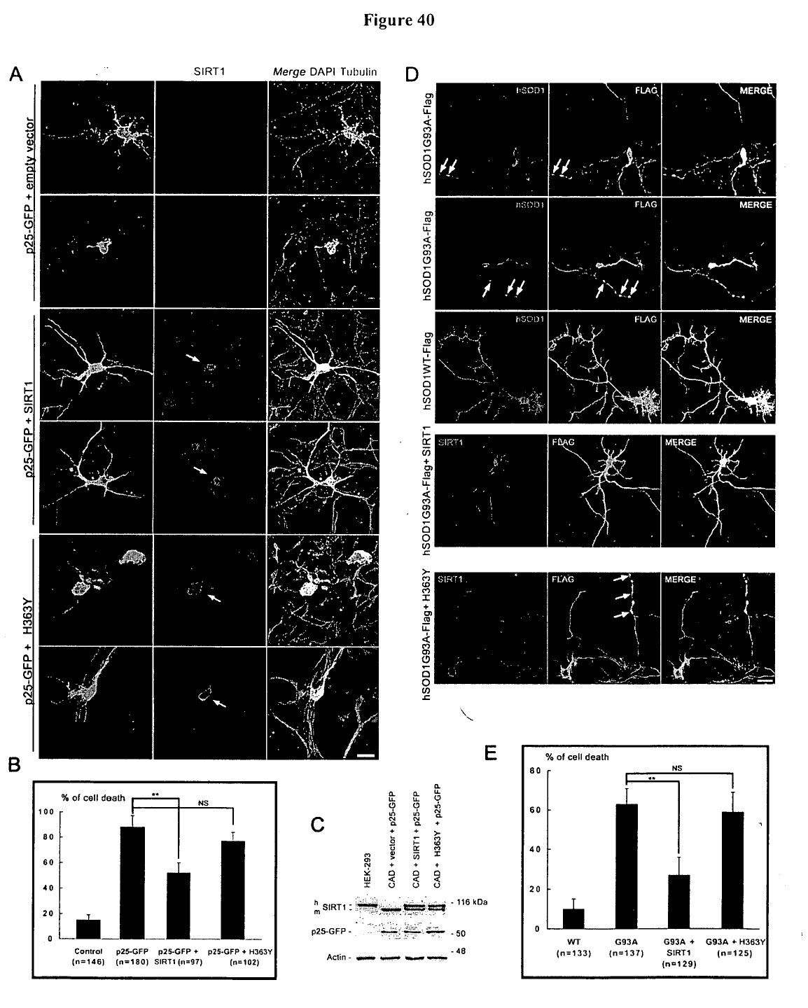

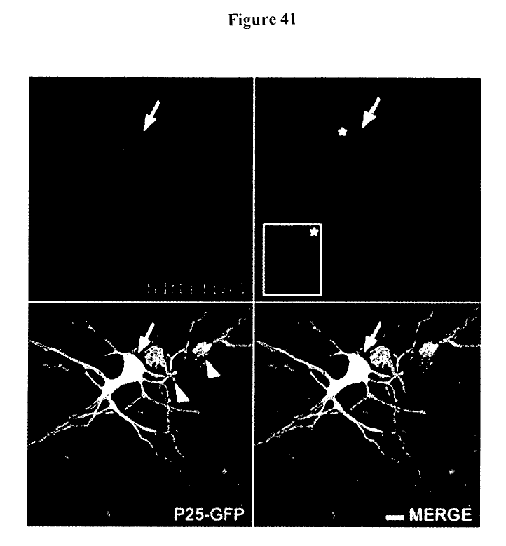

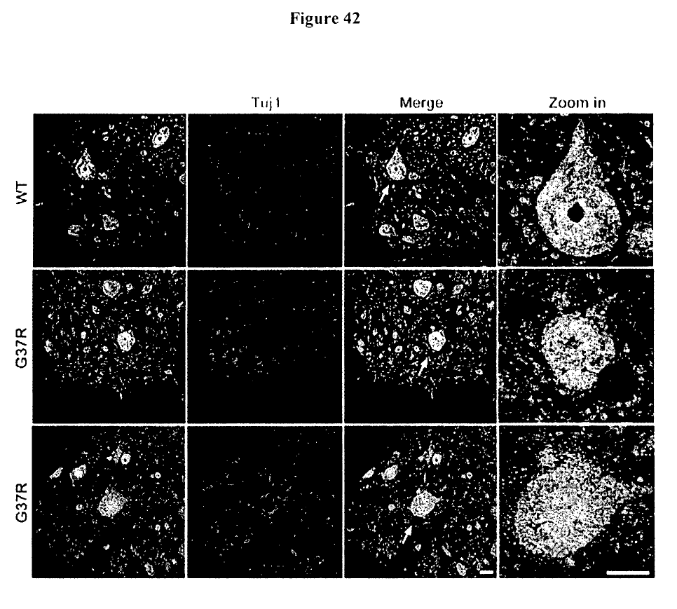

To directly verify the protective role of SIRT1 in neurodegeneration, we transfected primary neurons with p25-GFP or SOD1G93A together with either SIRT1 or SIRT1 lacking the catalytic deacetylase activity (H363Y). Forty eight hours after transfection, approximately 80% of neurons with p25-GFP underwent degeneration as scored by cell integrity and nuclear morphology, as described above (FIG. 40B). Overexpression of SIRT1, but not H363Y, rescued the rate of cell death from 80% to 50% (82.7+/−7% vs 53.0+/−6.2%; P (T<=t) two-tails: 0.001; 82.7+/−7% vs 78.0+/−5.8%; P (T<=t) two-tails: 0.27) (FIG. 40A-B; FIG. 41). The morphology of the p25-GFP/SIRT1-overexpressing neurons appeared remarkably healthy which is indistinguishable from control GFP-transfected neurons. This protective effect is not due to an effect of SIRT1 on the stability of p25-GFP as similar levels of p25-GFP were detected in the absence of presence of SIRT1 overexpression (FIG. 40C). We also sought to determine whether SIRT1 overexpression protects against mutant SOD1 induced neurotoxicty. Approximately, 60% (62.3+/−8.9%) of primary neurons, transfected with mutant SOD1G93A, but not wild-type SOD1, exhibited cytoskeletal disruption and SOD1 aggregates, hallmarks of ALS-linked SOD1 toxicity (FIG. 40D-E). The overexpression of SIRT1, but not H363Y, also protects against SOD1G93A toxicity with a 30% reduction of neurons displaying the degenerative phenotype (62.3+/−8.9% vs 30.1+/−10.2%; P (T<=t) two-tails: 0.001; 62.3+/−8.9% vs 78.0+/−11.0%; P (T<=t) two-tails: 0.33) (FIG. 40D-E). In primary neuronal cultures and human, mouse CNS neurons, SIRT1 is localized in the nucleus and, to a lesser extent, in the cell body (FIGS. 39, 42 and 43). Together, these results indicate that increased levels of SIRT1 in primary neurons confer potent protection against neurotoxicity induced by p25 or mutant SOD1. The observation that the H363Y mutant did not protect suggests that the deacetylase activity of SIRT1 is required for neuroprotection. The protection conferred by SIRT1 expression is remarkably stronger than resveratrol.

### Example 20

### SIRT1 Levels are Upregulated in Postmortem Brain Samples of Alzheimer's Disease

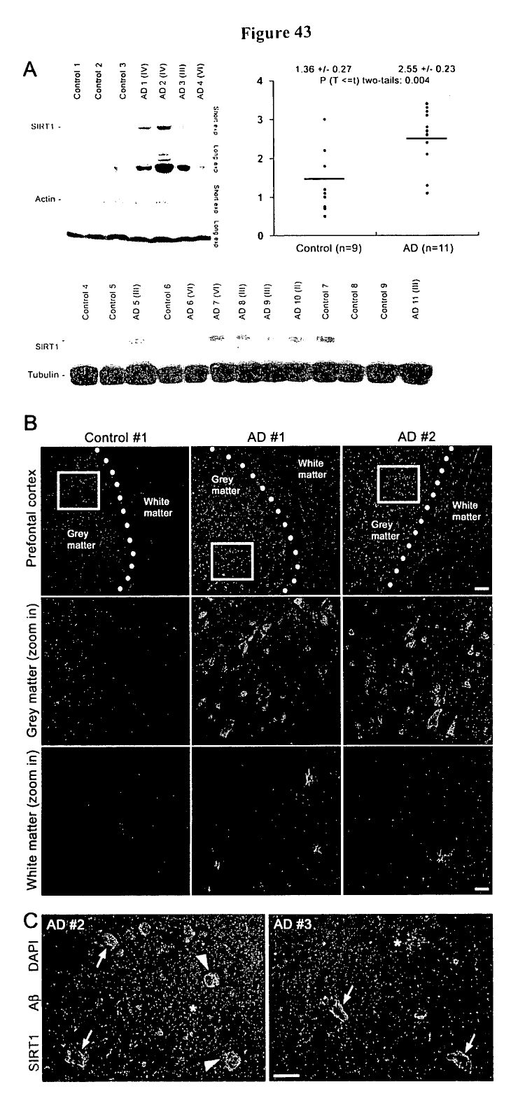

As an anti-aging molecule and player in neuronal survival, we hypothesized that SIRT1 levels may increase as a protective response to neurodegenerative disorders such as AD in human patients. We surveyed levels of SIRT1 in normal patients (n=9) and patients with AD pathology (n=11). For evaluation of human brains, we utilized Braak and Braak classification of progressive neuronal changes in AD (Braak, H. and Braak, E. (1991) Acta Neuropathol (Berl) 82:239-59). Using neurofibrillary tangles and a neuropil thread characteristic distribution pattern, Braak and Braak classification recognizes the following six stages in disease progression: I-II, transentorhinal (clinically silent cases); III-IV, limbic (incipient AD); V-VI, neocortical (fully-developed AD) (Braak, H. and Braak, E. (1998) J Neural Transm Suppl 53: 127-40). As detected by immunoblotting, AD samples exhibited higher levels of SIRT1 protein when compared to controls (2.55±0.23 vs 1.36±0.27; P (T<=t) two-tails: 0.004) (FIG. 43A), although no consistent correlation was found between levels of SIRT1 and the Braak and Braak stages. The localization of SIRT1 in paraffin-embedded prefontal cortex tissues from three individuals (control #1 and AD #1 and 2) was then determined using indirect immunofluorescence. Consistent with immunoblot data, there was a dramatically higher level of SIRT1 in neurons of gray matter of prefontal cortex from AD samples while the white matter regions of all samples rarely exhibited SIRT1-positive cells (FIG. 43B). The areas of intense SIRT1 staining were not confined to neurons adjacent to β-amyloid plaques, a hallmark of AD pathology (AD samples #2 and #3; FIG. 43C). Collectively, these results indicate that SIRT1 is upregulated in regions of the brain that are degenerating.

Equivalents

Those skilled in the art will recognize, or be able to ascertain using no more than routine experimentation, many equivalents of the specific embodiments described herein. Such equivalents are intended to be encompassed by the following claims.

1. A method for treating or preventing a neurodegenerative disease in a subject, comprising administering to a subject in need thereof a therapeutically effective amount of an agent that increases the activity or protein level of a sirtuin in a cell.

2. The method of claim 1, wherein the sirtuin is SIRT1.

3. The method of claim 2, wherein the agent is a nucleic acid encoding SIRT1.

4. The method of claim 1, wherein the agent is a sirtuin-activating compound, salt or prodrug thereof.

5. The method of claim 4, wherein the sirtuin-activating compound comprises the formula selected from the group consisting of formulas 1-25, 30 and 32-65.

6. The method of claim 1, wherein the subject is human.

7. The method of claim 1, wherein the neurodegenerative disease is a disease selected from the group consisting of Alzheimer's disease, amyotrophic lateral sclerosis (ALS), Parkinson's disease, Huntington's disease, and multiple sclerosis.

8. The method of claim 1, further comprising administering to the subject a therapeutically effective amount of a therapeutic agent for treating the neurodegenerative disease.

9. The method of claim 1, wherein the subject is a subject who has been diagnosed with a neurodegenerative disease.

10. The method of claim 9, wherein the diagnosis comprises determining the level or activity of a sirtuin in the subject.

11. The method of claim 10, wherein the neurodegenerative disease is Alzheimer's disease and the diagnosis comprises determining the level or activity of SIRT1 in a brain sample of the subject.

12. The method of claim 10, wherein the neurodegenerative disease is ALS and the diagnosis comprises determining the level or activity of SIRT1 in a spinal cord sample of the subject.

13. The method of claim 1, further comprising monitoring the progression of the neurodegenerative disease.

14. The method of claim 13, wherein monitoring the progression of the neurodegenerative disease comprises determining the level or activity of a sirtuin in the subject.

15. The method of claim 14, wherein the neurodegenerative disease is Alzheimer's disease and monitoring the progression of the disease comprises determining the level or activity of SIRT1 in a brain sample of the subject.

16. The method of claim 14, wherein the neurodegenerative disease is ALS and monitoring the progression of the disease comprises determining the level or activity of SIRT1 in a spinal cord sample of the subject.

17. A method for determining whether a subject has or is likely to develop a neurodegenerative disease, comprising (i) obtaining a biological sample from a subject; and (ii) determining the level or activity of a sirtuin in the biological sample, wherein a higher level or activity of the sirtuin the biological sample of the subject relative to a control level indicates that the subject has or is likely to develop a neurodegenerative disease.

18. The method of claim 17, wherein the sirtuin is SIRT1.

19. The method of claim 18, wherein the biological sample is a brain sample.

20. The method of claim 19, wherein the disease is Alzheimer's disease.

21. The method of claim 18, wherein the disease is ALS and the biological sample is a spinal cord sample.

22. A method for treating or preventing a neurodegenerative disease in a subject, comprising determining whether a subject has or is likely to develop a neurodegenerative disease according to the method of claim 17 and administering to a subject who has been diagnosed as having or as being likely to develop a neurodegenerative disease a therapeutically effective amount of a therapeutic agent for treating the neurodegenerative disease.

23. A composition comprising a compound having a formula selected from the group of formulas 1-25, 30 and 32-65 and a therapeutic agent for treating a neurodegenerative disease.
